SEQUENCE LISTING (756 sequences)

SEQ ID NO: 1 - DNA, 16 bp, synthetic construct
  source 1..16: mol_type = other DNA
gattcagcacggttta

SEQ ID NO: 2 - DNA, 651 bp, synthetic construct
  source 1..651: mol_type = other DNA
cttttcttatatggggagctctgagagaggagccttagccctggattccaaggcctatccacttggtgatcagcactgagcaccgaggattcaccatggaactggggctccgctgggttttccttgttgctattttagaaggtgtcaagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgtccctagcagctcgtcccttttggggccaagggacaatggtcaccgtctcttcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaa

SEQ ID NO: 3 - DNA, 16 bp, synthetic construct
  source 1..16: mol_type = other DNA
tcggtaaagatgcgac

SEQ ID NO: 4 - DNA, 684 bp, synthetic construct
  source 1..684: mol_type = other DNA
gctccctctgctgatgaaaaccagcccagccctgaccctgcagctctgggagaggagccccagtcctgagattcccaggtgtttccattcagtgagcagcactgaacacagaggactcaccatggagttgggactgagctggattttccttttggcttttttaaaaggtgtccagtgtgaagtgcggttggtggagtctgggggaggcttggtacagcctggcaggtccctgagactctcctgtgcagcctctggattcacctttgatgattatgccatgcactgggtccggcaagctccagggaggggcctggagtgggtctcaggtattagttggaaaggtgatgccttaggctatgcggactctgtgaagggccgattcaccatctccagagacaacgccaagaactccctttatctgcaaatgagcagtctgcgagctgaggacacggccttgtattactgtgcaaaagatactggcagctaccactactactacggtctggacgtctggggccaagggaccacggtcgccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtc

SEQ ID NO: 5 - DNA, 16 bp, synthetic construct
  source 1..16: mol_type = other DNA
gctgggtagccacgct

SEQ ID NO: 6 - DNA, 680 bp, synthetic construct
  source 1..680: mol_type = other DNA
tccctctgctgatgaaaaccagcccagccctgaccctgcagctctgggagaggagccccagccctgagattcccaggtgtttccattcagtgatcagcactgaacacagaggactcaccatggagttgggactgagctggattttccttttgactattttaaaaggtgtccagtgtgaagtgcagttggtggagtctgggggaggcttggtacagcctggcaggtccctgagactctcctgtgcagcctctggattcacctttgatgattatgccatgcactgggtccggcaagttacagggaagggcctggagtgggtctcaggtattaattggaatagtggtaacataggctatgcggactctgtgaggggccgattcaccatctccagagacaacgccaagaactccctgtatctgcaaatgaacagtctgagagctgaggacacggccttgtattactgtgcaaaagatagtggcagcagctggtacgggtacttcgatctctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 7 - DNA, 16 bp, synthetic construct
  source 1..16: mol_type = other DNA
ctgatcccacctatcc

SEQ ID NO: 8 - DNA, 646 bp, synthetic construct
  source 1..646: mol_type = other DNA
tggggagctctgggagaggagccccagccctgagattcccaggtgtttccattcagtgatcagcactgaacacagaggactcaccatggagttgggactgagctggattttccttttggctattttaaaaggtgtccagtgtgaagtgcaactggtggagtctgggggaggcttggtacagcctggcaggtccctgagactctcctgtgcagcctctggattcacctttgatgattttgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaagacacggccttgtattactgtacaaaagatagcagcagcagctggtacgggtacttcgatctctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 9 - DNA, 16 bp, synthetic construct
  source 1..16: mol_type = other DNA
acatacggtcacaagg

SEQ ID NO: 10 - DNA, 618 bp, synthetic construct
  source 1..618: mol_type = other DNA
ggtgccctgagagcatcacccagcaaccacatctgtcctctagagaatcccctgagagctccgttcctcaccatggactggacctggaggatcctcttcttggtggcagcagccacaggagcccactcccaggtgcagctggtacaatctggggctgaggtgaagaagcctggggcctcagtgaaggtctcctgcaaggcttctggatacaccttcaccggctattatatatactgggtgcgacaggcccctggacaagggcttgagtggatgggatggatcaaccctaacagtggtggcacaaactatgcacagaagtttcagggcagggtcaccatgaccagggacacgtccatcagcacagtctatatggagctgagcaggctgagatctgacgacgcggccgtgtattactgtgcgaagcggaaaaggggtctggacggctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 11 - DNA, 16 bp, synthetic construct
  source 1..16: mol_type = other DNA
tgcacctgtcaagcga

SEQ ID NO: 12 - DNA, 762 bp, synthetic construct
  source 1..762: mol_type = other DNA
aatcactctgggacacagcccactcagaggcatcccttccagaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccaatgtgaagtgcagctggtggagtctgggggggacttggtgcagcctggcaggtccctgagactctcctgtgcagcctctggattcacctttgatgattatgccatgcactgggtccggcaagctccagggaagggcctggagtgggtctcaggtattagttggaatggtaatagtttaggttatgtggactctgtgaagggccgattcaccgtctccagagacaacgccaagaattccctgtatctgcaaatgaacagtctgagggctgaggacacggccttgtattactgtacaaaagatactgggagctaccactcctactacggtttggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 13 - DNA, 16 bp, synthetic construct
  source 1..16: mol_type = other DNA
gatcgatagaacaatc

SEQ ID NO: 14 - DNA, 680 bp, synthetic construct
  source 1..680: mol_type = other DNA
tccctctgctgatgaaaaccagcccagccctgaccctgcagctctgggagaggagccccagccctgagattcccaggtgtttccattcagtgatcagcactgaacacagaggactcaccatggaattgggactgagctggattttccttttggctattttaaaaggtgtccagtgtgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatggtgctagcataggctatgcggactctctaaagggccgattcaccatctccagagacaacgccaagaactccctctatttgcaaatgaacagtctgagatctgaggacacggccttgttttactgtgcaagagattacagcagtgcctggtacgggtactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 15 - DNA, 16 bp, synthetic construct
  source 1..16: mol_type = other DNA
ttctacaagctgaacg

SEQ ID NO: 16 - DNA, 684 bp, synthetic construct
  source 1..684: mol_type = other DNA
tccctctgctgatgaaaaccagcccagccctgaccctgcagctctgggagaggagccccagccctgagattcccaggtgtttccattcagtgatcagcactgaacacagaggactcaccatggagttgggactgagctggattttccttctggctattttaaaaggtgtccagtgtgaagtgcagttggtggagtctgggggaggcttggtacaacctggcaggtccctgagactctcctgtgcggcctctggattcacctttgatgattatgccatgcactgggtccggcaagctccagggaagggcctggagtgggtctcaggtattaattggaatggtggtaatataggctatgcggactccgtgaagggccgattcaccatgtccagagacaatgccaagaactccctgtatctgcaaatgaaccgtctgagagctgaggacacgggcttgtattactgtacaaaagataggggcagctggtacgagggctggttcgacccctggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactgg

SEQ ID NO: 17 - DNA, 16 bp, synthetic construct
  source 1..16: mol_type = other DNA
agtgggacaggattgg

SEQ ID NO: 18 - DNA, 643 bp, synthetic construct
  source 1..643: mol_type = other DNA
ggccctgagagcatcactcaacaaccacatctgtcctctagagaaaaccctgtgagcacagctcctcaccatggactggacctggaggatcctcttcttggtggcagcagctacaagtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtgggtgggatggatgaaccctaacagtggtaacacaggctatgcacagaagttccagggcagagtcaccatgaccaggaacacctccataaccacagcctacatggagctgagcagcctgagatctgaggacacggccgtgtattactgtgcgaggccctacagtagagcaactggatacttcgatctctggggccgtggcaccctggtcactgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaat

SEQ ID NO: 19 - DNA, 16 bp, synthetic construct
  source 1..16: mol_type = other DNA
cgagccagtggtacag

SEQ ID NO: 20 - DNA, 666 bp, synthetic construct
  source 1..666: mol_type = other DNA
cataactgtttcttatatggggagctctgggagaggagccccagccctgagattcccaggtgtttccattcagtgatcagcactgaacacagaggactcaccatggaattgggactgagctggattttccttttggctattttaaaaggtgtccagtgtgaagtgcagctggtggagtctgggggaggcttggtacagcctggcaggtccctgagactctcctgtgcagcctctggattctcctttgatgattatgccatgcactgggtccggcaagttccagggaggggcctggagtgggtctcaggtattagttggaaaggtgatgccttaggctatgcggactctgtgaagggccgattcaccatctccagagacaacgccaagaactccctttatctgcaaatgaccagtctgcgagctgaagacacggccttatattactgtgcaaaagatactggtagttatcactactattacggtctggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 21 - DNA, 16 bp, synthetic construct
  source 1..16: mol_type = other DNA
ggatgttagtgggcta

SEQ ID NO: 22 - DNA, 657 bp, synthetic construct
  source 1..657: mol_type = other DNA
tctttcttatatggggagctctgggagaggagccccagccctgagattcccaggtgtttccgttcagtgctcagcactgaacacagaggactcaccatggaattgggactgagctggattttccttttggctattttaaaaggtgtccagtgtgaagtgcaactggtggagtctgggggaggcttggtacagcctggcaggtccctgagactctcctgtgcagcctctggattcacctttgatgattatgccatgcactgggtccggcaagctccagggaagggcctggaatgggtctcaggtattagttggaatagtggtagcataggctatgcggactctgtgaagggccgattcaccatctccagagacaacgccaagaactccctgtatctgcaaatgaacagtctgagagctgaggacacggccttatattactgtgcaagagattacagtagtgcctggtacgggtactttgactcctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 23 - DNA, 16 bp, synthetic construct
  source 1..16: mol_type = other DNA
gcgaccaagtgcaagc

SEQ ID NO: 24 - DNA, 653 bp, synthetic construct
  source 1..653: mol_type = other DNA
catctacatcatttcttatatggggagctctgggagaggagcccagcactagaagtcggcggtgtttccattcggtgatcagcactgaacacagaggactcaccatggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtgtcagttatatcatatgatggaagtaataaatactatgcagactccgtgaagggccgattcaccatctccagagacaattccaagaacacgctgtttctgcaagtgaacagcctgagagctgaggacacggctgtatattactgtgcgaaagaagggagaactgggtttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 25 - DNA, 16 bp, synthetic construct
  source 1..16: mol_type = other DNA
gtacgtaagtgcgtga

SEQ ID NO: 26 - DNA, 751 bp, synthetic construct
  source 1..751: mol_type = other DNA
tgggacacagcctactctgaggcatcccttccagaagtcactatatagtaggagacatgcaaatggggtcctccctctgccgatgaaaaccagcccagccctgaccctgcagctctgggagaggagccccagccctgagattcccacgtgtttccattcagtgatcagcactgaacacagaggactcgccatggagtttgggctgaactgggttttccttgttgctattttaaaaggtgtccagtgtgaggtgcaactggtggagtctgggggagaaatgatacggcctggggggtccctgagactctcctgtgcagcctctggattcatctttgatgattatggcatgagctggatccgccaagctccagggaaggggctggagtgggtctctggtattaattggaatggtggtcacacaggttattcagactctgtgaagggccgattcaccatctccagagacaacgccaagaactccctgtatctgcaaatgaacagtctgagagccgaggacacggccttatatcactgtgtgagagattcaagcagcagttggtacggggtttttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 27 - DNA, 16 bp, synthetic construct
  source 1..16: mol_type = other DNA
cttaggacaactggcc

SEQ ID NO: 28 - DNA, 649 bp, synthetic construct
  source 1..649: mol_type = other DNA
tggggagctctgggagaggagccccagccctgagattcccaggtgtttccattcagtgatcagcagtgaacacagaggactcaccatggagttgggactgagttggattttccttttggctattttaaaaggtgtccagtgtgaagtgcagctggtggagtctgggggaggcttggtacagcctggcaggtccctgagactctcctgtgcagcctctggattcaactttgatgattatgccatgcactgggtccggcaagctccagggaagggcctggagtgggtctcaggtattagttggaatggtggtagtttaggctatgcggactctgtaaagggccgattcaccatctccagagacaacgccaagaactccctgtatctgcaaatgaacagtctgagagctgaggacacggccttatattactgtgcaaaagatagtgggagctatcactactactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 29 - DNA, 16 bp, synthetic construct
  source 1..16: mol_type = other DNA
ttgactttctttaggg

SEQ ID NO: 30 - DNA, 661 bp, synthetic construct
  source 1..661: mol_type = other DNA
tcgtcctttcttatatggggagctctgggagaggagccccagccctgagattcccaggtgtttccattcagtgatcagcactgaacacagaggactcaccatggagttgggactgagctggattttccttttggctattttaaaaggtgtccagtgtgaagtgcagctggtggagtcagggggagccttggtacagcctggccggtccctgagactctcctgtgcagcctctggattcgcctttgatgattatgccatgcactgggtccggcaagctccagggaagggcctggagtgggtctcaggtattagttggaatggtgctagcttaggctatgcggactctctaaagggccgattcaccatctccagagacaacgccaagaactccctctatctgcaaatgaacagtctgagatctgaggacacggccttgttttactgtgcaagagattacagcagtgcctggtacgggtactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 31 - DNA, 16 bp, synthetic construct
  source 1..16: mol_type = other DNA
acagccggtgttcgat

SEQ ID NO: 32 - DNA, 682 bp, synthetic construct
  source 1..682: mol_type = other DNA
gctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagtgtgaagtgcagctggtggagtctgggggaggattggtacagcctggcaggtccctgagactctcctgtgcagcctctggattcacctttgatgattatggcatgcactgggtccggcaagctccagggaagggcctggagtgggtctctggtattagttggaatagtggtcacattggctatgcggactctgtgaagggccgattcaccatctccagagacaacgccaagaactccctgaatctgcaaatgaacagtctgagagctgaggacacggccttgtattactgtgcaaaagatataagcagcagctggtacgggtacttcgatctctggggccgtggcaccctggtcactgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 33 - DNA, 16 bp, synthetic construct
  source 1..16: mol_type = other DNA
tacagtgtcctgtaga

SEQ ID NO: 34 - DNA, 672 bp, synthetic construct
  source 1..672: mol_type = other DNA
cattcgctatttcttatatggggagctctcagagaggtgccttagccctggattccaaggcatttccacttggtgatcagcactgaacacagaggactcaccatggagttggggctgtgctgggttttccttgttgctattttagaaggtgtccagtgtgaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtaatgttaccatatactacgcagactctgtgaagggccgattcaccatctccagagacaacgccaagaactcactgtatctgcaaatgaacagcctgagagccgaggacacggctgtttattactgtgcgagagatggggttactatggttcggggagttattccccatgcttttgatatctggggccaagggacaatggtcaccgtctcttcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 35 - DNA, 16 bp, synthetic construct
  source 1..16: mol_type = other DNA
tgtgtttgtacaccgc

SEQ ID NO: 36 - DNA, 696 bp, synthetic construct
  source 1..696: mol_type = other DNA
acgctgtagactcctcccttcagggcagggtgacaactatgcaaatgcaagtgggggcctccccacttaaacccagggctcccctccacagtgagtctccctctctgcccagctgggatctcagggcttcattttctgtcctccaccatcatggggtcaaccgccatcctcgccctcctcctggctgttctccaaggagtctgtgccgaggtgcagctggtgcagtctggagcagaggtgaaaaagcccggggagtctctgaagatctcctgtaagggttctggatacaggtttactactaactggatcggctgggtgcgccagatgcccgggaaaggcctggaatggatggggatcatctatcctgatgactctgataccagatacagtccgtccttccaaggccaggtcaccatctcagccgacaagtccatcaacaccgcctatcttcactggaacagcctgaagtcctcggacaccgccatctattactgtgcgagaggggctaactggttcgacccctggggccagggaaccctggtcaccgtctcctcaaccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 37 - DNA, 16 bp, synthetic construct
  source 1..16: mol_type = other DNA
catcggggtctccatc

SEQ ID NO: 38 - DNA, 680 bp, synthetic construct
  source 1..680: mol_type = other DNA
tccctctgctgatgaaaaccagcccagccctgaccctgcagctctgggagaggagccccagccctgagattcccaggtgtttccattcagtgatcagcactgaacacagaggactcaccatggacttgggactgacctggattttccttttggctattttaaaaggtgtccagtgtgaagtgcagctggtggagtctgggggaggcttggtacagcctggcaggtccctgagactctcctgtgcagcctctggattcacctttgatgattttgccatgcactgggtccggcaagaaccagggaagggcctggagtgggtctcaggtattagttggaatggtggtaatataggctatgcggactctgtgaagggccgattcaccatttccagagacaacgccaagaactccctgtatctgcaaatgaacagtctgagagctgaggacacggccttctattactgtgcaaaagatgtaagcagtgcctggtacggctactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 39 - DNA, 16 bp, synthetic construct
  source 1..16: mol_type = other DNA
acgccgatcatgcatg

SEQ ID NO: 40 - DNA, 658 bp, synthetic construct
  source 1..658: mol_type = other DNA
ctttcttatatggggagctctgagagaggagccttagccctggattccaaggcctatccacttggtgatcagcactgagcaccgaggattcaccatggaactggggctccgctgggttttccttgttgctattttagaaggtgtccagtgtgaggtgcagctggtggagtctgggggaggcctggtcaagcctggggggtccctgagactctcctgtgcagcctctggattcaccttcagttcctatagcatgaactgggtccgccaggctccagggaagggactggagtgggtctcatccattagtagtagtagtatttacatatactatccagactcagtgaagggccgattcaccatctccagagacaacgccaagaactcactgtatctgcaaatgaacagcctgagagccgaggacacggctgtgtattactgtgcgagagatcgagggagtatagcagctcggggtgcttttgatatctggggccaagggacaatggtcaccgtctcttcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 41 - DNA, 16 bp, synthetic construct
  source 1..16: mol_type = other DNA
tgtgtttgtactcgcg

SEQ ID NO: 42 - DNA, 682 bp, synthetic construct
  source 1..682: mol_type = other DNA
gctccctctgccgatgaaaaccagcccagccctgaccctgcagctctgggagaggagccccagccctgagattcccacgtgtttccattcagtgatcagcactgaacacagaggactcgccatggagtttgggctgagctgggttttccttgttgctattttaaaaggtgtccagtgtgaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatgtccagagacaacgccaagaagtccctgtatctgcaaatgaacagtctgagagccgaggacacggccttttatcactgtgcgagagacgatagcagcagcttgtacggatacttccagcactggggccagggcaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 43 - DNA, 16 bp, synthetic construct
  source 1..16: mol_type = other DNA
tacggatcatgctggc

SEQ ID NO: 44 - DNA, 555 bp, synthetic construct
  source 1..555: mol_type = other DNA
gaggtgtttccattcagtgatcagcactgaacacagaggactcaccatggagttgggactgagctggattctccttttggctattttaaaaggtgtccagtgtgaagtgcagctggtggagtctgggggaggcttggtacagcctggcaggtccctgagactctcctgtgcagcctctggattcacttttgatgattatgccatgtactgggtccggcaaattccagggaagggcctggagtgggtctcaggtattacttggaatagtggtagcataggctatgcgggctctgtgcagggccgattcaccatctccagagacaacgccaagaactccctgtatctgcaaatgaacagtctgagagttgaggacacggccttgtattactgtgcaaaggatattggagggagctacttagggtttgactactggggccagggagccctggtcaccgtctcctcagccaaaacaacagccccatcggtctatccactggcccctgtgtgtggagatacaactggctcctcggtgactctaggatgcctggtcaaggg

SEQ ID NO: 45 - DNA, 16 bp, synthetic construct
  source 1..16: mol_type = other DNA
tgcacctgtctaggtt

SEQ ID NO: 46 - DNA, 589 bp, synthetic construct
  source 1..589: mol_type = other DNA
ggagagaggagcccagccctgggattttcaggtgttttcatttggtgatcaggactgaacagagagaactcaccatggagtttgggctgagctggctttttcttgtggctattttaaaaggtgtccagtgtgaggtgcagctgttggagtctgggggaggcttggtacagcctggggggtccctgaaactctcctgtgcagcctctggattcacctttagcagctatgccatgagctgggtccgccaggctccagggaaggggctggagtgggtctcaggtattagtggtagtggtggtagcacatattacgcagactccgtgaagggccggttcaccatatccagagacaattccaagaacacgctgtatctacaaatgaacagcctgagaaccgaggacacggccggatattactgtgcgaaagatcgggggagtatagcagttcgaggtgcttttgatatctggggccaagggacaatggtcaccgtctcttcagccaaaacaacagccccatcggtctatccactggcccctgtgtgtggagatacaactggctcctcggtgactctaggatgcctggtcaaggg

SEQ ID NO: 47 - DNA, 16 bp, synthetic construct
  source 1..16: mol_type = other DNA
tcgcgaggtggcccta

SEQ ID NO: 48 - DNA, 584 bp, synthetic construct
  source 1..584: mol_type = other DNA
ggggagaggagccccagccctgagattcccaggtgtttccattcagtgatcagcactgaacacagaggactcaccatggagttgggactgagctggattttccttttggctattttaaaaggtgtccagtgtgaagtgcagctggtggagtctgggggagacttggttcagcctggcacgtccctgagactctcctgtgcagcctctggattcacctttgatgattatgccatgcactgggtccggcaaactcctggtcagggcctggaatgggtctcaggtattaattggaatagtggtaccataggctatgcggactctgtgaagggccgattcaccatctccagagacaacgccaagaactccctctttctgcaaatgaacagtctgagagctgaggactcggccttttattactgcacaaaagatataggtgggagctactacgcctctgacttctggggccagggaaccctggtcaccgtctcctcagccaaaacaacagccccatcggtctatccactggcccctgtgtgtggagatacaactggctcctcggtgactctaggatgcctggtcaaggg

SEQ ID NO: 49 - DNA, 16 bp, synthetic construct
  source 1..16: mol_type = other DNA
atctgcctcacctcgt

SEQ ID NO: 50 - DNA, 632 bp, synthetic construct
  source 1..632: mol_type = other DNA
gagctctgggagaggagccccagccctgagattcccacgtgtttctattcagtgatcagcaccgaacacagaggactcgccatggagtttgggctgaactgggttttccttgttgctattttaaaaggtgcccagtgtgaggtgcaattggtggagtctgggggaactgtggaacggcctggggggtccctgagactctcctgtgcagcctctggattcacctttgatgattatggcatgagttgggtccgccaagctccagggaaggggctggagtgggtctctggtacaagttggaatggtggtcatataggttatgcagactctgtgaagggccgattcatcatctccagagacaacgccaagaactccctgtatctgcaaattaacagtctgagagtcgaggacacggccttgtattattgtacgagagattcaaccagtagttggtatggggtctttgacttctggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctgg

SEQ ID NO: 51 - DNA, 16 bp, synthetic construct
  source 1..16: mol_type = other DNA
ctcattatcgagagca

SEQ ID NO: 52 - DNA, 623 bp, synthetic construct
  source 1..623: mol_type = other DNA
gggggcagagcctgctgaattctggctgaccagggcagtcaccagagctccagacaatgtctgtctccttcctcatcttcctgcccgtgctgggcctcccatggggtgtcctgtcacaggtacagctgcagcagtcaggtccaggactggtgaagccctcgcagaccctctcactcacctgtgccatctccggggacagtgtctctagcaacattgctacttggaactggatcaggcagtccccctcgagaggccttgagtgcctgggaaggacatactacaggtccaagtggtatagtgattatgcagtatctgtgaaaagtcgaataaccatcaacccagacacatccaagaaccagttctccctgcagctgaactctgtgactcccgaggacacggctgtgtattactgtgcaagaggctggaactacgggtactactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 53 - DNA, 16 bp, synthetic construct
  source 1..16: mol_type = other DNA
agacgttcaaactgct

SEQ ID NO: 54 - DNA, 595 bp, synthetic construct
  source 1..595: mol_type = other DNA
gggggggagaggagccccagccctgagattcccaggtgtttccattcagtgatcagcactgaacacagaggactcaccatggagttgggactgagctggattttccttttggctattttaaaaggtgtccagtgtgaagtgcagctggtggagtctgggggagacttggtacagcctggcaggtccctgagactctcctgtgcagcctctggattcacctttgatgattatgccatgcactgggtccggcaagctccagggaagggcctggagtgggtctcaggtcttagttggaatagtggtggcataggctatgtggactctgtgaagggccgattcaccatctccagagacaacgccaagaactccctgtatctgcaaatgaacagtctgagacctgaggacacggccttgtattactgtgcaaaagatttcaacagctggccccatgaatttgatgtctggggccaagggacaatggtcaccgtctcttcagccaaaacaacagccccatcggtctatccactggcccctgtgtgtggagatacaactggctcctcggtgactctaggatgcctggtcaagggttatttcc

SEQ ID NO: 55 - DNA, 16 bp, synthetic construct
  source 1..16: mol_type = other DNA
ctgaagtagccagttt

SEQ ID NO: 56 - DNA, 577 bp, synthetic construct
  source 1..577: mol_type = other DNA
gggggagagaggagcccagccctgggattttcaggtgttttcatttggtgatcaggactgaacagagagaactcaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagttattagtggtagtggtggtagcacatactacgcagactccgtgaagggccggttcaccatctccagagacaattccaagaacacgctgtatctgcaaatgaacagcctgagagccgaagacacggccgtatattactgtgcgaaagatcggcggggatcctttgactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 57 - DNA, 16 bp, synthetic construct
  source 1..16: mol_type = other DNA
ccacctatcctcaacc

SEQ ID NO: 58 - DNA, 608 bp, synthetic construct
  source 1..608: mol_type = other DNA
ccacctatcctcaaccggataagtaatttcttatgggggagggaggagcccagccctgggattttcaggtgttttcatttggtgatcaggactgaacagagagagctcaccatggagtttgggctgatctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccatatattactgtgcgagatacgggaactggaactatatctggggccagggaaccctggtcaccgtctcctcagccaaaacgacacccccatctgtctatccactggcccctggatctgctgcccaaactaactccatggtgaccctgggatgcctggtcaaggg

SEQ ID NO: 59 - DNA, 16 bp, synthetic construct
  source 1..16: mol_type = other DNA
catcagaaggaatgga

SEQ ID NO: 60 - DNA, 586 bp, synthetic construct
  source 1..586: mol_type = other DNA
gctgggagaggagccccagccctgagaatcccaggtgtttccatttagtgatcagtactgaacacagaggactcaccatggagttgggactgagctggattttccttttggctattttaaaaggtgtccagtgtgaagtgcagttggtggagtctgggggaggcatggtgcagcctggcaggtccctgagactctcctgtgcagcctctggattcacctttgatgattatgccatgcactgggtccggcaagctccagggaagggcctggagtgggtctcaggtattaagtggaatggtggaaacataggctatgcggactctgtgaaggaccgattcaccatctccagagacaacgccaagaactccctgtttctgcaaatgaacagtctgagagctgaggacgcggccttgtattcctgtgcaaaggatataggggggagccaatacgctttggacgtctggggccaagggaccacggtcaccgtctcctcagccaaaacaacagccccatcggtctatccactggcccctgtgtgtggagatacaactggctcctcggtgactctaggatgcctggtcaaggg

SEQ ID NO: 61 - DNA, 16 bp, synthetic construct
  source 1..16: mol_type = other DNA
atctactagaaaccta

SEQ ID NO: 62 - DNA, 643 bp, synthetic construct
  source 1..643: mol_type = other DNA
gagctctgggagaggagccccagccctgagattcccaggtgtttccattcagtgatcagcactgaacacagaggactcaccatggagttgggactgagctggattttccttttggctattttaaaaggtgtccagtgtgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtaacataggctatgcggactctgtgaagggccgattcaccatctccagagacaacgccaataactccctgtatctgcaaatgaacagtctgagagctgaggacacggccttgtattactgtgcaaaagattatagcagtgcctggtacgggtactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcac

SEQ ID NO: 63 - DNA, 16 bp, synthetic construct
  source 1..16: mol_type = other DNA
tgggaagcagttcatg

SEQ ID NO: 64 - DNA, 592 bp, synthetic construct
  source 1..592: mol_type = other DNA
gctgggagaggagccccagccctgagattcccaggtgtttccattcagtgatcagcactgaacacagaggactcaccatggaattgggactgagctggattttccttttggctattttaaaaggtgtccagtgtgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtgttaattggaatggtgttagtttaggctatgcggactctgtgaagggccgattcaccatctccagagacaacgccaagaactccctgtatctgcaaatgaacagtctgagagctgaggacacggccttctattactgtgcaaaagatagtgggagttaccactattactacggtatggacgggtggggccaagggaccacggtcaccgtctcctcagccaaaacaacagccccatcggtctatccactggcccctgtgtgtggagatacaactggctcctcggtgactctaggatgcctggtcaaggg

SEQ ID NO: 65 - DNA, 16 bp, synthetic construct
  source 1..16: mol_type = other DNA
actatctgtgtgacga

SEQ ID NO: 66 - DNA, 628 bp, synthetic construct
  source 1..628: mol_type = other DNA
gagagaggtgccttagccctggattccaaggcatttccacttggtgatcagcactgaacacagaggactcaccatggagttggggctgtgctgggttttccttgttgctattttagaaggtgtccagtgtgaggtgcagttgctggagtctgggggaggcttggtacagcctggagggtccctgagactctcctgtgcagcctctagattcaccttcagtaattatgaaatgaactgggtccgccaggctccagggaagggactggagtggctttcatacattaatactagtggtactaccatatactacgcagactctgtgaagggccgattcaccatctccagagacaacgccaagaactcactgtatctgcaaatgaacagcctgagagccgaggacacggctgtttattactgtgcgagagtgaactggaactctgatgcttttgatatctggggccaagggacaatggtcaccgtctcttcagccaaaacgacacccccatctgtctatccactggcccctggatctgctgcccaaactaactccatggtgaccctgggatgcctggtcaagggctatttccctgagccagtgacagggatccagagttccaggtcactgtca

SEQ ID NO: 67 - DNA, 16 bp, synthetic construct
  source 1..16: mol_type = other DNA
cggacgtcacaccgca

SEQ ID NO: 68 - DNA, 636 bp, synthetic construct
  source 1..636: mol_type = other DNA
gggggagaagagccccagccctgagattcccaggtgtttccattcagtgatcagcactgaacacagaggactcaccatggacttgggactgagctggattttccttttggcaattttaaaagatgtccagtgtgaagttcagttggtggcctctgggggagacttggttcagcctggcaggtccctgagactctcctgtgcagcctccggattcacctttgatgattttggcatgcactgggtccggcaagctccagggaagggcctggagtgggtctcaggtattagttggaatggtggtcacatgggctatgcggcctctgtgaagggccgattcaccatgtccagagacaacgccaagaactccctgtatctgcaaatgaacagtctgagagttgaggacacggccttatattattgtgtaaaagagtttagcagcagctggtacgggttcttcgatctctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtc

SEQ ID NO: 69 - DNA, 16 bp, synthetic construct
  source 1..16: mol_type = other DNA
acgggcttcacttact

SEQ ID NO: 70 - DNA, 616 bp, synthetic construct
  source 1..616: mol_type = other DNA
tgggggagagaggagccttagccctggattccaaggcctatccacttggtgatcagcactgagcaccgaggattcaccatggaactggggctccgctgggttttccttgttgctattttagaaggtgtccagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagataggagcagctcccccttagaaaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaact

SEQ ID NO: 71 - DNA, 16 bp, synthetic construct
  source 1..16: mol_type = other DNA
tgagcatagagactta

SEQ ID NO: 72 - DNA, 629 bp, synthetic construct
  source 1..629: mol_type = other DNA
gctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagggtgaagtgcagctggtggagtctgggggcggcttggtacagcctggcaggtccctgagactctcctgtgcagcctctggattcacctttgatgattatgccatgcactgggtccggctagctccagggaagggcctggaatgggtctcaggtatcacttggaatagtggtagtatatcctatgcggactctgtgaagggccgattcaccatctccagagacaacgccaagaattccctgtatcttcaaatgaacagcctgagagctgaagacacggccttgtattactgtgcaaaagatatggggagcagtggctggtacgtgggcgaccactggggccagggaactctggtcaccgtctcctcagccaaaacgacacccccatctgtctatccactggcccctggatctgctgcccaaactaactccatggtgaccctgggatgcctggtcaaggg

SEQ ID NO: 73 - DNA, 16 bp, synthetic construct
  source 1..16: mol_type = other DNA
cacacctcacaggcct

SEQ ID NO: 74 - DNA, 595 bp, synthetic construct
  source 1..595: mol_type = other DNA
gaggtctgggagaggagcccagcactagaagtcggcggtgtttccattcggtgatcagcactgcacacagaggactcaccatggagtttgggctgagctgggttttcctcgttgctcttttaagaggtgtccagtgtcaagtgcacctggtggagtctgggggagacgtggtccagcctgggaggtccctgagactctcctgtgcagcgtctggattcaccttcagttactatggcatgcactgggtccgccaggctccattcaaggggctggagtgggtggcagttatatggtatgatggaagtaataaatactatgcagactccgtgaagggccgattcaccatcaccagagacaattccaagaacacggtgtatctgcaaatgaacagcctgagagccgaggacacggctatatattactgtgcacgctggggtggattcggtgtaactggaactcacttagactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 75 - DNA, 16 bp, synthetic construct
  source 1..16: mol_type = other DNA
gttctcgagccaggat

SEQ ID NO: 76 - DNA, 587 bp, synthetic construct
  source 1..587: mol_type = other DNA
tgggggagagaggagccttagccctggattccaaggcctatccacttggtgatcagcactgagcaccgaggattcaccatggaactggggctccgctgggttttccttgttgctattttagaaggtgtccagtgtgaggttcacctggtggagtctgggggaggcctggccaagcctggggggtccctgagactctcctgtacagcctctggattcaccttcagtacctataacttgaattgggtccgccaggctccagggaaggggctggagtgggtctcatacattagtcgtagtagtgtttacatatactacgcagactcactgaagggccgattcaccatctccagagacgacgccaagaattcactgtatctgcaaatgaacagcctgagagccgaggacacggctgtgtattactgtgtgagggatgactacggtgactacgttcctcttgactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 77 - DNA, 16 bp, synthetic construct
  source 1..16: mol_type = other DNA
tacggattcgtccgtt

SEQ ID NO: 78 - DNA, 641 bp, synthetic construct
  source 1..641: mol_type = other DNA
gggagctctgggagaggagccccagccctgagattcccaggtgtttccattcagtgatcagcactgaacacagaggactcaccatggagttgggactgagctggattctccttttggctattttaaaaggtgtccagtgtgaagtgcagctggtggagtctgggggagacttggtgcagcctggcaggtccctgagactctcctgtgcagcctctggattcacctttgatgattatgccatgtactgggtccggcaaactccagggaagggcctggagtgggtctcacgtataacttggaatagtggtagtataggctatgcgggctctgtgcagggccgattcaccatctccagagacaacgccaagaactccctgtatctgcaaatgaacagtctgagagctgaggactcggccttgtattattgtgcaaaagatattggtgggagctacttagggtttgactattggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 79 - DNA, 16 bp, synthetic construct
  source 1..16: mol_type = other DNA
gatgaggcaagcgagt

SEQ ID NO: 80 - DNA, 737 bp, synthetic construct
  source 1..737: mol_type = other DNA
gagggcgagcccaaaagactggggcctccctcctcctttttcacctctccatacaaaggcaccacccacatgcaaatcctcacttaagcacccacaggaaaccaccacacatttccttaaattcagggtccagctcacatgggaaataatttctgagagtcctggacctcctgtgcaagaacatgaaacatctgtggttcttccttctcctggtggcagctcccagatgggtcatgtcccaggtgcagctgcaggagtcgggcccaggactggtgaagccttcggagaccctgtccctcacctgcactatctctggtggctccatcagtacttactactggagctggatccggcagcccccagggaagggactggagtggattggatatatctattacagtggaaacaccaattacaacccctccctcaagagtcgagtcaccatatcagtagacacgtccaagaaccagttctccctgaagctgaactctgtgaccgccgcagacacggccgtgtattactgtgcgagacatcgtgggagttatgtctacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 81 - DNA, 16 bp, synthetic construct
  source 1..16: mol_type = other DNA
tggttagcaacacgcc

SEQ ID NO: 82 - DNA, 685 bp, synthetic construct
  source 1..685: mol_type = other DNA
gctccctctgctgatgaaaaccagcccagccctgaccctgcagctctgggagaggagccccagccctgagattcccaggtgtttccactcagtgatcagcactgaacacagaggactcaccatggagttgggactgacctggattttccttttggctattttaaaaggtgtccagtgtgaagtgcagctggtggagtctgggggaggcttggtacagcctggcaggtccctgagactctcctgtgcagcctctggattcacctttgatgattatgccatgcactgggtccggcaagctccagggaggggcctggagtgggtctcaggtattagttggaaaggtgatgccttaggctatgcggactctgtgaagggccgattcaccatctccagagacaacgccaagaactccctttatctacaaatgagcagtctgcgagctgaggacacggccttgtattactgtgcaaaagatactggtagttaccactattactacggtctggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 83 - DNA, 16 bp, synthetic construct
  source 1..16: mol_type = other DNA
ggtgttatcccaggtg

SEQ ID NO: 84 - DNA, 632 bp, synthetic construct
  source 1..632: mol_type = other DNA
tctggggagctctgagagaggagcccagccctgggattttcaggtgttttcatttggtgatcaggactgaacagagagaactcaccatggagtttgggctgagctggctttttcttgtggctattttaaaaggtgtccagtgtgaggtgcagctgttggaatctgggggaggcttggtacagcctggggggtccctgagactctcctgtgcagcctctggattcacctttatcagttataccctgagctgggtccgccaggctccagggaaggggctggagtgggtctcagctattagtggaagtggtgggagtacatactacgtagactccgtgaagggccggttcaccatctccagagacaattccaggaacacgctgtatctgcaaatgaatagtctgagagccgaggacacggccgtatattactgtgcgaaagatttgggatctacagtctactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 85 - DNA, 16 bp, synthetic construct
  source 1..16: mol_type = other DNA
cgcggtaagagttggc

SEQ ID NO: 86 - DNA, 722 bp, synthetic construct
  source 1..722: mol_type = other DNA
ggaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcaccatggagttgggactgacctggattttccttttggctattttaaaaggtgtccagtgtgaagtgcagctggtggagtctgggggaggcttggtacagcctggcaggtccctgagactctcctgtgcagcctctggattcacgtttgatgattatgccatgcactgggtccggcaagctccagggaggggcctggagtgggtctcaggtattagttggaaaggtgatgccttaggctatgcggactctgtgaagggccgattcaccatctccagagacaacgccaagaactccctttatctacaaatgagcagtctgcgagttgaggacacggccttgtattactgtggaaaagatactggtagttaccactactactacggtctggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 87 - DNA, 16 bp, synthetic construct
  source 1..16: mol_type = other DNA
ggtgaagcatcgatgt

SEQ ID NO: 88 - DNA, 649 bp, synthetic construct
  source 1..649: mol_type = other DNA
tggggagctctgggagaggagccccagccctgagattcccaggtgtttccattcagtgatcagcactgaacacagaggactcaccatggaattgggactgagctggattttccttttgtctattttaaaaggtgtccagagtgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtgttaatttaggctatgcggactctgtgaagggccgattcaccatctccagagacaacgccaagaactccctgtatctgcaaatgaacagtctgagagttgaggacacggccttgtatttctgtgcaaaagatagtgggagctaccactactattatggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 89 - DNA, 16 bp, synthetic construct
  source 1..16: mol_type = other DNA
gcatgatgtagcacga

SEQ ID NO: 90 - DNA, 836 bp, synthetic construct
  source 1..836: mol_type = other DNA
aacattgactgccacagaacatacttcatgtgcatctatacagtagaaatgctaataagaattaattgtttatgaagtgtaatcactctgggacacagcccactcagaggcatcccttccagaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagtgtgaagtgcagctggtggagtctgggggaggcttggtacagcctggcaggtccctgagactctcctgtgcagcctctggattcacctttgatgattatgccatgtactgggtccggcaaactccagggaagggcctggagtgggtctcaggtattacttggaatagtggtagcataggctatgcggcctctgtgcagggccgattcaccatctccagagacaacgccaagaactccctgtatctgcaaatgaacagtctgagagctgaggacacggccttgtattactgtacaaaagatcttggtgggagttacttagggtttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 91 - DNA, 16 bp, synthetic construct
  source 1..16: mol_type = other DNA
ctacccagttgctcct

SEQ ID NO: 92 - DNA, 656 bp, synthetic construct
  source 1..656: mol_type = other DNA
ctttcttatatggggagctctgggagaagagccccagccctgagattcccaggtgtttccattcagtgatcagcactgaacacagaggactcaccatggacttgggactgagctggattttccttttggctattttaaaagatgtccagtgtgaagttcagttggaggcctctgggggagacttggttcagcctggcaggtccctgagactctcctgtgcagcctctggattcatctttgatgattttggcatgcactgggtccggcaagctccagggaagggcctggagtgggtctcaggtattagttggaatggtggtcacataggttatgcggactctgtgaagggccgattcaccatctccagagacaacgccaagaactccctgtatctgcaaatgaacagtctgagagctgaggatacggccttgtattattgtgcaaaagaacatagcagcagctggtacgggttcttcgatttctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 93 - DNA, 16 bp, synthetic construct
  source 1..16: mol_type = other DNA
agcgtatcactgtgta

SEQ ID NO: 94 - DNA, 679 bp, synthetic construct
  source 1..679: mol_type = other DNA
gctccctctgctgatgaaaaccagcccagccctgaccctgcagctctgggagaggagccccagccctgagattcccaggtgtttccattcagtgatcagcactgaacacagaggactcaccatggaattgggactgaggtggatcctcctattggctattttaaaaggtgtccagtgtgaactacagctggtggagtctgggggaggcttggtacaacctggcaggtccctgagactctcctgtgctgcctctggattcccctttgatgcctatgccatgcactgggtccggcaagctccagggaagggcctgcagtgggtctcaggtattaggtggaatggtggaaatatagattatgtggactctgtgaagggccgattcaccatctccagagacaacgccaagaactccctgtatctgcaattgaacagtctgagaagtgaggacacggccttatattactgtgcaaaagatctgggagggagccactacgctttggacgtctggggccaggggaccatggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 95 - DNA, 16 bp, synthetic construct
  source 1..16: mol_type = other DNA
aaccatggtgtatggg

SEQ ID NO: 96 - DNA, 685 bp, synthetic construct
  source 1..685: mol_type = other DNA
gctccctctgctgatgaaaaccagcccagccctgaccctgcagctctgggagaggagccccagccctgagattcccaggtgtttccattcagtgatcagcactgaacacagaggactcaccatggagttgggactgaactggattttccttttggcaattttaaaaggtgtccagtgtgaagtgcagctggtggagtctgggggagtcttggtacagcctggcaggtccctgagactctcctgtgcagcctctggattcacctttgatgattttgccatgcactgggtccggcaagctccagggaagggcctggagtgggtctcaggtattagttggaatggtaatagcataggctatgcggactctgtgaagggccgattcaccatctccagagacaatgccaagaactccctgtatctgcaaatgaacagtctgagaactgaggacacggccttgtattactgtgcaaaagataccagcagctggagctactactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 97 - DNA, 16 bp, synthetic construct
  source 1..16: mol_type = other DNA
cagctggtcacctcgt

SEQ ID NO: 98 - DNA, 660 bp, synthetic construct
  source 1..660: mol_type = other DNA
cagcccagccctgaccctgcagctctgagagaggagcccagccctgggattttcaggtgttttcatttggtgatcaggactgaacagagagaactcaccatggagtttgggctgaactggcttttacttgtgactcttttaaaagctgtccagtgtgaggtgcacctgttggagtctgggggaggcttcatacagcctggggggtccctgagactctcctgtgcagcctctggattcacctttagtacctatgccatgaattgggtccgcctggctccagggaaggggctggagtgggtctcgactattagtggtagtggtggtttcacatcctacacagactccgtgcagggccgattcaccatctccagagacaattccaagaataaactgtatctgcaaataaacaacctgacagccgaggacacggccgtatattactgtgcgaaagatagacgtatagtgggagcgactggctttgactactggggccagggagccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 99 - DNA, 16 bp, synthetic construct
  source 1..16: mol_type = other DNA
gatgaggtcgaatgct

SEQ ID NO: 100 - DNA, 634 bp, synthetic construct
  source 1..634: mol_type = other DNA
atggggagctctgagagaggagcccagccctgggattttcaggtgttttcatttggtgatcaggactgaacagagagatctcaccatggagtttgggctgagctggctttttcttgtggcttttttaaaaggtgtccagtgtgaggtgaagctgttggagtcggggggaggcttggtacagcctggggggtccctgagactctcctgtgcagcctctggattcagctttagcacctatgccatgaactgggtccgccaggctccagggaaggggctggagtgggtctcagctattactggtagtggtggtagcacatactacgcagactccgtgaagggccggttcaccatgtccagagacaattccaagaacacgctgtatctgcaaatgaacagcctgagagccgaagacacggccgtatattactgcgcgaaagatcggagaggtgcttttgatatcgggggccaagggacaatggtcaccgtctcttcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 101 - DNA, 16 bp, synthetic construct
  source 1..16: mol_type = other DNA
tgcgcagtcctttctc

SEQ ID NO: 102 - DNA, 680 bp, synthetic construct
  source 1..680: mol_type = other DNA
gcctccctctcctgatgaaaaccagcccagccctgactccgcagctctgggagaggagcccccgccctgggattcccaggtgttttcatttggtgatcagcactgaacacagaagagtcatgatggagtttgggctgagctgggttttccttgttgctatttttaaaggtgtccagtgtgaggtgcagttggtggagtctgggggaggcttggtccagcctggggggtccctgagactctcctgtgcagcctctggattcaccttcagtagccatgctatgcactgggtccgccaggctccagggaagggactggaatatgtttcaggtattagtagtaatgggggtagcacatatcatgcagactctgtgaagggcagattcaccatctccagagacaattccaagaacacgctgtatcttcaaatgggcagcctgagagctgaggacatggctgtgtattactgtgcgaggtatagcagcagaaggtactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 103 - DNA, 16 bp, synthetic construct
  source 1..16: mol_type = other DNA
tcgaggcagtgaagag

SEQ ID NO: 104 - DNA, 679 bp, synthetic construct
  source 1..679: mol_type = other DNA
gctccctctgctgatgaaaaccagcccagccctgaccctgcagctctgggagaggagccccagccctgagattcccaggtgattccattcagtgatcagcactgaacacagaggactcaccatggagttgggactgagctggattttccttttggctattttaaaaggtgtccagtgtgaagtgcagctggtggagtctgggggaggcttggtacaaactggcaggtccctgagactctcctgtggtgcctctggattcacctttgatgattatgccatgcactgggtccggcaagctccagggaagggcctggagtgggtctcaggtattaggtggaatggtggtaccatagactatacggactctgtgaagggccgattcaccatctccagagacaacgccaagaataccctatatctgcaaatgaacagtctgagaactgaggacacggccttgtattactgtgcaaaagatatgggagggagcaactacggtttggacgtctggggccaagggaccacggtcaccgtttcttcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 105 - DNA, 16 bp, synthetic construct
  source 1..16: mol_type = other DNA
cttggctagtaggtgc

SEQ ID NO: 106 - DNA, 684 bp, synthetic construct
  source 1..684: mol_type = other DNA
ctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagtgtgaaatacaactggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaataatggtagcttaggctatgcggactctgtgaagggccgattcaccatctccagagacaacgccaagaactccctgtatctacaaatgaacagtctgagaactgaggacacggccttgtattactgtgtaaaagataccggcagctgggcctcctactacggcatggacgtctggggccacgggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 107 - DNA, 16 bp, synthetic construct
  source 1..16: mol_type = other DNA
ctaatggagcgtaata

SEQ ID NO: 108 - DNA, 634 bp, synthetic construct
  source 1..634: mol_type = other DNA
ataaagccctctttcttatatggggacccaaaaaccacacccctccttgggagaatcccctagatcacagctcctcaccatggactggacctggagcatccttttcttggtggcagcagcaacaggtgcccactcccaggttcagctggtgcagtctggagctgaggtgaagaaacctggggcctcagtgaaggtctcctgcaaggcttctggttacacctttaccaactatggtatcagctgggtgcgacaggcccctggacaagggcttgagtggatgggatggatcagcgtttacaatggtaacacaaactatgcacagaagctccagggcagagtcaccatgaccacagacacatccacgagcacagcctacatggaggtgaggagcctgagatctgacgacacggccgtgtattactgtgcgaggggggtcaactggaactacgtctttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 109 - DNA, 684 bp, synthetic construct
  source 1..684: mol_type = other DNA
gaggctgatcacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagctggttagcctggtatcagcagaaaccagggagagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataac

SEQ ID NO: 110 - DNA, 663 bp, synthetic construct
  source 1..663: mol_type = other DNA
gggaggaatcagtcccactcaggacacagcatggacatgagggtccccgctcagctcctggggctcctgctgctctggttcccaggtgccaggtgtgacatccagatgacccagtctccatcctccctgtctgcatctgtgggagacagagtcaccatcacttgccgggcaagtcagggcattagaactgatttaggctggtatcagcagaagccagggaaagcccctaagcgcctaatctatgctgcatccaatttgcacggtggggtcccatcaaggttcagcggcactggatctgggacagacttcactctcacaatcagcagcctgcagcctgaagattttgcaacttttttctgtctacagcattatagttacccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 111 - DNA, 700 bp, synthetic construct
  source 1..700: mol_type = other DNA
tgccctgaagccttattaataggctggtcacactttgtgcaggagtcagacccagtcaggacacagcatggacatgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtaggtggttggcctggtatcagcagaaaccagggaaagcccctaaattcctgatctataaggcgtctagtttagaaagtggggtcccatcaaggttcagcggcagtggatctgggacagaattcactctcaccatcagcagcctgcagcctgatgattttgcaacttattactgccaacagtataatagtgattctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 112 - DNA, 728 bp, synthetic construct
  source 1..728: mol_type = other DNA
gaggctggtcacactttgtgcaggagtcagacccagtcaggacacagcatggacatgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtagctggttggcctggtatcagcagaaaccagggaaagcccctaacctcctgatctataaggcgtctagtttagaaagtggggtcccatcaaggttcagcggcagtggatctgggacagaattcactctcaccatcagcagcctgcagcctgatgattttgcaacttattactgtcaacagtataatagtgattctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccat

SEQ ID NO: 113 - DNA, 720 bp, synthetic construct
  source 1..720: mol_type = other DNA
aggaacgatattttttcttatatggggactgatcaggactcctcagttcaccttctcacaatgaggctccctgctcagctcctggggctgctaatgctctgggtctctggatccagtggggatattatgatgactcagtctccactctccctgcccgtcacccctggagagccggcctccatctcctgcaggtctagtcagagcctcctgcatagtaatggatacaactatttggattggtatctgcagaagccagggcagtctccacagctcctgatctatttgggttctaatcgggcctccggggtccctgacaggttcagtggaagtggatcaggcacagattttacactgaaaatcagcagagtggaggctgaggatgttggggtttattactgcatgcaaggtctacaaactctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggcc

SEQ ID NO: 114 - DNA, 683 bp, synthetic construct
  source 1..683: mol_type = other DNA
cggacttcttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaggcccctaagcgcctgatctatgctgcatccagtttgccaagtggggtcccatcaaggttcagcggcagtggatctgggacagaattcactctcacaatcagcagcctgcagcctgaagattttgcaacttattactgtctacagcttaatacttaccctctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 115 - DNA, 681 bp, synthetic construct
  source 1..681: mol_type = other DNA
gaggctggtgacatcctgtgcagaagtctctctcagtcaggacacagcatggacatgagggtccctgctcagctcctgggactcctgctgctctggctcccagatgccagatgtgacatccagatgacccagtctccatcctccctgtctgcatctgtaggagacagagtcaccatcacttgccgggcgagtcaggacattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaactataacagtgtccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 116 - DNA, 522 bp, synthetic construct
  source 1..522: mol_type = other DNA
ggaggaatcagtcccactcaggacacagcatggacatgagggtccccgctcagctcctggggctcctgctgctctggttcccaggtgccaggtgtgacatccagatgacccagtctccatcctccctgtctgcatctgaaggagacagagtcaccatcacttgccgggcaagtcaggacattagaattgatttaggctggtatcagcagaaaccagggaaagcccctaagcgcctgatctatgctgcttccagtttgcaaagtggggtcccatcaaggttcagcggcagtggatctgggacagaattcactctcacaatcagcagcctgcagcctgaagattttgcaacttactactgtctacagcataatagttacccactcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaag

SEQ ID NO: 117 - DNA, 700 bp, synthetic construct
  source 1..700: mol_type = other DNA
gatccgttggttttcttatatggggaggagtcagacccagtcaggacacagcatggacatgagggtccccgctcagctcctggggctgctaatgctctgggtctctggatccagtggggatattgtgatgactcagtctccactctccctgcccgtcacccctggagagccggcctccatctcctgcaggtctagtcagagcctcctgcatagtaatggatacaactatttggattggttcctgcagaagccagggcagtctccacagctcctgatctatttggattctaatcgggcctccggggtccctgacaggttcagtggcagtggatcaggcacagattttacactgaaaatcagcagagtggaggctgaggatgttggggtttattactgcatgcaagctctacaaactccattcactttcggccctgggaccaaaatagatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 118 - DNA, 754 bp, synthetic construct
  source 1..754: mol_type = other DNA
tgccagaacaatcttttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaactgatttaggctggtatcagcagaaaccagggaaagcccctgagcgcctgatctatgctgcatccattttacaaggtggggtcccatcgaggttcagcggcagtggatctgggacagaattcactctcacaatcagcagcctgcagcctgaagattttgcaactttttactgtctacagcattatatttacccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaaca

SEQ ID NO: 119 - DNA, 724 bp, synthetic construct
  source 1..724: mol_type = other DNA
catgtcccacagagcaacgcctactgccctgaacatttatcaataggctggtgacatcctgtgcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgagtcagggcattaacaattatttagcctggtatcagcagaaaccagggaaaattcctaagctcctgatctttactgcatccactttgcaatcaggggtcccatctcggttcagtggcagtggatctgggacagatttcactctcaccatcaacagcctgcagcctgaagatgttgcaacttattactgtcaaaactataacagtgtccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 120 - DNA, 730 bp, synthetic construct
  source 1..730: mol_type = other DNA
catgtcccacagagcaacgcctactgccctgaacatttatcaataggctggtgacatcctgtgcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgcgtcagggcattagcaattatttagcctggtttcagcagaaaccaggaaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagc

SEQ ID NO: 121 - DNA, 676 bp, synthetic construct
  source 1..676: mol_type = other DNA
gtgtgacatcctgtgcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattctttagcctggtatcagcagaaaccagggacagttcctaaactcctgatctttgctgcatccactttgctatcagggatcccatctcggttcagtggcggtggatctgggacagatttcactctcaccatcagcagcctacagcctgaagatgttgcaacttattactgtcaaaactataacagtgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 122 - DNA, 690 bp, synthetic construct
  source 1..690: mol_type = other DNA
ctggcccctttctttttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcacggtggggtcccatcaaggttcagcggcagtggatctgggacagaattcactctcacaatcagcagcctgcagcctgaagattttgcaacttattactgtctacagcataatagttaccctctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 123 - DNA, 838 bp, synthetic construct
  source 1..838: mol_type = other DNA
tagcctgcagggagaaacacatgctctgcagactttggacacctgggaggcactgggcctgtgcagtgttattgagataagacatctttgcagctgtgcagatttgcatgtcccacagagcaacgcctactgccctgaacatttatcaataggctggtgacatcctgtgcagaagtctctctcagtcaggacacagcatggacatgagggtccctgctcagctcctgggactcctgctgctctggctcccagatgccagatgtgacatccagatgacccagtctccatcctccctgtctgcatctgtaggagacagagtcaccatcacttgccgggcgagtcaggacattagcaattatttagcctggtatcagcaaaaaccagggaaagttcctaagctcctgatctatgctgcatccactttgcaatcaggggtcccatctcggttcagtggcagtggatctgggacagatttcactctcaccatcagcagcctgcagccacaagatgttgcaacttattactgtcaaaactataacagtgtccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagcta

SEQ ID NO: 124 - DNA, 718 bp, synthetic construct
  source 1..718: mol_type = other DNA
gaggctggtgacatcctgtgcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgagtcagggcattagcaattttttagcctggtttcagcagaaaccagggaaagttcctaagctcctgatctatgctgcatccactttgcaatcaggggtcccctctcggttcagtggcagtggatctgggacagatttcactctcaccatcagcagcctgcagcctgaagatgttgcaacttattactgtcaagagtataacagtgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaa

SEQ ID NO: 125 - DNA, 662 bp, synthetic construct
  source 1..662: mol_type = other DNA
ggggaggagtcagtctcagtcaggacacagcatggacatgagggtccctgctcagctcctggggctcctgctgctctggctctcaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatactaatctcccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgac

SEQ ID NO: 126 - DNA, 847 bp, synthetic construct
  source 1..847: mol_type = other DNA
ctaccctcagactgagccaacggccttttctggcctgatcacctgggcatgggctgctgagagcagaaaggggaggcagattgtctctgcagctgcaagcccagcacccgccccagctgctttgcatgtccctcccagccgccctgcagtccagagcccatatcaatgcctgggtcagagctctggagaagagctgctcagttaggacccagagggaaccatggaaaccccagcgcagcttctcttcctcctgctactctggctcccagataccaccggagaaattgtgttgacgcagtcgccaggcattctgtctttgtctccagggaaaagagccaccctctcctgcagggccagtcagagtgttagtagcaatttcctagcctggtatcagcagaaacctggccaggctcccaggctcctcatctatggtacatccagcagggccaatggcatcccagacaggttcagtggcagtgggtctgggacagacttcactctcaccatcagcggtctggagcctgaagattttgcagaatattactgtcagcagtatggtagttcagtcaccttcggccaggggacacgactggagattaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 127 - DNA, 679 bp, synthetic construct
  source 1..679: mol_type = other DNA
ggctggtcacactttgtgcaggagtcagacccagtcaggacacagcatggacatgagggtccccgctcagctcctggggctcctgctgctctggctcccaggtgccaaatgtgacatccagatgacccagtctccttccaccctgtctgcatctgtaggagacagagtcaccatcacttgccgggccagtcagagtgttagtagctggttggcctggtatcagcagaaaccagggaaagcccctaagctcctgatctataaggcgtctagtttagaaagtggggtcccattaaggttcagcggcagtggatctgggacagaatttactctcaccatcagcagcctgcagcctgatgattttgcaacttattactgccaagtttataatagtgattctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 128 - DNA, 775 bp, synthetic construct
  source 1..775: mol_type = other DNA
gagccagccctgcagctgtgctcagcctgccccatgccctgctgattgatttgcatgttccagagcacagccccctgccctgaagacttttttatgggctggtcgcaccctgtgcaggagtcagtctcagtcagggcacagcttggacatgagggtccccgctcagctcctggggctcctgctactctggctccgaggtgccagatgtgacatccagatgacccagtctccatccgccctgtctgcatctgtaggagacagagtcaccatcacttgccggccaagtcagaccattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagttcccctctcaccttcggccaagggacacgactggagattaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 129 - DNA, 661 bp, synthetic construct
  source 1..661: mol_type = other DNA
ggggcagaagtctctctcagtcaggacacagcatggacatgagggtccctgctcagctcctgggactcctgctgctctggctcccagataccagatgtgacatccagatgacccagtctccatcctccctgtctgcttctgtaggagacagagtcaccatcacttgccgggcgagtcagggcattagcatttatttagcctggtatcagcagaaaccagggaaagttcctaaactcctgatctatgctgcatccactttgcaatcaggggtcccatctcggttcagtggcagtggatctgggacagatttcactctcaccatcagcagcctgcagcctgaagatgttgcaacttattactgtcaaaactataacagtgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacg

SEQ ID NO: 130 - DNA, 672 bp, synthetic construct
  source 1..672: mol_type = other DNA
gagagctctggagaagagctgctcagttaggacccagagggaaccatggaaaccccagcgcagcttctcttcctcctgctactctggctcccagataccaccggagaaactgtgttgacgcagtctccaggcaccctgtctttgtctccaggggaaagagtcaccctctcctgcagggccagtcagagtgttagcagcacctacttagcctggtaccagcagaaacctggccaggctcccaggctcctcatctatggcgcatccaacagggccactggcatcccagacagattcagtggcagtgggtctgggacagacttcactctcaccatcagcagactggagcctgaagattttgcagtgtattactgtcagcagtatggtagctcgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 131 - DNA, 531 bp, synthetic construct
  source 1..531: mol_type = other DNA
ggaggctgatcacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtggacgttcggccaagggaccaaggtggaaatcaaacgggctgatgctgcaccaactgtatccatcttcccaccatccagtgagcagttaacatctggaggtgcctcagtcgtgtgcttcttgaacaactt

SEQ ID NO: 132 - DNA, 659 bp, synthetic construct
  source 1..659: mol_type = other DNA
gggaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagaattagcaccgacttaaattggtatcagcagaaatcagggaaagcccctaatctcctgatctatggtgcatccagtttgcaaagtggggtcccatcaaggttcagtggcagtggatctgggacagatttcattctcaccatcagcactctacaacctgaagattttgcaacttactactgtcaacagagttacagtaccccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacg

SEQ ID NO: 133 - DNA, 677 bp, synthetic construct
  source 1..677: mol_type = other DNA
gtgacatcctgtgcagaagtctctctcagtcaggacacagcatggacatgagggtccctgctcagctcctgggactcctgctgctctggctcccagataccagatgtgacatccagatgacccagtctccctcctccctgtctgcatctgtaggagacagagtcaccatcacttgccgggcgagtcaggacattagcattaatttagcctggtatcagcaaaaaccggggaaaactcctaagctcctgatctatgctgctgcatccactttgcaatcaggggtcccatctcggttcagtggcagtggatctgggacagatttcactctcaccatcagcagcctgcagcctgaagatgttgcaacttattactgtcaaaactataacagtgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 134 - DNA, 701 bp, synthetic construct
  source 1..701: mol_type = other DNA
gcttccagagcccatatcaatgcctgggtcagagctctggggaggaactgctcagttaggacccagacggaaccatggaagccccagcgcagcttctcttcctcctgctactctggctcccagataccactggagaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccaaccaggttcagtggcagtgggtctgggacagagttcactctcaccatcagcagcctgcagtctgaagattttgcagtttattactgtcagcagtataataactggccgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 135 - DNA, 502 bp, synthetic construct
  source 1..502: mol_type = other DNA
ggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcttccagtttacaaagtggggtcccatcgaggttcagcggcagtggatctgggacagaattcactctcacaatcagcagcctgcagcctgaagattttgcaacttattactgtctacagcataatagttatttcaccttcggccaagggacacgactggagattaaacgggctgatgctgcaccaactgtatccatcttcccaccatccagtgagcagttaacatctggaggtgcctcagtcgtgtgcttcttgaa

SEQ ID NO: 136 - DNA, 681 bp, synthetic construct
  source 1..681: mol_type = other DNA
gaggctgatcacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagctggtttgcctggtatcagcagaaaccagggaaagcccctaaactcctgatctatgctgcatccagtttgctaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatttcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 137 - DNA, 673 bp, synthetic construct
  source 1..673: mol_type = other DNA
ggacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagttggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 138 - DNA, 802 bp, synthetic construct
  source 1..802: mol_type = other DNA
tggttgtggttttcttatatggggaggagtcagtcccaaccaggacacagcatggacatgagggtccctgctcagctcctggggctcctgctgctctggctctcaggtgccagatgtgacatccagatgacccagtctccatcctccctgtctgcatctgtaggagacagagtcaccatcacttgccgggcaagtctgagcattggcagctacttaaattggtatcagaagaaaccagggaaagcccctaaactcctgatctttgtcgcatccagtttgcaaggtggggtctcatcaaggttcagtggcagcggctctgggacagatttcactctcaccatcagcagtctgcaacctgaagattttgcaacttactactgtcaacagacttacagtactccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagccaagtgacca

SEQ ID NO: 139 - DNA, 502 bp, synthetic construct
  source 1..502: mol_type = other DNA
ggggaggagtcagacccagtcaggacacagcatggacatgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtagctggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacagtataatagttattctcggacgttcggccaagggaccaaggtggaaatcaaacgggctgatgctgcaccaactgtatccatcttcccaccatccagtgagcagttaacatctggaggtgcctcagtcgtgtgcttc

SEQ ID NO: 140 - DNA, 500 bp, synthetic construct
  source 1..500: mol_type = other DNA
ggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccattacttgccgggcaagtcagggcattagaaatgatttaggctggtatcaacagaaaccagggaaagaccctaagcgcctgatctttgctgcaaccaatttgcacggtggggtcccatcaaggttcagcggcggtgtatctgggacagaattcactctcacaatcagcagcctgcagcctgaagattttgcaacttattactgtctacagcataatagttacccgctcactttcggcggagggaccaaggtggagatcaaacgggctgatgctgcaccaactgtatccatcttcccaccatccagtgagcagttaacatctggaggtgcctcagtcgtgtgcttc

SEQ ID NO: 141 - DNA, 673 bp, synthetic construct
  source 1..673: mol_type = other DNA
gagagctctggggaggaactgctcagttaggacccagacggaaccatggaagccccagcgcagcttctcttcctcctgctactctggctcccagaaaccactggagaaatagtgatgacgcagtctccagccaccctgtctgtgtctccaggggaaagagccaccctctcctgcagggccagtcagattgttggcagcaacttagcctggtaccaccagaaacctggccaggctcccagactcctcatctatagtgcatccaccagggccactggtatcccagccaggttcagtggcagtgggtctgggacagagttcactctcaccatcagcagcctgcagtctgaagattttgcagtttattactgtcagcagtataataactggcctccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgac

SEQ ID NO: 142 - DNA, 531 bp, synthetic construct
  source 1..531: mol_type = other DNA
ggggcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgagtcagggcttaggcaattctgtagcctggtatcagcagaaaccagggcaaattcctaggctcctgatctctgctgcggcctctttacaatcaggggtcccatctcggttcagtggcagtggatctgggacagatttcactctcaccatcagcagcctgcagcctgaagatgttgcaacttattactgtcaaaactataacagtgcccctcggacgttcggccaagggaccaatgtagaaatcaaacgggctgatgctgcaccaactgtatccatcttcccaccatccagtgagcagttaacatctggaggtgcctcagtcgtgtgcttcttgaacaacttctaccccaaagacatca

SEQ ID NO: 143 - DNA, 529 bp, synthetic construct
  source 1..529: mol_type = other DNA
ggggaggctgatcacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgatcaccttcggccaagggacacgactggagattaaacgggctgatgctgcaccaactgtatccatcttcccaccatccagtgagcagttaacatctggaggtgcctcagtcgtgtgcttcttgaaca

SEQ ID NO: 144 - DNA, 671 bp, synthetic construct
  source 1..671: mol_type = other DNA
gacaccctgtgcaggagtcagacccactcaggacacagcatggacatgagggtccccgctcagctcctggggctcctgctgctctggctcccaggtgccagatgtgccatccagatgacccagtctccatcctccctgtctgcatctgtaggagacagagtcaccatcacttgccgggcaagtcagggcattagaaatgatttagactggtttcagcagaaaccagggaaagcccctaagctcctgatctatggtgcgtccaatttacaaagtggggtcccatcaaggttcggcggcagtggatctggcacagatttcactctcaccatcagcagcctgcagcctgaagattttgcaacttattcctgtctacaaaattacaattacccgtggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgaca

SEQ ID NO: 145 - DNA, 782 bp, synthetic construct
  source 1..782: mol_type = other DNA
ggtgcaggagtcagtcccagtcaggacacagcatggacatgagggtccccgctcagctcctggggctcctgctgctctggctcccaggtgccagatgtgacatccagttgacccagtctccatccctcctgtctgcatctgtaggagacagagtcaccatcacttgctgggccagtcaggacattgccggtcatttcgcctggtatcagcaaaaaccagggacagtccctaaactcctgatctacgctatatccactttggacagtggggtcccatcaaggttcagcggcagtagatctgggacagaattcactctcacaatcagcagcctgcagcctgaggattttgcaacttattactgtcaacaccttaatagttacccgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagccaagtgacc

SEQ ID NO: 146 - DNA, 519 bp, synthetic construct
  source 1..519: mol_type = other DNA
gaggctgatcacaccctgtgcaggagtcagacccactcaggacacagcatggacatgagggtccccgctcagctcctggggctcctgctgctctggctcccaggtgccagatgtgccatccagatgacccagtctccatcctccctgtctgcatctgtgggagacagaatcaccatcacttgccgggcaagtcagggcattagcaatgacttaggctggtatcagcagaaaccagggaaagcccctaaactcctgatctttgctgcatccagtctacaaagtggggtcctatcaaggttcagcggcagtggatctggcacagatttcactctcaccatcagcagcctgcagcctgaagatgttgcaacttatttctgtctacaagattacaattttcctcggacattcggccaagggaccaaggtggaaatcaaacgggctgatgctgcaccaactgtatccatcttcccaccatccagtgagcagttaacatctggaggtgcctcagtcgtgtgcttc

SEQ ID NO: 147 - DNA, 723 bp, synthetic construct
  source 1..723: mol_type = other DNA
ttggagttgagttttcttatatggggagaagagctgctcagttaggacccagagggaaccatggaaaccccagcgcagcttctcttcctcctgctactctggctcccagataccaccggagaaattgtgttgacgcagtctccaggcaccctgtctttgtctccaggggaaagagtcaccctctcctgcagggccagtcagagtgttagcgccacctactttgcctggtaccaacagaaacctggccagcctcccaggctcctcatctatggcgcatccaacagggccactggcatcccagacaggttcagtggcagtgggtctgggacagacttcactctcaccatcagcagactggagcctgaagattttgcagtgtattactgtcagcagaatggtaactcgtacacttttggccaggggaccatcctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatca

SEQ ID NO: 148 - DNA, 772 bp, synthetic construct
  source 1..772: mol_type = other DNA
gagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctttagtagacagagtcaccatcacttgccgggcaagtcagaccattagcagctatttaaattggtatcagcagaaaccaggaaaagcccctaagctcctgatctatggtgcatccattttgcaaagtggggtcccatcaaggttcagtggcagtggatctgggacagatttcactctcaccatcagcagtctgcaacctgaagatcatgcagtttacttctgtcaacagagttacagttcccccactttcggcggagggaccaaggtggagatcaaaagggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 149 - DNA, 682 bp, synthetic construct
  source 1..682: mol_type = other DNA
cccttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctgagcgccttatctatgctgcatccaatttgcaaagtggggtcccatcaaggttcagcggcagtggatctgggacagaattcactctcacaatcagcagcctgcagcctgaagattttgcaactttttactgtctacagcattatagttacccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataaca

SEQ ID NO: 150 - DNA, 686 bp, synthetic construct
  source 1..686: mol_type = other DNA
ccgtttcttatatggggaattaggactcctcaggtcaccttctcacaatgaggctccttgctcatcttctggggctgctaatgctctgggtccctggatccagtggggatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagcaacacaatttcccactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 151 - DNA, 689 bp, synthetic construct
  source 1..689: mol_type = other DNA
ccaccacgtattttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgttggagacagagtcaccatcacttgccgggcaagtcagggcattagagatgatttaggctggtatcagcagaaaccagggaaagcccctgagcgcctgatctatgctgcatccaatttgcaaagtggggtcccatcaaggttcagcggcagtggatctgggacagaattcactctcacaatcagcagcctgcagcctgaagattttgcaactttttactgtctacagcattataattacccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataaca

SEQ ID NO: 152 - DNA, 679 bp, synthetic construct
  source 1..679: mol_type = other DNA
gctttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcaacagaaaccagggaaagcccctaagcgcctgatctatgctgcatccaatttgcacggtggggtcccatcaaggttcagcggcagtggatctgggacagaattcactctcacaatcagcagcctgcagcctgaagattttgtaacttattactgtctacagcattataattacccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacata

SEQ ID NO: 153 - DNA, 841 bp, synthetic construct
  source 1..841: mol_type = other DNA
ctggcctgatcacctgggcatgggctgctgagagcagaaaggggaggcagattgtctctgcagctgcaagcccagcacccgccccagctgctttgcatgtccctcccagccgccctgcagtccagagcccatatcaatgcctgggtcagagctctggagaagagctgctcagttaggacccagagggaaccatggaaaccccagcgcagcttctcttcctcctgctactctggctcccagataccaccggagaaattgtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagaagggccactggcatcccagacaggttcagtggcagtgggtctgggacagacttcactctcaccatcagcagactggagcctgaagattttgcagtatattactgtcagcagtatggtagctcgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccac

SEQ ID NO: 154 - DNA, 832 bp, synthetic construct
  source 1..832: mol_type = other DNA
agcctgcagggagaaacacatgctctgcagactttggacacctgggaggcactgggcctgtgcagtgttattgagataagacatctttgcagctgtgcagatttgcatgtcccacagagcaacgcctactgccctgaacatttatcaaaaggctggtgacatcctgtgcagaagtctctctcagtcaggacacagcatggacatgagggtccctgctcagctcctgggactcctgctgctctggctcccagataccagatgtgacatccagatgacccagtctccatcctccctgtctgcatctgtaggagacagagtcaccatcacttgccgggcgagtcagggcttgagtaattctgtagcctggtatcagcagaaaccagggcaaattcctaggctcctgatctctgctgcggcctctttgcaatcaggggtcccatctcggttcagtggcagtggatctgggacagatttcactctcaccatcagcagactgcagcctgaagatgttgcaacttattactgtcaaaactataacagtgcccctcggacgttcggccaagggaccaatgtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataac

SEQ ID NO: 155 - DNA, 775 bp, synthetic construct
  source 1..775: mol_type = other DNA
gagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccatatgtgacatccagatgacccagtctccatcctccctgtctgcatctgtaggagccagagtcaccatcacttgccgggcaagtcagagcattaacaatgatttaaattggtatcagcagaagccagggaaagcccctaagttcttgatctatgctgcatccagtttgcaaagtggggtcccatcaaggttcagcggccgtggatctgggacagatttcactctcaccatcagaagtctgcaacctgaagactttgctgtttactactgtcaacagacttacagttcccccttcactttcggccctgggaccaaagtggatttgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 156 - DNA, 819 bp, synthetic construct
  source 1..819: mol_type = other DNA
ggggacacctggggacactgagctgctgctgagttactgagatgagccagccctgcagctgcgcccagcctgccccatcccctgctcatttgcatgttcccagagcacagcctcctgccctgaagccttattaataggctggacacacttcatgcaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccaatttgcaaagtggggtcccatcaagattcagcggcagtggatctgggacagaattcactttcacaatcagcagcctgcagcctgaagattttgcaacttattactgtctacagcataatagttacccgtatacttttggccaggggaccaaactggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataaca

SEQ ID NO: 157 - DNA, 756 bp, synthetic construct
  source 1..756: mol_type = other DNA
gctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgcaggcgacagagtcaccatcacttgccgggcaagtcagagcattagcagctatttaaattggtatcagcagaaaccagggaaagcccctaagctcctgatctatgctgcatccagtttgcaaagtggagtcccatcaaggttcagtggcagtggatctgggacagatttcactctcaccatcagcagtctccaacctgaagattctgcaacttactactgtcaacagagttatggttttcacaccttcggccaggggacacgactggagattaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataac

SEQ ID NO: 158 - DNA, 755 bp, synthetic construct
  source 1..755: mol_type = other DNA
gcgcccagcctgccccatcccctgctcatttgcatgttcccagagcacagtctcctgacctgaagacttattaacaggctgatcacaccctgtgcaggagtcagacccagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgtcgggcgagtcagggtattagcagctggttagcctggtatcagcagaaaccagggaaagtccctaagttcctgatctatgctgcatccaatttgcaaagtggggtcccatcaaggttcagcggcagtggatctgggacagatttcactctcaccatcagcagcctgcagcctgaagattttgcaacttactattgtcaacaggctaatagtttcccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 159 - DNA, 690 bp, synthetic construct
  source 1..690: mol_type = other DNA
tttcttatatggggagaagagctgctcagttaggacccagagggaaccatggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtacatccagcagggccactggcatcccagacaggttcagtggcagtgggtctgggacagacttcactctcaccatcagcagactggagcctgaagattttgcagtgtattactgtcagctgtatggtagctcaccgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacc

SEQ ID NO: 160 - DNA, 784 bp, synthetic construct
  source 1..784: mol_type = other DNA
tactaagatgagccagccctgcagctgtgctcagcctgccccatgccctgctgattgatttgcatgttcccgagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccagggaaagcccctaagttcctgatctatgctgcatccagtttgcaaagtggggtcccatcaaggttcagtggcggtggatctgggacagatttcactctcaccatcagcagtctgcagcctgaagattttgcaacttactcctgtcaacagagttacagttcccccttcactttcggccctgggaccaaagtggctatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 161 - DNA, 794 bp, synthetic construct
  source 1..794: mol_type = other DNA
ggtttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctataggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagccaagtgacca

SEQ ID NO: 162 - DNA, 677 bp, synthetic construct
  source 1..677: mol_type = other DNA
acttttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgttgtatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 163 - protein, 11 aa, synthetic construct [sequence omitted]

SEQ ID NO: 164 - protein, 10 aa, synthetic construct
CASLAARPFW

SEQ ID NO: 165 - DNA, 33 bp, synthetic construct
  source 1..33: mol_type = other DNA
tgtcaacaggctaacagtttccctctcactttc

SEQ ID NO: 166 - protein, 11 aa, synthetic construct [sequence omitted]

SEQ ID NO: 167 - protein, 17 aa, synthetic construct
CAKDTGSYHYYYGLDVW

SEQ ID NO: 168 - DNA, 33 bp, synthetic construct
  source 1..33: mol_type = other DNA
tgtctacagcattatagttacccgctcactttc

SEQ ID NO: 169 - protein, 11 aa, synthetic construct [sequence omitted]

SEQ ID NO: 170 - protein, 16 aa, synthetic construct
CAKDSGSSWYGYFDLW

SEQ ID NO: 171 - DNA, 33 bp, synthetic construct
  source 1..33: mol_type = other DNA
tgccaacagtataatagtgattctcggacgttc

SEQ ID NO: 172 - protein, 11 aa, synthetic construct [sequence omitted]

SEQ ID NO: 173 - protein, 16 aa, synthetic construct
CTKDSSSSWYGYFDLW

SEQ ID NO: 174 - DNA, 33 bp, synthetic construct
  source 1..33: mol_type = other DNA
tgtcaacagtataatagtgattctcggacgttc

SEQ ID NO: 175 - protein, 10 aa, synthetic construct [sequence omitted]

SEQ ID NO: 176 - protein, 11 aa, synthetic construct
CAKRKRGLDGW

SEQ ID NO: 177 - DNA, 30 bp, synthetic construct
  source 1..30: mol_type = other DNA
tgcatgcaaggtctacaaactctcactttc

SEQ ID NO: 178 - protein, 11 aa, synthetic construct [sequence omitted]

SEQ ID NO: 179 - protein, 17 aa, synthetic construct
CTKDTGSYHSYYGLDVW

SEQ ID NO: 180 - DNA, 33 bp, synthetic construct
  source 1..33: mol_type = other DNA
tgtctacagcttaatacttaccctctcactttc

SEQ ID NO: 181 - protein, 11 aa, synthetic construct
CQNYNSVPRTF

SEQ ID NO: 182 - protein, 16 aa, synthetic construct
CARDYSSAWYGYFDYW

SEQ ID NO: 183 - DNA, 33 bp, synthetic construct
  source 1..33: mol_type = other DNA
tgtcaaaactataacagtgtccctcggacgttc

SEQ ID NO: 184 - protein, 11 aa, synthetic construct [sequence omitted]

SEQ ID NO: 185 - protein, 16 aa, synthetic construct
CTKDRGSWYEGWFDPW

SEQ ID NO: 186 - DNA, 33 bp, synthetic construct
  source 1..33: mol_type = other DNA
tgtctacagcataatagttacccactcactttc

SEQ ID NO: 187 - protein, 11 aa, synthetic construct [sequence omitted]

SEQ ID NO: 188 - protein, 15 aa, synthetic construct
CARPYSRATGYFDLW

SEQ ID NO: 189 - DNA, 33 bp, synthetic construct
  source 1..33: mol_type = other DNA
tgcatgcaagctctacaaactccattcactttc

SEQ ID NO: 190 - protein, 11 aa, synthetic construct
CLQHYIYPLTF

SEQ ID NO: 191 - protein, 17 aa, synthetic construct
CAKDTGSYHYYYGLDVW

SEQ ID NO: 192 - DNA, 33 bp, synthetic construct
  source 1..33: mol_type = other DNA
tgtctacagcattatatttacccgctcactttc

SEQ ID NO: 193 - protein, 11 aa, synthetic construct
CQNYNSVPRTF

SEQ ID NO: 194 - protein, 16 aa, synthetic construct
CARDYSSAWYGYFDSW

SEQ ID NO: 195 - DNA, 33 bp, synthetic construct; identical to SEQ ID NO: 183
  source 1..33: mol_type = other DNA

SEQ ID NO: 196 - protein, 11 aa, synthetic construct [sequence omitted]

SEQ ID NO: 197 - protein, 12 aa, synthetic construct
CAKEGRTGFDYW

SEQ ID NO: 198 - DNA, 33 bp, synthetic construct
  source 1..33: mol_type = other DNA
tgtcaaaagtataacagtgccccgtggacgttc

SEQ ID NO: 199 - protein, 11 aa, synthetic construct [sequence omitted]

SEQ ID NO: 200 - protein, 16 aa, synthetic construct
CVRDSSSSWYGVFDYW

SEQ ID NO: 201 - DNA, 33 bp, synthetic construct
  source 1..33: mol_type = other DNA
tgtcaaaactataacagtgcccctcggacgttc

SEQ ID NO: 202 - protein, 11 aa, synthetic construct [sequence omitted]

SEQ ID NO: 203 - protein, 17 aa, synthetic construct
CAKDSGSYHYYYGMDVW

SEQ ID NO: 204 - DNA, 33 bp, synthetic construct
  source 1..33: mol_type = other DNA
tgtctacagcataatagttaccctctcactttc

SEQ ID NO: 205 - protein, 11 aa, synthetic construct
CQNYNSVPRTF

SEQ ID NO: 206 - protein, 16 aa, synthetic construct
CARDYSSAWYGYFDYW

SEQ ID NO: 207 - DNA, 33 bp, synthetic construct; identical to SEQ ID NO: 183
  source 1..33: mol_type = other DNA

SEQ ID NO: 208 - protein, 11 aa, synthetic construct [sequence omitted]

SEQ ID NO: 209 - protein, 16 aa, synthetic construct
CAKDISSSWYGYFDLW

SEQ ID NO: 210 - DNA, 33 bp, synthetic construct
  source 1..33: mol_type = other DNA
tgtcaagagtataacagtgcccctcggacgttc

SEQ ID NO: 211 - protein, 11 aa, synthetic construct
CQQYTNLPFTF

SEQ ID NO: 212 - protein, 20 aa, synthetic construct
CARDGVTMVRGVIPHAFDIW

SEQ ID NO: 213 - DNA, 33 bp, synthetic construct
  source 1..33: mol_type = other DNA
tgtcaacagtatactaatctcccattcactttc

SEQ ID NO: 214 - protein, 10 aa, synthetic construct [sequence omitted]

SEQ ID NO: 215 - protein, 11 aa, synthetic construct [sequence omitted]

SEQ ID NO: 216 - DNA, 30 bp, synthetic construct
  source 1..30: mol_type = other DNA
tgtcagcagtatggtagttcagtcaccttc

SEQ ID NO: 217 - protein, 11 aa, synthetic construct
CQVYNSDSRTF

SEQ ID NO: 218 - protein, 16 aa, synthetic construct
CAKDVSSAWYGYFDYW

SEQ ID NO: 219 - DNA, 33 bp, synthetic construct
  source 1..33: mol_type = other DNA
tgccaagtttataatagtgattctcggacgttc

SEQ ID NO: 220 - protein, 11 aa, synthetic construct [sequence omitted]

SEQ ID NO: 221 - protein, 17 aa, synthetic construct
CARDRGSIAARGAFDIW

SEQ ID NO: 222 - DNA, 33 bp, synthetic construct
  source 1..33: mol_type = other DNA
tgtcaacagagttacagttcccctctcaccttc

SEQ ID NO: 223 - protein, 11 aa, synthetic construct [sequence omitted]

SEQ ID NO: 224 - protein, 16 aa, synthetic construct
CARDDSSSLYGYFQHW

SEQ ID NO: 225 - DNA, 33 bp, synthetic construct; identical to SEQ ID NO: 201
  source 1..33: mol_type = other DNA

SEQ ID NO: 226 - protein, 10 aa, synthetic construct [sequence omitted]

SEQ ID NO: 227 - protein, 15 aa, synthetic construct
CAKDIGGSYLGFDYW

SEQ ID NO: 228 - DNA, 30 bp, synthetic construct
  source 1..30: mol_type = other DNA
tgtcagcagtatggtagctcgtacactttt

SEQ ID NO: 229 - protein, 11 aa, synthetic construct [sequence omitted]

SEQ ID NO: 230 - protein, 17 aa, synthetic construct
CAKDRGSIAVRGAFDIW

SEQ ID NO: 231 - DNA, 33 bp, synthetic construct
  source 1..33: mol_type = other DNA
tgtcaacaggctaacagtttcccgtggacgttc

SEQ ID NO: 232 - protein, 11 aa, synthetic construct [sequence omitted]

SEQ ID NO: 233 - protein, 15 aa, synthetic construct
CTKDIGGSYYASDFW

SEQ ID NO: 234 - DNA, 33 bp, synthetic construct
  source 1..33: mol_type = other DNA
tgtcaacagagttacagtaccccattcactttc

SEQ ID NO: 235 - protein, 11 aa, synthetic construct [sequence omitted]

SEQ ID NO: 236 - protein, 16 aa, synthetic construct
CTRDSTSSWYGVFDFW

SEQ ID NO: 237 - DNA, 33 bp, synthetic construct; identical to SEQ ID NO: 201
  source 1..33: mol_type = other DNA

SEQ ID NO: 238 - protein, 11 aa, synthetic construct [sequence omitted]

SEQ ID NO: 239 - protein, 14 aa, synthetic construct
CARGWNYGYYFDYW

SEQ ID NO: 240 - DNA, 33 bp, synthetic construct
  source 1..33: mol_type = other DNA
tgtcagcagtataataactggccgtacactttt

SEQ ID NO: 241 - protein, 10 aa, synthetic construct [sequence omitted]

SEQ ID NO: 242 - protein, 15 aa, synthetic construct
CAKDFNSWPHEFDVW

SEQ ID NO: 243 - DNA, 30 bp, synthetic construct
  source 1..30: mol_type = other DNA
tgtctacagcataatagttatttcaccttc

SEQ ID NO: 244 - protein, 11 aa, synthetic construct [sequence omitted]

SEQ ID NO: 245 - protein, 12 aa, synthetic construct
CAKDRRGSFDYW

SEQ ID NO: 246 - DNA, 33 bp, synthetic construct
  source 1..33: mol_type = other DNA
tgtcaacaggctaacagtttcccattcactttc

SEQ ID NO: 247 - protein, 11 aa, synthetic construct [sequence omitted]

SEQ ID NO: 248 - protein, 11 aa, synthetic construct
CARYGNWNYIW

SEQ ID NO: 249 - DNA, 33 bp, synthetic construct
  source 1..33: mol_type = other DNA
tgtcaacaggctaacagtttcccgctcactttc

SEQ ID NO: 250 - protein, 11 aa, synthetic construct [sequence omitted]

SEQ ID NO: 251 - protein, 15 aa, synthetic construct
CAKDIGGSQYALDVW

SEQ ID NO: 252 - DNA, 33 bp, synthetic construct
  source 1..33: mol_type = other DNA
tgtcaacagacttacagtactccattcactttc

SEQ ID NO: 253 - protein, 11 aa, synthetic construct [sequence omitted]

SEQ ID NO: 254 - protein, 16 aa, synthetic construct
CAKDYSSAWYGYFDYW

SEQ ID NO: 255 - DNA, 33 bp, synthetic construct
  source 1..33: mol_type = other DNA
tgccaacagtataatagttattctcggacgttc

SEQ ID NO: 256 - protein, 11 aa, synthetic construct [sequence omitted]

SEQ ID NO: 257 - protein, 17 aa, synthetic construct
CAKDSGSYHYYYGMDGW

SEQ ID NO: 258 - DNA, 33 bp, synthetic construct
  source 1..33: mol_type = other DNA
tgtctacagcataatagttacccgctcactttc

SEQ ID NO: 259 - protein, 12 aa, synthetic construct [sequence omitted]

SEQ ID NO: 260 - protein, 14 aa, synthetic construct
CARVNWNSDAFDIW

SEQ ID NO: 261 - DNA, 36 bp, synthetic construct
  source 1..36: mol_type = other DNA
tgtcagcagtataataactggcctccattcactttc

SEQ ID NO: 262 - protein, 11 aa, synthetic construct [sequence omitted]

SEQ ID NO: 263 - protein, 16 aa, synthetic construct
CVKEFSSSWYGFFDLW

SEQ ID NO: 264 - DNA, 33 bp, synthetic construct; identical to SEQ ID NO: 201
  source 1..33: mol_type = other DNA

SEQ ID NO: 265 - protein, 11 aa, synthetic construct [sequence omitted]

SEQ ID NO: 266 - protein, 13 aa, synthetic construct
CARDRSSSPLENW

SEQ ID NO: 267 - DNA, 33 bp, synthetic construct
  source 1..33: mol_type = other DNA
tgtcaacaggctaacagtttcccgatcaccttc

SEQ ID NO: 268 - protein, 11 aa, synthetic construct
CLQNYNYPWTF

SEQ ID NO: 269 - protein, 16 aa, synthetic construct
CAKDMGSSGWYVGDHW

SEQ ID NO: 270 - DNA, 33 bp, synthetic construct
  source 1..33: mol_type = other DNA
tgtctacaaaattacaattacccgtggacgttc

SEQ ID NO: 271 - protein, 11 aa, synthetic construct [sequence omitted]

SEQ ID NO: 272 - protein, 17 aa, synthetic construct
CARWGGFGVTGTHLDYW

SEQ ID NO: 273 - DNA, 33 bp, synthetic construct
  source 1..33: mol_type = other DNA
tgtcaacaccttaatagttacccgtacactttt

SEQ ID NO: 274 - protein, 11 aa, synthetic construct [sequence omitted]

SEQ ID NO: 275 - protein, 15 aa, synthetic construct
CVRDDYGDYVPLDYW

SEQ ID NO: 276 - DNA, 33 bp, synthetic construct
  source 1..33: mol_type = other DNA
tgtctacaagattacaattttcctcggacattc

SEQ ID NO: 277 - protein, 10 aa, synthetic construct
CQQNGNSYTF

SEQ ID NO: 278 - protein, 15 aa, synthetic construct
CAKDIGGSYLGFDYW

SEQ ID NO: 279 - DNA, 30 bp, synthetic construct
  source 1..30: mol_type = other DNA
tgtcagcagaatggtaactcgtacactttt

SEQ ID NO: 280 - protein, 10 aa, synthetic construct [sequence omitted]

SEQ ID NO: 281 - protein, 15 aa, synthetic construct
CARHRGSYVYGMDVW

SEQ ID NO: 282 - DNA, 30 bp, synthetic construct
  source 1..30: mol_type = other DNA
tgtcaacagagttacagttcccccactttc

SEQ ID NO: 283 - protein, 11 aa, synthetic construct [sequence omitted]

SEQ ID NO: 284 - protein, 17 aa, synthetic construct
CAKDTGSYHYYYGLDVW

SEQ ID NO: 285 - DNA, 33 bp, synthetic construct; identical to SEQ ID NO: 168
  source 1..33: mol_type = other DNA

SEQ ID NO: 286 - protein, 10 aa, synthetic construct [sequence omitted]

SEQ ID NO: 287 - protein, 11 aa, synthetic construct
CAKDLGSTVYW

SEQ ID NO: 288 - DNA, 30 bp, synthetic construct
  source 1..30: mol_type = other DNA
tgcatgcaagcaacacaatttcccactttc

SEQ ID NO: 289 - protein, 11 aa, synthetic construct [sequence omitted]

SEQ ID NO: 290 - protein, 17 aa, synthetic construct
CGKDTGSYHYYYGLDVW

SEQ ID NO: 291 - DNA, 33 bp, synthetic construct
  source 1..33: mol_type = other DNA
tgtctacagcattataattacccgctcactttc

SEQ ID NO: 292 - protein, 11 aa, synthetic construct [sequence omitted]

SEQ ID NO: 293 - protein, 17 aa, synthetic construct
CAKDSGSYHYYYGMDVW

SEQ ID NO: 294 - DNA, 33 bp, synthetic construct; identical to SEQ ID NO: 291
  source 1..33: mol_type = other DNA

SEQ ID NO: 295 - protein, 10 aa, synthetic construct [sequence omitted]

SEQ ID NO: 296 - protein, 15 aa, synthetic construct
CTKDLGGSYLGFDYW

SEQ ID NO: 297 - DNA, 30 bp, synthetic construct; identical to SEQ ID NO: 228
  source 1..30: mol_type = other DNA

SEQ ID NO: 298 - protein, 11 aa, synthetic construct [sequence omitted]

SEQ ID NO: 299 - protein, 16 aa, synthetic construct
CAKEHSSSWYGFFDFW

SEQ ID NO: 300 - DNA, 33 bp, synthetic construct; identical to SEQ ID NO: 201
  source 1..33: mol_type = other DNA

SEQ ID NO: 301 - protein, 11 aa, synthetic construct
CQQTYSSPFTF

SEQ ID NO: 302 - protein, 15 aa, synthetic construct
CAKDLGGSHYALDVW

SEQ ID NO: 303 - DNA, 33 bp, synthetic construct
  source 1..33: mol_type = other DNA
tgtcaacagacttacagttcccccttcactttc

SEQ ID NO: 304 - protein, 11 aa, synthetic construct [sequence omitted]

SEQ ID NO: 305 - protein, 17 aa, synthetic construct
CAKDTSSWSYYYGMDVW

SEQ ID NO: 306 - DNA, 33 bp, synthetic construct
  source 1..33: mol_type = other DNA
tgtctacagcataatagttacccgtatactttt

SEQ ID NO: 307 - protein, 10 aa, synthetic construct
CQQSYGFHTF

SEQ ID NO: 308 - protein, 16 aa, synthetic construct
CAKDRRIVGATGFDYW

SEQ ID NO: 309 - DNA, 30 bp, synthetic construct
  source 1..30: mol_type = other DNA
tgtcaacagagttatggttttcacaccttc

SEQ ID NO: 310 - protein, 11 aa, synthetic construct [sequence omitted]

SEQ ID NO: 311 - protein, 12 aa, synthetic construct
CAKDRRGAFDIG

SEQ ID NO: 312 - DNA, 33 bp, synthetic construct
  source 1..33: mol_type = other DNA
tgtcaacaggctaatagtttcccattcactttc

SEQ ID NO: 313 - protein, 11 aa, synthetic construct [sequence omitted]

SEQ ID NO: 314 - protein, 15 aa, synthetic construct
CARYSSRRYYGMDVW

SEQ ID NO: 315 - DNA, 33 bp, synthetic construct
  source 1..33: mol_type = other DNA
tgtcagctgtatggtagctcaccgtacactttt

SEQ ID NO: 316 - protein, 11 aa, synthetic construct [sequence omitted]

SEQ ID NO: 317 - protein, 15 aa, synthetic construct
CAKDMGGSNYGLDVW

SEQ ID NO: 318 - DNA, 33 bp, synthetic construct
  source 1..33: mol_type = other DNA
tgtcaacagagttacagttcccccttcactttc

SEQ ID NO: 319 - protein, 11 aa, synthetic construct [sequence omitted]

SEQ ID NO: 320 - protein, 17 aa, synthetic construct
CVKDTGSWASYYGMDVW

SEQ ID NO: 321 - DNA, 33 bp, synthetic construct
  source 1..33: mol_type = other DNA
tgtctacagcataatagttacccgtacactttt

SEQ ID NO: 322 - protein, 11 aa, synthetic construct [sequence omitted]

SEQ ID NO: 323 - protein, 14 aa, synthetic construct
CARGVNWNYVFDYW

SEQ ID NO: 324 - DNA, 33 bp, synthetic construct
  source 1..33: mol_type = other DNA
tgtctacagcataatagttacccgtggacgttc

SEQ ID NO: 325 - DNA, 30 bp, synthetic construct
  source 1..30: mol_type = other DNA
tgtgcgtccctagcagctcgtcccttttgg

SEQ ID NO: 326 - DNA, 51 bp, synthetic construct
  source 1..51: mol_type = other DNA
tgtgcaaaagatactggcagctaccactactactacggtctggacgtctgg

SEQ ID NO: 327 - DNA, 48 bp, synthetic construct
  source 1..48: mol_type = other DNA
tgtgcaaaagatagtggcagcagctggtacgggtacttcgatctctgg

SEQ ID NO: 328 - DNA, 48 bp, synthetic construct
  source 1..48: mol_type = other DNA
tgtacaaaagatagcagcagcagctggtacgggtacttcgatctctgg

SEQ ID NO: 329 - DNA, 33 bp, synthetic construct
  source 1..33: mol_type = other DNA
tgtgcgaagcggaaaaggggtctggacggctgg

SEQ ID NO: 330 - DNA, 51 bp, synthetic construct
  source 1..51: mol_type = other DNA
tgtacaaaagatactgggagctaccactcctactacggtttggacgtctgg

SEQ ID NO: 331 - DNA, 48 bp, synthetic construct
  source 1..48: mol_type = other DNA
tgtgcaagagattacagcagtgcctggtacgggtactttgactactgg

SEQ ID NO: 332 - DNA, 48 bp, synthetic construct
  source 1..48: mol_type = other DNA
tgtacaaaagataggggcagctggtacgagggctggttcgacccctgg

SEQ ID NO: 333 - DNA, 45 bp, synthetic construct
  source 1..45: mol_type = other DNA
tgtgcgaggccctacagtagagcaactggatacttcgatctctgg

SEQ ID NO: 334 - DNA, 51 bp, synthetic construct
  source 1..51: mol_type = other DNA
tgtgcaaaagatactggtagttatcactactattacggtctggacgtctgg

SEQ ID NO: 335 - DNA, 48 bp, synthetic construct
  source 1..48: mol_type = other DNA
tgtgcaagagattacagtagtgcctggtacgggtactttgactcctgg

SEQ ID NO: 336 - DNA, 36 bp, synthetic construct
  source 1..36: mol_type = other DNA
tgtgcgaaagaagggagaactgggtttgactactgg

SEQ ID NO: 337 - DNA, 48 bp, synthetic construct
  source 1..48: mol_type = other DNA
tgtgtgagagattcaagcagcagttggtacggggtttttgactactgg

SEQ ID NO: 338 - DNA, 51 bp, synthetic construct
  source 1..51: mol_type = other DNA
tgtgcaaaagatagtgggagctatcactactactacggtatggacgtctgg

SEQ ID NO: 339 - DNA, 48 bp, synthetic construct; identical to SEQ ID NO: 331
  source 1..48: mol_type = other DNA

SEQ ID NO: 340 - DNA, 48 bp, synthetic construct
  source 1..48: mol_type = other DNA
tgtgcaaaagatataagcagcagctggtacgggtacttcgatctctgg

SEQ ID NO: 341 - DNA, 60 bp, synthetic construct
  source 1..60: mol_type = other DNA
tgtgcgagagatggggttactatggttcggggagttattccccatgcttttgatatctgg

SEQ ID NO: 342 - DNA, 33 bp, synthetic construct
  source 1..33: mol_type = other DNA
tgtgcgagaggggctaactggttcgacccctgg

SEQ ID NO: 343 - DNA, 48 bp, synthetic construct
  source 1..48: mol_type = other DNA
tgtgcaaaagatgtaagcagtgcctggtacggctactttgactactgg

SEQ ID NO: 344 - DNA, 51 bp, synthetic construct
  source 1..51: mol_type = other DNA
tgtgcgagagatcgagggagtatagcagctcggggtgcttttgatatctgg

SEQ ID NO: 345 - DNA, 48 bp, synthetic construct
  source 1..48: mol_type = other DNA
tgtgcgagagacgatagcagcagcttgtacggatacttccagcactgg

SEQ ID NO: 346 - DNA, 45 bp, synthetic construct
  source 1..45: mol_type = other DNA
tgtgcaaaggatattggagggagctacttagggtttgactactgg

SEQ ID NO: 347 - DNA, 51 bp, synthetic construct
  source 1..51: mol_type = other DNA
tgtgcgaaagatcgggggagtatagcagttcgaggtgcttttgatatctgg

SEQ ID NO: 348 - DNA, 45 bp, synthetic construct
  source 1..45: mol_type = other DNA
tgcacaaaagatataggtgggagctactacgcctctgacttctgg

SEQ ID NO: 349 - DNA, 48 bp, synthetic construct
  source 1..48: mol_type = other DNA
tgtacgagagattcaaccagtagttggtatggggtctttgacttctgg

SEQ ID NO: 350 - DNA, 42 bp, synthetic construct
  source 1..42: mol_type = other DNA
tgtgcaagaggctggaactacgggtactactttgactactgg

SEQ ID NO: 351 - DNA, 45 bp, synthetic construct
  source 1..45: mol_type = other DNA
tgtgcaaaagatttcaacagctggccccatgaatttgatgtctgg

SEQ ID NO: 352 - DNA, 36 bp, synthetic construct
  source 1..36: mol_type = other DNA
tgtgcgaaagatcggcggggatcctttgactactgg

SEQ ID NO: 353 - DNA, 33 bp, synthetic construct
  source 1..33: mol_type = other DNA
tgtgcgagatacgggaactggaactatatctgg

SEQ ID NO: 354 - DNA, 45 bp, synthetic construct
  source 1..45: mol_type = other DNA
tgtgcaaaggatataggggggagccaatacgctttggacgtctgg

SEQ ID NO: 355 - DNA, 48 bp, synthetic construct
  source 1..48: mol_type = other DNA
tgtgcaaaagattatagcagtgcctggtacgggtactttgactactgg

SEQ ID NO: 356 - DNA, 51 bp, synthetic construct
  source 1..51: mol_type = other DNA
tgtgcaaaagatagtgggagttaccactattactacggtatggacgggtgg

SEQ ID NO: 357 - DNA, 42 bp, synthetic construct
  source 1..42: mol_type = other DNA
tgtgcgagagtgaactggaactctgatgcttttgatatctgg

SEQ ID NO: 358 - DNA, 48 bp, synthetic construct
  source 1..48: mol_type = other DNA
tgtgtaaaagagtttagcagcagctggtacgggttcttcgatctctgg

SEQ ID NO: 359 - DNA, 39 bp, synthetic construct
  source 1..39: mol_type = other DNA
tgtgcgagagataggagcagctcccccttagaaaactgg

SEQ ID NO: 360 - DNA, 48 bp, synthetic construct
  source 1..48: mol_type = other DNA
tgtgcaaaagatatggggagcagtggctggtacgtgggcgaccactgg

SEQ ID NO: 361 - DNA, 51 bp, synthetic construct
  source 1..51: mol_type = other DNA
tgtgcacgctggggtggattcggtgtaactggaactcacttagactactgg

SEQ ID NO: 362 - DNA, 45 bp, synthetic construct
  source 1..45: mol_type = other DNA
tgtgtgagggatgactacggtgactacgttcctcttgactactgg

SEQ ID NO: 363 - DNA, 45 bp, synthetic construct
  source 1..45: mol_type = other DNA
tgtgcaaaagatattggtgggagctacttagggtttgactattgg

SEQ ID NO: 364 - DNA, 45 bp, synthetic construct
  source 1..45: mol_type = other DNA
tgtgcgagacatcgtgggagttatgtctacggtatggacgtctgg

SEQ ID NO: 365 - DNA, 51 bp, synthetic construct
  source 1..51: mol_type = other DNA
tgtgcaaaagatactggtagttaccactattactacggtctggacgtctgg

SEQ ID NO: 366 - DNA, 33 bp, synthetic construct
  source 1..33: mol_type = other DNA
tgtgcgaaagatttgggatctacagtctactgg

SEQ ID NO: 367 - DNA, 51 bp, synthetic construct
  source 1..51: mol_type = other DNA
tgtggaaaagatactggtagttaccactactactacggtctggacgtctgg

SEQ ID NO: 368 - DNA, 51 bp, synthetic construct
  source 1..51: mol_type = other DNA
tgtgcaaaagatagtgggagctaccactactattatggtatggacgtctgg

SEQ ID NO: 369 - DNA, 45 bp, synthetic construct
  source 1..45: mol_type = other DNA
tgtacaaaagatcttggtgggagttacttagggtttgactactgg

SEQ ID NO: 370 - DNA, 48 bp, synthetic construct
  source 1..48: mol_type = other DNA
tgtgcaaaagaacatagcagcagctggtacgggttcttcgatttctgg

SEQ ID NO: 371 - DNA, 45 bp, synthetic construct
  source 1..45: mol_type = other DNA
tgtgcaaaagatctgggagggagccactacgctttggacgtctgg

SEQ ID NO: 372 - DNA, 51 bp, synthetic construct
  source 1..51: mol_type = other DNA
tgtgcaaaagataccagcagctggagctactactacggtatggacgtctgg

SEQ ID NO: 373 - DNA, 48 bp, synthetic construct
  source 1..48: mol_type = other DNA
tgtgcgaaagatagacgtatagtgggagcgactggctttgactactgg

SEQ ID NO: 374 - DNA, 36 bp, synthetic construct
  source 1..36: mol_type = other DNA
tgcgcgaaagatcggagaggtgcttttgatatcggg

SEQ ID NO: 375 - DNA, 45 bp, synthetic construct
  source 1..45: mol_type = other DNA
tgtgcgaggtatagcagcagaaggtactacggtatggacgtctgg

SEQ ID NO: 376 - DNA, 45 bp, synthetic construct
  source 1..45: mol_type = other DNA
tgtgcaaaagatatgggagggagcaactacggtttggacgtctgg

SEQ ID NO: 377 - DNA, 51 bp, synthetic construct
  source 1..51: mol_type = other DNA
tgtgtaaaagataccggcagctgggcctcctactacggcatggacgtctgg

SEQ ID NO: 378 - DNA, 42 bp, synthetic construct
  source 1..42: mol_type = other DNA
tgtgcgaggggggtcaactggaactacgtctttgactactgg

SEQ ID NO: 379 - DNA, 16 bp, synthetic construct
  source 1..16: mol_type = other DNA
cctaccaaggcaaaga

SEQ ID NO: 380 - DNA, 643 bp, synthetic construct
  source 1..643: mol_type = other DNA
tgcctacacatttcttatatgggggcagagcctgctgaattctggctgaccagggcagtcaccagagctccagacaatgcctgtctccttcctcatcttcctgcccgtgctgggcctcccatggggtgtcctgtcacaggtacagctgcagcagtcaggtccaggactggtgaagccctcgcagaccctctcactcacctgtgtcatatccggggacagtgtctctagcaacagtgttacttggaactggatcaggcagtccccatcgagaggccttgagtggctgggaaggacatactacaggtccaagtggtataatgaatatgcagtttctgtgaaaagtcgagtaatcatcaacccagacacatcgaagaaccagttttccctgcagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 381 - DNA, 16 bp, synthetic construct
  source 1..16: mol_type = other DNA
agagcgaagatacaca

SEQ ID NO: 382 - DNA, 571 bp, synthetic construct
  source 1..571: mol_type = other DNA
ggcagagcctgctgaattctggctgaccagggcagtcaccagagctccagacaatgtctgtctccttcctcatcttcctgcccgtgctgggcctcccatggggtgtcctgtcacaggtacagctgcagcagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtcccagtggtatcatgaatatgcagtttctgtgatgaaacgagtcaccatcaacccagacacatccaagaatcagttttccctccagatgaactctatgactcccgaggacacggctgtatatttctgtgcaagaggggctaactggggattctggttcgacccctggggccagggaaccctgatcaccgtctcctcagccaaaacaacagccccatcggtctatccactggcccctgtgtgtggagatacaactggctcctcggtgactctaggatgcctggtcaaggg

SEQ ID NO: 383 - DNA, 16 bp, synthetic construct
  source 1..16: mol_type = other DNA
agtgaggcatggaata

SEQ ID NO: 384 - DNA, 644 bp, synthetic construct
  source 1..644: mol_type = other DNA
atattccggattttcttatatgggggcagagcctgctgaattctggctgaccagggcagtcaccagagctccagacaatgtctgtctccttcctcatcttcctgcccgtgctgggcctcccatggggtgtcctgtcacaggtacagctgcagcagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtccaagtggtataatgattatgcggtttctgtgaaaagtcgagtaaccatcaacccagacacatccaagaaccagttttccctgcagatgaactctatgactcccgaggacacggctgtatatttctgtgcaagaggggctaactggggattctggttcgacccctggggccagggaatcctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 385 - DNA, 16 bp, synthetic construct
  source 1..16: mol_type = other DNA
caactagagtgcaagc

SEQ ID NO: 386 - DNA, 643 bp, synthetic construct
  source 1..643: mol_type = other DNA
tgaacttttctttcttatatgggggcagagcctgctgaattctggctgaccagggcagtcaccagagctccagacaatgtctgtctccttcctcatcttcctgcccgtgctgggcctcccatggggtgtcctgtcacaggtacagctgcaacagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtccaactggtataatgattatgcagtttctgtgaaaagtcgagtaaccatcaacccagacacatccaagaaccagttttccctgcagatgaactctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 387 - DNA, 16 bp, synthetic construct
  source 1..16: mol_type = other DNA
caccactagacaggct

SEQ ID NO: 388 - DNA, 592 bp, synthetic construct
  source 1..592: mol_type = other DNA
tgggggcagagcctgctgaattctggctgaccagggcagtcaccagagctccagacaatgcctgtctccttcctcatcttcctgcccgtgctgggcctcccatggggtgtcctgtcacaggtacagctgcagcagtcaggtccaggactggtgaagccctcgcagaccctctcactcacctgtgtcatatccggggacagtgtctctagcaacagtgttacttggaactggatcaggcagtccccatcgagaggccttgagtggctgggaaggacatactacaggtccaagtggtataatgaatatgcagtttctgtgaaaagtcgagtaatcatcaacccagacacatcgaagaaccagttttccctgcagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccag

SEQ ID NO: 389 - DNA, 16 bp, synthetic construct
  source 1..16: mol_type = other DNA
tcattaccataggata

SEQ ID NO: 390 - DNA, 571 bp, synthetic construct
  source 1..571: mol_type = other DNA
ggcagagcctgctgaattctggctgaccagggcagtcaccagagctccagacaatgtctgtctccttcctcatcttcctgcccgtgctgggcctcccatggggtgtcctgtcacaggtacacctgcagcagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtccaagtggtataatgaatatgcagtttctgtgcaaagtcgagttaccatcaaccctgacacatccaagaaccagttttccctgcagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaaggg

SEQ ID NO: 391 - DNA, 16 bp, synthetic construct
  source 1..16: mol_type = other DNA
tgttccgtcccaagta

SEQ ID NO: 392 - DNA, 581 bp, synthetic construct
  source 1..581: mol_type = other DNA
gagagcctgctgaattctggctgaccagggcagtcaccagagctccagacaatgtctgtctccttcctcatcttcctgcccgtgctgggcctcccatggggtgtcctgtcacaggtacacctgcagcagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtccaagtggtataatgaatatgcagtttctgtgcaaagtcgagttaccatcaaccctgacacatccaagaaccagttttccctgcagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctga

SEQ ID NO: 393 - DNA, 16 bp, synthetic construct
  source 1..16: mol_type = other DNA
ttaggcagtctcatcc

SEQ ID NO: 394 - DNA, 620 bp, synthetic construct
  source 1..620: mol_type = other DNA
gggggcagagcctgctgaattctggctgaccagggcagtcaccagagctccagacaatgtctgtctccttcctcatcttcctgcccgtgctgggcctcccatggggtgtcctgtcacaggtacagctgcagcagtcaggtccaggactggtgaagccctcgcagaccctctcactcacctgtgccatatccggggacagtgtctctagcaacagtgccacttggaactggatcaggcagtccccatcgagaggccttgagtggctgggaaggacatactacaggtccaagtggtataatgaatatgcagtttctgtgaaaagtcgagtaaccatcaacccagacacatccaagaaccagttttccctacagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctg

SEQ ID NO: 395 - DNA, 16 bp, synthetic construct
  source 1..16: mol_type = other DNA
ttcccagcattgagct

SEQ ID NO: 396 - DNA, 635 bp, synthetic construct
  source 1..635: mol_type = other DNA
gtggtttcttatatggggagagcctgctgaattctggctgaccagggcagtcaccagagctccagacaatgtctgtcgccttcctcatcttcctgcccgtgctgggcctcccatggggtgtcctgtcacaggtacagctgcagcagtcaggtccaggactggtgaagccctcgcagaccctctcactcacctgtgccatatccggggacagtgtctctagcaacagtgctacttggaactggatcaggcagtccccatcgagaggccttgagtggctgggaaggacatactacaggtccaagtggtataatgaatatgaagtttctgtgaaaagtcgagtaattatcaacccagacacatccaagaaccagttttccctgcagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 397 - DNA, 16 bp, synthetic construct
  source 1..16: mol_type = other DNA
ttggcaaagatatgca

SEQ ID NO: 398 - DNA, 623 bp, synthetic construct
  source 1..623: mol_type = other DNA
tgggggcagagcctgctgaattctggctgaccagggcagtcaccagagctccagacaatgtctgtctccttcctcatcttcctgcccgtgctgggcctcccatggggtgtcctgtcacaggtacagctgcagcagtcaggtccaggactggtgaagccctcgcagaccctctcactcacctgtgccatatccggggacagtgtctctagcaacagtgccacttggaactggatcaggcagtccccatcgagaggccttgagtggctgggaaggacatactacaggtccaagtggtataatgaatatgcagtttctgtgaaaagtcgagtaaccatcaacccagacacatccaagaaccagttttccctacagatgaattctatgactcccgaggacacggctgtatatttctgtgcaagaggggctaactggggattctggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtc

SEQ ID NO: 399 - DNA, 16 bp, synthetic construct
  source 1..16: mol_type = other DNA
acgtcaagtgttcttt

SEQ ID NO: 400 - DNA, 748 bp, synthetic construct
  source 1..748: mol_type = other DNA
gcattctcctcccagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgtagacacgtccaggaaccagttcaccctgaagctgagttctgtgaccgccgcagacacggctttgttttactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 401 - DNA, 16 bp, synthetic construct
  source 1..16: mol_type = other DNA
caacctccaagcgagt

SEQ ID NO: 402 - DNA, 736 bp, synthetic construct
  source 1..736: mol_type = other DNA
gagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgttgacacgtccaggaaccagttcaccctgaagctgagttctgtgaccgccgcagacacggctttgtattactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 403 - DNA, 16 bp, synthetic construct
  source 1..16: mol_type = other DNA
cgattgacatggtcta

SEQ ID NO: 404 - DNA, 573 bp, synthetic construct
  source 1..573: mol_type = other DNA
gaggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgtagacacgtccaggaaccagttcaccctgaagctgagttctgtgaccgccgcagacacggctttgtattactgtgcgagacaaaaaggttcggggagttttgacaactggggccagggaaccctggtcaccgtctcctcagccaaaacaacacccccatcagtctatccactggcccctgggtgtggagatacaactggttcctccgtgactctgggatgcctggtcaagggctacttccc

SEQ ID NO: 405 - DNA, 16 bp, synthetic construct
  source 1..16: mol_type = other DNA
gcgaccacatccgtgg

SEQ ID NO: 406 - DNA, 616 bp, synthetic construct
  source 1..616: mol_type = other DNA
ggggaggtccaactcataagggaaatgctttctgagagtcatggatctcatgtgcaagaaaatgaagcacctgtggttcttcctcctgctggtggcggctcccagatgggtcctgtcccaactgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacgtactacaacccgtccctcaagagtcgagtcaccatatccatagacacgtccaggaaccagttcaccctgaagctgagttctgtgaccgccgcagacacggctttgtattactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 407 - DNA, 16 bp, synthetic construct
  source 1..16: mol_type = other DNA
ggtattgcaacgatgg

SEQ ID NO: 408 - DNA, 692 bp, synthetic construct
  source 1..692: mol_type = other DNA
gactgctccgtacaaacgcaccacccccatgcaaatcctcacttaggcgcccacaggaagccaccacacatttccttaaattcaggtccaactcataagggaaatgctttctgagagtcatggatctcatgtgcaagaaaatgaagcacctgtggttcttcctcctgctggtggcggctcccagatgggtcctgtcccaactgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgaagacacgtccaggaaccagttcaccctgaagctgagttctgtgaccgccgcagacacggctttgtattactgtgcgagacaaaaaggttcggggagttttgacaactggggccagggagccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctg

SEQ ID NO: 409 - DNA, 16 bp, synthetic construct
  source 1..16: mol_type = other DNA
gtcgggtcagggattg

SEQ ID NO: 410 - DNA, 615 bp, synthetic construct
  source 1..615: mol_type = other DNA
gggaggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgtagacacgtccaggaaccagttcgccctgaacctgaattctgtgaccgccgcagacacggctttgtattactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 411 - DNA, 16 bp, synthetic construct
  source 1..16: mol_type = other DNA
tctcatacattggtac

SEQ ID NO: 412 - DNA, 619 bp, synthetic construct
  source 1..619: mol_type = other DNA
gaaattcaggtccaactcataagggaaatgctttctgagagtcatggatctcatgtgcaagaaaatgaagcacctgtggttcctcctcctgctggtggcggctcccagatgggtcctgtcccagctgcaggtgcaggagtcgggcccaggactggtgaagccttcggagaccctgtccctcacatgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgaagacacgtccaggaaccagttcaccctgaagctgagttctgtgaccgccgcagacacggctttgtattactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 413 - DNA, 16 bp, synthetic construct
  source 1..16: mol_type = other DNA
tcttcggaggctacga

SEQ ID NO: 414 - DNA, 608 bp, synthetic construct
  source 1..608: mol_type = other DNA
ggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccgtagacacgtccaggaaccagttcgccctgaacctgaattctgtgaccgccgcagacacggctttgtattactgtgcgagacaaaaaggttcggggagttttgacaactggggccagggaaccctggtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctg

SEQ ID NO: 415 - DNA, 16 bp, synthetic construct
  source 1..16: mol_type = other DNA
ttcttaggtcagtgga

SEQ ID NO: 416 - DNA, 638 bp, synthetic construct
  source 1..638: mol_type = other DNA
gagccaccacacatttccttaaattcaggtccaactcataagggaaatgctttctgagagtcatggatctcatgtgcaagaaaatgaagcacctgtggttcttcctcctgctggtggcggctcccagatgggtcctgtcccagctgcagctgcaggagtcgggcccaggactggtgaagccttcggagaccctgtccctcacctgcactgtctctggtgactccatcagcagtagtagtttccactgggtctggatccgccagcccccagggaaggggctggagtggattgggagtacccattatagtgggggcacctactacaacccgtccctcaagagtcgagtcaccatatccggagacacgtccaggaaccagttcaccctgaagctgagttctgtgaccgccgcagacacggctttgtattactgtgcgagacaaaaaggttcggggagttttgacaactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 417 - DNA, 16 bp, synthetic construct
  source 1..16: mol_type = other DNA
atgaggggttacgact

SEQ ID NO: 418 - DNA, 620 bp, synthetic construct
  source 1..620: mol_type = other DNA
gagggtccagctcacaggggaagtgctttctgagagtcatggacctcctgcacaagaacatgaaacacctgtggtgcttcctcctcctggtggcagctcccagatgggtcctgtcccaggtgcagctacagcagtggggcgcaggactgctgaagccctcggagaccctgtccctcacctgcgctgtctatggtgagtccttcagtggttactactggacctggatccgccagtccccagggaagggactggagtgggttggggacatcaaacatggtgagagcgccaactacaagccgtccctcaagagtcgagtcaccatatcacttgacgcgtccaacaaccagttctccctgaaactgaactctgtgaccgccgcggacacggctgtctactactgtgcgagaggaagggattgtggtggtgaatgttcacactttgactactggggccagggaaccctggtcaccatctcctcggccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 419 - DNA, 16 bp, synthetic construct
  source 1..16: mol_type = other DNA
cgagccagtagtacct

SEQ ID NO: 420 - DNA, 620 bp, synthetic construct
  source 1..620: mol_type = other DNA
gagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagccccccgggaaggggctggagtgggttggggacagcaaacatggtggaagcgccaactacaagccgtccctcaagagtcgagtcaccatatcactggacacgtccaacaaccagttctccctgaaactgagctctgtgactgccgcggacacggctgtgtattactgtgcgagaggaagggattgtggtggtgaatgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 421 - DNA, 16 bp, synthetic construct
  source 1..16: mol_type = other DNA
acaccaagtgttgagg

SEQ ID NO: 422 - DNA, 581 bp, synthetic construct
  source 1..581: mol_type = other DNA
ggagagtcatggacctcctgcacaagaacatgaaacacctgtggttcttcctcctcctggtggcagctcccagatgggtcctgtcccaggtgcagctacagcagtggggcgcaggactgctgaggccttcggagaccctgtccctcacctgcgctgtctatggtgggtccttcagtggttactactggagctggatccgccagccccccgggaaggggctggagtggattggggacaacaaacatggtggaaccgccaactacaagccgtccctcaagagtcgagtcaccatatcactggacacgtccaacaaccagttctccctgaaactgatctctgtgactgccgcggacacggctgtgtatttctgtgcgagaggacgggattgtggtggtgaatgctcccactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctgga

SEQ ID NO: 423 - DNA, 16 bp, synthetic construct
  source 1..16: mol_type = other DNA
acatggttcaacgcta

SEQ ID NO: 424 - DNA, 772 bp, synthetic construct
  source 1..772: mol_type = other DNA
agcaagcacctcccagggtgagcccaaaagactggggcctccctcatcccttcttgcctatccatacaaaggcaccacccacatgcaaatcctcacttaggcacccacaggaaatgacttcacatttccttacattcagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagccccccgggaaggggctggagtggcttggggacagcaaacatggtggaagcgccaactataagccgtccctcaagagtcgaatcaccatatcacttgacacgtccaacaaccagttctccctgaaactgaactctgtgactgccgcggacacggctgtgtattactgtgcgagaggaagggattgtggtggtgaatgctcccactttgactactggggccagggaaccctggtcaccgtttcttcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaata

SEQ ID NO: 425 - DNA, 16 bp, synthetic construct
  source 1..16: mol_type = other DNA
atcgagtagacgacgt

SEQ ID NO: 426 - DNA, 631 bp, synthetic construct
  source 1..631: mol_type = other DNA
gagggtccagctcacaggggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcactgtctatggtgggtccttcagtggttactactggacctggatccgccagtccccagggaagggactggaatggattggggacatcaaacatggtgagagcgccaactacaagccgtccctcaagagtcgagtcaccatatcacttgacgcgtccaacaaccagttctccctgaaactgaattctgtgaccgccgcggacacggctgtctactactgtgcgagaggaagggattgtggtggtgagtgttcacactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcaggg

SEQ ID NO: 427 - DNA, 16 bp, synthetic construct
  source 1..16: mol_type = other DNA
cacagtatcgatagaa

SEQ ID NO: 428 - DNA, 577 bp, synthetic construct
  source 1..577: mol_type = other DNA
ggagttttcttatatggggacaggggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggacctggatccgccagtccccagggaagggactggagtggattggggacatcaaacatggtgagagcgccaactacaagccgtccctcaagagtcgagtcaccatatcacttgacgcgtccaacaaccagttctccctaaaactgaattctgtgaccgccgcggacacggctgtctactactgtgcgagaggaagggattgtggtggtgagtgttcacactttgactactggggccagggaaccctggtcaccgtctcctcagccaaaacgacacccccatctgtctatccactggcccctggatctgctgcccaaactaactccatggtgaccctgggatgcctggtcaaggg

SEQ ID NO: 429 - DNA, 16 bp, synthetic construct
  source 1..16: mol_type = other DNA
catcagacacctcgtt

SEQ ID NO: 430 - DNA, 707 bp, synthetic construct
  source 1..707: mol_type = other DNA
gggcttgcctatccatacaaaggcaccacccacatgcaaatcctcacttaggcacccacaggaaatgacttcacatttccttacattcagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagccccccgggaaggggctggagtggcttggggacagcaaacatggtggaagcgccaactacaagccgtccctcaagagtcgagtcaccatatcactggacacgtccaacaaccagttctccctgaaactgaactctgtgactgccgcggacacggctgtgtattactgtgcgagaggaagggattgtggtggtgaatgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 431 - DNA, 16 bp, synthetic construct
  source 1..16: mol_type = other DNA
gcagttacagacaagc

SEQ ID NO: 432 - DNA, 898 bp, synthetic construct
  source 1..898: mol_type = other DNA
ccttgggctgctgcgcctccactgagccgcctgggtgtgaggacctcgcgccggcgcctccgggaaggacgcggagccgccgcggccgagcccagggctgcagctgctgcccgacgcccgcgggaaggcccttccgccaccgcccgggggttcgaacttggagcagctgctgccagctgaggcgccagcggcgtcgggagctcctcagagtcagtgttttgtgctggattcggcatgtcctctgaaaactcgggaatttgtcactgaaatggtgacaggagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagccccccgggaaggggctggagtggcttggggacagcaaacatggtggaagcgccaactataagccgtccctcaagagtcgaatcaccatatcacttgacacgtccaacaaccagttctccctgaaactgaactctgtgactgccgcggacacggctgtgtattactgtgcgagaggaagggattgtggtggtgaatgctcccactttgactactggggccagggaaccctggtcaccgtttcttcagccaaaacgacacccccatctgtctatccactggcccctggatctgctgcccaaactaactccatggtgaccctgggatgcctggtcaagggctatttccctgagccagtgacagggatccagagttccaggtcactgtca

SEQ ID NO: 433 - DNA, 16 bp, synthetic construct
  source 1..16: mol_type = other DNA
tttggtttctttacgt

SEQ ID NO: 434 - DNA, 583 bp, synthetic construct
  source 1..583: mol_type = other DNA
caagcacctcccaggggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagccccccgggaaggggctggagtgggttggggacagcaaacatggtggaagcgccaactacaagccatccctcaagagtcgagtcaccatatcactggacacgtccaacaaccagttctccctgagactgagctctgtgactgccgcggacacggctgtgtattactgtgcgagaggaagggattgtggtggtgaatgctcccactttgactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 435 - DNA, 16 bp, synthetic construct
  source 1..16: mol_type = other DNA
gcaaactcacattagc

SEQ ID NO: 436 - DNA, 636 bp, synthetic construct
  source 1..636: mol_type = other DNA
cacatttccttaaattcagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctatggtggatccttcagtggttactactggacctggatccgccagcccccagggaaggggctggagtgggttggggacatcaagcatggtggaagcgccaactacaacccgtccctcaagagtcgagtcaccatatcactagacacgtccaagaaccagttcaccctgaaactgacctctgtgaccgccgcggacacggctgtatattactgtgcgcgaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 437 - DNA, 16 bp, synthetic construct
  source 1..16: mol_type = other DNA
aaacggggtgtttggt

SEQ ID NO: 438 - DNA, 638 bp, synthetic construct
  source 1..638: mol_type = other DNA
ggggagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctatggtggatccttcagtggttactactggacctggatccgccagcccccagggaaggggctggagtgggttggggacatcaagcatggtggaagcgccaactacaacccgtccctcaagagtcgagtcaccatatcactagacacgtccaagaaccagttcaccctgaaactgacctctgtgaccgccgcggacacggctgtatatttctgtgcgcgaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaat

SEQ ID NO: 439 - DNA, 16 bp, synthetic construct
  source 1..16: mol_type = other DNA
aaggttctccgaatgt

SEQ ID NO: 440 - DNA, 637 bp, synthetic construct
  source 1..637: mol_type = other DNA
gagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctatggtgggtccttcagtggttactactggagctggatccgccagcccccagggaagggactggagtgggttggggacatcaaacatggtgggagtgccaactacaagccgtccctcaagagtcgagtcaccatatcacttgacgcgtccaacaaccagttctccctgaaactgaactctgtgaccgccgcggacacggctgtgtactactgtgcgagaggaagggattgtggtggtgattgctcccactttgactactggggccagggaaccctggtcatcgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaatag

SEQ ID NO: 441 - DNA, 16 bp, synthetic construct
  source 1..16: mol_type = other DNA
atttctgtcaatctct

SEQ ID NO: 442 - DNA, 637 bp, synthetic construct
  source 1..637: mol_type = other DNA
gagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctatggtggatccttcagtggttactactggacctggatccgccagcccccagggaaggggctggagtgggttggggacatcaagcatggtggaagcgccaactacaacccgtccctcaagagtcgagtcaccatatcactagacacgtccaagaaccagttctccctgaaaatgacctctgtgaccgccgcggacacggctgtgtattactgtgcgcgaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaatag

SEQ ID NO: 443 - DNA, 16 bp, synthetic construct
  source 1..16: mol_type = other DNA
cacagtatcatagcac

SEQ ID NO: 444 - DNA, 623 bp, synthetic construct
  source 1..623: mol_type = other DNA
ggggagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagtccccagggaaggggctggagtgggttggggacatcaaacatggtgggagcgccaactacaagccgtccctcaagagtcgagtcaccatatcacttgacgcgtccaacaaccagttctccctgaaactgaattctgtgaccgccgcggacacggctgtgtactactgtgcgagaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 445 - DNA, 16 bp, synthetic construct
  source 1..16: mol_type = other DNA
catcaagagtggtccc

SEQ ID NO: 446 - DNA, 559 bp, synthetic construct
  source 1..559: mol_type = other DNA
gacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctatggtggatccttcagtggttactactggacctggatccgccagcccccagggaaggggctggagtgggttggggacatcaagcatggtggaagcgccaactacaacccgtccctcaagagtcgagtcaccatatcactagacacgtccaagaaccagttcaccctgaaactgacctctgtgaccgccgcggacacggctgtatatttctgtgcgcgaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaaggg

SEQ ID NO: 447 - DNA, 16 bp, synthetic construct
  source 1..16: mol_type = other DNA
ctcggagagtaggtgc

SEQ ID NO: 448 - DNA, 641 bp, synthetic construct
  source 1..641: mol_type = other DNA
ggagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctatctatggtgggtccttcagtggttactactggagctggatccgccagtccccagggaaggggctggagtgggttggggacatcaaacatggtgggagcgccaacttcaagccgtccctcaagagtcgagtcaccatatcacttgacgcgtccaacaaccagttctccctgaaactgaattctgtgaccgccgcggacacggctgtgtactactgtgcgagaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaatagccc

SEQ ID NO: 449 - DNA, 16 bp, synthetic construct
  source 1..16: mol_type = other DNA
cttaccggtatagtag

SEQ ID NO: 450 - DNA, 769 bp, synthetic construct
  source 1..769: mol_type = other DNA
agggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgttgaagccttcggagaccctgtccctcacctgcgctgtctctggtggatccttcagcggttactactggacctggatccgccagcccccagggaagggactggagtggattggggacatcaagcatggtggaagcgccaactacaacccgtccctcaagagtcgagtcaccatatcactagacacgtccaagaaccagttcaccctgaaactgacctctgtgaccgccgcggacacggctttatatttctgtgcgcgaggaagggattgtggtggtgattgctcccactttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaatagcccttgacca

SEQ ID NO: 451 - DNA, 16 bp, synthetic construct
  source 1..16: mol_type = other DNA
gaccaatagtcaagcg

SEQ ID NO: 452 - DNA, 643 bp, synthetic construct
  source 1..643: mol_type = other DNA
ggggagggtccagctcacatgggaagtgctttctgagagtcatggacctcctgcacaagaacatgaaacacctgtggttcttcctcctcctggtggcagctcccagatgggtcctgtcccaggtgcagctacagcagtggggcgcaggactgctgaagccttcggagaccctgtccctcacctgcgctgtctatggtgggtccttcagtggttactactggagctggatccgccagcccccagggaagggactggagtgggttggggacatcaaacatggtggaagcgccaactacaagccggccctcaagagtcgagtcaccatttcactagacccgtccaacaaccagttctccctgaaactgagctctgtgaccgccgcggacacggctgtttactactgtgcgagaggaagggattgtggtggtgattgctcccactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgacagtggcctg

SEQ ID NO: 453 - DNA, 16 bp, synthetic construct
  source 1..16: mol_type = other DNA
caccaggtcgtcacgg

SEQ ID NO: 454 - DNA, 651 bp, synthetic construct
  source 1..651: mol_type = other DNA
tggggagcgctgagagaggagcccagccctgggattttctggtgttttcatttggtgatcaggactgaacagagagaattcaccatggagactggcctgaactggctttttcttgttgctattttaaaaggtgtccagtgtgaggttcaattgttagaatctgggggaggcttggttcagcctggggagtccctgagactctcctgtgcaacctctggattcatctttaatttctacgccatgaactgggtccgccaggctccagggaaggggctggagtggatctcaactattagtggtaatggtggtaccacattctacacagactccgtgaagggccgattcaccatctccagagacaattccaagaacatcctgtatctgcagatgaacagcctgagagccgaggacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 455 - DNA, 16 bp, synthetic construct
  source 1..16: mol_type = other DNA
aacgttggtattaccg

SEQ ID NO: 456 - DNA, 368 bp, synthetic construct
  source 1..368: mol_type = other DNA
tacgccatgaactgggtccgccaggctccagggaaggggctggagtggatctcaactattagtggtaatggtggtaccacattctacacagactccgtgaagggccggttcaccatctccagagacaattccaagaacatgctatatctgcagatgaacagcctgagagccgatgacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 457 - DNA, 16 bp, synthetic construct
  source 1..16: mol_type = other DNA
cagtcctcacatttct

SEQ ID NO: 458 - DNA, 640 bp, synthetic construct
  source 1..640: mol_type = other DNA
gccctccgtctgctgatgaaaaccagcccagccctgaccctgcagcgctgagagaggagcccagccctgggattttctggtgttttcatttggtgatcaggactgaacagagagaactcaccatggagtttgggctgagctggcttcttcttgttgctactttaaaaggtgtccagtgtgaggtgcaattattggaatctgggggaggcttggttcagcctggggagtccctgagactctcctgtgcaacctctggattcacctttaatttctatgccatgaactgggtccgccaggctccagggaaggggctggagtggatctcaactattagtggtaatggtggtaccacattctacacagactccgtgaagggccggttcaccatctccagagacaattccaagaacatactgtatctacaaatgaacagcctgagagccgatgacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaaggg

SEQ ID NO: 459 - DNA, 16 bp, synthetic construct
  source 1..16: mol_type = other DNA
cattatctcgatgagg

SEQ ID NO: 460 - DNA, 558 bp, synthetic construct
  source 1..558: mol_type = other DNA
gggtgatcaggactgaacagagagaactcaccatggagtttgggctgagctggctttttcttgttgctattttaaaaggtgtccagtgtgaggtgcagctattggaatctgggggaggcttggttcagcctggggagtccctgagactctcctgtgcaacctctggattcacctttaatttctatgccatgaactgggtccgccaggctccagggaaggggctggagtggatctcaactattagtggtaatggtggtaccacattctacacagactccgtgaagggccggttcaccatctccagagacaattccaagaacatactgtatctgcaaatgaacagcctgagagccgatgacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaagggttatttcc

SEQ ID NO: 461 - DNA, 16 bp, synthetic construct
  source 1..16: mol_type = other DNA
gtcctcaagtgttgaa

SEQ ID NO: 462 - DNA, 651 bp, synthetic construct
  source 1..651: mol_type = other DNA
tggggagcgctgagagaggagcccagccctgggattttctggtgttttcatttggtgatcaggactgcacagagagaactcaccatggagtctgggctgagctggctttttcttgttgctattttacaaggtgtccagtgtgaggtgcagctgttggaatctgggggaggcttggttcagcctggggagtccctgagactctcctgtgcaacgtctggattcacctttaatttctatgccatgaactgggtccgccagactccagggaaggggctggagtggatctcaactattagtggtaatggtggcaccacattctacgcagactccgtgaagggccggttcaccatctccagagacaattccaagaacatgctgtatctgcaaatgaacagcctgagagccgaggacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 463 - DNA, 16 bp, synthetic construct
  source 1..16: mol_type = other DNA
tcgggacagcagatcg

SEQ ID NO: 464 - DNA, 652 bp, synthetic construct
  source 1..652: mol_type = other DNA
ctggggagcgctgagagaggagcccagccctgggattttctggtgttttcatttggtgatcaggactgaacagagagaactcaccatggagtttgggctgagctggctttttcttgttgctattttaaaaggtgtccagtgtgaggtgcagctgttggaatctgggggaggcttggttcagcctggggagtccctgagactctcctgtgcaacctctggattcacctttaatttctatgccatgaactgggtccgccaggcgccagggaaggggctggagtggatatcaactattagtggtaatggtggtaccacattctacacaaactccgtgaagggccggttcaccatctccagagacaattccaagaacatgctgtatctgcaaatgaacagcctgagagccgaggacacggccgtttattactgtgcgaaatggggaataacgtttggggaagttgtcgtccactttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 465 - DNA, 16 bp, synthetic construct
  source 1..16: mol_type = other DNA
atagacccatcgggtc

SEQ ID NO: 466 - DNA, 682 bp, synthetic construct
  source 1..682: mol_type = other DNA
gctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagtgtgaagtgcagttggtggagtctgggggaggcttggttcagcctggcaggtccctgagactctcctgtgcagcctctggattcacctttgatgactatggcatacactgggtccggcaagttccagggaagggcctggagtgggtctcaggtagtagttggaataatggtcatataggctatgcggactctgtgaagggccgattcaccatctccagagacaacgccaagaactccctgtatctgcaaatgaacagtctgagaactgaggacacggccttgtattactgtgtaaaagatattagcagcagctggtacgggtatttcgatctctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 467 - DNA, 16 bp, synthetic construct
  source 1..16: mol_type = other DNA
acgaggagtcatactg

SEQ ID NO: 468 - DNA, 590 bp, synthetic construct
  source 1..590: mol_type = other DNA
gggggggagaggagccccagccctgagattcccaggtgtttccattcagtgatcagcacggaacacagaggactcaccatggagttgggactgagctggattttccttttggctattttaaaaggtgtccagtgtgaagtgcagttggtggagtctgggggaggcttggtacagcctggcaggtccctgagactctcctgtgcagcctctggattcacctttgatgactatggcatacactgggtccggcaaactccagagaagggcctggagtgggtcgcaggtagtagttggaataatggtcatataggctatgcggactctgtgaagggccgattcaccatctccagagacaacgccaagaactccctgtatctgcaaatgaacagtctgagaactgaggacacggccttgtattactgtgtaaaagatattagcagcagctggtacgggtatttcgatctctggggccgtggcaccctggtcactgtctcctcagccaaaacaacagccccatcggtctatccactggcccctgtgtgtggagatacaactggctcctcggtgactctaggatgcctggtcaaggg

SEQ ID NO: 469 - DNA, 16 bp, synthetic construct
  source 1..16: mol_type = other DNA
gaccaataggtagctg

SEQ ID NO: 470 - DNA, 645 bp, synthetic construct
  source 1..645: mol_type = other DNA
ggggagctctgggagaggagccccagccctgagattcccaggtgtttccattcagtgatcagcactgaacacagaggactcaccatggagttgggactgagctggattttccttttggctattttaaaaggtgtccagtgtgaagtgcagttggtggagtctgggggaggcttggtacagcctggcaggtccctgagactctcctgtgcagcctctggattcacctttgatgactatggcatacactgggtccggcaagctccagggaagggcctggagtgggtctcaggtagtagttggaataatggtcatataggctatgcggactctgtgaagggccgattcaccatctccagagacaacgccaagaactccctgtatctgcaaatgaacagtctgagaactgaggacacggccttgtattactgtgtaaaagatattagcagcagctggtacgggtatttcgatctctggggccgtggcaccctggtcactgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 471 - DNA, 16 bp, synthetic construct
  source 1..16: mol_type = other DNA
gggcactagtctcggc

SEQ ID NO: 472 - DNA, 590 bp, synthetic construct
  source 1..590: mol_type = other DNA
gggggggagaggagccccagccctgagattcccaggtgtttccattcagtgatcagcactgaacacagaggactcaccatggagttgggactgagctggattttccttttggctattttaaaaggtgtccagtgtgaagtgcagttggtggagtctgggggaggcttggtacagcctggcaggtccctgagactctcctgtgcagcctctggattcacctttgatgactatggcatacactgggtccggcaagctccagggaagggcctggagtgggtctcaggtagtagttggaataatggtcatataggctatgcggactctgtgaagggccgattcaccatctccagagacaacgccaagaactccctgtatctgcaaatgaacagtctgagaactgaggacacggccttgtattactgtgtaaaagatattagcagcagttggtacgggtatttcgatctctggggccgtggcaccctggtcactgtctcctcagccaaaacaacagccccatcggtctatccactggcccctgtgtgtggagatacaactggctcctcggtgactctaggatgcctggtcaaggg

SEQ ID NO: 473 - DNA, 16 bp, synthetic construct
  source 1..16: mol_type = other DNA
gtcctcacagggtatg

SEQ ID NO: 474 - DNA, 591 bp, synthetic construct
  source 1..591: mol_type = other DNA
tgggggggagaggagccccagccctgagattcccaggtgtttccattcagtgatcagcactgaacatagaggactcaccatggagttgggactgagctggattttccttttggctattttaaaaggtgtccagtgtgaagtgcagttggtggagtctgggggaggcttggtacagcctggcaggtccctgagactctcctgtgcagcctctggattcacctttgatgattatggcatacattgggtccggcaagttccagggaagggcctggagtgggtctcaggtagtagttggaataatggtcatttaggctatgcggactctgtgaggggccgattcaccatttccagagacaacgccaagaactccctgtatctgcaaatgaacagtctgagaactgaggacacggccttatattactgtgtaaaagacattagcagcagctggtacgggtatttcgatctttggggtcgtggcaccctggtcactgtctcctcagccaaaacaacagccccatcggtctatccactggcccctgtgtgtggagatacaactggctcctcggtgactctaggatgcctggtcaaggg

SEQ ID NO: 475 - DNA, 16 bp, synthetic construct
  source 1..16: mol_type = other DNA
tgtgtttcacaacgcc

SEQ ID NO: 476 - DNA, 634 bp, synthetic construct
  source 1..634: mol_type = other DNA
gcctgaccctgcagctctgggagaggagccccagccctgagattcccaggtgtttccattcagtgatcagcacggaacacagaggactcaccatggagttgggactgagctggattttccttttggctattttaaaaggtgtccagtgtgaagtgcagttggtggagtctgggggaggcttggtacagcctggcaggtccctgagactctcctgtgcagcctctggattcacctttgatgactatggcatacactgggtccggcaaactccagagaagggcctggagtgggtcgcaggtagtagttggaataatggtcatataggctatgcggactctgtgaagggccgattcaccatctccagagacaacgccaagaactccctgtatctgcaaatgaacagtctgagaactgaggacacggccttgtattactgtgtaaaagatattagcagcagctggtacgggtatttcgatctctggggccgtggcaccctggtcactgtctcctcagagtctgcgagaaatcccaccatctacccactgacactcccaccagctctgtcaagtgacccagtgataatcggctgcctgattcacgattacttcccttccggcacgatgaatgtgacctg

SEQ ID NO: 477 - DNA, 16 bp, synthetic construct
  source 1..16: mol_type = other DNA
acgatgtcagtataag

SEQ ID NO: 478 - DNA, 664 bp, synthetic construct
  source 1..664: mol_type = other DNA
catgcctagtttcttatatggggagctctgggagaggagccccagccctgagattcccaggtgtttccattcagtgatcagcactgaacacagaggactcaccatggagttgggactgagctggattttccttttggctattttaaaaggtgtccagtgtgaagtacaattggtggagtctgggggagccttggtacagcctggcaggtccctgagactctcctgtgaagcctctggattcacctttgatgattatggcatgcactgggtccgacaaactccagggatgggcctggagtgggtctcagggattagttggaatggtggtcacataggcttggcggactctgtgaagggccgattcaccatctccagagacaacgccaagaattccctctttcttcaaatgaacagtctgagacctgaggatacggccttgtattactgtacaagagatataagtagcagctggtacggatattttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 479 - DNA, 16 bp, synthetic construct
  source 1..16: mol_type = other DNA
acgatgtcatgatcca

SEQ ID NO: 480 - DNA, 668 bp, synthetic construct
  source 1..668: mol_type = other DNA
atgtagctgcttttcttatatgggagctctgggagaggagccccagccctgagattcccaggtgtttccattcagtgatcagcactgaacacagaggactcaccatggagttgggactgagctggattttccttttggctattttaaaaggtgtccagtgtgaagtacagctggtggagtctgggggaggcttggtacagcctggcaggtccctgagactctcctgtgcagcctctggattcacctttgatgattatggcatgcactgggtccggcaaactccagggaagggcctggagtgggtctcaggtcttagctggaatggagctcacctaggcttggcgggctctgtgaggggccgattcaccatctccagagacaacgccaagagttccctgtatctgcaaatgaacagtctgagagctgaggacacggccttctattactgtacaagagatataagtagcagttggtatgggtattttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagt

SEQ ID NO: 481 - DNA, 16 bp, synthetic construct
  source 1..16: mol_type = other DNA
cggacaccaagtctgt

SEQ ID NO: 482 - DNA, 646 bp, synthetic construct
  source 1..646: mol_type = other DNA
tggggagctctgggagaggagccccagccctgagattcccaggtgtttccattcagtgatcagcactgaacacagaggactcaccatggagttgggactgagctggattttccttttggctattttaaaaggtgtccagtgtgaagtacagctggtggagtctgggggaggcttggtacagcctggcaggtccctgagactctcctgtgcagcctctggattcacctttgatgattatggcatgcactgggtccggcaaactccagggaagggcctggagtgggtctcaggtcttagctggaatggagctcacctaggcttggcgggctctgtgaggggccgattcaccatctccagagacaacgccaagagttccctgtatctgcaaatgaacagtctgagagctgaggacacggccttctattactgtacaagagatataagtagcagttggtatgggtattttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 483 - DNA, 16 bp, synthetic construct
  source 1..16: mol_type = other DNA
cctctgacatgtaaga

SEQ ID NO: 484 - DNA, 682 bp, synthetic construct
  source 1..682: mol_type = other DNA
gctccctctgctgatgaaaaccagcccagccctgaccctgcagctctgggagaggagccccagccctgagattcccaggtgtttccattcagtgatcagcactgaacacagaggactcaccatggagttgggactgagctggattttccttttggctattttaaaaggtgtccagtgtgaagtacaactggtggagtctgggggaggcttggtacagcctggcaggtccctgagactctcctgtgcggcctctggattcacctttgatgattttggcatgcactgggtccgacaaactccagggaagggcctggagtgggtctcagggattaattggaatggtggtcacataggcttggcggactctgtgaagggccgattcaccatctccagagacaacgccaagaattccctgtttctgcaaatgaacagtctgagagctgaggatacggccttgtattattgtacaagagatataagtagcagctggtacgggtattttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 485 - DNA, 16 bp, synthetic construct
  source 1..16: mol_type = other DNA
gtcacggtcacaggcc

SEQ ID NO: 486 - DNA, 633 bp, synthetic construct
  source 1..633: mol_type = other DNA
gggggggagaggagccccagccctgagattcccaggtgtttccaatcagtgatcagcactgaacacagaggactcaccatggagttgggactgagctggattttccttttggctattttaaaaggtatccaatgtgaagtacagctggtggaatctgggggagccttggttcagcctggcagctccctgagactctcctgtacagcctctggattcacctttgatgattatggcatgcactgggtccggcaaactccagggaagggcctggagtgggtctcaggtattaattggaatggtgctcacttaggcttggcggcctctgtgaagggccgattcaccatctccagagacaacgccaagaattccctgtatctgcaaatgaacagtctgagagctgaggacacggccttctattactgtacaagagatataagtagcagctggtacgggtattttgactactggggccagggaaccctggtcaccgtctcctcagccaaaacaacagccccatcggtctatccactggcccctgtgtgtggagatacaactggctcctcggtgactctaggatgcctggtcaagggttatttcccagagttccaggtcaaggtcactggctcagggaaa

SEQ ID NO: 487 - DNA, 16 bp, synthetic construct
  source 1..16: mol_type = other DNA
agtgggatcaacacca

SEQ ID NO: 488 - DNA, 803 bp, synthetic construct
  source 1..803: mol_type = other DNA
tcgtcagcaaacatctgcagggcaccgtcttattatctgggtacagcctattcctccagcgtcccaccctagagcttgttatatagtaggagatatgcaaaaagggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgtttctattttagaaggtgtccagtgtgaggtgcaattggtggagtctgggggaggcctggtcaaccctggggggtccctgagactctcctgtgcagcctctggaatcaccttcagttcctctaccatggcctgggtccgccaggctccggggaagggactggagtgggtctcatccattagttcttttagtaattacataaattactcagactcagtgaagggccgattcaccatctccagagacaacgtcaagaactcactgtttctgcagatgaacagcctgagagccgaggacacggctgtctattactgtgcgagcggtataggaggtcgtccggtctttgactactggggccagggaaccctggtcaccgtctcctcaaccaaaacgacacccccatctgtctatccactggcccctggatctgctgcccaaactaactccatggtgaccctgggatgcctggtcaagggctatttccctgagccagtgacagggatccagagttccaggtcactgtcactggctcagggaaatagccct

SEQ ID NO: 489 - DNA, 16 bp, synthetic construct
  source 1..16: mol_type = other DNA
attggacaggaattac

SEQ ID NO: 490 - DNA, 809 bp, synthetic construct
  source 1..809: mol_type = other DNA
tattttccaaaaaccatcgtcagcaaacatctgcagggcaccgtcttattatctgggtacagcctattcctccagcgtcccaccctagagcttgttatatagtaggagatatgcaaaaagggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgttgctattttagaaggtgtccagtgtgaggtacaactggtggagtctgggggaggcctggtcaagcctggggggtccctgagactctcctgtgcagcctctggaatcaccttcagttcctgtaccatgacctgggtccgccaggctccagggaaggggctggagtgggtctcatccattagtacttatagtaattacataaattactcagactcagtgaagggccgattcaccatctccagagacaacgccaagaactcactgtttctgcaaatgaacagcctgagagccgaggacacggctgtctattactgtgcgagcggtataggaggtcgtccggtctttgactactggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactggctcaggg

SEQ ID NO: 491 - DNA, 16 bp, synthetic construct
  source 1..16: mol_type = other DNA
cattcgccatagtaag

SEQ ID NO: 492 - DNA, 636 bp, synthetic construct
  source 1..636: mol_type = other DNA
tgggggagagaggagccttagccctggattccaaggcctatccacttggtgatcagcactgagcaccgaggattcaccatggaactggggctccgctgggttttccttgtttctattttagaaggtgtccagtgtgaggtgcaattggtggagtctgggggaggcctggtcaaccctggggggtccctgagactctcctgtgcagcctctggaatcaccttcagttcctctaccatggcctgggtccgccaggctccggggaagggactggagtgggtctcatccattagtacttttagtaattacataaattactcagactcagtgaagggccgattcaccatctccagagacaacgtcaagaactcactgtttctgcagatgaacagcctgagagccgaggacacggctgtctattactgtgcgagcggtataggaggtcgtccggtctttgactactggggccagggaaccctggtcaccgtctcctcaaccaaaacgacacccccatctgtctatccactggcccctggatctgctgcccaaactaactccatggtgaccctgggatgcctggtcaagggctatttccctgagccagtgacagggatccagagttccaggtcactgtcactg

SEQ ID NO: 493 - DNA, 16 bp, synthetic construct
  source 1..16: mol_type = other DNA
cctattagttaaagac

SEQ ID NO: 494 - DNA, 681 bp, synthetic construct
  source 1..681: mol_type = other DNA
tggggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgtttctattttagaaggtgtccagtgtgaggtgcaattggtggagtctgggggaggcctggtcaatccgggggggtccctgagactctcctgtgcagcctctggaatcaccttcagttcctctaccatggcctgggtccgccaggctccggggaagggactggagtgggtctcatccattagttcttttagtaattacataaattattcagactcagtgaagggccgattcaccatctccagagacaatgtcaagaactcactgtttctgcagatgaacagcctgagagccgaggacacggctgtctattactgtgcgagcggtataggaggtcgtccggtctttgactactggggccagggaaccctggtctccgtctcctcaaccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 495 - DNA, 16 bp, synthetic construct
  source 1..16: mol_type = other DNA
ctgtttacaaattgcc

SEQ ID NO: 496 - DNA, 678 bp, synthetic construct
  source 1..678: mol_type = other DNA
ggacctccctctactgatgaaaaccaaccgaaccctgaccctgcagctctgagagaggagccttagccctggattccaaggcctatccacttggtgatcagcactgagcaccgaggattcaccatggaactggggctccgctgggttttccttgtttctattttagaaggtgtccagtgtgaggtgcaattggtggagtctgggggaggcctggtcaatccgggggggtccctgagactctcctgtgcagcctctggaatcaccttcagttcctctaccatggcctgggtccgccaggctccggggaagggactggagtgggtctcatccattagttcttttagtaattacataaattattcagactcagtgaagggccgattcaccatctccagagacaatgtcaagaactcactgtttctgcagatgaacagcctgagagccgaggacacggctgtctattactgtgcgagcggtataggaggtcgtccggtctttgactactggggccagggaaccctggtctccgtctcctcaaccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 497 - DNA, 16 bp, synthetic construct
  source 1..16: mol_type = other DNA
ccttcccgtgagtgac

SEQ ID NO: 498 - DNA, 636 bp, synthetic construct
  source 1..636: mol_type = other DNA
tgcacatcttttcttatatggggagggtccagctcacatgggaaatactttctgagagtcctggacctcctgtgcaagaacatgaaacatctgtggttcttccttctcctggtggcagctcccagatgggtcctgtcccaggtccaactgcaggagtcgggcccaggactggtgaggccttcggagaccctgtccctcacctgcactgtctctggtggctccttcagtttttactcctggagctggatccggcagcccccagggaagggactggactggattgggtatatctataacagtgggagcaccaactacaacccctcactcaagagtcgggtcaccatatcagtagacacgtccaagaaccagttctccctgaagctgagctctgtgaccgccgcagacacggccgtgtattactgtgcgagacatcgtgggagctacgtctacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 499 - DNA, 16 bp, synthetic construct
  source 1..16: mol_type = other DNA
catcgaagtctctctg

SEQ ID NO: 500 - DNA, 609 bp, synthetic construct
  source 1..609: mol_type = other DNA
gagggtccagctcacatgggaaatactttctgagagtcctggacctcctgtgcaagaacatgaaacatctgtggctcttccttctcctggtggcagctcccagatgggtcctgtcccaggtgcagctgcaggagtcgggcccaggactggtgaagccttcggagaccctgtccctcacctgcactgtctctggtggctccttcagtttttactcctggagctggatccggcagcccccagggaagggactggactggattggatatatctataacagtgggagcaccaactacaacccctcactcaagagtcgggtcaccatatcagtagacacgtccaagaaccagttatccctgaagctgagctctgtgaccgccgcagacacggccgtgtattactgtgcgagacatcgtgggagctacgtctacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccc

SEQ ID NO: 501 - DNA, 16 bp, synthetic construct
  source 1..16: mol_type = other DNA
cgccaaggtccatcct

SEQ ID NO: 502 - DNA, 565 bp, synthetic construct
  source 1..565: mol_type = other DNA
gagggtccagctcacatgggaaatactttctgagagtcctggacctcctgtgcaagaacatgaaacatctgtggttcttccttctcctggtggcagctcccagatgggtcctgtcccaggtgcagctgcaggagtcgggcccaggactggtgaaggcttcggagaccctgtccctcacctgcactgtctctggtggctccttcagtacttactactggagctggatccggcagcccccaggaaagggactggaatggattggatatatctattacagtgggaacaccaactacaacccctccctcaagagtcgagtcaccatatcagtagacacgtccaagaaccagttctccctgaagctgaactctgtgaccgccgctgacacggccgtatattactgtgcgagacatcgtgggagctacgtctacggtatggacgtctggggccaagggaccacggtcaccgtctcctcagccaaaacgacacccccatctgtctatccactggcccctggatctgctgcccaaactaactccatggtgaccctgggatgcctggtcaaggg

SEQ ID NO: 503 - DNA, 16 bp, synthetic construct
  source 1..16: mol_type = other DNA
gtgggtccagctgtgc

SEQ ID NO: 504 - DNA, 596 bp, synthetic construct
  source 1..596: mol_type = other DNA
gatactttctgagagtcctggacctcctgtgcaagaacatgaaacatctgtggttcttccttctcctggtggcagctcccagatgggtcctgtcccaggtgcagctgcaggagtcgggcccaggactggtgaagccttcggagaccctgtccctcacctgcactggctctggtgactccatcagtaattactactggagctggatccggcagtccccagggaagggactggagtggattgggtatatcaatttcagtgggaacatcaactacaatccctccctcaagagtcgagtcaccatatcagtagacacgtccaagaaccagttctccctgaaactgaggtctgtgaccgccgcagacacggccgtgtattactgtgcgagacatcgtgggagctacgtctacggtatggacgtctggggccaagggaccacggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactg

SEQ ID NO: 505 - DNA, 16 bp, synthetic construct
  source 1..16: mol_type = other DNA
ggcgtgtcacctcgtt

SEQ ID NO: 506 - DNA, 640 bp, synthetic construct
  source 1..640: mol_type = other DNA
tctaggatttcttatatgggggcagagcctgctgaattctggctgaccaggacagtcaccagagctccagacaatgtctgtctccttcctcatcttcctgcccgtgctgggcctcccatggggtgtcctgtcacaggtacagctgcagcagtcaggtccaggactggtacagccctcgcagaccctctcactcacctgtgccatctccggggacagtgtctctagtaacagtgctggttggaattgggtcaggcagtccccatcgagaggccttgagtggctgggaaggacattctacaggtccgagtggtataatgattatgcagtgtctgtgaaaagtcgaataaccatcaatgtcgacacatccaagaaccagttctccctgcagctgaaatctgtgactcccgaggacacggctgtatattactgtgcaagagtagataagtggaattattggttcgacccctggggccagggaaccctggtcaccgtctccacagccaaaacgacacccccatctgtctatccactggcccctggatctgctgcccaaactaactccatggtgaccctgggatgcctggtcaagggctatttccctgagccagtgacagtgacctggaactctggatccctgtcc

SEQ ID NO: 507 - DNA, 16 bp, synthetic construct
  source 1..16: mol_type = other DNA
gcctctagtcgtggct

SEQ ID NO: 508 - DNA, 627 bp, synthetic construct
  source 1..627: mol_type = other DNA
tgggggcagagcctgctgaattctggctgaccagggcagtcaccagagctccagacaatgtctgtctccttcctcatcttcctgcccgtgctgggcctcccatggggtgtcctgtcacaggtacagttgcagcagtcaggtccagggctggtgcagccctcgcagaccctctctctcacctgtgccatctccggggacagtgtctctagcaacagtgctggttggaactggatcaggcagtccccatcgagaggccttgagtggctgggaaggacattctacaggtccgagtggtattatgattatgcagtgtctgtgaaaagtcgaataaccatcaacccagacacatccaagaaccagttctccctgcagctgaactctgtgactcccgacgacacggctatatattactgtgcaagagtagataagtggaactactggttcgacccctggggccagggaaccctggtcaccgtctcctcagccaaaacgacacccccatctgtctatccactggcccctggatctgctgcccaaactaactccatggtgaccctgggatgcctggtcaagggctatttccctgagccagtgacagggatccagagttccaggtcactgtcactg

SEQ ID NO: 509 - DNA, 16 bp, synthetic construct
  source 1..16: mol_type = other DNA
ttggaactcatcaccc

SEQ ID NO: 510 - DNA, 633 bp, synthetic construct
  source 1..633: mol_type = other DNA
tttcttctctgggggcagagcctgctgaattctggctgaccagggcagtcaccagagctccagacaatgtctgtctccttcctcatcttcctgcccgtgctgggcctcccatggggtgtcctgtcacaggtacagttgcggcagtcaggtccaggactggtgcagccctcgcagaccctctcactcacctgtgccatctccggggacagtgtctctagcaacagtgctggttggaactggatcaggcagtccccatcgagaggccttgagtggctgggaaggacattctacaggtccgagtggtattatgattatgcagtgtctgtgaaaagtcgaataaccatcaacccagacacatccaagaaccagttctccctgcaactgaactctgtgactcccgacgacacggctatatattactgtgcaagagtagataagtggaactactggttcgacccctggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtcc

SEQ ID NO: 511 - DNA, 693 bp, synthetic construct
  source 1..693: mol_type = other DNA
agcccatatcaatgcctgggtcagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctccaggggaaagagccaccctctcctgcagggccagtcagagtattagcagtaatttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 512 - DNA, 510 bp, synthetic construct
  source 1..510: mol_type = other DNA
gagagctctggggaggaactgctcagttaggacccagacggaaccatggaagccccagcgcagcttctcctcctcctgctactctggctcccagaaaccactggagaaatagtgatgacgcagtctccaggcaccctgtctgtgtctccaggggaaagagccaccctctcctgcagggccagtcagagtattagcagcaacttagcctggtaccaacagaaacttggccaggctcccaggctcctcatctatgctgcatccaccagggccactggttgccccgccaggttcagtggcagtgggtctgggacagagttcactctcaccatcagcagcctacagtctgaagattttgcattttattactgtcagcaatatattgagaggcctctcactttcggcggagggaccaaggtggcgatcaaacgggctgatgctgcaccaactgtatccatcttcccaccatccagtgagcagttaacatctggaggtgcctcagtcgtgtgcttc

SEQ ID NO: 513 - DNA, 695 bp, synthetic construct
  source 1..695: mol_type = other DNA
ttccacttggtttcttatatggggagagctctggggaggaactgctcagttaggacccagacggaaccatggaagccccagcgcagcttctcctcctcctgctactctggctcccagaaaccactggagaaatagtgatgacgcagtctccagccaccctgtctgtgtctccaggggagagagccaccctctcctgcagggccagtcagagtattagcagcaacttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 514 - DNA, 799 bp, synthetic construct
  source 1..799: mol_type = other DNA
gaggatgctcagtgcagagaggaagaagcaggtggtctttgcagctgaaagctcagctcccaccccagctgctttgcatgtccctcccagctgccctaccttccagagcccatatcaatgcctgggtcagagctctggggaggaactgctcagttaggacccagacggaaccatggaagccccagcgcagcttctcctcctcctgctactctggctcccagaaaccactggagaaatagtgttgacgcagtctccagccaccctgtctgtgtctccaggggaaagagccaccctctcctgcagggccagtcagagtattagcagcaacttagcctggtaccaacagaaacttggccaggctcccaggctcctcatctatgctgcatccaccagggccactggtatcccagccagattcagtggcagtgggtctgggacagagttcactctcaccatcagcagcctgcagtctgaagattttgcatcttatcattgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 515 - DNA, 551 bp, synthetic construct
  source 1..551: mol_type = other DNA
gatatatcaatgcctgggtcagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctccaggggaaagagccaccctctcctgcagggccagtcagagtattagcagtaatttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaag

SEQ ID NO: 516 - DNA, 510 bp, synthetic construct
  source 1..510: mol_type = other DNA
gagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctcctggggaaagagccaccctctcctgcagggccagtcagattattagcagcaacttagcctggtaccaacagaaacttggccaggctcccaggctcctcatctatgctgcatccaccagggccactgatatcccagccaggttcagtggcagtgggtctgggacagagttcactctcaccatcagcagcctgcagtctgaagattttgcattttattattgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttc

SEQ ID NO: 517 - DNA, 637 bp, synthetic construct
  source 1..637: mol_type = other DNA
ggatgctcagtgcagagaggaagaagcaggtggtctttgcagctgaaagctcagctcccaccccagctgctttgcatgtccctcccagctgccctaccttccagagcccatatcaatgcctgggtcagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctcctggggaaagagccaccctctcctgcagggccagtcagattattagcagcaacttagcctggtaccaacagaaacttggccaggctcccaggctcctcatctatgctgcatccaccagggccactgatatcccagccaggttcagtggcagtgggtctgggacagagttcactctcaccatcagcagcctgcagtctgaagattttgcattttattattgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttctt

SEQ ID NO: 518 - DNA, 549 bp, synthetic construct
  source 1..549: mol_type = other DNA
gtaaaatcttttcttatatgggggggtcagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctccaggggaaagagccaccctctcctgcagggccagtcagagtattagcagcaacttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttc

SEQ ID NO: 519 - DNA, 673 bp, synthetic construct
  source 1..673: mol_type = other DNA
gggagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctccaggggaaagagccaccctctcctgcagggccagtctgagtattagcagcaacttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgaca

SEQ ID NO: 520 - DNA, 538 bp, synthetic construct
  source 1..538: mol_type = other DNA
gagagctctggggaggaactgctcagttaggacccagacggaaccatggaagccccagcgcagcttctcctcctcctgctactctgcctcccagaaaccactggagaaatagtgatgacgcagtctccagccaccctgtctgtgtctccaggggaaagagccaccctctcctgcagggccagtcagagtattagcagcaacttagcctggtaccaacagaaacttggccaggctcccaggctcctcatctatgctgcatccaccagggccactggtatcccagccaggttcagtggcagtgggtctgggacagagttcactctcaccatcagcagcctgcagtctgaagattttgcattttattactgtcagcagtatattgagaggcctctcactttcggcggagggaccaaggtggcgatcaaacgggctgatgctgcaccaactgtatccatcttcccaccatccagtgagcagttaacatctggaggtgcctcagtcgtgtgcttcttgaacaacttctaccccaaagacatca

SEQ ID NO: 521 - DNA, 663 bp, synthetic construct
  source 1..663: mol_type = other DNA
ggcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 522 - DNA, 793 bp, synthetic construct
  source 1..793: mol_type = other DNA
gtgctgagttactaagatgagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 523 - DNA, 688 bp, synthetic construct
  source 1..688: mol_type = other DNA
gagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaggattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggact

SEQ ID NO: 524 - DNA, 824 bp, synthetic construct
  source 1..824: mol_type = other DNA
ctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccctcaggattcagtggcagtggatctgggacagatttcactctcaccatcagcagtctgcaacctgacgattttgcgacttactactgtcaacagacttacagtaccccattcactttcggccctgggaccacagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaat

SEQ ID NO: 525 - DNA, 665 bp, synthetic construct
  source 1..665: mol_type = other DNA
ggtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacctacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 526 - DNA, 774 bp, synthetic construct
  source 1..774: mol_type = other DNA
agccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 527 - DNA, 834 bp, synthetic construct
  source 1..834: mol_type = other DNA
gctgcgctggtctgacctgggaccctggggacattgcccctgtgctgagttactaagatgagccagccctgcagctgtgctcagcctgccccatgccctgctgattgatttgcatgttccagagcacagccccctgccctgaagacttttttatgggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 528 - DNA, 665 bp, synthetic construct
  source 1..665: mol_type = other DNA
tggggaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcagcagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 529 - DNA, 799 bp, synthetic construct
  source 1..799: mol_type = other DNA
gggggctggtcgcaccctgtgcaggagtcagtctcagtcaggacacagcatggacatgagggtccccgctcagctcctggggctcctgctactctggctccgaggtgccagatgtgacatccagatgacccagtctccatcctccctgtctgcatctgtaggagacagagtcaccatcacttgccgggcaagtcagaacattatcacctatttaacttggtatcaacagagaccagggaaagcccctaggctcctgatctatactgcatccagtttgcaaagtggagtcccatcagggttcagtggcagtggatctgggacagatttcactctcaccatcagcagtctgcaacctgaagattttgcgacttactactgtcaacagacttacagtaccccattcactttcggccctgggaccatagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagccaagtgacc

SEQ ID NO: 530 - DNA, 807 bp, synthetic construct
  source 1..807: mol_type = other DNA
cgtggggacactgagctggtgctgagttactgagatgcgccagctctgcagctgtgcccagcctgccccatcccctgctcatttgcatgttcccagagcacaacctcctgccctgaagccttattaataggctggtcagactttgtgcaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagcaattatttagcctggtttcagcagaaaccagggaaagcccctaagtccctgatctttgctgcatccagtttgctaagtgggatcccatcaaagttcagcggcagtggatctgggacagatttcactctcaccatcagcagcctgcagcctgaagattttgcaacttattactgccaacattattatacttaccctcggacgttcggccaagggaccacggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgaca

SEQ ID NO: 531 - DNA, 684 bp, synthetic construct
  source 1..684: mol_type = other DNA
ggtgatgttttttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagtaattatttagcctggtttcagcagaaaccagggaaagcccctgagtccctgatctatgctgcacacagtttgctaagtggggtcccatcaaggttcagcggcagtggatctgggacagatttcactctcaccatcagcagcctgcagcctgaagatactgcaacttattactgccaacattattatacttaccctcggacgttcggccaagggaccgaggtggatatcggacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 532 - DNA, 522 bp, synthetic construct
  source 1..522: mol_type = other DNA
ggctggtcagactttgtgcaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcaggacattagtaattatttagcctggtttcagcagaaaccagggaaagcccctgactccctgatctttgctgcacgcactttgctaagtggggtcccatcaaggttcagcggcagtggatctgggacagatttcactctcaccatcaacagcctgcagcctgaagatgctgcgacttattactgccaacattattatacttaccctcggacgttcggccaagggaccgaggtggatatcggacgggctgatgctgcaccaactgtatccatcttcccaccatccagtgagcagttaacatctggaggtgcctcagtcgtgtgcttcttgaa

SEQ ID NO: 533 - DNA, 711 bp, synthetic construct
  source 1..711: mol_type = other DNA
tacgtttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcaggacattagtaattatttagcctggtttcagcagaaaccagggaaagcccctgactccctgatctttgctgcacgcactttgttaagtggggtcccatcaaggttcagcggcagtggatctgggacagatttcactctcaccatcagcagccttcagcctgaagatactgcaacttattactgccaacattattatacttaccctcggacgttcggccaagggaccgaggtggatctcggacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagaca

SEQ ID NO: 534 - DNA, 682 bp, synthetic construct
  source 1..682: mol_type = other DNA
ggaggctggtcagactttgtgcaggaatcagacccagtcaggacacagcatggacatgagagtcctcgctcagctcctggggctcctgctgctctgtttcccaggtgccagaggtgacatccagatgacccagtctccttcttcactgtctgcatctataggagaccgagtcaccatcacttgtcgggcgagtcagggcattagcaattatttagcctggtttcagcagaaaccagggaaagcccctaagtccctgatctttgctgcatccagtttgttaagtggggtcccatcaaagttcagcggcagtggatctgggacagatttcactctcaccatcagcagcctgcagcctgaagattttgcaacttattactgccaacattattatacttaccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 535 - DNA, 519 bp, synthetic construct
  source 1..519: mol_type = other DNA
gaggctggtcagactttgtgcaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagcaattatttagcctggtttcagcagaaaccagggaaagcccctaagtccctgatctttgctgcatccagtttgctaagtggggtcccatcaaagttcagcggcagtggatctgggacagatttcactctcaccatcagcagcctgcagcctgaagattttgcaacttattactgccaacattattatacttaccctcggacgttcggccaagggaccacggtggaaatcaaacgggctgatgctgcaccaactgtatccatcttcccaccatccagtgagcagttaacatctggaggtgcctcagtcgtgtgcttc

SEQ ID NO: 536 - DNA, 677 bp, synthetic construct
  source 1..677: mol_type = other DNA
ctatttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcaggacattagtaattatttagcctggtttcagcagaaaccagggaaagcccctgactccctgatctttgctgcacgcactttgttaagtggggtcccatcaaggttcagcggcagtggatctgggacagatttcactctcaccatcagcagcctgcagcctgaagatactgcaacttattactgccaacattattatacttaccctcggacgttcggccaagggaccgaggtggatatcggacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 537 - DNA, 520 bp, synthetic construct
  source 1..520: mol_type = other DNA
atatcagttttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcaggacattagtaattatttagcctggtttcagcagaaaccagggaaagcccctgactccctgatctttgctgcacgcactttgttaagtggggtcccatcaaggttcagcggcagtggatctgggacagatttcactctcaccatcagcagccttcagcctgaagatactgcaacttattactgccaacattattatacttaccctcggacgttcggccaagggaccgaggtggatctcggacgggctgatgctgcaccaactgtatccatcttcccaccatccagtgagcagttaacatctggaggtgcctcagtcgtgtgcttc

SEQ ID NO: 538 - DNA, 665 bp, synthetic construct
  source 1..665: mol_type = other DNA
tggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagtaattatttagcctggtttcagcagaaaccagggaaagcccctgagtccctgatctatgctgcacacagtttgctaagtggggtcccatcaaggttcagcggcagtggatctgggacagatttcactctcaccatcagcagcctgcagcctgaagatactgcaacttattactgccaacattattatacttaccctcggacgttcggccaagggaccgaggtggatatcggacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 539 - DNA, 681 bp, synthetic construct
  source 1..681: mol_type = other DNA
catacgatttcttatatggggaggaatcagacccagtcaggacacagcatggacatgagagtcctcgctcagctcctggggctcctgctgctctgtttcccaggtgccagatgtgacatccagatgacccagtctccttcctcactgtctgcatctataggagacagagtcaccatcacttgtcgggcgagtcagggcattagcaattatttagcctggtttcagcagaaaccagggaaagcccctaagtccctgatctatgctgcatccagtttacatactggggtcccgtcaaagttcagcggcagtggatctgggacagatttcactctcaccatcagcagcctgcagcctgaagattttgcaacttattactgccaacattattatagttaccctcggacgttcggccaagggaccaaggtggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 540 - DNA, 681 bp, synthetic construct
  source 1..681: mol_type = other DNA
gaggctggtcagactttgtgcaggaatcagacccagtcaggacacagcatggacatgagagtcctcgctcagctcctggggctcctgctgctctgtttcccaggtgccagatgtgacatccagatgacccagtctccttcctcactgtctgcatctataggagacagagtcaccatcacttgtcgggcgagtcagggcattagcaattatttagcctggtttcagcagaaaccagggaaagcccctaagtccctgatctatgctgcatccagtttacatactggggtcccgtcaaagttcagcggcagtggatctgggacagatttcactctcaccatcagcagcctgcagcctgaagattttgcaacttattactgccaacattattatagttaccctcggacgttcggccaagggaccaaggtggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 541 - DNA, 681 bp, synthetic construct
  source 1..681: mol_type = other DNA
gaggctggtcagactttgtgcaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagcaattatttagcctggtttcagcagaaaccagggaaagcccctaagtccctgatctttgctgcatccagtttactaagtggggtcccatcaaagttcagcggcagtggatctgggacagatttcactctcaccatcagcagcctgcagcctgaagattttgcaacttattactgccaacattattatagttaccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 542 - DNA, 684 bp, synthetic construct
  source 1..684: mol_type = other DNA
gaggctggtcagactttgtgcaggaatcagacccagtcaggacacagcatggacatgagagtcctcgctcagctcctggggctcctgctgctctgtttcccaggtgccagatgtgacatccagatgacccagtctccttcctcactgtctgcatctataggagacagagtcaccatcacttgtcgggcgagtcagggcattagcaattatttagcctggtttcagcagaagccagggaaagcccctaagtccctgatctatgctgcatccagtttgcatagtggggtcccgtcaaagttcagcggcagtggatctgggacagatttcactctcaccatcagcagcctgcagcctgaagattttgcaacttattactgccaacattattatagttaccctcggacgttcggccaagggaccaaggtggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataac

SEQ ID NO: 543 - DNA, 675 bp, synthetic construct
  source 1..675: mol_type = other DNA
ctttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagcaattatttagcctggtttcagcagaaaccagggaaagcccctaagtccctgatctttgctgcatccagtttgctaagtggggtcccatcaaagttcagcggcagtggatctgggacagatttcactctcaccatcagcagcctgcagcctgaagattttgcaacttattactgccaacattattatagttaccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 544 - DNA, 511 bp, synthetic construct
  source 1..511: mol_type = other DNA
gagactttgtgcaggaatcagacccagtcaggacacagcatggacatgagagtcctcgctcagctcctggggctcctgctgctctgtttcccaggtgccagatgtgacatccagatgacccagtctccttcctcactgtctgcatctataggagacagagtcaccatcacttgtcgggcgagtcagggcattagcaattatttagcctggtttcagcagaaaccagggaaagcccctaagtccctgatctatgctgcatccagtttacatactggggtcccgtcaaagttcagcggcagtggatctgggacagatttcactctcaccatcagcagcctgcagcctgaagattttgcaacttattactgccaacattattatagttaccctcggacgttcggccaagggaccaaggtggaaatcaagcgggctgatgctgcaccaactgtatccatcttcccaccatccagtgagcagttaacatctggaggtgcctcagtcgtgtgcttct

SEQ ID NO: 545 - DNA, 689 bp, synthetic construct
  source 1..689: mol_type = other DNA
actttcttatatggggaggaatcagacccagtcaggacacagcatggacatgagagtcctcgctcagctcctggggctcctgctgctctgtttcccaggtgccagaggtgacatccagatgacccagtctccttcttcactgtctgcatctataggagacagagtcaccatcacttgtcgggcgagtcagggcattagcaattatttagcctggtttcagcagaaaccagggaaagcccctaagtccctgatctttgctgcatccagtttgctaagtggggtcccatcaaagttcagcggcagtggatctgggacagatttcactctcaccatcagcagcctgcagcctgaagattttgcaacttattactgccaacattattatagttaccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacct

SEQ ID NO: 546 - DNA, 681 bp, synthetic construct
  source 1..681: mol_type = other DNA
gaggctggtcagactttgtgcaggaatcagacccagtcaggacacagcatggacatgagagtcctcgctcagctcctggggctcctgctgctctgtttcccaggtgccagatgtgacatccagatgacccagtctccttcctcactgtctgcatctataggagacagagtcagcatcacttgtcgggcgagtcagggcattagcaattatttagcctggtttcagcagaaaccagggaaagcccctaagtccctgatctatgctgcatccactttgaatagtggggtcccgtcaaagttcagcggcagtggatctgggacagatttcactctcaccatcagcagcctgcagcctgaagattttgcaacttattactgccaacattattatagttaccctcggacgttcggccaagggaccaaggtggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 547 - DNA, 690 bp, synthetic construct
  source 1..690: mol_type = other DNA
ggggaggctggtcagactttgtgcaggaatcagacccagtcaggacacagcatggacatgagagtcctcgctcagctcctggggctcctgctgctctgtttcccagcctcaggtgccagaggtgacatccagctgacccagtctccttcttcactgtcttcatctataggagacagagtcaccatcacttgtcgggcgagtcagggcattagcaattatttagcctggtttcagcagaaaccagggaaagcccctaagtccctgatctatgctgcatccagtttgctaagtggggtcccatcaaagttcagcggcagtggatctgggacagatttcactctcaccatcagcagcctgcagcctgaagattttgcaacttattactgccaacattattatagttatcctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 548 - DNA, 676 bp, synthetic construct
  source 1..676: mol_type = other DNA
atttcttatatggggaggaatcagtcccactcaggacacagcgtggacatgagggtccccgctcagctcctggggctcctgctgctctggttcccaggtgccacgtgtgacatccagttgacccagtctccatcctccctgtctgcatctgtaggagacagagtcaccatcacttgccgggcaagtcaggccattagaaatgatttaggctggtatcagcagaacccagggaaagcccctaagcgcctgatctatgctgcatccagtttgcaaagtggggtcccatcaaggttcagcggcagtggatctgggacagaattcactctcacgatcagcagcctgcagcctgaagattttgcaacttattactgtctgcaacatagtagttacccattcactttcggccctgggaccaaagtggatatcagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacata

SEQ ID NO: 549 - DNA, 668 bp, synthetic construct
  source 1..668: mol_type = other DNA
ggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaccccgggaaagcccctaagcgcctgatctatgctgcatccagtttgcaaagtggggtcccatcaaggttcagcggcagtggatctgggacagaattcactctcacgatcagcagcctgcagcctgaagattttgcaacttattactgcctacagcatagtagttacccattcactttcggccctgggaccaaagtggatatcagacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataaca

SEQ ID NO: 550 - DNA, 519 bp, synthetic construct
  source 1..519: mol_type = other DNA
gaggctggacacacttcatgcaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgcagcatccagtttgcaaagtggggtcccatcaaggttcagcggcagtggatctgggacagaattcactctcacaatcagcagcctgcagcctgaagattttgcaacttattactgtctacagcatagtagttacccattcactttcggccctgggaccaaagtggatatcaaacgggctgatgctgcaccaactgtatccatcttcccaccatccagtgagcagttaacatctggaggtgcctcagtcgtgtgcttc

SEQ ID NO: 551 - DNA, 565 bp, synthetic construct
  source 1..565: mol_type = other DNA
tgtgtttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtgggagacagagtcaccatcacttgccgggcaagtcagggcattagaaatgatttaggctggtatcaacagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcatagtagttacccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatg

SEQ ID NO: 552 - DNA, 676 bp, synthetic construct
  source 1..676: mol_type = other DNA
ggtttcttatatggggaggaatcagtcccactcaggacacagcatggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttacaaagtggggtcccgtcaaggttcagcggcagtggatctgggacagaattcactctcacaatcagcagcctgcagcctgaagattttgcaacttattactgtctacagcatagtagttacccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 553 - DNA, 680 bp, synthetic construct
  source 1..680: mol_type = other DNA
gttttcttatatggggaggaatcagtcccactcaggacacagcatggacatgggggtccccgctcagctcctggggctcctgctgctctggttcccagttgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcatagtagttacccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataaca

SEQ ID NO: 554 - DNA, 722 bp, synthetic construct
  source 1..722: mol_type = other DNA
tgtcccacagagcaacgcctactgccctgaacatttatcaataggctggtgacatcctgtgcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcaatcaggggtcccatctcggttcagtggcagtggatctgggacagatttcactctcaccatcagcagcctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 555 - DNA, 518 bp, synthetic construct
  source 1..518: mol_type = other DNA
gggcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcaatcaggggtcccatctcggttcagtggcagtggatctgggacagatttcactctcaccatcagcagtctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctacc

SEQ ID NO: 556 - DNA, 681 bp, synthetic construct
  source 1..681: mol_type = other DNA
gaggctggtgacatcctgtgcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcaatcaggggtcccatctcggttcagtggcagtggatctgggacagatttcactctcaccatcagcagcctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 557 - DNA, 514 bp, synthetic construct
  source 1..514: mol_type = other DNA
gggtgacatcctgtgcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcaatcaggggtcccatctcggttcagtggcagtggatctggggcagatttcactctcaccatcagcagcctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttc

SEQ ID NO: 558 - DNA, 645 bp, synthetic construct
  source 1..645: mol_type = other DNA
gggggcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcattcaggggtcccatctcggttcagtggcagtggatctgggacagatttcactctcaccatcagcagcctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgacc

SEQ ID NO: 559 - DNA, 663 bp, synthetic construct
  source 1..663: mol_type = other DNA
ggcagaagtctctctcagtcagggcacagcatggacatgagggtccctgctcagctcctgggactcctgctgctctggctcccagataccagatgtggcatccagatgacccagtctccatcctccctgtctgcatctgttggagacagagtcaccatcacttgccgggcgagtcatgacattagcaattatttagcctggtatcagcagaaaccagggaaagttcctgaactcctgatctatgctgcatccactttgcaatcaggggtcccatctcggttcagtggcagtggatctgggacagatttcactctcaccatcagcagtctgcagcctgaagatgttggaacttattactgtctagattataacaatgcccctcggacgttcggccaagggaccaaggtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 560 - DNA, 681 bp, synthetic construct
  source 1..681: mol_type = other DNA
gaggctggtcacactttgtgcaggagtcagacccagtcaggacacagcatggacatgagggtccccgctcagctcctggggctcctgctgctctggctcccaggtgccaaatgtgacatccggatgacccagtctccttccatcctgtctgcatctgtaggagacagagtcaccatcacttgccgggccagtcagagtatcagtagctgggtggcctggcttcagcagaaaccagggaaagcccctaagctcctgatctataaggcgtcgagtttagaaagtggggtcccatcaaggttcagcggcagtggatctgggacagaattcactctcaccatcagcagcctgcagcctgatgattttgcaacttattactgccaacagtataatagtgattctcggacgttcggccaagggaccaaggtggaaatcatacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 561 - DNA, 681 bp, synthetic construct
  source 1..681: mol_type = other DNA
gaggctggtcacactttgtgcaggagtcagacccagtcaggacacagcatggacatgagggtccccgctcagctcctggggctcctgctgctctggctcccaggtgccaaatgtgacatccagatgacccagtctccttccatcctgtctgcatctgtaggagacagagtcaccatcacttgccgggccagtcagagtatcagtagctggttggcctggcatcagcagaaaccagggaaagcccctaggctcctgatctataaggcgtctagtttagaaagaggggtcccatcaaggttcagcggcagtggatctgggacagaattcactctcaccatcagcagcctgcagcctgatgattttgcaacttattactgccaacagtataatagtgattctcggacgttcggccaagggaccaaggtggagatcatacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 562 - DNA, 680 bp, synthetic construct
  source 1..680: mol_type = other DNA
aggctggtcacactttgtgcaggagtcagacccagtcaggacacagcatggacatgagggtccccgctcagctcctggggctcctgctgctctggctcccaggtgccaaatgtgacatccagatgacccagtctccttccatcctgtctgcatctgtaggagacagagtcaccatcacttgccgggccagtcagagtatcagtagctggttggcctggcatcagcagaaaccagggaaagcccctaggctcctgatctataaggcgtctagtttagaaagaggggtcccatcaaggttcagcggcagtggatctgggacagaattcactctcaccatcagcagcctgcagcctgatgattttgcaacttattactgccaacagtataatagtgattctcggacgttcggccaagggaccaaggtggagatcatacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 563 - DNA, 685 bp, synthetic construct
  source 1..685: mol_type = other DNA
ggaggctggtcacactttgtgcaggagtcagacccagtcaggacacagcatggacatgagggtccccgctcagctcctggggctcctgctgctctggctcccaggtgccaaatgtgacatccggatgacccagtctccttccatcctgtctgcatctgtaggagacagagtcaccatcacttgccgggccagtcagagtatcagtagctgggtggcctggtttcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaactttttactgccaacagtataatagtgattctcggacgttcggccaagggaccaaggtggaaatcatacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataac

SEQ ID NO: 564 - DNA, 503 bp, synthetic construct
  source 1..503: mol_type = other DNA
gggggaggagtcagacccagtcaggacacagcatggacatgagggtccccgctcagctcctggggctcctgctgctctggctcccaggtgccaaatgtgacatccagatgacccagtctccttccatcctgtctgcgtctgtaggagacagagtcaccatcacttgccgggccagtcagagtatcagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagtgattctcggacgttcggccaagggaccaaggtggaaatcatacgggctgatgctgcaccaactgtatccatcttcccaccatccagtgagcagttaacatctggaggtgcctcagtcgtgtgcttc

SEQ ID NO: 565 - DNA, 695 bp, synthetic construct
  source 1..695: mol_type = other DNA
tgggggtatcttaaaagaggttctttctctgggatgtggcatgagcaaaactgacaagtcaaggcagggagatgttgccatcacaactcattgggtttctgctgctctgggttccagcctccaggggtgaagttgtgctgactcagtctccagactttcagtctgtgactccaaaggacaaagtcgccatctcctgccgggccagtcagaacattggtattaacttacactggtaccagcggaaaccagatcagtctccaaagctcctcatcaaatttgcttcccagtccttctcaggagtcccctcgaggttcagtggcagtggatctgggacagatttcaccctcaccatcaatagcctggaagctgaagatgctgcgacgtatttctgtcatcagagtagcagtttaccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 566 - DNA, 694 bp, synthetic construct
  source 1..694: mol_type = other DNA
gggggtatcttaaaagaggttctttctctgggatgtggcatgagcaaaactgacaagtcaaggcaggaagatgttgccatcacaactcattgggtttctgctgttctgggttccagcctccaggggtgaaattgtgctgactcagtctccagactttcagtctgtgactccaaaggagaaagtcgccatcacctgccgggccagtcagagcattggtagtaacttacactggtaccagcagaaaccagatcagtctccaaaactcctcatcaagtttgcctcccagtccttctcaggggtcccctcgaggttcagtggcagtggatctgggacagatttcaccctcaccatcaatagcctggaagctgaagatgctgcgacgtattactgtcatcagagtagcagtttaccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 567 - DNA, 572 bp, synthetic construct
  source 1..572: mol_type = other DNA
ggccctgcctctcggtatcttaaaagaggttctttctctgggatgtggcatgagcaaaactgacaagtcaaggcagggagatgttgccatcacaactcattgggtttctgctgctctgggttccagcctccaggggtgaagttgtgctgactcagtctccagactttcagtctgtgactccaaaggacaaagtcgccatctcctgccgggccagtcagaacattggtattaacttacactggtaccagcggaaaccagatcagtctccaaagctcctcatcaaatttgcttcccagtccttctcaggggtcccctcgaggttcagtggcagtggatctgggacagatttcaccctcaccatcaatagcctggaagctgaagatgctgcgacgtatttctgtcatcagagtagcagtttaccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaat

SEQ ID NO: 568 - DNA, 810 bp, synthetic construct
  source 1..810: mol_type = other DNA
ggggtatcttaaaagaggttctttctctgggatgtggcatgagcaaaactgacaagtcaaggcagggagatgttgccatcacaactcattgggtttctgctgctctgggttccagcctccaggggtgaagttgtgctgactcagtctccagactttcagtctgtgactccaaaggacaaagtcgccatctcctgccgggccagtcagaacattggtattaacttacactggtaccagcggaaaccagatcagtctccaaaactcctcatcaaatttgcttcccagtccttctcaggggtcccctcgaggttcagtggcagtggatctgggacagatttcaccctcaccatcaatagcctggaagctgaagatgctgcgacgtatttctgtcatcagagtagcagtttaccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaaaggtcctgagacgccaccaccagccaagtgacc

SEQ ID NO: 569 - DNA, 778 bp, synthetic construct
  source 1..778: mol_type = other DNA
gggggtatcttaaaagaggttctttctctgggatgtggcatgagcaaaactgacaagtcaaggcagggagatgttgccatcacaactcattgggtttctgctgctctgggttccagcctccaggggtgaagttgtgctgactcagtctccagactttcagtctgtgactccaaaggacaaagtcgccatctcctgccgggccagtcagaacattggtattaacttacactggtaccagcggaaaccagatcagtctccaaaactcctcatcaaatttgcttcccagtccttctcaggggtcccctcgaggttcagtggcagtggatctgggacagatttcaccctcaccatcaatagcctggaagctgaagatgctgcgacgtatttctgtcatcagagtagcagtttaccattcactttcggccctgggaccaaagtggatatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagagacaa

SEQ ID NO: 570 - DNA, 597 bp, synthetic construct
  source 1..597: mol_type = other DNA
ggggctggtcgcaccctgtgcaggagtcagtctcagtcaggacacagcatggacatgagggtccccgctcagctcctggggctcctgctactctggctccgaggtgccagatgtgacatccagatgacccagtctccatcctctctgtctgcatctgttggagacagattcaccatcacttgccgggcaagtcagagtattagaagttatttaaattggtatcagcagaagccagggaaagcccctaacctcctgatctatgctgcatccagcttgcaaagtggggtcccatcacggttcagtggcagtggatctgggacagatttcactctcaccatcagcagtctgcaacctgaagattttgcaacttactactgtcaacagagttacagtaccccattcactttcggccctgggaccaaagtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgatc

SEQ ID NO: 571 - DNA, 545 bp, synthetic construct
  source 1..545: mol_type = other DNA
tggggaggagtcagtctcagtcaggacacagcatggacatgagggtccccgctcagctcctggggctcctgctactctggctccgaggtgccagatgtgacatccagatgacccagtctccatcctctctgtctgcatctgttggagacagattcaccatcacttgccgggcaagtcagaatattagaagctatttaaattggtatcagcagaagccagggaaagcccctaacctcctgatctatgctgcatccagcttgcaaagtggggtcccatcacggttcagtggcagtggatctgggacagatttcactctcaccatcagcagtctgcaacctgaagattttgcaacttactactgtcaacagagttacagtaccccattcactttcggccctgggaccaaagtggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaag

SEQ ID NO: 572 - DNA, 502 bp, synthetic construct
  source 1..502: mol_type = other DNA
gtgcaggagtcagtctcagtcaggacacagcatggacatgagggtccccgctcagctcctggggctcctgctactctggctccgaggtgccagatgtgacatccagatgacccagtctccatcctccctggctgcatctgtgggagacagagtcaccatcacttgccgggcaagtcagagcattaccagctatttaaattggtatcagcagaaaccagggaaagcccctaagctcctgatctatgctgcatcttattttcaaagtggggtcccatcaaggttcagtggcagtggatctgggacagatttcactctcaccatcagcagtctggaacctgaagattttgcaacttactactgtcagcagagttacagtaccccattcactttcggccctgggaccaaagtggatatcaaacgggctgatgctgcaccaactgtatccatcttcccaccatccagtgagcagttaacatctggaggtgcctcagtcgtgtgcttc

SEQ ID NO: 573 - DNA, 509 bp, synthetic construct
  source 1..509: mol_type = other DNA
gaggagtcagtctcagtcaggacacagcatggacatgagggtccccgctcagctcctggggctcctgctgctctggttcccaggttccagatgcgacatccagatgacccagtctccatcttccgtgtctgcatctgtaggagacagagtcaccatcacttgccgggcaagtcaaaatattagaagttatttaaattggtatcagcagaaattagggaaagcccctaagctcctgatctatgctgcatcctatttacaaagtgggatcccttcaaggttcagtggcagtggatctgggacagatttcactctcaccatcagcagtctgcaacctgaagattttgcaactttctattgtcaacagagttacagtacccctttcactttcggccctgggaccaaagtggatatcaaacgggctgatgctgcaccaactgtatccatcttcccaccatccagtgagcagttaacatctggaggtgcctcagtcgtgtgcttcagatcggaag

SEQ ID NO: 574 - DNA, 686 bp, synthetic construct
  source 1..686: mol_type = other DNA
tttataccgcagtttcttatatggggaggagtcagacccagtcaggacacagcatggacatgagggtccccgctcagctcctggggctcctgctgctctggttcccaggtaccagatgcgacatccagatgacccaatctccatcttccgtgtctgcgtctgtaggagacagagtcaccatcacttgtcgggcgagtcagggcatttacagctggttggcctggtatcagcagaaaccagggaaagcccctaacctcctgatctatgctgcatccagtttgcaaagtggggtcccatcaaggttcagcggcagtggatctgggacagatttcactctcaccatcagcaatttgcaccctgaagattgtgcaacttactattgtcaacaggctaacagtctcccgctcactttcggcggagggaccaaggttgagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 575 - DNA, 685 bp, synthetic construct
  source 1..685: mol_type = other DNA
tggggaggctgatcacaccctgtgcaggagtcagacccagtcaggacacagcatggacatgagggtccccgctcagctcctggggctcctgctgctctggttcccaggtaccagatgcgacatccagatgacccagtctccatcttccgtgtctgcgtctgtaggagacagagtcaccatcacttgtcgggcgagtcagggcatttacagctggttggcctggtatcagcagaaaccagggaaagcccctaacctcctgatctatgctgcatccagcttgcaaactggggtcccatcaaggttcagcggcagtggatctgggacagatttcattctcaccatcagtagtctgcaccctgaagattgtgcaacttactattgtcaacaggctaacagtctcccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 576 - DNA, 681 bp, synthetic construct
  source 1..681: mol_type = other DNA
gaggctgatcacaccctgtgcaggagtcagacccagtcaggacacagcatggacatgagggtccccgctcagctcctggggctcctgctgctctggttcccaggtaccagatgcgacatccagatgacccagtctccatcttccgtgtctgcgtctgtaggagacagagtcaccatcacttgtcgggcgagtcagggcatttacagctggttggcctggtatcagcagaaaccagggaaagcccctaacctcctgatctatgctgcatccagcttgcaaactggggtcccatcaaggttcagcggcagtggatctgggacagatttcattctcaccatcagcagtctgcaccctgaagattgtgcaacttactattgtcaacaggctaacagtctcccgctcactttcggcggagggaccaaggtggagatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacat

SEQ ID NO: 577 - protein, 11 aa, synthetic construct
CQQYIERPLTF

SEQ ID NO: 578 - protein, 14 aa, synthetic construct
CARGANWGFWFDPW

SEQ ID NO: 579 - protein, 13 aa, synthetic construct
CARQKGSGSFDNW

SEQ ID NO: 580 - protein, 11 aa, synthetic construct [sequence omitted]

SEQ ID NO: 581 - protein, 17 aa, synthetic construct
CARGRDCGGECSHFDYW

SEQ ID NO: 582 - protein, 11 aa, synthetic construct [sequence omitted]

SEQ ID NO: 583 - protein, 17 aa, synthetic construct
CARGRDCGGDCSHFDYW

SEQ ID NO: 584 - protein, 11 aa, synthetic construct [sequence omitted]

SEQ ID NO: 585 - protein, 18 aa, synthetic construct
CAKWGITFGEVVVHFDYW

SEQ ID NO: 586 - protein, 11 aa, synthetic construct
CLDYNNAPRTF

SEQ ID NO: 587 - protein, 16 aa, synthetic construct
CVKDISSSWYGYFDLW

SEQ ID NO: 588 - protein, 16 aa, synthetic construct
CTRDISSSWYGYFDYW

SEQ ID NO: 589 - protein, 11 aa, synthetic construct [sequence omitted]

SEQ ID NO: 590 - protein, 14 aa, synthetic construct
CASGIGGRPVFDYW

SEQ ID NO: 591 - protein, 11 aa, synthetic construct [sequence omitted]

SEQ ID NO: 592 - protein, 14 aa, synthetic construct
CARVDKWNYWFDPW

SEQ ID NO: 593 - DNA, 33 bp, synthetic construct
  source 1..33: mol_type = other DNA
tgtcagcagtatattgagaggcctctcactttc

SEQ ID NO: 594 - DNA, 42 bp, synthetic construct
  source 1..42: mol_type = other DNA
tgtgcaagaggggctaactggggattctggttcgacccctgg

SEQ ID NO: 595 - DNA, 33 bp, synthetic construct
  source 1..33: mol_type = other DNA
tgtcaacagacttacagtaccccattcactttc

SEQ ID NO: 596 - DNA, 39 bp, synthetic construct
  source 1..39: mol_type = other DNA
tgtgcgagacaaaaaggttcggggagttttgacaactgg

SEQ ID NO: 597 - DNA, 33 bp, synthetic construct
  source 1..33: mol_type = other DNA
tgtcaacagacctacagtaccccattcactttc

SEQ ID NO: 598 - DNA, 33 bp, synthetic construct
  source 1..33: mol_type = other DNA
tgccaacattattatacttaccctcggacgttc

SEQ ID NO: 599 - DNA, 51 bp, synthetic construct
  source 1..51: mol_type = other DNA
tgtgcgagaggaagggattgtggtggtgaatgttcacactttgactactgg

SEQ ID NO: 600 - DNA, 51 bp, synthetic construct
  source 1..51: mol_type = other DNA
tgtgcgagaggaagggattgtggtggtgaatgctcccactttgactactgg

SEQ ID NO: 601 - DNA, 33 bp, synthetic construct
  source 1..33: mol_type = other DNA
tgtcagcaatatattgagaggcctctcactttc

SEQ ID NO: 602 - DNA, 51 bp, synthetic construct
  source 1..51: mol_type = other DNA
tgtgcgagaggacgggattgtggtggtgaatgctcccactttgactactgg

SEQ ID NO: 603 - DNA, 51 bp, synthetic construct
  source 1..51: mol_type = other DNA
tgtgcgagaggaagggattgtggtggtgagtgttcacactttgactactgg

SEQ ID NO: 604 - DNA, 33 bp, synthetic construct
  source 1..33: mol_type = other DNA
tgccaacattattatagttaccctcggacgttc

SEQ ID NO: 605 - DNA, 51 bp, synthetic construct
  source 1..51: mol_type = other DNA
tgtgcgcgaggaagggattgtggtggtgattgctcccactttgactactgg

SEQ ID NO: 606 - DNA, 51 bp, synthetic construct
  source 1..51: mol_type = other DNA
tgtgcgagaggaagggattgtggtggtgattgctcccactttgactactgg

SEQ ID NO: 607 - DNA, 33 bp, synthetic construct
  source 1..33: mol_type = other DNA
tgccaacattattatagttatcctcggacgttc

SEQ ID NO: 608 - DNA, 33 bp, synthetic construct
  source 1..33: mol_type = other DNA
tgtctgcaacatagtagttacccattcactttc

SEQ ID NO: 609 - DNA, 54 bp, synthetic construct
  source 1..54: mol_type = other DNA
tgtgcgaaatggggaataacgtttggggaagttgtcgtccactttgactactgg

SEQ ID NO: 610 - DNA, 33 bp, synthetic construct
  source 1..33: mol_type = other DNA
tgcctacagcatagtagttacccattcactttc

SEQ ID NO: 611 - DNA, 33 bp, synthetic construct
  source 1..33: mol_type = other DNA
tgtctacagcatagtagttacccattcactttc

SEQ ID NO: 612 - DNA, 33 bp, synthetic construct
  source 1..33: mol_type = other DNA
tgtctagattataacaatgcccctcggacgttc

SEQ ID NO: 613 - DNA, 48 bp, synthetic construct
  source 1..48: mol_type = other DNA
tgtgtaaaagatattagcagcagctggtacgggtatttcgatctctgg

SEQ ID NO: 614 - DNA, 48 bp, synthetic construct
  source 1..48: mol_type = other DNA
tgtgtaaaagatattagcagcagttggtacgggtatttcgatctctgg

SEQ ID NO: 615 - DNA, 48 bp, synthetic construct
  source 1..48: mol_type = other DNA
tgtgtaaaagacattagcagcagctggtacgggtatttcgatctttgg

SEQ ID NO: 616 - DNA, 48 bp, synthetic construct
  source 1..48: mol_type = other DNA
tgtacaagagatataagtagcagctggtacggatattttgactactgg

SEQ ID NO: 617 - DNA, 48 bp, synthetic construct
  source 1..48: mol_type = other DNA
tgtacaagagatataagtagcagttggtatgggtattttgactactgg

SEQ ID NO: 618 - DNA, 48 bp, synthetic construct
  source 1..48: mol_type = other DNA
tgtacaagagatataagtagcagctggtacgggtattttgactactgg

SEQ ID NO: 619 - DNA, 33 bp, synthetic construct
  source 1..33: mol_type = other DNA
tgtcatcagagtagcagtttaccattcactttc

SEQ ID NO: 620 - DNA, 42 bp, synthetic construct
  source 1..42: mol_type = other DNA
tgtgcgagcggtataggaggtcgtccggtctttgactactgg

SEQ ID NO: 621 - DNA, 45 bp, synthetic construct
  source 1..45: mol_type = other DNA
tgtgcgagacatcgtgggagctacgtctacggtatggacgtctgg

SEQ ID NO: 622 - DNA, 33 bp, synthetic construct
  source 1..33: mol_type = other DNA
tgtcagcagagttacagtaccccattcactttc

SEQ ID NO: 623 - DNA, 33 bp, synthetic construct
  source 1..33: mol_type = other DNA
tgtcaacagagttacagtacccctttcactttc

SEQ ID NO: 624 - DNA, 33 bp, synthetic construct
  source 1..33: mol_type = other DNA
tgtcaacaggctaacagtctcccgctcactttc

SEQ ID NO: 625 - DNA, 42 bp, synthetic construct
  source 1..42: mol_type = other DNA
tgtgcaagagtagataagtggaattattggttcgacccctgg

SEQ ID NO: 626 - DNA, 42 bp, synthetic construct
  source 1..42: mol_type = other DNA
tgtgcaagagtagataagtggaactactggttcgacccctgg

SEQ ID NO: 627 - DNA, 16 bp, synthetic construct
  source 1..16: mol_type = other DNA
gactgcgaggaccaca

SEQ ID NO: 628 - DNA, 16 bp, synthetic construct
  source 1..16: mol_type = other DNA
cctaaaggtctaaacc

SEQ ID NO: 629 - DNA, 16 bp, synthetic construct
  source 1..16: mol_type = other DNA
aaccgcgtcccaagat

SEQ ID NO: 630 - DNA, 16 bp, synthetic construct
  source 1..16: mol_type = other DNA
tcggtaaaggacacca

SEQ ID NO: 631 - DNA, 16 bp, synthetic construct
  source 1..16: mol_type = other DNA
cttctctgtctcaaca

SEQ ID NO: 632 - DNA, 16 bp, synthetic construct
  source 1..16: mol_type = other DNA
cggctagcatcccatc

SEQ ID NO: 633 - DNA, 648 bp, synthetic construct
  source 1..648: mol_type = other DNA
gacataccagcaagggagtgaccagttagtcttaaggcaccactgagcccaagtcttagacatcatgggttggctgtggaacttgctattcctgatggcagctgcccaaagtgcccaagcacagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggataaacacctactctggagtgccaacatatgctgatgacttcaagggacggtttgtcttctctttggaaacctctgccagcactgcctatttgcagatcaacaacctcaaaaatgaggacacggctacatatttctgtgcaagagcgtattactacggtagtagctacttgatctggtacttcgatgtctggggcacagggaccacggtcaccgtctcctcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 634 - DNA, 552 bp, synthetic construct
  source 1..552: mol_type = other DNA
gatataacagcccaggcttctttaagggcagctgccaggagcctaagaaagcatcctctcttccagctctcagagatggagacagacacactcctgctatgggtgctgctgctctgggttccaggttccacaggtaacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtacacgttcggaggggggaccaagctggaaataaaacgggctgatgctgcaccaactgtatccatcttcccaccatccagtgagcagttaacatctggaggtgcctcagtcgtgtgcttc

SEQ ID NO: 635 - DNA, 611 bp, synthetic construct
  source 1..611: mol_type = other DNA
accattattcatctttcttatatggggatcctcttctcatagagcctccatcagaccatggctgtcctggcactgctcctctgcctggtgacattcacaagctgtgtcctgtcccaggtgcttctgaaggagtcaggacctggccttgtggcgccctcacagagcctgtccatcacatgcactgtctcagggttctcattaaccagctatggtgttagctgggttcgccagcctccaggaaagggtctggagtggctgggagtaatatggggtgacgggaacacagattatcattcagctctcatatccagactgagcatcaggaaggataactccaagagccaagttttcttaaaactgaacagtctgcaaactgatgacacagccacgtactactgtgccatcggtagtccttttgcttactggggccaagggactctggtcactgtctctgcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 636 - DNA, 525 bp, synthetic construct
  source 1..525: mol_type = other DNA
gggactgatcagtctcctcaggctgtctcctcaggttgcctcctcaaaatgaagttgcctgttaggctgttggtgctgatgttctggattcctgcttccagcagtgatgttttgatgacccaaactccactctccctgcctgtcagtcttggagatcaagtctccatctcttgcagatcaagtcagaacattgtacatagtactggaaacacctatttagaatggtacctgcagaaaccaggccagtctccaaagctcctgatcttcaaagtctccaaccgactttctggggtcccagacaggttcagtggcagtggatcagggacagatttcacactcaacatcagcagggtggaggctgcggatctgggaatttattcctgctttcaaggttcacatgttccattcacgttcggctcggggacaaagttggaaataaaacgggctgatgctgcaccaactgtatccatcttcccaccatccagtgagcagttaacatctggaggtgcctcagtcgtgtgcttc

SEQ ID NO: 637 - DNA, 635 bp, synthetic construct
  source 1..635: mol_type = other DNA
caccatgaaaacaacctatgatcagtgtcctctccacagtccctgaagacactgactctaaccatgggattcagcaggatctttctcttcctcctgtcagtaactacaggtgtccactcccaggcttatctacagcagtctggggctgagctggtgaggcctggggcctcagtgaagatgtcctgcaaggcttctggctacacatttatcagttacaatatgcactgggtaaagcagacacgtagacagggcctggaatggattggagctatttatccaggaaatggtgatacttcctacaatcagaagttcaagggcaaggccacactgactgtagacaaatcctccagcacagcctacatgcagctcagcagcctgacatctgaagactctgcggtctatttctgtgcaagatgggattactatggtagtagctacggggcttactggggccaagggactctggtcactgtctctgcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 638 - DNA, 509 bp, synthetic construct
  source 1..509: mol_type = other DNA
cacaccatgtgctggagtcagcctcacactgatcacacacagacatgagtgtgcccactcaggtcctggggttgctgctgctgtggcttacagatgccagatgtgacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtggacgttcggtggaggcaccaagctggaaatcaaacgggctgatgctgcaccaactgtatccatcttcccaccatccagtgagcagttaacatctggaggtgcctcagtcgtgtgcttc

SEQ ID NO: 639 - DNA, 633 bp, synthetic construct
  source 1..633: mol_type = other DNA
ccctgaaaacaacatatgtacaatgtcctcaccacagacactgaacacactgactctaaccatgggatggagctggatctttctctttctcctgtcaggaactgcaggtgtcctctctgaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcgtacggtcccccccggggacccccctggtttgcttactggggccaagggactctggtcactgtctctgcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 640 - DNA, 523 bp, synthetic construct
  source 1..523: mol_type = other DNA
gagctgccaggagcctaagaagcatcctctcttccagctctcagagatggagacagacacactcctgttatgggtactgctgctctgggttccaggttccactggtgacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtggacgttcggtggaggcaccaagctggaaatcaaacgggctgatgctgcaccaactgtatccatcttcccaccatccagtgagcagttaacatctggaggtgcctcagtcgtgtgcttc

SEQ ID NO: 641 - DNA, 621 bp, synthetic construct
  source 1..621: mol_type = other DNA
tgggatcaacatatgtccaatgtcctctccacaatccctgaagacacggactctaactatgggatggaactggatctttattttaatcctgtcagtaactacaggtgtccactctgaggtccacctgcagcagtctggacctgacctggtgaagcctggggcttcagtgatgatttcctgccaggcttctggttactcattcactgcctattacataaactgggtgaaacaaagtcctgaaaagagccttgagtggattggagagattaatcctggcagtggaggtgttatctataaccggaaattcgaggccaaggccacattgattgtggacaaatcctccaacacagccttcatgctactcacgagactgacatctgaggactctgcaatttattattgtgcaagaaagggcttctaccagtttgactactggggccaaggcaccactctcacagtctcctcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 642 - DNA, 559 bp, synthetic construct
  source 1..559: mol_type = other DNA
gtagattcttgcaggtcagcccagctgcccatgttttataaaccaggcctttgctgtaagaacagaaatacatcaggcaggcaagggcatcaagatgaagtcacagacccaggtcttcgtatttctactgctctgtgtgtctggtactcatgggagtgttgtgatgacccagattcccaagttcctggttgtatcagcaggagacagggttaccataacctgtaaggccagtcagagtgtgaatgatggtgttttttggtaccaacagaagacagggcagtctcctaaactggtaatatatcatacatccaatcgctacactggagtctctgatcgcttcactggcagtgggtatgggacggagttcactttcaccatcagcgctgtgcagtctgaagacctggcaatttatttctgtcagcaggattatacctctccgttgacgttcggtggaggcaccaggctggaaatcaaacgggctgatgctgcaccaactgtatccatcttcccaccatccagtgagcagttaacatctggaggtgcctcagtcgtgtgcttc

SEQ ID NO: 643 - DNA, 636 bp, synthetic construct
  source 1..636: mol_type = other DNA
gggtgaaaacaacatatgactcctgtcttctccacagtcccagaacacactcactctaaccatggaatggccttgtatctttctcttcctcctgtcagtaactgaaggtgtccactcccaggttcagctgcagcagtctggacctgagctggtgaagcctggggcctcagtgaagatttcctgcaaggctcctggcaacgcattcagttactcctggatgaactgggtgaagcagaggcctggaaagggtcttgagtgggttggacggatttatcctggagatggagatactaattacaatgggaagttcatgggcaaggccacactgactgcagacaagtcctccaacactgcctacatacaactcagcagcctgacatctgaggactctgcggtctatttctgtgcaagatcggggggtgggaccctctactggtacttcgatgtctggggcacagggaccacggtcaccgtctcgtcagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgc

SEQ ID NO: 644 - DNA, 510 bp, synthetic construct
  source 1..510: mol_type = other DNA
acagcatcagacaggctgggcagcaagatggaatcacagacccaggtcctcatgtttcttctgctctgggtatctggtgcctgtgcagacattgtgatgacccagtctccatcctccctggctttgtcagtaggacagaaggtcactatgaactgcaggtccagtcagagccttttaaatagtttcaatcaaaagaactatttggcctggttccagcagaaaccaggacagtctcctagacttctgatatactttgcatccactagggaatctggggtccctgatcgcttcataggcagtggatctgggacagatttcactcttaccatcagcagtgtgcaggctgaagacctggcagattacttctgtcagcaacattataatactccgctcacgttcggtgctgggaccacgctggagctgaaacgggctgatgctgcaccaactgtatccatcttcccaccatccagtgagcagttaacatctggaggtgcctcagtcgtgtgcttc

SEQ ID NO: 645 - protein, 11 aa, synthetic construct
CQQNNEDPYTF

SEQ ID NO: 646 - protein, 11 aa, synthetic construct
CFQGSHVPFTF

SEQ ID NO: 647 - protein, 11 aa, synthetic construct
CQHFWGTPWTF

SEQ ID NO: 648 - protein, 11 aa, synthetic construct
CQHSRELPWTF

SEQ ID NO: 649 - protein, 11 aa, synthetic construct
CQQDYTSPLTF

SEQ ID NO: 650 - protein, 11 aa, synthetic construct
CQQHYNTPLTF

SEQ ID NO: 651 - protein, 19 aa, synthetic construct
CARAYYYGSSYLIWYFDVW

SEQ ID NO: 652 - DNA, 33 bp, synthetic construct
  source 1..33: mol_type = other DNA
tgtcagcaaaataatgaggatccgtacacgttc

SEQ ID NO: 653 - DNA, 57 bp, synthetic construct
  source 1..57: mol_type = other DNA
tgtgcaagagcgtattactacggtagtagctacttgatctggtacttcgatgtctgg

SEQ ID NO: 654 - protein, 10 aa, synthetic construct
CAIGSPFAYW

SEQ ID NO: 655 - DNA, 33 bp, synthetic construct
  source 1..33: mol_type = other DNA
tgctttcaaggttcacatgttccattcacgttc

SEQ ID NO: 656 - DNA, 30 bp, synthetic construct
  source 1..30: mol_type = other DNA
tgtgccatcggtagtccttttgcttactgg

SEQ ID NO: 657 - protein, 15 aa, synthetic construct
CARWDYYGSSYGAYW

SEQ ID NO: 658 - DNA, 33 bp, synthetic construct
  source 1..33: mol_type = other DNA
tgtcaacatttttggggtactccgtggacgttc

SEQ ID NO: 659 - DNA, 45 bp, synthetic construct
  source 1..45: mol_type = other DNA
tgtgcaagatgggattactatggtagtagctacggggcttactgg

SEQ ID NO: 660 - protein, 15 aa, synthetic construct
CAYGPPRGPPWFAYW

SEQ ID NO: 661 - DNA, 33 bp, synthetic construct
  source 1..33: mol_type = other DNA
tgtcagcacagtagggagcttccgtggacgttc

SEQ ID NO: 662 - DNA, 45 bp, synthetic construct
  source 1..45: mol_type = other DNA
tgtgcgtacggtcccccccggggacccccctggtttgcttactgg

SEQ ID NO: 663 - protein, 12 aa, synthetic construct
CARKGFYQFDYW

SEQ ID NO: 664 - DNA, 33 bp, synthetic construct
  source 1..33: mol_type = other DNA
tgtcagcaggattatacctctccgttgacgttc

SEQ ID NO: 665 - DNA, 36 bp, synthetic construct
  source 1..36: mol_type = other DNA
tgtgcaagaaagggcttctaccagtttgactactgg

SEQ ID NO: 666 - protein, 16 aa, synthetic construct
CARSGGGTLYWYFDVW

SEQ ID NO: 667 - DNA, 33 bp, synthetic construct
  source 1..33: mol_type = other DNA
tgtcagcaacattataatactccgctcacgttc

SEQ ID NO: 668 - DNA, 48 bp, synthetic construct
  source 1..48: mol_type = other DNA
tgtgcaagatcggggggtgggaccctctactggtacttcgatgtctgg

SEQ ID NO: 669 - DNA, 16 bp, synthetic construct
  source 1..16: mol_type = other DNA
ggaataatcagtccct

SEQ ID NO: 670 - DNA, 666 bp, synthetic construct
  source 1..666: mol_type = other DNA
cagccctttcttatatggggagctctgggagaggagccccagccctgagattcccaggtgtttccattcagtgatcagcactgaacacagaggactcaccatggagttgggactgagctggattttccttttggctattttaaaaggtgtcaagtgtgaagtgaagttggtggagtctgggggaggcttggtccagcctggcaggtccctgagactctcctgtgcagcctctggattcatctttgatgattatgtcatgcattgggtccgtcaacgtcctgggaagggcctggagtgggtcgcacgtatcacgtggaatagtagtacattggcctatgcggactgtgttaagggccgattcaccatctccagagacaaagccaagaaaaccctgtatctgcaaatgggcagtctgagagatgaggacacgggcttatattactgtgcaaaaggcggcgactatgtcactgttggctacttctactactctatggccgtctggggccaagggaccacggtcaccgtctcctcagcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 671 - DNA, 16 bp, synthetic construct
  source 1..16: mol_type = other DNA
aaacgggcacggatag

SEQ ID NO: 672 - DNA, 320 bp, synthetic construct
  source 1..320: mol_type = other DNA
atctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 673 - DNA, 16 bp, synthetic construct
  source 1..16: mol_type = other DNA
acaccaagtctcacct

SEQ ID NO: 674 - DNA, 320 bp, synthetic construct; identical to SEQ ID NO: 672
  source 1..320: mol_type = other DNA

SEQ ID NO: 675 - DNA, 16 bp, synthetic construct
  source 1..16: mol_type = other DNA
acttactcaatctgca

SEQ ID NO: 676 - DNA, 319 bp, synthetic construct
  source 1..319: mol_type = other DNA
tctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 677 - DNA, 16 bp, synthetic construct
  source 1..16: mol_type = other DNA
attggaccaccgaatt

SEQ ID NO: 678 - DNA, 303 bp, synthetic construct
  source 1..303: mol_type = other DNA
ctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcagga

SEQ ID NO: 679 - DNA, 16 bp, synthetic construct
  source 1..16: mol_type = other DNA
cacaggcagtaagtac

SEQ ID NO: 680 - DNA, 698 bp, synthetic construct
  source 1..698: mol_type = other DNA
gggagctctgggagaggagccccagccctgagattcccaggtgtttgcattcagtgatcagcactgaacacagaggactcaccatggagttgggactgagctggattttccttttggctcttttaaaaggtgtcaagtgtgaagtggaactggtggagtctgggggaggcttggtacagcctggcaggtccctcagactctcctgtgcggcctctggattcatctttgatgatcatgtcatgttttgggtccggcaacctcctgggaagggcctggagtgggtcgcacggatctcttggaatagtattagattggcctatgcggactgtgtgaagggccgattcaccatctccagagacaaagccaagaacaccctatatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 681 - DNA, 16 bp, synthetic construct
  source 1..16: mol_type = other DNA
cacaggccacctcgtt

SEQ ID NO: 682 - DNA, 401 bp, synthetic construct
  source 1..401: mol_type = other DNA
ggaatagtattagattggcctatgcggactgtgtgaagggccgattcaccatctccagagacaaagccaagaacaccctatatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 683 - DNA, 16 bp, synthetic construct
  source 1..16: mol_type = other DNA
cttggctagaccacga

SEQ ID NO: 684 - DNA, 322 bp, synthetic construct
  source 1..322: mol_type = other DNA
atatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 685 - DNA, 16 bp, synthetic construct
  source 1..16: mol_type = other DNA
gatcgatgtgatgccc

SEQ ID NO: 686 - DNA, 347 bp, synthetic construct
  source 1..347: mol_type = other DNA
tccctcagactctcctgtgcggcctctggattcatctttgatgatcatgtcatgttttgggtccggcaacctcctgggaagggcctggagtgggtcgcacggatctcttggaatagtattagattggcctatgcggactgtgtgaagggccgattcaccatctccagagacaaagccaagaacaccctatatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatc

SEQ ID NO: 687 - DNA, 16 bp, synthetic construct
  source 1..16: mol_type = other DNA
gggcactagtgtctca

SEQ ID NO: 688 - DNA, 322 bp, synthetic construct
  source 1..322: mol_type = other DNA
atatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 689 - DNA, 16 bp, synthetic construct
  source 1..16: mol_type = other DNA
ggtgaagtcctagaac

SEQ ID NO: 690 - DNA, 317 bp, synthetic construct
  source 1..317: mol_type = other DNA
ggcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 691 - DNA, 16 bp, synthetic construct
  source 1..16: mol_type = other DNA
gtaactgagtatctcg

SEQ ID NO: 692 - DNA, 310 bp, synthetic construct
  source 1..310: mol_type = other DNA
ggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 693 - DNA, 16 bp, synthetic construct
  source 1..16: mol_type = other DNA
gtgttaggtggcaaac

SEQ ID NO: 694 - DNA, 393 bp, synthetic construct
  source 1..393: mol_type = other DNA
attagattggcctatgcggactgtgtgaagggccgattcaccatctccagagacaaagccaagaacaccctatatctgcaaatggacagtctgagacatgaggacacggccttatattactgtgcaaaaggcggcgactacgtcactgttggctacttctactactctatggatgtctggggtcaagggacagcggtcaccgtctcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcagga

SEQ ID NO: 695 - DNA, 16 bp, synthetic construct
  source 1..16: mol_type = other DNA
tacttaccatccttgc

SEQ ID NO: 696 - DNA, 322 bp, synthetic construct; identical to SEQ ID NO: 684
  source 1..322: mol_type = other DNA

SEQ ID NO: 697 - DNA, 16 bp, synthetic construct
  source 1..16: mol_type = other DNA
ttaggacgtctcaaca

SEQ ID NO: 698 - DNA, 322 bp, synthetic construct; identical to SEQ ID NO: 684
  source 1..322: mol_type = other DNA

SEQ ID NO: 699 - DNA, 16 bp, synthetic construct
  source 1..16: mol_type = other DNA
attctaccattcactt

SEQ ID NO: 700 - DNA, 343 bp, synthetic construct
  source 1..343: mol_type = other DNA
acaatccctccctcatgagtcgagccaccatgtcagtggacgcgtccaaaaatcagttctccctgacgttgaaatatgtgaccgccgcggacacggccgtgtattattgtgcgagatccgaaggactacaattcgcagggcgggtcctcggtacacgcaaatatgacttcgacgtctggggccaagggaccacggtcatcgtctcctcagcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 701 - DNA, 16 bp, synthetic construct
  source 1..16: mol_type = other DNA
catcagaagccacgct

SEQ ID NO: 702 - DNA, 301 bp, synthetic construct
  source 1..301: mol_type = other DNA
gagtcaccatttcattggacacgtccaagaaccaactctccctgaacctgaggtctctgaccgctgcggacacggccgtgtattattgtgcgagagagtgggtggtttcaccctccactgatgcttttgatgtgtggggccaagggacaatggtcaccgtctcttccgcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 703 - DNA, 16 bp, synthetic construct
  source 1..16: mol_type = other DNA
gacgcgtagacaagcc

SEQ ID NO: 704 - DNA, 619 bp, synthetic construct
  source 1..619: mol_type = other DNA
gaaattcaggttccagctcacatgggaaatcctttctgagagtcctggacctcctgtgcaagaacatgaaacatctgtggatcttccttctcctggtggcagctcccagatgggtcctgtcccaggtgcagctacagcagtcgggcccgggactggtgaagccttcggagaccctgtccctcacctgcacagtgtctggaggctccatcagtgggttctattggacctggatccggcagcccccagggaagggactggaatggattggctatatctctcacagggggagcacccacttcaacccctccttaaagagtcgagtcaccatttcattggacacgtccaagaaccaactctccctgaacctgaggtctctgaccgctgcggacacggccgtgtattattgtgcgagagagtgggtggtttcaccctccactgatgcttttgatgtgtggggccaagggacaatggtcaccgtctcttccgcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 705 - DNA, 16 bp, synthetic construct
  source 1..16: mol_type = other DNA
ttaggcaagcgagaaa

SEQ ID NO: 706 - DNA, 301 bp, synthetic construct; identical to SEQ ID NO: 702
  source 1..301: mol_type = other DNA

SEQ ID NO: 707 - DNA, 16 bp, synthetic construct
  source 1..16: mol_type = other DNA
ccgggatcaatagcaa

SEQ ID NO: 708 - DNA, 479 bp, synthetic construct
  source 1..479: mol_type = other DNA
gggtccctgagactctcctgtgcaacctctggattcacctttagtggtttttggatgacctgggtccgccaggctccggggaagggactggaatgtgtggctaacataaaccaagatggaagtgagaaataccatgtggactcagtgaagggccgattcaccatctccagagacaacgccaagaactctgtctatctgcaaatgaacagcctgagagccgaggacacggctctctattattgtgcgagagtcgggagtttttgtggtagtgacagctgcaatacgtggcggccctactggtacttcgatctctggggccgtggcacccaggtcactgtctcctccgcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 709 - DNA, 16 bp, synthetic construct
  source 1..16: mol_type = other DNA
ccttacggtgtctgat

SEQ ID NO: 710 - DNA, 340 bp, synthetic construct
  source 1..340: mol_type = other DNA
ggactcagtgaagggccgattcaccatctccagagacaacgccaagaactctgtctatctgcaaatgaacagcctgagagccgaggacacggctctctattattgtgcgagagtcgggagtttttgtggtagtgacagctgcaatacgtggcggccctactggtacttcgatctctggggccgtggcacccaggtcactgtctcctccgcctccacacagagcccatccgtcttccccttgacccgctgctgcaaaaacattccctccaatgccacctccgtgactctgggctgcctggccacgggctacttcccggagccggtgatggtgacctgggac

SEQ ID NO: 711 - DNA, 16 bp, synthetic construct
  source 1..16: mol_type = other DNA
gcctctaagatgccag

SEQ ID NO: 712 - DNA, 447 bp, synthetic construct
  source 1..447: mol_type = other DNA
ggattcacctttagtggtttttggatgacctgggtccgccaggctccggggaagggactggaatgtgtggctaacataaaccaagatggaagtgagaaataccatgtggactcagtgaagggccgattcaccatctccagagacaacgccaagaactctgtctatctgcaaatgaacagcctgagagccgaggacacggctctctattattgtgcgagagtcgggagtttttgtggtagtgacagctgcaatacgtggcggccctactggtacttcgatctctggggccgtggcacccaggtcactgtctcctccgcctccacacagagcccatccgtcttccccttgacccgctgctgcaaaaacattccctccaatgccacctccgtgactctgggctgcctggccacgggctacttcccggagccggtgatggtgacctgggac

SEQ ID NO: 713 - DNA, 16 bp, synthetic construct
  source 1..16: mol_type = other DNA
tgttccggtaacgttc

SEQ ID NO: 714 - DNA, 434 bp, synthetic construct
  source 1..434: mol_type = other DNA
ggtttttggatgacctgggtccgccaggctccggggaagggactggaatgtgtggctaacataaaccaagatggaagtgagaaataccatgtggactcagtgaagggccgattcaccatctccagagacaacgccaagaactctgtctatctgcaaatgaacagcctgagagccgaggacacggctctctattattgtgcgagagtcgggagtttttgtggtagtgacagctgcaatacgtggcggccctactggtacttcgatctctggggccgtggcacccaggtcactgtctcctccgcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 715 - DNA, 16 bp, synthetic construct
  source 1..16: mol_type = other DNA
tgcgtggagatccgag

SEQ ID NO: 716 - DNA, 353 bp, synthetic construct
  source 1..353: mol_type = other DNA
atcagtgcccgcaccggccgaacatattatgcagactccgtcaggggccggttcaccatctccagagacaactccaacagcactctgtctcttcagatgaacatcctgagggccgaggacacggccatttattactgtgcgaaaaactaccaacccacaagcatcttggtggactggttcgacccctggggccagggaaccctggtcaccgtctcctcagcctccacacagagcccatccgtcttccccttgacccgctgctgcaaaaacattccctccaatgccacctccgtgactctgggctgcctggccacgggctacttcccggagccggtgatggtgacctgggacac

SEQ ID NO: 717 - DNA, 515 bp, synthetic construct
  source 1..515: mol_type = other DNA
cgtctctgggggctggggtctcaggaggcagcgctctcgggacgtctccaccatggcctgggctctgctattcctcgccctcctcactcagggcacagggtcctgggcccagtctgccctgactcagcctgcctccgtgtctgggtctcctggacagtcgatcaccatctcctgcactgggaccagcggtgacgttggtggttacaaatatgtctcctggtatcaacaacacccaggcaaagcccccaaactcctcatttatgatgtcacttatcgaccctcaggggtttctgatcgcttctctggctccaagtctggcaacacggcctccctgaccatctctgggctccaggctgacgacgaagctgattattactgttgttcatatacaattagagacactctcgttttcgggactgggaccagggtcaccgtcctaggtcagcccaaggccaaccccactgtcactctgttcccgccctcctctgaggagctccaagccaacaaggccacac

SEQ ID NO: 718 - DNA, 556 bp, synthetic construct
  source 1..556: mol_type = other DNA
gagaagagctgctcagttaggacccagagggaaccatggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcacatacttagcctggtaccagcagaaacctggccaggctcccaggctcctcatctatgattcatccaccagggccactggcatcccagacaggttcagtggcagtgggtctgggacagacttcactctcaccatcagcagactggagcctgaagattttgcactgtattactgtcagcagtatggtagctcacctggcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 719 - DNA, 388 bp, synthetic construct
  source 1..388: mol_type = other DNA
atcacgaccggccctcagggatccctgagcgtttctctggctccaactctgggaacacggccaccctgaccatcagcagggtcgaagccggggatgaggccgattattactgccaggtgtggcataaccttagtgatgaagtggtgttcggcggagggaccaggttgaccgtccttggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 720 - DNA, 658 bp, synthetic construct
  source 1..658: mol_type = other DNA
gccttttcttatatgggggctggggtctccggaggcggcgctctcgggacgtctccaccatggcctgggctctgctattcctcgccctcctcactcagggcacagggtcctgggcccagtctgccctgactcagcctgcctccgtgtctgggtctcctggacagtcggtcactatctcctgttttggaagcagtggtgacgttggtgctttcaaatatgtctcctggtatcaacaatatccaggcaaagtccccagactcctcctttatgatgtcactaatcggccctcaggagtttctgatcgcttctctggctccaagtctggcaacacggcctccctgaccatctctgggctccaaactgacgacgaggctgactactactgttcctcatatacaattagcgacactgtcctgttcgggactgggacccaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagcta

SEQ ID NO: 721 - DNA, 628 bp, synthetic construct
  source 1..628: mol_type = other DNA
gagtctgggcctaaggaagcagcactggtggtgcctcagccatggcctggaccgttctcctcctcggcctcctctctcactgcacaggctctgtgacctccttcgtgctgactcagccaccctcggtgtcagtggccccaggaaagatggccacaatttcctgtgggggaaacaatcttggatataaaagtgtgcactggtaccggcagaaggcaggccaggcccctgtggtggtcctccatgatgttcgcgagcggccctcagggatccctgagcgattctctggctccaattctgggaacatagccactctgaccatcagcgggacccaggctatggatgaggctgactattactgtcaggtgtgggacagcagcactgtggtgttcggcggagggaccaaattgaccgtcctaggtcagcccaaggccaaccccactgtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 722 - DNA, 545 bp, synthetic construct
  source 1..545: mol_type = other DNA
tgggaggaactgctcagttaggacccagagggaaccatggaagccccagctcagcttctcttcctcctgctactctggctcccagatatcaccggagaaattgtgttgacacagtctccagccaccctgtctttgtctccagggcagagagccaccctcttctgcagggccagtcagagtgttggcagcaccctggcctggtatcagcagaaacctggccaggctcccaggctcgtcatctatgatgcatccaacaaggccactggcatcccagccaggttcagtggcagtgggtctgggacagacttcactctcaccatcagcagcctagagcctgaagattttgcagtttattactgtcagcagcgtgcgctcactttcggcggagggacgaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 723 - DNA, 445 bp, synthetic construct
  source 1..445: mol_type = other DNA
atgtgtactggtatcagcaattcccaggaacggccccccaactcctcatcgataagaatgatcaacggccctcaggggtccctgaccggttctctggctccaagtctggcacctcagcctccctgaccatcagtggactccggtccgaggatgaggctgattattactgtgcgatatggcatgacagtgagactggttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 724 - DNA, 641 bp, synthetic construct
  source 1..641: mol_type = other DNA
agctcagcttcagctgtgggtagagaagacaggactcaggacaatctccagcatggccagcttccctctcctcctcaccctcctcactcactgtgcagggtcctgggcccagtctgtgctgactcagccaccctcaacgtctgggacccccgggcagaaggtcaccatctcttgttctggaagcagttccaacatcggagggcctattgtcaactggtaccagcagatcccaggaacggcccccaaattaatcatctatactaatgatcagcggccctcaggggtccctgaccgattctctggctccaagtctggcacctcagcctccctggccatcagtggactccagtctgaggatgaggctgagtattactgtgcagcatgggatgacagactgaatggatgggtgttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcgg

SEQ ID NO: 725 - DNA, 647 bp, synthetic construct
  source 1..647: mol_type = other DNA
gagcttcagctgtgggtagagaagacaggactcaggacaatctccagcatggccagcttccctctcctcctcaccctcctcactcactgtgcagggtcctgggcccagtctgtgctgactcagccaccctcaacgtctgggacccccgggcagaaggtcaccatctcttgttctggaagcagttccaacatcggagggcctattgtcaactggtaccagcagatcccaggaacggcccccaaattaatcatctatactaatgatcagcggccctcaggggtccctgaccgattctctggctccaagtctggcacctcagcctccctggccatcagtggactccagtctgaggatgaggctgagtattactgtgcagcatgggatgacagactgaatggatgggtgttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 726 - DNA, 307 bp, synthetic construct
  source 1..307: mol_type = other DNA
catccagtttgagaattggggtcccatcaagattcagtggcagtggatttgggacagagttcactctcaccatcagcagcctgcagtctgaagattttatactttattactgtcaacagtatgataagtggttgattaccttcgggcaagggacacgactgcagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 727 - protein, 12 aa, synthetic construct
CCSYTIRDTLVF

SEQ ID NO: 728 - protein, 20 aa, synthetic construct
CAKGGDYVTVGYFYYSMAVW

SEQ ID NO: 729 - protein, 20 aa, synthetic construct
CAKGGDYVTVGYFYYSMDVW

SEQ ID NO: 730 - protein, 11 aa, synthetic construct [sequence omitted]

SEQ ID NO: 731 - protein, 13 aa, synthetic construct
CQVWHNLSDEVVF

SEQ ID NO: 732 - protein, 12 aa, synthetic construct
CSSYTISDTVLF

SEQ ID NO: 733 - protein, 11 aa, synthetic construct [sequence omitted]

SEQ ID NO: 734 - protein, 8 aa, synthetic construct [sequence omitted]

SEQ ID NO: 735 - protein, 13 aa, synthetic construct
CAIWHDSETGWVF

SEQ ID NO: 736 - protein, 24 aa, synthetic construct
CARSEGLQFAGRVLGTRKYDFDVW

SEQ ID NO: 737 - protein, 17 aa, synthetic construct
CAREWVVSPSTDAFDVW

SEQ ID NO: 738 - protein, 13 aa, synthetic construct [sequence omitted]

SEQ ID NO: 739 - protein, 25 aa, synthetic construct
CARVGSFCGSDSCNTWRPYWYFDLW

SEQ ID NO: 740 - protein, 11 aa, synthetic construct
CQQYDKWLITF

SEQ ID NO: 741 - protein, 18 aa, synthetic construct
CAKNYQPTSILVDWFDPW

SEQ ID NO: 742 - DNA, 36 bp, synthetic construct
  source 1..36: mol_type = other DNA
tgttgttcatatacaattagagacactctcgttttc

SEQ ID NO: 743 - DNA, 60 bp, synthetic construct
  source 1..60: mol_type = other DNA
tgtgcaaaaggcggcgactatgtcactgttggctacttctactactctatggccgtctgg

SEQ ID NO: 744 - DNA, 60 bp, synthetic construct
  source 1..60: mol_type = other DNA
tgtgcaaaaggcggcgactacgtcactgttggctacttctactactctatggatgtctgg

SEQ ID NO: 745 - DNA, 33 bp, synthetic construct
  source 1..33: mol_type = other DNA
tgtcagcagtatggtagctcacctggcactttc

SEQ ID NO: 746 - DNA, 39 bp, synthetic construct
  source 1..39: mol_type = other DNA
tgccaggtgtggcataaccttagtgatgaagtggtgttc

SEQ ID NO: 747 - DNA, 36 bp, synthetic construct
  source 1..36: mol_type = other DNA
tgttcctcatatacaattagcgacactgtcctgttc

SEQ ID NO: 748 - DNA, 33 bp, synthetic construct
  source 1..33: mol_type = other DNA
tgtcaggtgtgggacagcagcactgtggtgttc

SEQ ID NO: 749 - DNA, 24 bp, synthetic construct
  source 1..24: mol_type = other DNA
tgtcagcagcgtgcgctcactttc

SEQ ID NO: 750 - DNA, 39 bp, synthetic construct
  source 1..39: mol_type = other DNA
tgtgcgatatggcatgacagtgagactggttgggtgttc

SEQ ID NO: 751 - DNA, 72 bp, synthetic construct
  source 1..72: mol_type = other DNA
tgtgcgagatccgaaggactacaattcgcagggcgggtcctcggtacacgcaaatatgacttcgacgtctgg

SEQ ID NO: 752 - DNA, 51 bp, synthetic construct
  source 1..51: mol_type = other DNA
tgtgcgagagagtgggtggtttcaccctccactgatgcttttgatgtgtgg

SEQ ID NO: 753 - DNA, 39 bp, synthetic construct
  source 1..39: mol_type = other DNA
tgtgcagcatgggatgacagactgaatggatgggtgttc

SEQ ID NO: 754 - DNA, 75 bp, synthetic construct
  source 1..75: mol_type = other DNA
tgtgcgagagtcgggagtttttgtggtagtgacagctgcaatacgtggcggccctactggtacttcgatctctgg

SEQ ID NO: 755 - DNA, 33 bp, synthetic construct
  source 1..33: mol_type = other DNA
tgtcaacagtatgataagtggttgattaccttc

SEQ ID NO: 756 - DNA, 54 bp, synthetic construct
  source 1..54: mol_type = other DNA
tgtgcgaaaaactaccaacccacaagcatcttggtggactggttcgacccctgg

(19) United States
(12) Patent Application Publication
(10) Pub. No.: US 2023/0349919 A1
(43) Pub. Date: Nov. 2, 2023

(54) Methods Of Mapping Antigen Specificity To Antibody-Secreting Cells

(71) Applicant: Regeneron Pharmaceuticals, Inc., Tarrytown, NY (US)

(72) Inventors: Brian Klotz, Tarrytown, NY (US); Seblewongel Asrat, Tarrytown, NY (US); Marion Francis Setliff, Tarrytown, NY (US); Andrea Vecchione, Tarrytown, NY (US); Joseph Cooper Devlin, Tarrytown, NY (US); Wei Keat Lim, Tarrytown, NY (US); Samuel Davis, Tarrytown, NY (US); Hang Song, Tarrytown, NY (US); Jamie Orengo, Tarrytown, NY (US); Gurinder Atwal, Tarrytown, NY (US); Matthew Sleeman, Tarrytown, NY (US); Wen-yi Lee, Tarrytown, NY (US); Gang Chen, Tarrytown, NY (US); Kristel Velez, Tarrytown, NY (US)

(21) Appl. No.: 18/184,054

(22) Filed: Mar. 15, 2023

Related U.S. Application Data
(60) Provisional application No. 63/319,883, filed on Mar. 15, 2022.
(60) Provisional application No. 63/433,728, filed on Dec. 19, 2022.

(51) Int. Cl. G01N 33/68

(52) U.S. Cl. CPC G01N 33/6857

(57) ABSTRACT

The present disclosure provides antibody capture complexes and methods of capturing a target antibody secreted by an antibody secreting cell.

### REFERENCE TO SEQUENCE LISTING

This application includes a Sequence Listing filed electronically as an XML file named 381203732SEQ, created on Mar. 10, 2023, with a size of 824 kilobytes. The Sequence Listing is incorporated herein by reference.

### FIELD

The present disclosure is directed, in part, to antibody capture complexes and methods of mapping antigen specificity to antibody-secreting cells.

### BACKGROUND

B cells express the B cell receptor (BCR) on their surface, thereby allowing the determination of an antigen-specific B cell repertoire profiling. While multiple platforms are available for antibody discovery from B cells expressing cell-surface BCR, the antibodies isolated vary from low to high affinity. Following activation by cognate antigen, B cells undergo fine-tuning of their BCRs and may ultimately differentiate into antibody-secreting cells (ASCs). Antibody secreting cells are a specialized cell type that represents the end-stage of the B-cell differentiation program and comprise plasmablasts, short-lived plasma cells, and long-lived plasma cells (or, more commonly, simply “plasma cells” (PCs); Tellier and Nutt, Eur. J. Immunol., 2019, 49, 30-37). These ASCs can produce high-affinity antibodies with therapeutic or prophylactic potential. However, ASCs can also be the source of antibody-mediated pathologies.

While antigen-specific antibodies from B cells expressing BCRs on their cell surface can be readily cloned and sequenced following flow cytometric isolation, PCs, which are ASCs that do not express cell-surface BCRs, cannot currently be easily profiled in a high-throughput way. And while PCs may be the source of very high-affinity antibody, the specificity of any given secreted antibody cannot rapidly be determined. Because there are limitations as to the application of current platforms to PCs, there is a long-felt but unsolved need to efficiently pair a given antibody with the BCR⁻ PC that secreted it as well as to efficiently determine the antigen specificity of that antibody.

### SUMMARY

The present disclosure provides an antibody capture complex comprising a first component of a binding pair linked to an antibody-capture molecule; wherein the first component of the binding pair is capable of binding a second component of the binding pair; wherein the antibody-capture molecule is capable of binding to a target antibody, wherein the target antibody is secreted by an antibody secreting cell. In some embodiments, when the second component of the binding pair is biotin and the first component of the binding pair is streptavidin, the antibody-capture molecule does not bind to a kappa light chain of the target antibody.

The present disclosure also provides methods of capturing a target antibody secreted by an antibody secreting cell, the methods comprising: contacting a population of antibody secreting cells with a second component of a binding pair to allow the second component of the binding pair to bind to the cell surface of the population of antibody secreting cells, wherein the population of antibody secreting cells comprises the antibody secreting cell that secretes the target antibody; contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the antibody secreting cells, and whereby the antibody-capture molecule captures the target antibody secreted by the antibody secreting cell; and contacting the population of antibody secreting cells with an antigen, whereby the target antibody secreted by the antibody secreting cell and captured by the antibody capture complex binds the antigen.

The present disclosure also provides methods of capturing a target antibody secreted by an antibody secreting cell, the methods comprising: contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell; and contacting the population of antibody secreting cells with an antigen, whereby the target antibody secreted by the antibody secreting cell and captured by the antibody capture complex binds the antigen.

The present disclosure also provides methods of capturing an IgE antibody secreted by an antibody secreting cell, wherein the IgE antibody is directed to an allergen, the methods comprising: contacting a population of antibody secreting cells with NHS-biotin to allow biotin to bind to the cell surface, wherein the population of antibody secreting cells comprises the antibody secreting cell that secretes the IgE antibody; contacting the population of antibody secreting cells with an antibody capture complex, wherein the antibody capture complex comprises streptavidin linked to an ectodomain of a high affinity IgE receptor (FcεRIa), whereby the antibody-capture molecule binds to the IgE antibody secreted by the antibody secreting cell; and contacting the population of antibody secreting cells with an antigen, wherein the antigen is an antigenic portion or a mixture of a plurality of antigenic portions of the allergen, whereby the IgE antibody captured by the antibody capture complex binds to the antigen.

In some embodiments of the methods disclosed herein, the methods further comprise, after contacting the population of antibody secreting cells with an antigen, sorting the antibody secreting cells to collect a pool of antibody secreting cells, wherein the antibody secreting cells in the pool each secrete an antibody that is captured by the antibody capture molecule and is bound by the antigen, wherein the collected pool of antibody secreting cells comprises the antibody secreting cell that secretes the target antibody. In some embodiments of the methods disclosed herein, the methods further comprise separating the collected pool of antibody secreting cells into single cells and isolating the antibody secreting cell that secretes the target antibody. In some embodiments, antibody-encoding nucleic acids are isolated from the antibody secreting cell that secretes the target antibody.

In some embodiments of the methods disclosed herein, the step of contacting the population of antibody secreting cells with an antigen comprises: (a) contacting the population of antibody secreting cells with a first labeled form of the antigen to allow the antigen to bind to antibodies captured at the cell surface of the population of antibody secreting cells including the target antibody captured on the cell surface of the antibody secreting cell, wherein the antigen of the first labeled form is conjugated to a first detectable label; (b) washing the population of antibody secreting cells to remove unbound antigen; (c) contacting the population of antibody secreting cells with (i) an unlabeled form of the antigen, (ii) a second labeled form of the antigen, or (iii) the unlabeled form of the antigen and the second labeled form of the antigen; (d) washing the population of antibody secreting cells to remove unbound antigen, and (e) collecting a pool of antibody secreting cells remaining bound to the first labeled form of the antigen, wherein the collected pool of antibody secreting cells comprises the antibody secreting cell that secretes the target antibody, and wherein the target antibody secreted by the antibody secreting cell and captured by the antibody capture complex remains bound to the first labeled form of the antigen.

In some embodiments of the methods disclosed herein, after collecting a pool of antibody secreting cells remaining bound to the first labeled form of the antigen, the methods further comprise separating the collected pool of antibody secreting cells into single cells and isolating the antibody secreting cell that secretes the target antibody. In some embodiments, antibody-encoding nucleic acids are isolated from the antibody secreting cell that secretes the target antibody.

The present disclosure also provides methods of identifying a region of a gene encoding an antigen-binding fragment of a target antibody wherein the target antibody is secreted by a cell comprising a modified cell surface, and wherein the target antibody is subsequently captured on the modified cell surface, the methods comprising: a) providing an antibody secreting cell comprising a modified cell surface, wherein the antibody secreting cell secretes a target antibody, wherein the target antibody is subsequently captured on the modified cell surface, wherein the target antibody captured on the modified cell surface is bound to an antigen, wherein the antigen is linked to a barcode nucleic acid molecule, and wherein the antibody secreting cell further comprises a nucleic acid molecule comprising the region of the gene encoding the antigen-binding fragment of the target antibody captured on the modified cell surface; b) hybridizing a portion of the barcode nucleic acid molecule to a portion of a first nucleic acid molecule attached to a solid surface; c) hybridizing a portion of the nucleic acid molecule comprising the region of the gene encoding the antigen-binding fragment of the target antibody captured on the modified cell surface to a portion of a second nucleic acid molecule attached to the solid surface; d) preparing a first library of amplicons of gene expression in the antibody secreting cell, a second library of amplicons of variable regions, diversity regions, and joining regions (VDJ) in the antibody secreting cell, and a third library of amplicons of antigen barcode nucleic acid molecules; e) sequencing each of the three libraries of amplicons; and f) identifying the region of the gene encoding the antigen-binding fragment of the target antibody captured on the modified cell surface.

The present disclosure also provides methods of identifying a region of a gene encoding an antigen-binding fragment of a target antibody, wherein the target antibody is secreted by an antibody secreting cell, the methods comprising: a) contacting a population of antibody secreting cells with a second component of a binding pair to allow the second component of the binding pair to bind to the cell surface, wherein the population of antibody secreting cells comprises the antibody secreting cell that secretes the target antibody; b) contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the population of antibody secreting cells, and whereby the antibody-capture molecule captures the target antibody secreted by the antibody secreting cell; c) contacting the population of antibody secreting cells with a secondary anti-Ig antibody, whereby the target antibody secreted by the antibody secreting cell and captured by the antibody capture complex binds the secondary anti-Ig antibody; d) contacting the population of antibody secreting cells with an antigen comprising a barcode nucleic acid molecule, whereby the target antibody secreted by the antibody secreting cell and captured by the antibody capture complex binds the antigen; e) collecting a pool of antibody secreting cells, wherein each cell in the pool secretes an antibody that is captured by the antibody capture complex, bound by the secondary anti-Ig antibody and by the antigen comprising the barcode nucleic acid molecule, and wherein the pool of antibody secreting cells comprises the antibody secreting cell that secretes the target antibody; f) separating the pool of antibody secreting cells into single antibody secreting cells, and for each single antibody secreting cell: 1) hybridizing a portion of the barcode nucleic acid molecule to a portion of a first nucleic acid molecule attached to a solid surface, wherein the solid surface and the first nucleic acid molecule attached thereto are unique to each single antibody secreting cell; 2) hybridizing a portion of the nucleic acid molecule comprising the region of the gene encoding the antigen-binding fragment of the target antibody to a portion of a second nucleic acid molecule attached to the solid surface; 3) preparing a first library of amplicons of gene expression, a second library of amplicons of variable regions, diversity regions, and joining regions (VDJ), and a third library of amplicons of antigen barcode nucleic acid molecules; and 4) sequencing each of the three libraries of amplicons; and g) identifying the region of the gene encoding the antigen-binding fragment of the target antibody.

The present disclosure also provides methods of identifying a region of a gene encoding an antigen-binding fragment of a target antibody, wherein the target antibody is secreted by an antibody secreting cell, the methods comprising: a) contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell; b) contacting the population of antibody secreting cells with a secondary anti-Ig antibody, whereby the target antibody secreted by the antibody secreting cell and captured by the antibody capture complex binds the secondary anti-Ig antibody; c) contacting the population of antibody secreting cells with an antigen comprising a barcode nucleic acid molecule, whereby the target antibody secreted by the antibody secreting cell and captured by the antibody capture complex binds the antigen; d) collecting a pool of antibody secreting cells, wherein each cell in the pool secretes an antibody that is captured by the antibody capture complex, bound by the secondary anti-Ig antibody and by the antigen comprising the barcode nucleic acid molecule, and wherein the pool of antibody secreting cells comprises the antibody secreting cell that secretes the target antibody; e) separating the pool of antibody secreting cells into single antibody secreting cells, and for each single antibody secreting cell: 1) hybridizing a portion of the barcode nucleic acid molecule to a portion of a first nucleic acid molecule attached to a solid surface, wherein the solid surface and the first nucleic acid molecule attached thereto are unique to each single antibody secreting cell; 2) hybridizing a portion of the nucleic acid molecule encoding the region of the gene encoding the antigen-binding fragment of the target antibody to a portion of a second nucleic acid molecule attached to the solid surface; 3) preparing a first library of amplicons of gene expression, a second library of amplicons of variable regions, diversity regions, and joining regions (VDJ), and a third library of amplicons of antigen barcode nucleic acid molecules; and 4) sequencing each of the three libraries of amplicons; and f) identifying the region of the gene encoding the antigen-binding fragment of the target antibody.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a representative workflow of an Ig secretion capture method. Step 1 shows the biotinylation of cells with N-hydroxysuccinimido biotin (NHS-biotin). Step 2 shows the Ig secretion-capture reagent comprising streptavidin coupled with anti-Fc. αlgK was used for antibodies that express kappa light chain or FcεRIα ecto-domain for specific IgE capture. Step 3 shows a secreted αlgE or αlgG antibody bound to an oligo-barcoded and fluorescently-tagged antigen bound by an anti-IgG or anti-IgE antibody.

FIG. 2 shows a representative workflow of an IgE secretion capture method. Step 1 shows the biotinylation of a PC using NHS-biotin. Step 2 shows a reagent comprising the ectodomain of the high-affinity IgE receptor (ectoFcεRIα) conjugated to streptavidin (the “secreted IgE capture reagent”) and the streptavidin bound to biotin on the PC. Step 2 also shows the ectoFcεRIα of the secreted IgE capture reagent capturing an IgE that the PC has secreted. Step 3 shows the captured IgE bound to a barcoded antigen and bound by an anti-IgE antibody.

FIG. 3 shows a representative workflow of an IgG secretion capture method. Step 1 shows the biotinylation of cells with NHS-biotin. Step 2 shows the IgG secretion-capture reagent comprising streptavidin-conjugated anti-mouse IgK assembled on the biotinylated cells. Step 3 shows a secreted IgG antibody bound to a barcoded antigen and bound by an anti-IgG antibody.

FIGS. 4A-4F show, in FIG. 4A, the detection of secreted IgG captured on the surface of ARH77 cells. FIG. 4B shows the detection of secreted IgE captured on the surface of U266 cells. FIG. 4C shows that, while secreted IgE captured on the surface of U266 cells was detected, surface IgE as a B-cell receptor was not. FIG. 4D shows a titration of streptavidin-αlgk (StAv-αlgk) on ARH77 cells. FIG. 4E shows a titration of streptavidin-FceRlα (StAv-FcεRIα) on U266 cells. FIG. 4F shows high specificity of detection of secreted IgE captured on the surface of U266 cells but not the IgM secreted by Ramos cells.

FIGS. 5A-5G show, in FIG. 5A, a representative workflow of a procedure to determine the sequences of V(D)J regions of an antibody specific for a barcoded antigen and that of the barcode followed by confirmation of antigen specificity of the antibody by an enzyme-linked immunosorbent assay (ELISA). FIG. 5B shows a flow cytometry of antigen-specific IgG captured on the surface of PCs in hIL-4Rα immunized mice. FIG. 5C shows a visualization of antigen specificity based on a barcode signal of hIL-6Rα (non-specific) and hIL-4Rα (specific) in bone marrow (BM) PCs and spleen draining lymph node (dLN) B cells and PCs. FIG. 5D shows a UMAP of B cells and PCs isolated from spleen/dLN and bone marrow of challenged mice. FIG. 5E shows a graph indicating the ELISA antibody levels of total IgG, anti-hIL-4Rα and the ratio of anti-hIL-4Rα to total IgG in BM-PCs, SP-PCs and SP-B cells. FIG. 5F and FIG. 5G show graphs from two separate experiments illustrating the binding affinity of antibodies cloned from BM-PCs, SP-PCs and SP-B cells.

FIGS. 6A-6F show, in FIG. 6A, a graph illustrating the percent similarity to the germline V region between hIL-4Rα-specific and non-specific cells isolated from BM-PCs, spleen/dLN-PCs, and spleen/dLN-B cells from challenged mice. FIG. 6B shows a Venn diagram illustrating clonal overlap based on CDR3 nucleotide sequences from heavy and light chains of clones isolated from BM-PCs, spleen/dLN-PCs, and spleen/dLN-B cells. FIG. 6C shows a Volcano plot comparison of differential gene expression between hIL-4Rα-specific and non-antigen specific BM-PCs. FIG. 6D shows a heatmap illustrating the transcriptional profile of differentially expressed genes expressed in hIL-4Rα-specific and non-antigen specific BM-PCs. FIG. 6E shows a UMAP illustrating the location and binding affinity (Kd) of hIL-4Rα-specific antibodies from BM-PCs on total BM-PCs. FIG. 6F shows a UMAP illustrating scaled gene expression of Cd24a, Ppob, Fkbp11, Ssr2, Tmem176a, Tmem176b, Ly6d and CD74 in BM-PCs.

FIGS. 7A-7H show, in FIG. 7A, a representative workflow of a procedure to determine the sequences of V(D)J regions of an antibody specific for a barcoded antigen and that of the barcode followed by confirmation of antigen specificity of the antibody by an enzyme-linked immunosorbent assay (ELISA). FIG. 7B shows from left to right, the gating of PCs as cells, single cells, live cells, DUMP⁻ cells, and CD138⁺Blimp-1⁺ cells. FIG. 7C shows IgE-secreting, IgE^{Venus+} PCs derived from the BM and LDLNs of mice that had been challenged with house dust mite (HDM) extract. FIG. 7D shows a UMAP of IgE PCs based on VDJ-BCR expression over non-IgE PCs. FIG. 7E shows the fraction of IgE, IgG, IgA and IgM PCs and mean expression of the top 4 genes expressed in different isotype from both dLN and BM. FIG. 7F shows a visualization of antigen specificity based on barcode expression of Der p1, Der p2, Der f1 versus the negative control Ole e1. FIG. 7G shows representative binding curves of IgE antibodies to Der p 1, Der p 2, Der f 1 and to the negative control antigen Ole e 1. FIG. 7H shows antigen specificity validated by ELISA for a subset of IgE antibodies generated from IgE-secreting cells.

FIGS. 8A-8G show, in FIG. 8A, flow plots of BM from an allergic bone marrow donor, BM from a non-allergic bone marrow control donor and a non-streptavidin FcεR1α capture control. FIG. 8B shows the results of a test performed on serum to detect levels of IgE specific to cat dander e1, dog dander e5, common silver birch t3, dermatoph pteronyssinus d1, timothy g6, and alternaria alternaria m6. FIG. 8C shows an expression dot plot illustrating scaled expression of immunoglobulin genes, and PC and B cell markers within total PCs. FIG. 8D shows a graph illustrating scaled expression of IGHE transcript in IgE, IgG, IgA and IgM PCs. FIG. 8E shows a table of clustered BCR clonotypes indicating 5 IgE clonotypes, their clone size, and the number of IgE versus non-IgE cells within each clonotype. FIG. 8F shows an alignment plot providing a comparison between IgE sequence from an IgE PC to IgG sequences in clonotype 21. FIG. 8G shows antigen specificity tested by ELISA for one human IgE antibody isolated by the IgE capture complex of the disclosure from an allergic BM. Representative binding curves of human IgE antibody to Fel d1, Can e1, Der p1 and DNP-IgE are shown.

FIG. 9 shows single cell capture as well as library preparation for mRNA, V(D)J regions, and antigen barcode. On the lower row of FIG. 9: (i) the light-blue rectangles represent template switch oligos; (ii) the beige rectangles represent sequences of three contiguous riboguanosine residues; (iii) the orange rectangles represent unique molecular identifiers (UMIs); (iv) the purple rectangles represent surface barcodes; and (iv) the black squares immediately to the right of the purple rectangles represent sequencing primers.

FIG. 10 shows a representative flowchart of the analysis of single-cell mRNA, V(D)J region, and antigen barcode libraries.

FIG. 11 shows a visualization of antigen specificity. Cells with higher antigen specificity scores, i.e., a strong signal for target antigens and a low signal for control antigens, were prioritized to select antibody candidates. The target antigens are the HDM allergens Der p 1, Der p 2, and Der f 1 and the negative control was olive antigen Ole e 1. The antigen specificity score for each target antigen was determined by subtracting the quantile rank of the control antigen (qC) raised to the x-th power from the quantile rank of the target antigen (qT), i.e., qT−qC^{x}. Cells with the highest antigen specificity score were selected as antigen specific candidates.

FIG. 12 shows a table of IgE candidates based on antigen specificity score. For each candidate the table records the sample from which the cell was obtained, the raw antigen barcode counts for each of the target antigens, the normalized antigen barcode counts and the antigen specificity score. Data were acquired for 1,069 IgE⁺ cells.

FIGS. 13A-13B show the binding of IgE antibodies derived from PC-secreted IgE captured with the secreted IgE capture reagent where the PCs were derived from mice challenged with HDM extract. FIG. 13A shows representative binding curves of IgE antibodies to Der p 1 (left-hand chart), Der p 2 (middle chart), and Der f 1 (right-hand chart). FIG. 13B shows representative binding curves of IgE antibodies to the negative control antigen Ole e 1.

FIGS. 14A-14C show that antigen-specific, secreted IgG can be captured on the surfaces of PCs derived from challenged VelocImmune® mice. FIG. 14A shows a successive flow cytometric gating strategy to detect antigen-specific IgG splenic B cells. FIG. 14B shows a successive flow cytometric gating strategy to identify BM-derived PCs upon which antigen-specific, secreted IgG had been captured. FIG. 14C shows, from left to right, four controls used to accurately define antigen-specific PC populations derived from the BM, and a splenic positive control.

FIGS. 15A-15D show the normalized value of a target antigen barcode counts on the y axis against the normalized barcode counts of a control antigen barcode counts in all PCs derived from immunized mice and colored by isotype. FIG. 15A and FIG. 15B show data for cells from the bone marrow while FIG. 15C and FIG. 15D show data for splenic cells.

FIGS. 16A-16C show, in FIG. 16A, a flow cytometry plot of ARH77 cells stained with αlgG. FIG. 16B shows a flow cytometry plot of U266 cells stained with αlgE. FIG. 16C shows a flow cytometry analysis of Ramos, U266 or a 100:1 mix of these cells in the presence of an embodiment of the StAv-FceRlα capture complex.

FIGS. 17A-17D show, in FIG. 17A, the immunization regimen mice underwent prior to tissue collection for downstream antibody specificity mapping through antibody secretion TRAP and Sequencing work. FIG. 17B shows IgG-secreting PCs gated as live cells, lymphocytes, single cells, DUMP⁻ cells (TCRβ, CD200R3, Ly6G, CD49b, CD11b, and IgM), CD98+TACI+, and IgG+Ag+ cells. FIG. 17C shows Ag+IgG+ cells from spleen and dLN gated as live cells, lymphocytes, single cells, CD19+CD3−, IgD−IgM−, and IgG+Ag+ cells. FIG. 17D shows gating controls to identify Ag+IgG+BMPCs.

FIGS. 18A-18D show, in FIG. 18A, a combined transcriptional analysis and unbiased clustering analysis of spleen plasma cells, BM plasma cells and splenic B cells. FIG. 18B and FIG. 18C show the three distinct cell populations yielded by scRNA-seq: bone marrow plasma cells (BMPCs), spleen/dLN plasmablast/plasma cell-like (Sp/dLN PBs/PCs), and spleen/dLN B cells (Sp/dLN B cells). FIG. 18D shows a Biacore analysis of supernatants and purified antibodies of the disclosure showing the affinity of the BMPCs, Sp/dLN PBs/PCs and Sp/dLN B cells as measured by negative log 10 of the binding Kds.

FIGS. 19A-19B show, in FIG. 19A, representative binding curves of IgE antibodies to Der p1, Der p2, Der f1 and to the negative control Ole e1. FIG. 19B shows an Fel d1 specific ELISA to test binding of a human IgE mAb 12_2 and its cross-reactivity to Fel d1.

FIGS. 20A-20B show, in FIG. 20A, a gating strategy to sort plasma cells from human bone marrow using CD38 staining. FIG. 20B shows the results of an exemplary serum ImmunoCAP® test for an allergic donor.

FIGS. 21A-21D show an antibody secretion TRAP and sequencing methodology disclosed herein allows detection of vaccine-specific IgG-secreting plasma cells from bone marrow donor. FIG. 21A, a representative workflow of a procedure to determine the gating of PCs as cells, single cells, live cells, DUMP⁻ cells, and CD20-CD38++ cells. FIG. 21B shows IgG-secreting and antigen-positive cells found within IgG-secreting cells. FIG. 21C shows lack of detection of IgG-secreting PCs without NHS-biotin component, or StAv-Igkappa, or anti-IgG antibody. FIG. 21D shows lack of detection of antigen-specific IgG-secreting cells without antigen staining.

FIG. 22 depicts the workflow of secretion trap combined with antigen chase (STAC). Step 1: antibody secreting cells were biotinylated with amine-reactive NHS-biotin. Step 2: Secreted antibodies were captured on the surface of antibody secreting cells using streptavidin coupled with anti-hlgG. Anti-hlgG was used here since all antibodies have human constants. Step 3: Cells were incubated with monomeric hIL-4Rα conjugated to AlexaFluor647 for 30 min at RT. Cells were washed and then incubated 2×45 min at RT with hIL-4Rα antigen pre-incubated with StAv-PE.

FIGS. 23A-23D show flow cytometry analysis of cells undergoing the STAC protocol. FIG. 23A shows a table for antibodies used in experiments. Antibodies 1-3 are specific for hIL-4Rα with differing affinities (KD), and antibody 4 is specific to a different target and serves as a negative control for the experiment. FIG. 23B shows a model B cell line with no surface BCR (A25 cell line) was incubated with NHS-biotin and an anti-hlgG trap reagent (Step 1 and 2 of FIG. 22). These cells were then incubated with antibodies from the table in FIG. 23A followed by the antigen chase incubations (Step 3 of FIG. 22). FIG. 23C shows flow cytometry analysis of cells undergoing STAC protocol shows that antibody secreting cells can be separated based on affinity. Antibodies 1-4 are color coded on the flow cytometry plots according to the colors in FIG. 23A. The higher affinity antibodies (ab_1, ab_2) skew towards the monomeric hIL-4Rα-AlexaFluor647 (y-axis), the lower affinity antibody (ab_3) skews toward the tetrameric hIL-4Rα-biotin-StAv-PE (x-axis), and the negative control antibody (ab_4) is negative for both antigens. FIG. 23D shows flow Cytometry analysis of cells undergoing STAC protocol. All cells are hlgK+ indicating all antibodies, including the negative control (ab_4), are bound to the surface by the secretion trap. An anti-hlgK FMO is shown in grey, all other colors coordinate with the colors in FIG. 23A.

### DESCRIPTION OF EMBODIMENTS

The terminology used herein is for the purpose of describing particular embodiments only and is not intended to be limiting.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “antibody” can comprise an intact immunoglobulin molecule, a fragmented immunoglobulin molecule comprising an antigen-binding fragment, an antigen-binding fragment without any other fragment of an immunoglobulin molecule.

The present disclosure provides antibody capture complexes comprising a first component of a binding pair linked to an antibody-capture molecule; wherein the first component of the binding pair is capable of binding a second component of the binding pair; wherein the antibody-capture molecule is capable of binding to a target antibody, wherein the target antibody is secreted by an antibody secreting cell. In some embodiments, when the second component of the binding pair is biotin and the first component of the binding pair is streptavidin, the antibody-capture molecule does not bind to a kappa light chain of the target antibody.

In some embodiments, a first component of a binding pair comprises a first reagent and a second component comprises a second reagent, wherein the first reagent and the second reagent form a bond.

In some embodiments, the first component of the binding pair comprises avidin, streptavidin, or anti-biotin, and the second component of the binding pair comprises biotin. In some embodiments, the first component of the binding pair comprises biotin and the second component of the binding pair comprises avidin and/or streptavidin. In some embodiments, the first component of the binding pair comprises biotin and the second component of the binding pair comprises anti-biotin. In some embodiments, the first component of the binding pair comprises one of jun and fos and the second component of the binding pair comprises the other of jun and fos. In some embodiments, the first component of the binding pair comprises one of mad and max and the second component of the binding pair comprises the other of mad and max. In some embodiments, the first component of the binding pair comprises one of myc and max and the second component of the binding pair comprises the other of myc and max. In some embodiments, the first component of the binding pair comprises one of an azide and an alkyne and the second component of the binding pair comprises the other of an azide and an alkyne. In some embodiments, the first component of the binding pair comprises one of an azide and a phosphine and the second component of the binding pair comprises the other of an azide and a phosphine.

In some embodiments, the second component of the binding pair comprises a surface marker of the antibody secreting cell and the first component of the binding pair comprises an antibody that binds to the surface marker. In some embodiments, the cell surface marker comprises CD27, CD38, CD45, CD138, CD98, CD78, CD319, CXCR4, BCMA, GPRC5D, FCRL5, CD19, Ly6D, CD52, or transmembrane activator calcium modulator and cyclophilin ligand interactor (TACI). In some embodiments, the cell surface marker comprises CD27. In some embodiments, the cell surface marker comprises CD38. In some embodiments, the cell surface marker comprises CD45. In some embodiments, the cell surface marker comprises CD138. In some embodiments, the cell surface marker comprises CD98. In some embodiments, the cell surface marker comprises CD78. In some embodiments, the cell surface marker comprises CD319. In some embodiments, the cell surface marker comprises CXCR4. In some embodiments, the cell surface marker comprises BCMA. In some embodiments, the cell surface marker comprises GPRC5D. In some embodiments, the cell surface marker comprises FCRL5. In some embodiments, the cell surface marker comprises CD19. In some embodiments, the cell surface marker comprises Ly6D. In some embodiments, the cell surface marker comprises CD52. In some embodiments, the cell surface marker comprises TACI.

In some embodiments, the second component of the binding pair comprises an interleukin-6 receptor (IL-6R), and the first component of the binding pair comprises an anti-IL-6R antibody, interleukin-6 (IL-6), and/or a fragment of IL-6 capable of binding to IL-6R. In some embodiments, the second component of the binding pair comprises CD27, and the first component of the binding pair comprises an anti-CD27 antibody, CD70, and/or a fragment of CD70 capable of binding to CD27.

In some embodiments, the second component of the binding pair comprises fluorescein isothiocyanate (FITC) and the first component of the binding pair comprises an anti-FITC antibody. In some embodiments, the second component of the binding pair comprises fluorescein and the first component of the binding pair comprises an anti-fluorescein antibody. In some embodiments, the second component of the binding pair comprises phycoerythrin (PE), and the first component of the binding pair comprises an anti-PE antibody. In some embodiments, the second component of the binding pair comprises APC, and the first component of the binding pair comprises an anti-APC antibody. In some embodiments, the second component of the binding pair comprises BV421, and the first component of the binding pair comprises an anti-BV421 antibody. In some embodiments, the second component of the binding pair comprises BV510, and the first component of the binding pair comprises an anti-BV510 antibody. In some embodiments, the second component of the binding pair comprises BV605, and the first component of the binding pair comprises an anti-BV605 antibody. In some embodiments, the second component of the binding pair comprises BV650, and the first component of the binding pair comprises an anti-BV650 antibody. In some embodiments, the second component of the binding pair comprises BV711, and the first component of the binding pair comprises an anti-BV711 antibody. In some embodiments, the second component of the binding pair comprises BV786, and the first component of the binding pair comprises an anti-BV786 antibody.

In some embodiments, the antibody-capture molecule comprises a capture antibody.

In some embodiments, the capture antibody comprises an anti-Fc antibody. In some embodiments, the capture antibody comprises an anti-Fcγ antibody, an anti-Fcα antibody, or an anti-Fcε antibody. In some embodiments, the capture antibody comprises an anti-Fcγ antibody. In some embodiments, the capture antibody comprises an anti-Fcα antibody. In some embodiments, the capture antibody comprises an anti-Fcε antibody. In some embodiments, the anti-Fcγ antibody comprises an anti-FcγRI antibody, an anti-FcγRIIA antibody, an anti-FcγRIIB antibody, an anti-FcγRIIB1 antibody, an anti-FcγRIIB2 antibody, an anti-FcγRIIIA antibody, and/or an anti-FcγRIIIB antibody. In some embodiments, the anti-Fcγ antibody comprises an anti-FcγRI antibody. In some embodiments, the anti-Fcγ antibody comprises an anti-FcγRIIA antibody. In some embodiments, the anti-Fcγ antibody comprises an anti-FcγRIIB antibody. In some embodiments, the anti-Fcγ antibody comprises an anti-FcγRIIB1 antibody. In some embodiments, the anti-Fcγ antibody comprises an anti-FcγRIIB2 antibody. In some embodiments, the anti-Fcγ antibody comprises an anti-FcγRIIIA antibody. In some embodiments, the anti-Fcγ antibody comprises an anti-FcγRIIIB antibody. In some embodiments, the anti-Fcα antibody comprises an anti-FcαRI antibody. In some embodiments, the anti-Fcε antibody comprises an anti-FcεRI antibody or an anti-FcεRIII antibody. In some embodiments, the anti-Fcε antibody comprises an anti-FcεRI antibody. In some embodiments, the anti-Fcε antibody comprises an anti-FcεRII antibody.

In some embodiments, the capture antibody comprises an anti-light chain kappa antibody and/or an anti-light chain lambda antibody. In some embodiments, the capture antibody comprises an anti-light chain kappa antibody. In some embodiments, the capture antibody comprises an anti-light chain lambda antibody.

In some embodiments, the capture antibody comprises an anti-Ig antibody.

In some embodiments, the capture antibody comprises an anti-IgM antibody.

In some embodiments, the capture antibody comprises an anti-IgG antibody. In some embodiments, the anti-IgG antibody comprises an anti-IgG1 antibody, an anti-IgG2 antibody, an anti-IgG2a antibody, an anti-IgG2b antibody, an anti-IgG3 antibody, and/or an anti-IgG4 antibody. In some embodiments, the anti-IgG antibody comprises an anti-IgG1 antibody. In some embodiments, the anti-IgG antibody comprises an anti-IgG2 antibody. In some embodiments, the anti-IgG antibody comprises an anti-IgG2a antibody. In some embodiments, the anti-IgG antibody comprises an anti-IgG2b antibody. In some embodiments, the anti-IgG antibody comprises an anti-IgG3 antibody. In some embodiments, the anti-IgG antibody comprises an anti-IgG4 antibody.

In some embodiments, the capture antibody comprises an anti-IgA antibody. In some embodiments, the anti-IgA antibody comprises an anti-IgA1 antibody, an anti-IgA2 antibody, an anti-secretory IgA antibody, or a polymeric anti-IgA antibody. In some embodiments, the anti-IgA antibody comprises an anti-IgA1 antibody. In some embodiments, the anti-IgA antibody comprises an anti-IgA2 antibody. In some embodiments, the anti-IgA antibody comprises an anti-secretory IgA antibody. In some embodiments, the anti-IgA antibody comprises a polymeric anti-IgA antibody.

In some embodiments, the capture antibody comprises an anti-IgE antibody.

In some embodiments, the antibody-capture molecule comprises an Fc receptor or an ectodomain of the Fc receptor. In some embodiments, the Fc receptor comprises an Fcγ receptor, an Fcα receptor, and/or an Fcε receptor. In some embodiments, the Fc receptor comprises an Fcγ receptor. In some embodiments, the Fc receptor comprises an Fcα receptor. In some embodiments, the Fc receptor comprises an Fcε receptor. In some embodiments, the Fcγ receptor comprises an FcγRI receptor, an FcγRIIA receptor, an FcγRIIB receptor, an FcγRIIB1 receptor, an FcγRIIB2 receptor, an FcγRIIIA receptor, an FcγRIIIB receptor, and/or an FcRn receptor. In some embodiments, the Fcγ receptor comprises an FcγRI receptor. In some embodiments, the Fcγ receptor comprises an FcγRIIA receptor. In some embodiments, the Fcγ receptor comprises an FcγRIIB receptor. In some embodiments, the Fcγ receptor comprises an FcγRIIB1 receptor. In some embodiments, the Fcγ receptor comprises an FcγRIIB2 receptor. In some embodiments, the Fcγ receptor comprises an FcγRIIIA receptor. In some embodiments, the Fcγ receptor comprises an FcγRIIIB receptor. In some embodiments, the Fcγ receptor comprises an FcRn receptor. In some embodiments, the Fcα receptor comprises an FcαRI receptor and/or an Fcα/μR receptor. In some embodiments, the Fcα receptor comprises an FcαRI receptor. In some embodiments, the Fcα receptor comprises an Fcα/μR receptor. In some embodiments, the Fcε receptor comprises an FcεRI receptor and/or an FcεRII receptor. In some embodiments, the Fcε receptor comprises an FcεRI receptor. In some embodiments, the Fcε receptor comprises an FcεRII receptor. In some embodiments, the Fc receptor comprises a fragment of an Fc receptor capable of binding Fc.

In some embodiments, the antibody-capture molecule comprises protein A, a fragment of protein A capable of binding to a portion of an Fc region, protein G, and/or a fragment of protein G capable of binding to a portion of an Fc region. In some embodiments, the antibody-capture molecule comprises protein A. In some embodiments, the antibody-capture molecule comprises a fragment of protein A capable of binding to a portion of an Fc region. In some embodiments, the antibody-capture molecule comprises protein G. In some embodiments, the antibody-capture molecule comprises a fragment of protein G capable of binding to a portion of an Fc region. In some embodiments, the antibody-capture molecule comprises protein L and/or a fragment of protein L capable of binding to a portion of a light chain. In some embodiments, the antibody-capture molecule comprises protein L. In some embodiments, the antibody-capture molecule comprises a fragment of protein L capable of binding to a portion of a light chain.

In some embodiments, the antibody capture complex comprises avidin or streptavidin linked to an ectodomain of a high affinity IgE receptor (FcεRIa). In some embodiments, the antibody capture complex comprises avidin linked to FcεRIa. In some embodiments, the antibody capture complex comprises streptavidin linked to FcεRIa.

### Target Antibody

In some embodiments, the target antibody comprises an IgM antibody.

In some embodiments, the target antibody comprises an IgG antibody. In some embodiments, the anti-IgG antibody comprises an IgG1 antibody, an IgG2 antibody, an IgG2a antibody, an IgG2b antibody, an IgG3 antibody, and/or an IgG4 antibody. In some embodiments, the anti-IgG antibody comprises an IgG1 antibody. In some embodiments, the anti-IgG antibody comprises an IgG2 antibody. In some embodiments, the anti-IgG antibody comprises an IgG2a antibody. In some embodiments, the anti-IgG antibody comprises an IgG2b antibody. In some embodiments, the anti-IgG antibody comprises an IgG3 antibody. In some embodiments, the anti-IgG antibody comprises an IgG4 antibody.

In some embodiments, the target antibody comprises an IgA antibody. In some embodiments, the IgA antibody comprises an IgA1 antibody, an IgA2 antibody, a secretory IgA antibody, or a polymeric IgA antibody. In some embodiments, the IgA antibody comprises an IgA1 antibody. In some embodiments, the IgA antibody comprises an IgA2 antibody. In some embodiments, the IgA antibody comprises a secretory IgA antibody. In some embodiments, the IgA antibody comprises a polymeric IgA antibody.

In some embodiments, the target antibody comprises an IgE antibody.

### Target Antigen

In some embodiments, the target of the target antibody, i.e., the antigen to which the target antibody binds, is an allergen. In some embodiments, the allergen comprises a component of an animal product, a drug, a foodstuff, a substance present in a venom, a substance present in saliva of a biting insect, a mold spore, a cosmetic, a metal, latex, a wood, and/or a plant pollen. In some embodiments, the allergen comprises a component of an animal product. In some embodiments, the allergen comprises a drug. In some embodiments, the allergen comprises a foodstuff. In some embodiments, the allergen comprises a substance present in a venom. In some embodiments, the allergen comprises a substance present in saliva of a biting insect. In some embodiments, the allergen comprises a mold spore. In some embodiments, the allergen comprises a cosmetic. In some embodiments, the allergen comprises a metal. In some embodiments, the allergen comprises latex. In some embodiments, the allergen comprises a wood. In some embodiments, the allergen comprises a plant pollen.

In some embodiments, the animal product comprises Bla g, Bla g 1, Bla g 2, Bla g 3, Bla g 3, Bla g 4, Bla g 5, Bla g 6, Bla g 7, Bla g 8, Bla g 9, Bla g 11, Per a, Per a 1, Per a 2, Per a 3, Per a 6, Per a 7, Per a 9, Per a 10, Per a 11, Per a 12, Der p 1, Der p 2, Der f 1, Eur m 1, Pso o 1, Equ c 1, Fel d 1, Fel d 4, Mus m 1, Rat n 1, dust mite tropomyosin, shellfish tropomyosin, and/or cockroach tropornyosin. In some embodiments, the animal product comprises Bla g. In some embodiments, the animal product comprises Bla g 1. In some embodiments, the animal product comprises Bla g 2. In some embodiments, the animal product comprises Bla g 3. In some embodiments, the animal product comprises Bla g 3. In some embodiments, the animal product comprises Bla g 4. In some embodiments, the animal product comprises Bla g 5. In some embodiments, the animal product comprises Bla g 6. In some embodiments, the animal product comprises Bla g 7. In some embodiments, the animal product comprises Bla g 8. In some embodiments, the animal product comprises Bla g 9. In some embodiments, the animal product comprises Bla g 11. In some embodiments, the animal product comprises Per a. In some embodiments, the animal product comprises, Per a 1. In some embodiments, the animal product comprises Per a 2. In some embodiments, the animal product comprises Per a 3. In some embodiments, the animal product comprises Per a 6. In some embodiments, the animal product comprises Per a 7. In some embodiments, the animal product comprises Per a 9. In some embodiments, the animal product comprises Per a 10. In some embodiments, the animal product comprises Per a 11. In some embodiments, the animal product comprises Per a 12. In some embodiments, the animal product comprises Der p 1. In some embodiments, the animal product comprises Der p 2. In some embodiments, the animal product comprises Der f 1. In some embodiments, the animal product comprises Eur m 1. In some embodiments, the animal product comprises Pso o 1. In some embodiments, the animal product comprises Equ c 1. In some embodiments, the animal product comprises Fel d 1. In some embodiments, the animal product comprises Fel d 4. In some embodiments, the animal product comprises Mus m 1. In some embodiments, the animal product comprises Rat n 1. In some embodiments, the animal product comprises dust mite tropomyosin. In some embodiments, the animal product comprises shellfish tropomyosin. In some embodiments, the animal product comprises cockroach tropomyosin.

In some embodiments, the animal product comprises animal fur, animal dander, cockroach calyx, wool, and/or a dust mite secretion. In some embodiments, the animal product comprises animal fur. In some embodiments, the animal product comprises animal dander. In some embodiments, the animal product comprises cockroach calyx. In some embodiments, the animal product comprises wool. In some embodiments, the animal product comprises a dust mite secretion.

In some embodiments, the drug comprises penicillin, a sulfonamide, a salicylate, and/or neomycin. In some embodiments, the drug comprises penicillin. In some embodiments, the drug comprises a sulfonamide. In some embodiments, the drug comprises a salicylate. In some embodiments, the drug comprises neomycin.

In some embodiments, the foodstuff comprises celery, celeriac, corn, maize, egg, fruit, pumpkin, eggplant, legume, milk, seafood, sesame, soy, tree nut, wheat, and/or balsam of Peru. In some embodiments, the foodstuff comprises celery. In some embodiments, the foodstuff comprises celeriac. In some embodiments, the foodstuff comprises corn. In some embodiments, the foodstuff comprises maize. In some embodiments, the foodstuff comprises egg. In some embodiments, the foodstuff comprises fruit. In some embodiments, the foodstuff comprises pumpkin. In some embodiments, the foodstuff comprises eggplant. In some embodiments, the foodstuff comprises legume. In some embodiments, the foodstuff comprises milk. In some embodiments, the foodstuff comprises seafood. In some embodiments, the foodstuff comprises sesame. In some embodiments, the foodstuff comprises soy. In some embodiments, the foodstuff comprises tree nut. In some embodiments, the foodstuff comprises wheat. In some embodiments, the foodstuff comprises balsam of Peru. In some embodiments, the legume comprises a bean, a pea, a peanut, or a soybean. In some embodiments, the legume comprises a bean. In some embodiments, the legume comprises a pea. In some embodiments, the legume comprises a peanut. In some embodiments, the legume comprises a soybean. In some embodiments, the tree nut comprises a pecan and/or an almond. In some embodiments, the tree nut comprises a pecan. In some embodiments, the tree nut comprises an almond.

In some embodiments, the substance present in a venom comprises a substance present in bee sting venom and/or a substance present in wasp sting venom. In some embodiments, the substance present in a venom comprises a substance present in bee sting venom. In some embodiments, the substance present in a venom comprises a substance present in wasp sting venom.

In some embodiments, the biting insect is a mosquito or tick. In some embodiments, the biting insect is a mosquito. In some embodiments, the biting insect is a tick. In some embodiments, the allergen in the biting insect is present in the saliva. In some embodiments, the allergen comprises an oligosaccharide. In some embodiments, the allergen comprises galactose-alpha-1,3-galactose or galactose-alpha-1,3-galactose-beta-1,4-N-acetyl glucosamine.

In some embodiments, the cosmetic comprises a fragrance and/or quaternium-15. In some embodiments, the cosmetic comprises a fragrance. In some embodiments, the cosmetic comprises quaternium-15.

In some embodiments, the metal comprises nickel and/or chromium. In some embodiments, the metal comprises nickel. In some embodiments, the metal comprises chromium.

In some embodiments, the plant pollen comprises a grass pollen, a weed pollen, and/or a tree pollen. In some embodiments, the plant pollen comprises a grass pollen. In some embodiments, the plant pollen comprises a weed pollen. In some embodiments, the plant pollen comprises a tree pollen. In some embodiments, the grass pollen comprises ryegrass pollen and/or timothy-grass pollen. In some embodiments, the grass pollen comprises ryegrass pollen. In some embodiments, the grass pollen comprises timothy-grass pollen. In some embodiments, the weed pollen comprises ragweed pollen, plantago pollen, nettle pollen, pollen of the species Artemisia vulgaris, pollen of the species Chenopodium album, or sorrel pollen. In some embodiments, the weed pollen comprises ragweed pollen. In some embodiments, the weed pollen comprises plantago pollen. In some embodiments, the weed pollen comprises nettle pollen. In some embodiments, the weed pollen comprises pollen of the species Artemisia vulgaris. In some embodiments, the weed pollen comprises pollen of the species Chenopodium album. In some embodiments, the weed pollen comprises sorrel pollen. In some embodiments, the tree pollen comprises birch pollen, alder pollen, hazel pollen, hornbeam pollen, pollen of the genus Aesculus, willow pollen, poplar pollen, pollen of the genus Platanus, pollen of the genus Tilia, pollen of the species Olea, pollen of Ashe juniper, or pollen of the species Alstonia scholaris. In some embodiments, the tree pollen comprises birch pollen. In some embodiments, the tree pollen comprises alder pollen. In some embodiments, the tree pollen comprises hazel pollen. In some embodiments, the tree pollen comprises hornbeam pollen. In some embodiments, the tree pollen comprises pollen of the genus Aesculus. In some embodiments, the tree pollen comprises willow pollen. In some embodiments, the tree pollen comprises poplar pollen. In some embodiments, the tree pollen comprises pollen of the genus Platanus. In some embodiments, the tree pollen comprises pollen of the genus Tilia. In some embodiments, the tree pollen comprises pollen of the species Olea. In some embodiments, the tree pollen comprises pollen of Ashe juniper. In some embodiments, the tree pollen comprises pollen of the species Alstonia scholaris. In some embodiments, the plant pollen comprises olive tree pollen. In some embodiments, the allergen comprises Ole e 1.

In some embodiments, the allergen comprises is one from an oak tree (Quercus albus), Japanese cedar tree (Cryptomeria japonica), or from an Elm tree or Hickory tree.

In some embodiments, the allergen comprises a dog allergen, such as Can f proteins. In some embodiments, the allergen comprises a cow's milk allergen or a beef protein, such as Bos d 2-13. In some embodiments, the allergen comprises a chicken allergen (e.g., Gal d proteins), pig allergens (i.e., Sus s proteins), or a serum albumin. In some embodiments, the allergen comprises a fish allergen (e.g., Gad c/Gad m/Pan h/Sals s proteins) In some embodiments, the allergen comprises a parvalbumin.

In any of the embodiments described herein, the allergen can be replaced with a non-allergen compound such as, for example, a foreign antigen. In some embodiments, the foreign antigen comprises a spike protein of a coronavirus. In some embodiments, the foreign antigen comprises a mutated peptide from a host. In some embodiments, the foreign antigen comprises a protein from another species. In some embodiments, the foreign antigen comprises a compound from an infectious disease, such as SARS-CoV-2 or influenza virus.

In some embodiments, the antibody secreting cell is a plasma cell. In some embodiments, the antibody secreting cell is a plasmablast. In some embodiments, the plasma cell is a short-lived plasma cell. In some embodiments, the plasma cell is a long-lived plasma cell.

### Methods of Capturing a Target Antibody Secreted by an Antibody Secreting Cell

The present disclosure also provides methods of capturing a target antibody secreted by an antibody secreting cell, the method comprising: contacting a population of antibody secreting cells with a second component of a binding pair to allow the second component of the binding pair to bind to the cell surface, wherein the population of antibody secreting cells comprises the antibody secreting cell that secretes the target antibody; contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the antibody secreting cells, and whereby the antibody-capture molecule captures the target antibody secreted by the antibody secreting cell; and contacting the population of antibody secreting cells with an antigen to allow the target antibody secreted by the antibody secreting cell and captured by the antibody capture complex to bind the antigen. In some embodiments, the method further comprises, after contacting the population of antibody secreting cells with an antibody capture complex, contacting the population of antibody secreting cells with a secondary anti-Ig antibody. The step of contacting with a secondary anti-Ig antibody can be performed before, with, or after the step of contacting the population of antibody secreting cells with an antigen.

The present disclosure also provides methods of capturing a target antibody secreted by an antibody secreting cell, the method comprising: contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell; and contacting the population of the antibody secreting cells with an antigen, whereby the target antibody secreted by the antibody secreting cell and captured by the antibody capture complex binds the antigen. In some embodiments, the method further comprises, after contacting the population of antibody secreting cells with an antibody capture complex, contacting the population of antibody secreting cells with a secondary anti-Ig antibody. The step of contacting with a secondary anti-Ig antibody can be performed before, with, or after the step of contacting the population of antibody secreting cells with an antigen.

In some embodiments, after the population of antibody secreting cells is contacted with an antigen, the method may further comprise sorting the population of antibody secreting cells to collect a pool of antibody secreting cells, wherein the antibody secreting cells in the pool each secrete an antibody that is captured by the antibody capture molecule and is bound by the antigen, wherein the collected pool of antibody secreting cells comprises the antigen secreting cell that secretes the target antibody; and in some such embodiments, the method may further comprise separating the collected pool of antibody secreting cells into single cells and isolating the antigen secreting cell that secretes the target antibody.

In some embodiments, the antigen is a barcoded antigen. In some embodiments, the antigen comprises an allergen. In some embodiments, the barcoded antigen comprises a barcode nucleic acid molecule. In some embodiments, a portion of the nucleotide sequence of the barcode nucleic acid molecule is unique to the antigen. In some embodiments, a portion of the barcode nucleic acid molecule comprises a sequencing primer. In some embodiments, the sequencing primer is upstream of the portion of the nucleotide sequence of the barcode nucleic acid molecule that is unique to the antigen. In some embodiments, a portion of the barcode nucleic acid molecule is complementary to a template switch oligonucleotide (TSO). In some embodiments, the portion of the nucleotide sequence of the barcode nucleic acid molecule is unique to the antigen, is upstream of the portion of the barcode nucleic acid molecule, and is complementary to the ISO. In some embodiments, the 3′ terminus of the barcode nucleic acid molecule comprises three contiguous cytidine or ribocytidine residues. In some embodiments, the barcode nucleic acid molecule comprises DNA. In some embodiments, the barcode nucleic acid molecule comprises RNA.

In some embodiments, the population of antibody secreting cells is contacted with a plurality of barcoded antigens, wherein each barcoded antigen of the plurality of barcoded antigens comprises a unique antigen linked to a unique nucleic acid molecule. In some embodiments, the unique antigen comprises an allergen. In some embodiments, the unique nucleic acid molecule comprises a barcode nucleic acid molecule.

In some embodiments, each barcoded antigen of the plurality of barcoded antigens is detectably labeled. In some embodiments, each barcoded antigen of the plurality of barcoded antigens is labeled with a radioactive compound, a fluorescent compound, or an enzyme. In some embodiments, each barcoded antigen of the plurality of barcoded antigens is labeled with a radioactive compound. In some embodiments, each barcoded antigen of the plurality of barcoded antigens is labeled with a fluorescent compound. In some embodiments, each barcoded antigen of the plurality of barcoded antigens is labeled with an enzyme. In some embodiments, the radioactive compound comprises ³H, ¹⁴C, ¹⁹F, ³⁵S, ¹²⁵I, ¹³¹I, ¹¹¹In, or ⁹⁹Tc. In some embodiments, the radioactive compound comprises 3H. In some embodiments, the radioactive compound comprises ¹⁴C. In some embodiments, the radioactive compound comprises ¹⁹F. In some embodiments, the radioactive compound comprises ³⁵S. In some embodiments, the radioactive compound comprises ¹²⁵I. In some embodiments, the radioactive compound comprises ¹³¹I. In some embodiments, the radioactive compound comprises ¹¹¹In. In some embodiments, the radioactive compound comprises ⁹⁹Tc. In some embodiments, the fluorescent compound comprises fluorescein, fluorescein isothiocyanate, rhodamine, 5-dimethylamine-1-naphthalenesulfonyl chloride, phycoerythrin, or a fluorescent protein. In some embodiments, the fluorescent compound comprises fluorescein. In some embodiments, the fluorescent compound comprises fluorescein isothiocyanate. In some embodiments, the fluorescent compound comprises rhodamine. In some embodiments, the fluorescent compound comprises 5-dimethylamine-1-naphthalenesulfonyl chloride. In some embodiments, the fluorescent compound comprises phycoerythrin. In some embodiments, the fluorescent compound comprises a fluorescent protein. In some embodiments, the enzyme comprises alkaline phosphatase, horseradish peroxidase, luciferase, or glucose oxidase. In some embodiments, the enzyme comprises alkaline phosphatase. In some embodiments, the enzyme comprises horseradish peroxidase. In some embodiments, the enzyme comprises luciferase. In some embodiments, the enzyme comprises glucose oxidase.

In embodiments where a secondary anti-Ig antibody is employed, in some such embodiments, the secondary anti-Ig antibody comprises an anti-IgM antibody, an anti-IgG antibody, an anti-IgA antibody, and/or an anti-IgE antibody. In some embodiments, the secondary anti-Ig antibody comprises an anti-IgM antibody. In some embodiments, the secondary anti-Ig antibody comprises an anti-IgG antibody. In some embodiments, the secondary anti-Ig antibody comprises an anti-IgA antibody. In some embodiments, the secondary anti-Ig antibody comprises an anti-IgE antibody.

In some embodiments, the secondary anti-Ig antibody is detectably labeled. In some embodiments, the secondary anti-Ig antibody is labeled with a radioactive compound, a fluorescent compound, or an enzyme. In some embodiments, the secondary anti-Ig antibody is labeled with a radioactive compound. In some embodiments, the secondary anti-Ig antibody is labeled with a fluorescent compound. In some embodiments, the secondary anti-Ig antibody is labeled with an enzyme. In some embodiments, the radioactive compound comprises ³H, ¹⁴C, ¹⁹F, ³⁵S, ¹²⁵I, ¹³¹I, ¹¹¹In, or ⁹⁹Tc. In some embodiments, the radioactive compound comprises 3H. In some embodiments, the radioactive compound comprises ¹⁴C. In some embodiments, the radioactive compound comprises ¹⁹F. In some embodiments, the radioactive compound comprises ³⁵S. In some embodiments, the radioactive compound comprises ¹²⁵I. In some embodiments, the radioactive compound comprises ¹³¹I. In some embodiments, the radioactive compound comprises ¹¹¹In. In some embodiments, the radioactive compound comprises ⁹⁹Tc. In some embodiments, the fluorescent compound comprises fluorescein, fluorescein isothiocyanate, rhodamine, 5-dimethylamine-1-naphthalenesulfonyl chloride, phycoerythrin, or a fluorescent protein. In some embodiments, the fluorescent compound comprises fluorescein. In some embodiments, the fluorescent compound comprises fluorescein isothiocyanate. In some embodiments, the fluorescent compound comprises rhodamine. In some embodiments, the fluorescent compound comprises 5-dimethylamine-1-naphthalenesulfonyl chloride. In some embodiments, the fluorescent compound comprises phycoerythrin. In some embodiments, the fluorescent compound comprises a fluorescent protein. In some embodiments, the enzyme comprises alkaline phosphatase, horseradish peroxidase, luciferase, or glucose oxidase. In some embodiments, the enzyme comprises alkaline phosphatase. In some embodiments, the enzyme comprises horseradish peroxidase. In some embodiments, the enzyme comprises luciferase. In some embodiments, the enzyme comprises glucose oxidase.

In some embodiments, the population of antibody secreting cells is obtained by enriching a population of cells obtained from a human for immune cells. In some embodiments, the population of cells obtained from the human is enriched for plasma cells or plasmablasts.

In some embodiments, the population of antibody secreting cells is obtained from a lymph node, lung, bone marrow, and/or blood of a human. In some embodiments, the population of antibody secreting cells is obtained from a lymph node. In some embodiments, the population of antibody secreting cells is obtained from a lung. In some embodiments, the population of antibody secreting cells is obtained from bone marrow. In some embodiments, the population of antibody secreting cells is obtained from blood.

In some embodiments, the antigen and/or the secondary anti-Ig antibody are/is detected. In some embodiments, the antibody secreting cells are sorted to collect a pool of antibody secreting cells that are bound by the antigen and/or by the secondary anti-Ig antibody, wherein the collected pool of antibody secreting cells comprises the antibody secreting cell that secretes the target antibody.

In some embodiments, the antibody secreting cells are contacted with an Fc block prior to contacting the population of antibody secreting cells with the second component of the binding pair.

The present disclosure also provides methods of capturing an IgE antibody secreted by an antibody secreting cell, wherein the IgE antibody is directed to an allergen, the method comprising: contacting a population of antibody secreting cells with NHS-biotin to allow biotin to bind to the cell surface, wherein the population of antibody secreting cells comprises the antibody secreting cell that secretes the IgE antibody; contacting the population of antibody secreting cells with an antibody capture complex, wherein the antibody capture complex comprises streptavidin linked to an ectodomain of FcεRIa, and whereby the antibody-capture molecule binds to the IgE antibody secreted by the antibody secreting cell; and contacting the population of antibody secreting cells with an antigen, wherein the antigen is an antigenic portion or a mixture of a plurality of antigenic portions of the allergen, whereby the IgE antibody secreted by the antibody secreting cell and captured by the antibody capture complex binds to the antigen. In some embodiments, the antigen comprises a plurality of barcoded antigens, wherein each of the plurality of barcoded antigens is a different antigenic portion of the allergen. In some embodiments, the method further comprises sorting the population of antibody secreting cells to collect a pool of antibody secreting cells, wherein the antibody secreting cells in the pool each secrete an IgE antibody that is captured by the antibody capture complex and is bound by the antigen, wherein the collected pool of antibody secreting cells comprises the antigen secreting cell that secretes the IgE antibody.

In some embodiments, the method further comprises contacting the population of antibody secreting cells with an anti-IgE antibody to allow the IgE antibody secreted by the antibody secreting cell and captured by the antibody capture complex to bind to the anti-IgE antibody. In some embodiments, the method may further comprise sorting the population of antibody secreting cells to collect a pool of antibody secreting cells, wherein the antibody secreting cells in the pool each secrete an IgE antibody that is captured by the antibody capture complex and is bound by the antigen and the anti-IgE antibody, wherein the collected pool of antibody secreting cells comprises the antigen secreting cell that secretes the IgE antibody.

In some embodiments, the method further comprises separating the collected pool of antibody secreting cells into single cells and isolating the antigen secreting cell that secretes the IgE antibody.

### Antigen Chase

A population of antibody secreting cells may comprise a plurality of antibody secreting cells each secreting an antibody that is captured by the antibody capture complex, wherein the plurality of antibody secreting cells includes the antibody secreting cell that secretes the target antibody. Some of the antibodies secreted by the plurality of antibody secreting cells bind an antigen to which the target antibody binds, although the antibodies may have varying affinities to the antigen. An antigen chase can be employed to obtain a cell population enriched in antibody secreting cells that secrete antibodies having high affinity for the antigen, which allows for isolation of an antibody secreting cell that secretes a target antibody having high affinity. Therefore, in some embodiments of the methods disclosed herein, the step of contacting the population of antibody secreting cells with an antigen includes an antigen chase. In these embodiments, as an initial binding step, the population of antibody secreting cells is contacted with a first labeled form of the antigen to allow the antigen to bind to antibodies including the target antibody captured at the surface of antibody secreting cells and form an antigen-antibody complex, followed by a “chase” step where the cells are contacted with one of several forms of the antigen: (i) an unlabeled form of the antigen (“cold chase”), (ii) a second labeled form of the antigen (“hot chase”), or a combination of an unlabeled form of the antigen and a second labeled form of the antigen (“combination chase”). The cells that remain bound to the first labeled form of the antigen after the chase represent cells secreting antibodies with high binding affinity. Accordingly, an antigen chase permits selection among cells secreting antibodies with different affinities to enrich for cells secreting antibodies with high affinities and subsequent isolation of an antibody secreting cell that secretes a target antibody of high affinity.

The term “enriching” means increasing the frequency or percentage of desired cells in a cell population, e.g., increasing the percentage of antibody secreting cells secreting high affinity antibodies within an antibody secreting cell population containing cells secreting antibodies having various affinities (e.g., high affinity, medium affinity, and low affinity). Thus, an antibody secreting cell population enriched in cells secreting high affinity antibodies encompasses a cell population having a higher frequency and/or higher percentage of antibody secreting cells secreting high affinity antibodies as a result of an enrichment process. In the present context, the enrichment process is a process that includes an antigen chase, thereby selecting cells that secrete high affinity antibodies to an antigen from a population of cells that secrete antibodies of various affinities to the antigen, and separating cells that secrete high affinity antibodies from cells that secrete antibodies not of high affinity.

The cell population obtained as a result of a chase is enriched in antibody-secreting cells secreting antibodies with high binding affinity to an antigen of interest. In other words, the enriched population of cells contains a greater percentage of cells that secretes an antibody that binds to the antigen with high binding affinity, as compared to a cell population before or without a chase. In some embodiments, at least 40% of the cells collected secrete a high affinity antibody, e.g., antibody having a KD from 0.1 pM to 25 nM. In some embodiments, the enriched cell population may be a population having at least 50% of cells within the population secreting an antibody that binds to the antigen of interest with high binding affinity, e.g., antibody having a KD from 0.1 pM to 25 nM. In some embodiments, the enriched cell population may be a population having at least 60% of cells within the population secreting an antibody that binds to the antigen of interest with high binding affinity, e.g., antibody having a KD from 0.1 pM to 25 nM. In some embodiments, the enriched cell population may be a population having at least 70% of cells within the population secreting an antibody that binds to the antigen of interest with high binding affinity, e.g., antibody having a KD from 0.1 pM to 25 nM. In some embodiments, the enriched cell population may be a population having at least 80% of cells within the population secreting an antibody that binds to the antigen of interest with high binding affinity, e.g., antibody having a KD from 0.1 pM to 25 nM. In some embodiments, the enriched cell population may be a population having at least 90% of cells within the population secreting an antibody that binds to the antigen of interest with high binding affinity, e.g., antibody having a KD from 0.1 pM to 25 nM. In some embodiments, the enriched cell population may be a population having at least 95% of cells within the population secreting an antibody that binds to the antigen of interest with high binding affinity, e.g., antibody having a KD from 0.1 pM to 25 nM. In some embodiments, the frequency of cells secreting high affinity antibodies (e.g., antibody having a KD from 0.1 pM to 25 nM) in the cell population after a chase is increased by at least 30% as compared to the frequency in the cell population before or without the chase. In some embodiments, the frequency of cells secreting high affinity antibodies (e.g., antibody having a KD from 0.1 pM to 25 nM) in the cell population after a chase is increased by at least 40% as compared to the frequency in the cell population before or without the chase. In some embodiments, the frequency of cells secreting high affinity antibodies (e.g., antibody having a KD from 0.1 pM to 25 nM) in the cell population after a chase is increased by at least 50% as compared to the frequency in the cell population before or without the chase. In some embodiments, the frequency of cells secreting high affinity antibodies (e.g., antibody having a KD from 0.1 pM to 25 nM) in the cell population after a chase is increased by at least 75% as compared to the frequency in the cell population before or without the chase. In some embodiments, the frequency of cells secreting high affinity antibodies (e.g., antibody having a KD from 0.1 pM to 25 nM) in the cell population after a chase is increased by at least 100% as compared to the frequency in the cell population before or without the chase. In some embodiments, the frequency of cells secreting high affinity antibodies (e.g., antibody having a KD of 0.1 pM to 25 nM) in the cell population after a chase is increased by at least 200% as compared to the frequency in the cell population before or without the chase. In some embodiments, the frequency of cells secreting high affinity antibodies having a KD of less than 1 nM (e.g., antibody having a KD from 0.1 pM to 1 nM) in the cell population after a chase is increased by at least 40% as compared to the frequency in the cell population before or without the chase. In some embodiments, the frequency of cells secreting high affinity antibodies having a KD of less than 1 nM (e.g., antibody having a KD from 0.1 pM to 1 nM) in the cell population after a chase is increased by at least 50% as compared to the frequency in the cell population before or without the chase. In some embodiments, the frequency of cells secreting high affinity antibodies having a KD of less than 1 nM (e.g., antibody having a KD from 0.1 pM to 1 nM) in the cell population after a chase is increased by at least 75% as compared to the frequency in the cell population before or without the chase. In some embodiments, the frequency of cells secreting high affinity antibodies having a KD of less than 1 nM (e.g., antibody having a KD from 0.1 pM to 1 nM) in the cell population after a chase is increased by at least 100% as compared to the frequency in the cell population before or without the chase. In some embodiments, the frequency of cells secreting high affinity antibodies having a KD of less than 1 nM (e.g., antibody having a KD from 0.1 pM to 1 nM) in the cell population after a chase is increased by at least 200% as compared to the frequency in the cell population before or without the chase. In some embodiments, the frequency of cells secreting high affinity antibodies having a KD of less than 0.1 nM (e.g., antibody having a KD from 0.1 pM to 0.1 nM) in the cell population after a chase is increased by at least 40% as compared to the frequency in the cell population before or without the chase. In some embodiments, the frequency of cells secreting g high affinity antibodies having a KD of less than 0.1 nM (e.g., antibody having a KD from 0.1 pM to 0.1 nM) in the cell population after a chase is increased by at least 50% as compared to the frequency in the cell population before or without the chase. In some embodiments, the frequency of cells secreting high affinity antibodies having a KD of less than 0.1 nM (e.g., antibody having a KD from 0.1 pM to 0.1 nM) in the cell population after a chase is increased by at least 75% as compared to the frequency in the cell population before or without the chase. In some embodiments, the frequency of cells secreting high affinity antibodies having a KD of less than 0.1 nM (e.g., antibody having a KD from 0.1 pM to 0.1 nM) in the cell population after a chase is increased by at least 100% as compared to the frequency in the cell population before or without the chase. In some embodiments, the frequency of cells secreting high affinity antibodies having a KD of less than 0.1 nM (e.g., antibody having a KD from 0.1 pM to 0.1 nM) in the cell population after a chase is increased by at least 200% as compared to the frequency in the cell population before or without the chase.

“Binding affinity,” as that term is known in the art, generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule for its binding partner can generally be represented by the dissociation equilibrium constant (KD or K_{D}). There is an inverse relationship between K_{D} (molar) value and binding affinity, therefore the smaller the K_{D} value (M), the higher the affinity. Thus “higher affinity” refers to antibodies that generally bind antigen stronger and/or faster and/or remain bound longer. Generally, a lower concentration (M) of antigen is needed to achieve the desired effect due to its stronger binding interaction.

The term “kd” (sec−1 or 1/s) refers to the dissociation rate constant of a particular antibody-antigen interaction, or the dissociation rate constant of an antibody, Ig, antibody-binding fragment, or molecular interaction. This value is also referred to as the k_{off} value.

The term “ka” (M−1×sec−1 or 1/M) refers to the association rate constant of a particular antibody-antigen interaction, or the association rate constant of an antibody, Ig, antibody-binding fragment, or molecular interaction.

The term “KD” or “K_{D}” (M) refers to the equilibrium dissociation constant of a particular antibody-antigen interaction, or the equilibrium dissociation constant of an antibody, Ig, antibody-binding fragment, or molecular interaction. The equilibrium dissociation constant is obtained by dividing the ka with the kd.

A variety of methods of measuring binding affinity are known in the art, any of which can be used for purposes of the present invention. Binding affinities obtained using the method are typically in the range of about 0.1 pM to about 25 nM as determined by surface plasmon resonance. In some embodiments, binding affinities are less than about 10 nM as determined by surface plasmon resonance.

The term “high affinity” antibody refers to those antibodies having a binding affinity, expressed as KD, of 25 nM or less, e.g., having a numerical value of about 0.1 pM to about 25 nM. To this end, high affinity antibodies may have a measured KD about 25×10⁻⁹ M (25 nM) or less, about 10×10⁻⁹ M (10 nM) or less, about 1×10⁻⁹ M (1 nM) or less, about 1×10⁻¹⁰ M (0.1 nM) or less, about 0.5×10⁻¹⁰ M (0.05 nM) or less, about 0.05×10⁻¹⁰ M (5 pM) or less, about 1 pM or less, or about 0.5 pM or less, as measured by surface plasmon resonance, e.g., BIACORE™ or solution-affinity ELISA. Those of skill in the art will recognize that values for KD of antibodies may be represented numerically either as nE^{-z}, or as n×10^{-z}, for example, 3.2E⁻¹² is equivalent to 3.2×10⁻¹² and indicates a KD of 3.2 picomolar (pM). In some embodiments, the high affinity antibodies have a measured KD in a range of from about 0.1 pM to about 25 nM. In some embodiments, the high affinity antibodies have a measured KD in a range of from about 0.1 pM to about 20 nM. In some embodiments, the high affinity antibodies have a measured KD in a range of from about 0.1 pM to about 15 nM. In some embodiments, the high affinity antibodies have a measured KD in a range of from about 0.1 pM to about 10 nM. In some embodiments, the high affinity antibodies have a measured KD in a range of from about 0.1 pM to about 5 nM. In some embodiments, the high affinity antibodies have a measured KD in a range of from about 0.1 pM to about 1 nM. In some embodiments, the high affinity antibodies have a measured KD in a range of from about 0.1 pM to about 0.5 nM. In some embodiments, the high affinity antibodies have a measured KD in a range of from about 0.1 pM to about 0.1 nM. In some embodiments, the high affinity antibodies have a measured KD of less than about 20 nM. In some embodiments, the high affinity antibodies have a measured KD of less than about less than about 15 nM. In some embodiments, the high affinity antibodies have a measured KD of less than about 10 nM. In some embodiments, the high affinity antibodies have a measured KD of less than about 5 nM. In some embodiments, the high affinity antibodies have a measured KD of less than about 1 nM. In some embodiments, the high affinity antibodies have a measured KD of less than about 0.1 nM. In some embodiments, the high affinity antibodies have a measured KD of less than about 0.5 nM. In some embodiments, the high affinity antibodies have a measured KD of less than about 0.01 nM. In some embodiments, the high affinity antibodies have a measured KD of less than about 0.001 nM (or 1 pM). In some embodiments, the high affinity antibodies have a measured KD of less than about 0.5 pM.

In some embodiments, the antigen is a protein that is present in a monomeric form. Examples of proteins that exist in monomers include interleukin molecules, such as IL-13. In some embodiments, the antigen is a protein that is present in a multimeric form, including homomers and heteromers. In some embodiments, the antigen is a protein that is present in both monomeric and multimeric forms, in which case a mixture of protein monomers and multimers can be used in the method described herein.

Whether the antigen is a protein that is present in a monomeric form, multimeric form, or a mixture there, the antigen may be employed in the methods described herein in a monovalent form or a multivalent form. The terms “monovalent” and “multivalent” are used to refer to the number of units of antigen being presented and to differentiate from the antigen itself being a protein in a monomer form, a multimer form or a mixture thereof. Thus, a monovalent form of an antigen refers to a single unit form of the antigen, where the antigen itself may be a protein in a monomeric form, a multimeric form or a mixture thereof. A multivalent form of an antigen refers to multiple units of the antigen being presented, typically by way of a multivalent molecule to which the antigen is bound or linked. A multivalent molecule can be a dimer, trimer, tetramer, pentamer, hexamer, and the like, or a mixture thereof. In some embodiments, the multivalent molecule is a streptavidin multimer (e.g., tetramer), which can be complexed with biotin which is then linked to the antigen, thereby providing a multivalent (e.g., tetravalent form) of the antigen. In some embodiments, a streptavidin multimer includes tetramer and may additionally include trimer and/or dimer. In some embodiments, a streptavidin multimer is conjugated with a fluorophore such as phycoerythrin. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

In some embodiments, the step of contacting the population of antibody secreting cells with an antigen comprises: (a) contacting the population of antibody-secreting cells with a first labeled form of the antigen to allow the antigen to bind to antibodies including the target antibody captured on the cell surface, wherein the antigen of the first labeled form is conjugated to a first detectable label; (b) washing the cells to remove unbound antigen; (c) contacting the cells with either (i) an unlabeled form of the antigen, (ii) a second labeled form of the antigen, or (iii) the unlabeled form of the antigen and the second labeled form of the antigen; (d) washing the cells to remove unbound antigen; (e) collecting a pool of antibody secreting cells remaining bound to the first labeled form of the antigen, wherein the collected pool of antibody secreting cells comprises the antibody secreting cell that secretes the target antibody, wherein the target antibody secreted by the antibody secreting cell and captured by the antibody capture complex remains bound to the first labeled form of the antigen.

In some embodiments, the first labeled form of the antigen is at a concentration between 0.001 nM and 1 uM. In some embodiments, the first labeled form of the antigen is at a concentration between 0.01 nM and 100 nM. In some embodiments, the first labeled form of the antigen is at a concentration between 0.05 nM to 10 nM. In some embodiments, the first labeled form of the antigen is at a concentration between 0.05 nM to 9 nM, 0.05 nM to 8 nM, 0.05 nM to 7 nM, 0.05 nM to 6 nM, 0.05 nM to 5 nM, 0.05 nM to 4 nM, 0.05 nM to 3 nM, 0.05 nM to 2 nM, or 0.05 nM to 1 nM. In some embodiments, the first labeled form of the antigen is at a concentration between 0.1 nM to 7.5 nM. In some embodiments, the first labeled form of the antigen is at a concentration between 0.1 nM to 7 nM, 0.1 nM to 6 nM, 0.1 nM to 5 nM, 0.1 nM to 4 nM, 0.1 nM to 3 nM, 0.1 nM to 2 nM, or 0.1 nM to 1 nM. In some embodiments, the first labeled form of the antigen is at a concentration between 0.2 nM to 7.5 nM. In some embodiments, the first labeled form of the antigen is at a concentration between 0.2 nM to 7 nM, 0.2 nM to 6 nM, 0.2 nM to 5 nM, 0.2 nM to 4 nM, 0.2 nM to 3 nM, 0.2 nM to 2 nM, 0.2 nM to 1 nM, 0.3 nM to 7 nM, 0.3 nM to 6 nM, 0.3 nM to 5 nM, 0.3 nM to 4 nM, 0.3 nM to 3 nM, 0.3 nM to 2 nM, 0.3 nM to 1 nM, 0.5 nM to 7 nM, 0.5 nM to 6 nM, 0.5 nM to 5 nM, 0.5 nM to 4 nM, 0.5 nM to 3 nM, 0.5 nM to 2 nM, 0.5 nM to 1 nM. In some embodiments, the first labeled form of the antigen is at a concentration between 1.0 nM to 10 nM, 1.0 nM to 9 nM, 1.0 nM to 8.0 nM, 1.0 nM to 7 nM, 1.0 nM to 6 nM, 1.0 nM to 5 nM, 1.0 nM to 4 nM, 1.0 nM to 3 nM, 1.0 nM to 2 nM, 2.0 nM to 10.0 nM, or 5.0 nM to 10.0 nM. In specific embodiments, the antibody-secreting cells can be contacted with a first labeled form of the antigen where the first labeled form of the antigen is at a concentration of 0.2 nM. In specific embodiments, the antibody-secreting cells can be contacted with a first labeled form of the antigen where the first labeled form of the antigen is at a concentration of 5.0 nM. In specific embodiments, the antibody-secreting cells can be contacted with a first labeled form of the antigen where the first labeled form of the antigen is at a concentration of 7.5 nM. In specific embodiments, the antibody-secreting cells can be contacted with a first labeled form of the antigen where the first labeled form of the antigen is at a concentration of 10 nM. In specific embodiments, the antibody-secreting cells can be contacted with a first labeled form of the antigen where the first labeled form of the antigen is at a concentration of 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 8.0 nM, 8.5 nM, 9.0 nM, 9.5 nM, or greater.

In some embodiments, the contacting of the antibody secreting cells with a first labeled form of the antigen occurs from about 5 to about 60 minutes, e.g., about 20 minutes, about 30 minutes, about 40 minutes, or about 50 minutes.

In some embodiments, the first labeled form of antigen is a monovalent form of the antigen. In some embodiments, the first labeled form of antigen is a multivalent form of the antigen. In some embodiments, the first labeled form of antigen is a mixture of monovalent and multivalent forms of the antigen. Whether a monovalent form, a multivalent form or a mixture thereof, the antigen itself can be a protein that is a monomer, multimer, or a mixture of monomer and multimer.

In some embodiments, the first labeled form of the antigen is the antigen conjugated to a first detectable label. The antigen can be labeled with small molecules, radioisotopes, enzymatic proteins and fluorescent dyes. In some embodiments, the detectable label is a small molecule. Detectable small molecule labels allow for easy labeling of proteins and can be used in a number of regularly deployed detection assays known in the art.

In some embodiments, the detectable label is an enzyme reporter. Enzyme labels are larger than biotin, however, they rarely disrupt antibody function. Commonly used enzyme labels are horseradish peroxidase (HRP), alkaline phosphatase (AP), glucose oxidase and β-galactosidase. To use enzyme-labeled antibodies, samples are incubated with an enzyme-specific substrate that is catalyzed by the enzyme to produce a colored product (chromogenic assays) or light (chemiluminescent assays). Each enzyme has a set of substrates and detection methods that can be employed. For example, HRP can be reacted with diaminobenzidine to produce a brown-colored product or with luminol to produce light. In contrast, AP can be reacted with para-Nitrophenylphosphate (pNPP) to produce a yellow-colored product detected by a spectrophotometer or with 5-bromo-4-chloro-3-indolyl phosphate (BCIP) and nitroblue tetrazolium (NBT) to produce a purple-colored precipitate.

In some embodiments, the detectable label is a fluorescent label. Fluorescent labels are directly conjugated to the antibody, no enzyme/substrate or binding interactions are required for detection. Therefore, the amount of fluorescent signal detected is directly proportional to the amount of target protein in the sample. Fluorescent tags can be covalently attached to antibodies through primary amines or thiol.

After incubation of the population of antibody-secreting cells with a first labeled form of the antigen (the initial binding step), any unbound antigen can be removed from the primary antibody-producing cells. In some embodiments, the unbound antigen is removed through washing. As is known in the art, washing is a technique where a wash buffer is used to remove unwanted components including unbound antigen. Wash buffers are known in the art. In some embodiments, the wash buffer is a phosphate buffered saline (PBS) based wash buffer. In some embodiments, the wash buffer is a Tris buffered saline (TBS) wash buffer. In some embodiments, the wash buffer comprises a detergent. In some embodiments, the detergent is Tween-20. The cells are washed with wash buffer for an allotted time in order to remove unbound antigen. This allotted amount of time will be an amount of time sufficient to remove unbound antigen. In some embodiments, this allotted time can be from about 10 minutes to about 60 minutes to remove unbound antigen; multiple washes that total from 10 to 60 minutes may be used, e.g., 3 washes of 10 minutes or one 30-minute wash; 2-4 washes of 5-15 minutes each, etc. After washing and aspirating the supernatant comprising the wash buffer and unbound antigen, the pellet comprising cells bound with antigen can be used in subsequent steps. In some embodiments, the pellet comprising the bound antigen can be resuspended in a buffer and used in subsequent steps. In some embodiments, the buffer used to resuspend the pellet can be the same wash buffer. In some embodiments, the buffer used to resuspend the pellet can be a different buffer than the wash buffer.

After contacting a population of antibody secreting cells with the first labeled form of antigen (initial binding/contacting step) and once the unbound first labeled form of antigen is removed, the cells are subjected to antigen chase, i.e., the cells are contacted again, or “chased,” with the antigen to selectively enrich for cells secreting antibodies with high affinities to the antigen. This chase can be performed using any of the several forms of the antigen: (i) an unlabeled form of the antigen (“cold” chase), (ii) a second labeled form of the antigen (“hot” chase); or (iii) an unlabeled form of the antigen and a second labeled form of the antigen (a “combination chase”). The chase allows the chase antigen to bind to the antibody initially bound by the first labeled form of the antigen, thereby chasing the first labeled form of the antigen off the antibody, unless the antibody has high affinity for the antigen and remains bound to the first labeled form of the antigen after the chase.

In some embodiments, the chase is performed using an unlabeled form of the antigen (cold chase). In some embodiments, the unlabeled form of the antigen is a monovalent form of the antigen—that is, while the antigen itself can be a protein in a monomeric form, a multimeric form, or a mixture of monomeric and multimeric forms, a monovalent form of the antigen is the antigen itself without further, secondary multimerization. In some embodiments, the unlabeled form of the antigen is a multivalent form of the antigen, where the antigen itself can be a protein in a monomeric form, a multimeric form, or a mixture of monomeric and multimeric forms. In some embodiments, the unlabeled form of the antigen is a mixture of a monovalent form and a multivalent form of the antigen. In embodiments where a multivalent form of the antigen is used, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer may include trimer and/or dimer in addition to tetramer. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

In some embodiments, the chase is performed using a second labeled form of the antigen (hot chase). In such embodiments, after the initial binding and washing steps, the antibody secreting cells are chased with a second labeled form of the antigen. The second labeled form of the antigen has a label that provides a different detectable signal than the label on the first labeled form of the antigen. Suitable choices of the label have been described herein, as long as the label on the second labeled form of the antigen is different from the label on the first labeled form of antigen. Such labels include small molecules, radioisotopes, enzymatic proteins and fluorescent dyes. In some embodiments, the first label is AlexaFluor647, and the second label is phycoerythrin.

In some embodiments, the second labeled form of the antigen is a monovalent form of the antigen. In some embodiments, the second labeled form of the antigen is a multivalent form of the antigen. In some embodiments, the second labeled form of the antigen comprises a mixture of a monovalent and a multivalent form of the antigen. In embodiments where a multivalent form of the antigen is used, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer may include trimer and/or dimer in addition to tetramer. In some embodiments, a streptavidin multimer is conjugated with a fluorophore such as phycoerythrin. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

The first labeled form of the antigen and the second labeled form of the antigen can be the same or different valent form of the antigen but must have labels that emit different detectable signals from each other. In some embodiments, the first labeled form of the antigen is a monovalent form of the antigen and the second labeled form of antigen is also a monovalent form. In some embodiments, the first labeled form of the antigen is a monovalent form of the antigen whereas the second labeled form of the antigen is a multivalent form.

In some embodiments, the chase is performed with an unlabeled form of an antigen (cold) and a second labeled form of the antigen (hot), also referred herein as a combination chase. In such embodiments, after the initial binding and washing steps, the antibody-secreting cells are chased with an unlabeled form of the antigen and a second labeled form of the antigen. The two forms of chase antigen: the unlabeled form of the antigen (“cold chase antigen”) and the second labeled form of the antigen (“hot chase antigen”) can be brought into contact with the cells at the same time or sequentially (e.g., with the cold chase antigen being added first, followed by the hot chase antigen, or vice versa). In some embodiments, the unlabeled form of the antigen is a monovalent form. In some embodiments, the unlabeled form of the antigen is a multivalent form. In some embodiments, the unlabeled form of the antigen is a mixture of monovalent form and multivalent form. In some embodiments, the second labeled form of the antigen is a monovalent form. In some embodiments, the second labeled form of the antigen is a multivalent form. In some embodiments, the second labeled form of the antigen comprises a mixture of a monovalent form and a multivalent form. In embodiments where a multivalent form of the antigen is used in a combination chase, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer may include trimer and/or dimer in addition to tetramer. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

For any format of the chase, the chase antigen concentration used is in excess as compared to the concentration of the first labeled form of the antigen, irrespective of the form of antigen used for the chase (i.e., unlabeled form, a second labeled form, or an unlabeled form and a second labeled form). In embodiments where an unlabeled form of the antigen is used (in a cold chase or in a combination chase), the antigen in the unlabeled form is at least 2 fold, i.e., 2 fold to up to, e.g., 2500 fold, in molar ratio relative to the first labeled form of the antigen. In some embodiments, the antigen in the unlabeled form is 2-fold in molar ratio relative to the first labeled form of the antigen. In some embodiments, the antigen in the unlabeled form is 3-fold in molar ratio relative to the first labeled form of the antigen. In some embodiments, the antigen in the unlabeled form is 4-fold in molar ratio relative to the first labeled form of the antigen. In some embodiments, the antigen in the unlabeled form is 5-fold in molar ratio relative to the first labeled form of the antigen. In some embodiments, the antigen in the unlabeled form is 6-fold, 7-fold, 8-fold, or 9-fold in molar ratio relative to the first labeled form of the antigen. In some embodiments, the antigen in the unlabeled form is 10-fold, 15-fold, 20-fold, or 25-fold in molar ratio relative to the first labeled form of the antigen. In some embodiments, the antigen in the unlabeled form is 30-fold, 40-fold, 50-fold, 60-fold, 70-fold, 80-fold, 90-fold, 100-fold, 150-fold, or greater in molar ratio relative to the first labeled form of the antigen. In some embodiments, depending on the concentration of the first labeled form of the antigen, the unlabeled form of the antigen may be at a concentration between 0.4 nM to 1 uM, or 10 to 600 nM. In some embodiments, depending on the concentration of the first labeled form of the antigen, the unlabeled form of the antigen is at a concentration between 10 nM to 600 nM, 10 nM to 500 nM, 10 nM to 400 nM, 10 nM to 300 nM, 10 nM to 200 nM, 10 nM to 150 nM, 10 nM to 100 nM, 10 nM to 75 nM, 10 nM to 65 nM, 10 nM to 50 nM, 10 nM to 40 nM, 10 nM to 30 nM, 10 nM to 25 nM, or 10 nM to 20 nM. In embodiments where a second labeled form of the antigen is used (in a hot chase or a combination chase), the antigen in the second labeled form is at least 2 fold, i.e., 2 fold to up to, e.g., 2500 fold, in molar ratio relative to the first labeled form of the antigen. In some embodiments, the antigen in the second labeled form is 2-fold in molar ratio relative to the first labeled form of the antigen. In some embodiments, the antigen in the second labeled form is 3-fold in molar ratio relative to the first labeled form of the antigen. In some embodiments, the antigen in the second labeled form is 4-fold in molar ratio relative to the first labeled form of the antigen. In some embodiments, the antigen in the second labeled form is 5-fold in molar ratio relative to the first labeled form of the antigen. In some embodiments, the antigen in the second labeled form is 6-fold, 7-fold, 8-fold, or 9-fold in molar ratio relative to the first labeled form of the antigen. In some embodiments, the antigen in the second labeled form is 10-fold, 15-fold, 20-fold, or 25-fold in molar ratio relative to the first labeled form of the antigen. In some embodiments, the antigen in the second labeled form is 30-fold, 40-fold, 50-fold, 60-fold, 70-fold, 80-fold, 90-fold, 100-fold, 150-fold, or greater in molar ratio relative to the first labeled form of the antigen. In some embodiments, depending on the concentration of the first labeled form of the antigen, the second labeled form of the antigen may be at a concentration between 0.4 nM to 1 uM, or 10 nM to 600 nM. In some embodiments, depending on the concentration of the first labeled form of the antigen, the second labeled form of the antigen may be at a concentration between 10 nM to 600 nM, 10 nM to 500 nM, 10 nM to 400 nM, 10 nM to 300 nM, 10 nM to 200 nM, 10 nM to 150 nM, 10 nM to 100 nM, 10 nM to 75 nM, 10 nM to 65 nM, 10 nM to 50 nM, 10 nM to 40 nM, 10 nM to 30 nM, 10 nM to 25 nM, or 10 nM to 20 nM.

In some embodiments where a combination chase is performed, the cold chase antigen and hot chase antigen can be at the same concentration or at different concentrations.

In some embodiments of a combination chase, the cells are contacted with a cold chase antigen and a hot chase antigen sequentially; and in some such embodiments, a washing step can be included between the two chase antigens. In such embodiments, the cells are washed with wash buffer for an allotted time sufficient to remove unbound antigen. In some embodiments, washing the cells for a period of time of about 10 minutes to about 60 minutes, in one or more washes.

The chase is performed for a period of time sufficient to allow the chase antigen to bind to antibodies. In some embodiments, the chase is performed with an unlabeled form or a second labeled form of the antigen for a time of about 5 to about 60 minutes, e.g., about 10 minutes, about 20 minutes, about 30 minutes, about 40 minutes, about 45 minutes, or about 50 minutes. In some embodiments of a combination chase, the chase is performed with an unlabeled form of the antigen for a time of about 5 to about 60 minutes followed by incubating with a second labeled form of the antigen for a time of about 5 to about 60 minutes. The time of incubation with the unlabeled form of antigen and with the second labeled form of antigen can be the same or different length. As a non-limiting example, the unlabeled form of antigen may contact the cells for about 30 minutes while the second labeled form of the antigen may subsequently contact the cells for about 45 minutes, and vice versa. In some embodiments of a combination chase, the chase is performed with a second labeled form of the antigen for a time of about 5 to about 60 minutes. In some embodiments of a combination chase, the chase is performed with an unlabeled form of the antigen and a second labeled form of the antigen at the same time for a time of about 5 to about 60 minutes.

In some embodiments, the chase is performed more than once. In some embodiments, the cold chase is performed more than once. In some embodiments, the hot chase is performed more than once. In some embodiments, the cold chase is performed once followed by a hot chase performed more than once. In some embodiments, a cold chase is performed more than once followed by a hot chase performed once. In some embodiments, a cold chase is performed more than once followed by a hot chase performed more than once. In some embodiments, a combination chase is performed more than once. In some embodiments, a wash step is included after each chase step. In some embodiments, more than one wash step is included after each chase step, e.g., 2 washes or 3 washes.

After chase has been completed, unbound antigen is again removed through washing and the cells remaining bound to the first labeled form of the antigen are collected. In some embodiments where the first detectable label is a first fluorescent label, fluorescence-activated cell sorting (FACS) can be utilized to collect the cells remaining bound to the first labeled form of the antigen. In some embodiments where the first detectable label is a first fluorescent label, and the second detectable label is a second fluorescent label that differentiates from the first fluorescent label, two-dimensional FACS is used to collect cells that remain bound to the first labeled form of the antigen. In specific embodiments, the first detectable label is A647 and the second detectable label is Phycoerythrin.

### Isolation of Antibody-Encoding Nucleic Acids

A pool of antibody secreting cells can be sorted or separated into single cells. Protocols for single cell isolation by flow cytometry are well-known (Huang, J. et al, 2013, supra). Single cells may be sorted and collected by alternative methods known in the art, including but not limited to manual single cell picking, limited dilution, microfluidics, laser capture microdissection, and Gel Bead Emulsions (GEMs), which are all well-known in the art. See, for example, Rolink et al., J. Exp Med (1996) 183:187-194; Lightwood, D. et al, J. Immunol. Methods (2006) 316(1-2):133-43; Gross et al., Int. J. Mol. Sci. (2015) 16: 16897-16919; and Zheng et al., Nature Communications (2017) 8: 14049. Gel Bead Emulsions (GEMs) are also commercially available (e.g., 10×Chromium System from 10× Genomics, Pleasanton, CA).

Once obtained, single antibody-secreting cells may be propagated by common cell culture techniques for subsequent DNA preparation. Alternatively, antibody genes may be amplified from single antibody-producing cells directly and subsequently cloned into DNA vectors.

A nucleic acid encoding an antibody or a fragment thereof can be isolated from single antibody-secreting cells obtained herein. For example, genes or nucleic acids encoding immunoglobulin variable heavy and variable light chains (i.e., VH, VL, VL can be VK or VA) can be recovered using RT-PCR protocols with nucleic acids isolated from antibody-secreting cells. For example, a nucleic acid encoding an antibody fragment is first reverse-transcribed (RT) to complementary DNA (cDNA). The cDNA is subsequently amplified in PCR reactions using primers specific to antibody gene sequences, e.g., constant regions of an antibody chain. All chains can be amplified using chain-specific primers in multiplex or separately. The amplicons are then sequenced to obtain the nucleic acid sequence encoding the antibody fragment. These RT-PCR protocols are well known and conventional techniques, as described for example, by Wang et al., J. Immunol. Methods (2000) 244:217-225 and described herein.

In some embodiments, the nucleic acid encodes a fragment of an antibody, such as a variable domain, constant domain or combination thereof. In certain embodiments, the nucleic acid isolated from an antibody-producing cell encodes a variable domain of an antibody. In some embodiments, the nucleic acid encodes an antibody heavy chain or a fragment thereof. In other embodiments, the nucleic acid encodes an antibody light chain or a fragment thereof.

Once recovered, antibody-encoding genes or nucleic acids can be cloned into IgG heavy- and light-chain expression vectors and expressed via transfection of host cells. For example, antibody-encoding genes or nucleic acids can be inserted into a replicable vector for further cloning (amplification of the DNA) or for expression (stably or transiently) in cells. Many vectors, particularly expression vectors, are available or can be engineered to comprise appropriate regulatory elements required to modulate expression of an antibody encoding gene or nucleic acid.

An expression vector in the context of the present disclosure can be any suitable vector, including chromosomal, non-chromosomal, and synthetic nucleic acid vectors (a nucleic acid sequence comprising a suitable set of expression control elements) as described herein. Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

In some embodiments, a nucleic acid molecule is included in a naked DNA or RNA vector, including, for example, a linear expression element (as described in, for instance, Sykes and Johnston, Nat Biotech (1997) 12:355-59), a compacted nucleic acid vector (as described in for instance U.S. Pat. No. 6,077,835), or a plasmid vector such as pBR322 or pUC 19/18. Such nucleic acid vectors and the usage thereof are well known in the art. See, for example, U.S. Pat. Nos. 5,589,466 and 5,973,972.

In certain embodiments, the expression vector can be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH. See, F. Ausubel et al., ed. Current Protocols in Molecular Biology, Greene Publishing and Wiley InterScience New York (1987); and Grant et al., Methods in Enzymol 153, 516-544 (1987).

In some embodiments, an expression vector carrying a nucleic acid isolated from antibody-secreting cell and encoding an antibody or fragment thereof is introduced into a host cell for expression of the antibody or the fragment thereof. Host cells include, e.g., mammalian cells, yeast cell, bacterial cells, or insect cells. In some embodiments, the host cells are cultured under conditions that express the nucleic acid, and the antibody or portion thereof can then be produced and isolated for further use. In some embodiments, host cells comprising one or more of the above nucleic acids are cultured under conditions that express a full-length antibody, and the antibody can then be produced and isolated for further use. In certain embodiments, the host cell comprises a nucleic acid that encodes a variable domain of an antibody, and the cell is cultured under conditions that express the variable domain. In other embodiments, the host cell comprises a nucleic acid that encodes a variable heavy chain (VH) domain of an antibody, and the cell is cultured under conditions that express the VH domain. In another embodiment, the host cell comprises a nucleic acid that encodes a variable light chain (VL) domain of an antibody, and the cell is cultured under conditions that express the VL domain. In specific embodiments, the host cell comprises a nucleic acid that encodes a VH domain of an antibody and nucleic acid that encodes a VL domain of an antibody, and the cell is cultured under conditions that express the VH domain and the VL domain.

In some embodiments, the host cell is a bacterial or yeast cell. In some embodiments, the host cell is a mammalian cell. In other embodiments, the host cell can be, for example, a Chinese hamster ovarian cells (CHO) such as, CHO K1, DXB-11 CHO, Veggie-CHO cells; a COS (e.g., COS-7); a stem cell; retinal cells; a Vero cell; a CV1cell; a kidney cell such as, for example, a HEK293, a 293 EBNA, an MSR 293, an MDCK, aHaK, a BHK21 cell; a HeLa cell; a HepG2 cell; W138; MRC 5; Colo25; HB 8065; HL-60; a Jurkat or Daudi cell; an A431 (epidermal) cell; a CV-1, U937, 3T3 or L-cell; a C127 cell, SP2/0, NS-0 or MMT cell, a tumor cell, and a cell line derived from any of the aforementioned cells. In a particular embodiment, the host cell is a CHO cell. In a specific embodiment, the host cell is a CHO K1 cell.

The present disclosure also provides methods of identifying a region of a gene encoding an antigen-binding fragment of a target antibody, wherein the target antibody is secreted by an antibody secreting cell comprising a modified cell surface, and wherein the target antibody is subsequently captured on the modified cell surface, the method comprising: a) providing an antibody secreting cell comprising a modified cell surface, wherein the cell secretes a target antibody wherein the target antibody is subsequently captured on the modified cell surface, wherein the target antibody captured on the modified cell surface is bound to an antigen, wherein the antigen is linked to a barcode nucleic acid molecule, and wherein the antibody secreting cell further comprises a nucleic acid molecule comprising the region of the gene encoding the antigen-binding fragment of the target antibody captured on the modified cell surface; b) hybridizing a portion of the barcode nucleic acid molecule to a portion of a first nucleic acid molecule attached to a solid surface; c) hybridizing a portion of the nucleic acid molecule comprising the region of the gene encoding the antigen-binding fragment of the target antibody captured on the modified cell surface to a portion of a second nucleic acid molecule attached to the solid surface; d) preparing a first library of amplicons of gene expression in the antibody secreting cell, a second library of amplicons of variable regions, diversity regions, and joining regions (VDJ) in the antibody secreting cell, and a third library of amplicons of antigen barcode nucleic acid molecules; e) sequencing each of the three libraries of amplicons; and f) identifying the region of the gene encoding the antigen-binding fragment of the target antibody captured on the modified cell surface.

In the above method, regarding step b) hybridizing a portion of the barcode nucleic acid molecule to a portion of a first nucleic acid molecule attached to a solid surface, in the partition (e.g., droplet), the partition (e.g., bead) dissolves and the cell barcode nucleic acid molecule/TSO oligos are free floating.

In the above method, regarding step c) hybridizing a portion of the nucleic acid molecule encoding the region of the gene encoding the antigen-binding fragment of the target antibody captured on the modified cell surface to a portion of a second nucleic acid molecule attached to the solid surface, the nucleic acid molecule comprising the region of the gene encoding the antigen-binding fragment of the target antibody captured on the modified cell surface (e.g., the BCR mRNA) does not directly hybridize. Rather, it is first made into first strand cDNA, and then the reverse transcriptase adds CCC to the end of the cDNA. The CCC then hybridizes to the rGrGrG part of the TSO. In addition, regarding the second nucleic acid molecule attached to the solid surface, in the partition (e.g., droplet), the partition (e.g., bead) dissolves and the second nucleic acid molecule is free floating.

In some embodiments, the modification of the modified cell surface comprises avidin, streptavidin, anti-biotin, biotin, the jun protein or a portion thereof, the fos protein or a portion thereof, the mad protein or a portion thereof, the max protein or a portion thereof, the myc protein or a portion thereof, an azide, an alkyne, and/or a phosphine. In some embodiments, the modification of the modified cell surface comprises avidin. In some embodiments, the modification of the modified cell surface comprises streptavidin. In some embodiments, the modification of the modified cell surface comprises biotin. In some embodiments, the modification of the modified cell surface comprises the jun protein or a portion thereof. In some embodiments, the modification of the modified cell surface comprises the fos protein or a portion thereof. In some embodiments, the modification of the modified cell surface comprises the mad protein or a portion thereof. In some embodiments, the modification of the modified cell surface comprises the max protein or a portion thereof. In some embodiments, the modification of the modified cell surface comprises the myc protein or a portion thereof. In some embodiments, the modification of the modified cell surface comprises an azide. In some embodiments, the modification of the modified cell surface comprises an alkyne. In some embodiments, the modification of the modified cell surface comprises a phosphine.

In some embodiments, the portion of the barcode nucleic acid molecule is complementary to a first template switch oligonucleotide (TSO). In some embodiments, the portion of the barcode nucleic acid molecule can be ligated to a first TSO. In some embodiments, a 3′ barcoded system can be used. In this case, the antigen barcode will have polyA. VDJ enrichment and sequencing may be performed differently.

In some embodiments, the portion of the first nucleic acid molecule attached to the solid surface comprises a first template switch oligo (TSO). In some embodiments, the 3′ terminus of the first nucleic acid molecule attached to the solid surface comprises a first sequence of three contiguous riboguanosine residues. In some embodiments, the first nucleic acid molecule attached to the solid surface further comprises a first unique molecular identifier (UMI). In some embodiments, the first nucleic acid molecule attached to the solid surface further comprises a first surface barcode. In some embodiments, the first nucleic acid molecule attached to the solid surface further comprises a first sequencing primer. In some embodiments, the first nucleic acid molecule attached to the solid surface comprises, from 3′ to 5′, the first sequence of three riboguanosine residues, the first TSO, the first UMI, the first surface barcode, and the first sequencing primer. In some embodiments, the first nucleic acid molecule attached to the solid surface is attached by the 5′ terminus of the first nucleic acid molecule attached to the solid surface.

In some embodiments, the first nucleic acid molecule attached to the solid surface comprises a first DNA molecule attached to the solid surface. In some embodiments, the first DNA molecule attached to the solid surface comprises a first single-stranded DNA molecule attached to the solid surface. In some embodiments, the portion of the first single-stranded DNA molecule attached to the solid surface comprises a first ISO and the first single-stranded DNA molecule attached to the solid surface beginning is reverse transcribed from the 3′ terminus of the portion of the barcode nucleic acid molecule which is complementary to the first TSO. Reverse transcription also occurs in the other strand as well, wherein the antigen barcode nucleic acid is extended from its 3′ and the first nucleic acid molecule attached to the solid surface is also extended from its 3′.

In some embodiments, the barcode nucleic acid molecule is a single-stranded DNA barcode nucleic acid molecule, wherein the portion of the first nucleic acid molecule attached to the solid surface comprises a first TSO, and the single-stranded DNA barcode nucleic acid molecule is reverse transcribed beginning from the 3′ terminus of the first TSO.

In some embodiments, the 3′ terminus of the portion of the barcode nucleic acid molecule comprises three contiguous cytidine or ribocytidine residues.

In some embodiments, an mRNA molecule encoding the region of the gene encoding the antigen-binding fragment of the target antibody captured on the modified cell surface is reverse transcribed, thereby generating a single-stranded DNA molecule encoding the region of the gene encoding the antigen-binding fragment of the target antibody captured on the modified cell surface. The actual chemistry occurs in solution in the partition (e.g., droplet).

In some embodiments, the nucleotide sequence of the single-stranded DNA molecule encoding the region of the gene encoding the antigen-binding fragment of the target antibody captured on the modified cell surface differs from a complement of the mRNA molecule encoding the region of the gene encoding the antigen-binding fragment of the target antibody captured on the modified cell surface in that the 3′ terminus of the single-stranded DNA molecule encoding the region of the gene encoding the antigen-binding fragment of the target antibody captured on the modified cell surface comprises three additional, contiguous cytidine or ribocytidine residues. In some embodiments, the 3′ terminus of the mRNA molecule encoding the region of the gene encoding the antigen-binding fragment of the target antibody captured on the modified cell surface comprises a plurality of contiguous adenine residues. In some embodiments, the 5′ terminus of the single-stranded DNA molecule encoding the region of the gene encoding the antigen-binding fragment of the target antibody captured on the modified cell surface comprises a plurality of contiguous thymine residues. In some embodiments, the 5′ terminus of the second nucleic acid molecule attached to the solid surface comprises a second sequence of three contiguous riboguanosine residues.

In some embodiments, the second nucleic acid molecule attached to the solid surface further comprises a second UMI. In some embodiments, the second nucleic acid molecule attached to the solid surface further comprises a second surface barcode. In some embodiments, the second surface barcode is the same surface (cell) barcode as the first nucleic acid molecule, in order to match antigen to BCR. In some embodiments, the second nucleic acid molecule attached to the solid surface further comprises a second sequencing primer. In some embodiments, the second nucleic acid molecule attached to the solid surface comprises, from 3′ to 5′, the second sequence of three riboguanosine residues, the second TSO, the second UMI, the second surface barcode, and the second sequencing primer. In some embodiments where a 10×genomics system is used, only the UMI is different for each of the nucleic acid molecules on the same surface. As long as the cell barcode is the same, the other components (e.g., ISO and sequencing primer) can be different. In some embodiments, the second nucleic acid molecule attached to the solid surface is attached by the 5′ terminus of the second nucleic acid molecule attached to the solid surface.

In some embodiments, the second nucleic acid molecule attached to the solid surface comprises a second DNA molecule attached to the solid surface. In some embodiments, the second DNA molecule attached to the solid surface comprises a second single-stranded DNA molecule attached to the solid surface. In some embodiments, the 3′ terminus of the portion of the second single-stranded DNA molecule attached to the solid surface comprises a second sequence of three contiguous riboguanosine residues. In some embodiments, the 3′ terminus of the portion of the nucleic acid molecule encoding the region of the gene encoding the antigen-binding fragment of the target antibody captured on the modified cell surface comprises three contiguous cytidine residues. In some embodiments, the second single-stranded DNA molecule attached to the solid surface is reverse transcribed beginning from the 3′ terminus of the three contiguous cytidine residues.

In some embodiments, the nucleic acid molecule encoding the region of the gene encoding the antigen-binding fragment of the target antibody captured on the modified cell surface comprises a second single-stranded DNA molecule encoding the region of the gene encoding the antigen-binding fragment of the antibody captured on the modified cell surface. In some embodiments, the 3′ terminus of the portion of the single-stranded DNA molecule encoding the region of the gene encoding the antigen-binding fragment of the target antibody captured on the modified cell surface comprises three contiguous cytidine or ribocytidine residues, and wherein the 5′ terminus of the portion of the second single-stranded DNA molecule attached to the solid surface comprises a second sequence of three contiguous riboguanosine residues. In some embodiments, the second single-stranded DNA molecule attached to the solid surface is reverse transcribed beginning from the 3′ terminus of the portion of the single-stranded DNA molecule encoding the region of the gene encoding the antigen-binding fragment of the target antibody captured on the modified cell surface. In some embodiments, the single-stranded DNA molecule encoding the region of the gene is reverse transcribed beginning from the 3′ terminus of the second single-stranded DNA molecule attached to the solid surface.

In some embodiments, the antibody secreting cell is disposed within a partition with the solid surface. In some embodiments, the partition contains a single antibody secreting cell. In some embodiments, the partition contains a single plasma cell or plasmablast. In some embodiments, the solid surface comprises a bead. In some embodiments, the antibody secreting cell within the partition is lysed. In some embodiments, the solid surface (e.g., bead) is dissolved upon partitioning and its surface-attached DNA molecules are released into solution within the partition. In some embodiments, the partition is an oil. In some embodiments, the partition is in the form of a droplet.

In some embodiments, the barcoded nucleic acid molecule further comprises a third sequencing primer.

In some embodiments, each amplicon is produced from a first library of amplicons that comprises the gene that encodes the antigen-binding fragment of the target antibody captured on the modified cell surface.

In some embodiments, the first library of amplicons is sample de-multiplexed, aligned, filtered, and UMI counted. In some embodiments, the sequencing of the first library of amplicons comprises next-generation sequencing.

In some embodiments, each amplicon of the first library of amplicons is mapped and aligned to a standard reference genome. In some embodiments, the standard reference genome comprises a human standard reference genome. In some embodiments, the human standard reference genome is GRCh38. In some embodiments, the standard reference genome comprises a murine standard reference genome. In some embodiments, the murine standard reference genome is mm10.

In some embodiments, each amplicon of the first library of amplicons is mapped to the standard reference genome with a single cell alignment software, such as STAR or kallisto (Bray et al., Nat. Biotechnol., 2016, 34, 525-527; Erratum in: Nat. Biotechnol., 2016, 34, 888), wherein each amplicon of the first library of amplicons comprises a unique molecular identifier, wherein all amplicons of the first library of amplicons which map to annotated genes of the standard reference genome are binned, and wherein the binned amplicons are counted for each unique molecular identifier (UMI), thereby generating a single cell count matrix comprising a mapped count for each annotated gene and a count for each UMI.

In some embodiments, the single cell count matrix is filtered for high quality cells and extensively profiled genes by calculating the ratio of the total number of annotated genes divided by the login of the total number of UMIs counted. In some embodiments, cells with a gene to UMI ratio below about 0.1 are filtered out. In some embodiments, cells with more than about four times the interquartile range of the total number of UMIs counted are filtered out. In some embodiments, cells with more than about 80% of reads map to a mitochondrial gene are filtered out. In some embodiments, cells with a gene to UMI ratio below about 0.1, cells with more than about four times the interquartile range of the total number of UMIs counted, and cells with more than about 80% of reads map to a mitochondrial gene are filtered out. In some embodiments, any threshold can be set for gene to UMI ratios and mitochondrial threshold. These values are highly dependent on the tissue being used. More stable tissues, such as blood and lymph nodes, require less stringent thresholds whereas tissue such as the gut, lung, or skin would have higher numbers of dead/dying cells with high mitochondrial contamination.

In some embodiments, the single cell count matrix is normalized such that the total number of UMIs counted is about 1,000, 5,000, 10,000, 20,000, 50,000, or 100,000. In some embodiments, the single cell count matrix is normalized such that the total number of UMIs counted is about 10,000. In some embodiments, the single cell count matrix is normalized such that the total number of UMIs counted is about 5,000. In some embodiments, the single cell count matrix is normalized such that the total number of UMIs counted is about 20,000.

In some embodiments, the principle component embeddings of the single cell count matrix are computed by principle component analysis (PCA) and are used as input to compute a uniform manifold approximation and projection (UMAP). Alternatively, tSNE projection could also be calculated from the PCA embeddings (van der Maaten et al., J. Mach. Learning Res., 2008, 9, 2579-2605). Additional iterative clustering of major cell types can be performed to identify similarities across batches. In some embodiments, the UMAP is generated using the methods disclosed in Korsunsky et al., Nat. Methods, 2019, 16, 1289-1296.

In some embodiments, cluster-specific centroids are used to determine a linear adjustment function per cell, applying the linear adjustment function per cell to correct for differences in batches, thereby generating batch corrected embeddings, and using the batch corrected embeddings to generate a uniform manifold approximation and projection (UMAP) in two dimensions. The method is called harmony (Korsunsky et al., Nat. Methods, 2019, 16, 1289-1296), however, several alternatives exist including the calculation of integration anchors from Seurat (Hao et al., Cell, 2021, 184, 3573-3587).

In some embodiments, a weighted k-neighbor graph is determined and the Leiden algorithm applied to the weighted k-neighbor graph with resolution values of about 0.1, about 0.2, about 0.5, and about 1.0 to determine cell type clusters in an unsupervised manner. In some embodiments, a weighted k-neighbor graph is determined and the Leiden algorithm applied to the weighted k-neighbor graph with resolution values of about 0.1 to determine cell type clusters in an unsupervised manner. In some embodiments, a weighted k-neighbor graph is determined and the Leiden algorithm applied to the weighted k-neighbor graph with resolution values of about 0.2 to determine cell type clusters in an unsupervised manner. In some embodiments, a weighted k-neighbor graph is determined and the Leiden algorithm applied to the weighted k-neighbor graph with resolution values of about 0.5 to determine cell type clusters in an unsupervised manner. In some embodiments, a weighted k-neighbor graph is determined and the Leiden algorithm applied to the weighted k-neighbor graph with resolution values of about 1.0 to determine cell type clusters in an unsupervised manner. Leiden is a community-detection algorithm that performs clustering iteratively by splitting nodes of similar cells to generate well-connected clusters (Traag et al., Scientific Reports, 2019, 9, 5233). Alternative algorithms include Louvain, K-means and NMF clustering.

In some embodiments, a pairwise Wilcox test is performed to all of the cells in one cluster and comparing the result of the first pairwise Wilcox test, and another pairwise Wilcox test is performed to all of the cells in every other cluster to quantify a p-value and fold-change for each gene in each cluster. In some embodiments, when a gene has a low p-value and the high fold-change, the cells are labeled by a common marker of antibody secreting cells and the cells are labeled by clustered subtypes that are named by the top genes from the differential expression result. This can be repeated for the cells with and without antigen specificity in each isotype (primarily IgE). In some embodiments, a test such as DESeq2 can be used for differential expression.

In some embodiments, sample de-multiplexing is performed, de novo assembly of read pairs into contigs accomplished, and the contigs against germline segment V(D)J reference sequences are aligned and annotated for the second family of amplicons.

In some embodiments, the alignment of the contigs against germline segment V(D)J reference sequences comprises aligning against a human germline reference database or a murine germline reference database using the software IgBlast (Ye et al., Nuc. Acids Res., 2013, 41, W34-W40). In some embodiments, the human germline reference database comprises the IMGT database of human germline immunoglobulin sequences (world wide web at “imgt.org/IMGTrepertoire/LocusGenes/”). In some embodiments, the murine germline reference database comprises the IMGT germline reference database.

In some embodiments, VDJ sequences are compared against sequences in the human germline reference database or the murine germline reference database, thereby labeling the VDJ sequences for quality alignments by checking for in-frame alignments, length of the CDR3, and/or the absence of stop codons in the variable region sequence and the VDJ sequences filtered based on the labels. In some embodiments, VDJ sequences are compared against sequences in the human germline reference database or the murine germline reference database, thereby labeling the VDJ sequences for quality alignments by checking for in-frame alignments. In some embodiments, VDJ sequences are compared against sequences in the human germline reference database or the murine germline reference database, thereby labeling the VDJ sequences for length of the CDR3. In some embodiments, VDJ sequences are compared against sequences in the human germline reference database or the murine germline reference database, thereby labeling the VDJ sequences for the absence of stop codons in the variable region sequence. Alternately, this step can be omitted to retain all possible VDJ sequences, which can be aligned to IgBlast to confirm high quality sequences that are in frame for human or mouse variable regions. This step may not be possible for less well-studied organisms if the database of germline VDJ sequences is not available.

In some embodiments, the VDJ sequences are mapped to a reference database (e.g., IMGT) of immunoglobulin chains.

In some embodiments, the immunoglobulin isotype, the variable region mapping, the joining region mapping, the diversity region mapping, the accuracy of full-length variable regions for both heavy and light chain sequence per cell are confirmed by the alignment. In some embodiments, the immunoglobulin isotype is confirmed by the alignment. In some embodiments, the variable region mapping is confirmed by the alignment. In some embodiments, the joining region mapping is confirmed by the alignment. In some embodiments, the diversity region mapping is confirmed by the alignment. In some embodiments, the accuracy of full-length variable regions for both heavy and light chain sequence per cell is confirmed by the alignment. In some embodiments, the VDJ sequences are mapped and annotated to the germline through IgBlast.

In some embodiments, the third library of amplicons is sample de-multiplexed, aligned, filtered, and UMI counted. In some embodiments, a single cell alignment software, such as STAR or kallisto, can be used.

In some embodiments, the third library of amplicons are mapped to a custom short-read reference which comprises the barcode nucleic acid molecule reference associated with each antigen. In some embodiments, the counts for each uniquely mapped barcode nucleic acid molecule are summed for each cell in a barcoded antigen single cell matrix.

In some embodiments, the barcode nucleic acid molecules across all cells are quantified and normalized by taking the centered log-ratio of each barcode nucleic acid molecule of the plurality of barcode nucleic acid molecules across each sample capture. Alternately, either centered log-ratio or denoising and scaling by background (DSB) can be used (Mule et al., bioRxiv, 2020.02.24.963603).

In some embodiments, the background antigen signal is removed by DSB for each barcode nucleic acid molecule of the plurality of barcoded nucleic acid molecules. Alternately, centered log-ratio can be used.

In some embodiments, an antigen signal distribution is determined, wherein when there is a well separated bimodal distribution in the antigen signal distribution, z-transformed values are used to compute antigen specificity, and wherein when there is not a well separated bimodal distribution in the antigen signal distribution, a quantile value is used. In some embodiments, rank order can be used rather than quantile value.

In some embodiments, the analysis of the first library of amplicons, the second library of amplicons, and the third library of amplicons is simultaneous to obtain at least one candidate sequence of the antigen-positive target antibody captured on the modified cell surface.

In some embodiments, antibody secreting cells with a valid antibody constant region are subset using the analysis of the first library of amplicons and the analysis of the second library of amplicons. In some embodiments, the antibody constant region comprises an IgE constant region. In some embodiments, the subsetted antibody secreting cell comprises detectable levels of IgE heavy chain and CD79a but lacking detectable levels of Ms4a1 and CD19, wherein the subsetted antibody secreting is an IgE plasma cell or plasmablast.

In some embodiments, the differential expression of the IgE antibody secreting cell is assessed against an IgA-secreting cell isotype, an IgG-secreting cell isotype, and/or an IgM-secreting cell isotype. In some embodiments, the assessment of differential expression further characterizes the unique transcriptional signature of the IgE antibody secreting cell.

In some embodiments, the antigen specificity of the IgE antibody secreting cell is assessed. In some embodiments, the assessment comprises comparing the antigen specificity of the IgE antibody secreting cell against a plurality of antigens and a control antigen, and wherein the plurality of antigens comprises the target antigen. In some embodiments, the comparing comprises calculating an empirical score for each antigen of the plurality of antigens and the control antigen by subtracting a quantile value of a signal associated with the antigen (qT) from a quantile value of a signal associated with the control antigen (qC) with a penalty factor (x) to determine an antigen specificity score where the antigen specificity score=qT−qC^{x}. In some embodiments, the penalty factor is the same for all antigens. In some embodiments, the penalty factor is different for different antibody isotypes. In some embodiments, the penalty factor is different when other antigens are used.

In some embodiments, the antigen specificity score for each of the plurality of antigens and the control antigen is determined. In some embodiments, a control antigen specificity score is calculated, wherein when the antigen specificity score is high and the control antigen specificity score is low, selecting the cell. In some embodiments, the VDJ regions identify antigen-specific antibodies.

In some embodiments, antibody candidates are selected from a ranked list of antigen signals using the paired V_{H}:V_{L} antibody sequence of the cell.

In some embodiments, differential expression between isotypes, conditions, and/or antigen specificity is determined, wherein determining the differential expression between isotypes comprises performing a pairwise Wilcox test for all of the cells in one isotype and performing a pairwise Wilcox test for all of the cells in another isotype, thereby quantifying a p-value and fold-change of each gene for each cluster; and wherein determining the differential expression between antigen specificity comprises performing a pairwise Wilcox test for all of the cells in one isotype and performing a pairwise Wilcox test for all of the cells in another isotype, thereby quantifying a p-value and fold-change of each gene for each cluster.

There are many models/statistics for differential expression analysis. Some examples include: 1) “wilcox”, which identifies differentially expressed genes between two groups of cells using a Wilcoxon Rank Sum test (as described herein); 2) “bimod”, which is a likelihood-ratio test for single cell gene expression, (McDavid et al., Bioinformatics, 2013); 3) “roc”, which identifies “markers” of gene expression using ROC analysis; for each gene, evaluates (using AUC) a classifier built on that gene alone, to classify between two groups of cells; an AUC value of 1 means that expression values for this gene alone can perfectly classify the two groupings (i.e., each of the cells in cells. 1 exhibit a higher level than each of the cells in cells. 2); an AUC value of 0 also means there is perfect classification, but in the other direction; a value of 0.5 implies that the gene has no predictive power to classify the two groups; it returns a “predictive power” (abs(AUC-0.5)*2) ranked matrix of putative differentially expressed genes; 4) “t”, which identifies differentially expressed genes between two groups of cells using the Student's t-test; 5) “negbinom”, which identifies differentially expressed genes between two groups of cells using a negative binomial generalized linear model; 6) “poisson”, which identifies differentially expressed genes between two groups of cells using a poisson generalized linear model; 7) “LR”, which uses a logistic regression framework to determine differentially expressed genes; constructs a logistic regression model predicting group membership based on each feature individually and compares this to a null model with a likelihood ratio test; 8) “MAST”, which identifies differentially expressed genes between two groups of cells using a hurdle model tailored to scRNA-seq data; utilizes the MAST package in R to run the DE testing; 9) “DESeq2”, which identifies differentially expressed genes between two groups of cells based on a model using DESeq2 which uses a negative binomial distribution (Love et al., Genome Biol., 2014); and 10) “detection rate”, which identifies differentially expressed genes between two groups of cells based on percent detection rate between cell groups.

In some embodiments, VDJ sequence similarity in an antigen positive target cell are clustered, wherein the clustering VDJ sequence similarity comprises sequence-based alignment, grouping of similar variable regions by amino acid sequence.

In some embodiments, the antigen positive target cell is IgE⁺.

In some embodiments, redundant sequences are trimmed.

The present disclosure also provides methods of identifying a region of a gene encoding an antigen-binding fragment of a target antibody, wherein the target antibody is secreted by an antibody secreting cell, the method comprising: a) contacting a population of antibody secreting cells with a second component of a binding pair to allow the second component of the binding pair to bind to the cell surface, wherein the population of antibody secreting cells comprises the antibody secreting cell that secretes the target antibody; b) contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the population of antibody secreting cells, and whereby the antibody-capture molecule captures the target antibody secreted by the antibody secreting cell; c) contacting the population of antibody secreting cells with a secondary anti-Ig antibody, whereby the target antibody secreted by the antibody secreting cell and captured by the antibody capture complex binds the secondary anti-Ig antibody; d) contacting the population of antibody secreting cells with an antigen comprising a barcode nucleic acid molecule, whereby the target antibody secreted by the antibody secreting cell and captured by the antibody capture complex binds the antigen; e) collecting a pool of antibody secreting cells, wherein each cell in the pool secretes an antibody that is captured by the antibody capture complex, bound by the secondary anti-Ig antibody and by the antigen comprising the barcode nucleic acid molecule, and wherein the pool of antibody secreting cells comprises the antibody secreting cell that secretes the target antibody; f) separating the pool of antibody secreting cells into single antibody secreting cells, and for each single antibody secreting cell: 1) hybridizing a portion of the barcode nucleic acid molecule to a portion of a first nucleic acid molecule attached to a solid surface, wherein the solid surface and the first nucleic acid molecule attached thereto are unique to each single antibody secreting cell; 2) hybridizing a portion of the nucleic acid molecule comprising the region of the gene encoding the antigen-binding fragment of the target antibody to a portion of a second nucleic acid molecule attached to the solid surface; 3) preparing a first library of amplicons of gene expression, a second library of amplicons of variable regions, diversity regions, and joining regions (VDJ), and a third library of amplicons of antigen barcode nucleic acid molecules; and 4) sequencing each of the three libraries of amplicons; and g) identifying the region of the gene encoding the antigen-binding fragment of the target antibody.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Materials and Methods

### Reagents Used

Mouse flow antibodies (fluorescently conjugated)

| | AbPID or | | REGN# or | |
|---|---|---|---|---|
| Antibody | Clone # | Supplier | Catalog # | Lot# |
| Fc Block | 2.4G2 | Tonbo | 70-0161-M001 | P0161032315705 |
| (CD16/32) | | Biosciences | | |
| B220 | RA3-6B2 | BD | 563793 | 121784 |
| CD138 | 281-2 | BD | 563193 | 9309295 |
| IgG1 | A85-1 | BD | 740121 | 0030366 |
| IgE | R35-72 | BD | 564207 and | 44493 and |
| | | | 744281 | 1082998 |
| IgM | II/41 | Invitrogen | 47-5790-82 | 2297386 |
| IgD | 11-26c.2a | BD | 565348 | 9023728 |
| TCRβ | H57-597 | Biolegend | 109220 | B249533 |
| Ly6G | 1A8-Ly6g | Biolegend | 127624 | B264760 |
| CD49b | DX5 | Biolegend | 108920 | B241908 |
| CD11b | M1/70 | Biolegend | 101226 | B259668 |
| TACI | 8F10 | BD | 742840 | 1260588 |
| CD98 | 4F2 | Biolegend | 128214 | B347216 |
| CD200R3 | Ba13 | Biolegend | 142208 | B301161 |
| CD19 | 1D3 | BD | 560143 | 1022418 |
| IgG2a | SB84a | Southern | 1155-02 | F1320-WE90 |
| | | Biotech | | |
| CD3 | 17A2 | Biolegend | 100214 | B316738 |

Human flow antibodies (fluorescently conjugated)

| | AbPID or | | REGN# or | |
|---|---|---|---|---|
| Antibody | Clone # | Supplier | Catalog # | Lot# |
| Human | polyclonal | eBioscience | 14-9161-73 | 2235896 |
| FC Block | | | | |
| CD20 | 2H7 | BD | 560631 | 6273736 |
| CD38 | HB7 | BD | 562666 | 8004645 |
| IgE | G7-26 | BD | 566324 | 7339778 |
| IgD | IA6-2 | BD | 555778 | 357891 |
| IgM | MHM88 | BioLegend | 314534 | B276950 |
| CD3 | UHT1 | BD | 555916 | 8066963 |
| CD11b | ICRF44 | BD | 562793 | 8067863 |
| CD14 | M5E2 | BioLegend | 301804 | B309751 |
| CD16 | 3G8 | BioLegend | 302006 | B256873 |
| CD123 | 7G3 | BD | 558663 | 8032894 |

Reagents used and lot numbers

| Reagent | Brand | Catalog/Lot |
|---|---|---|
| House Dust | Greer | XPB70D3A25/348717 |
| Mite (HDM) extract | | |
| Saline | Sigma | S8776/RNBF2733 |
| Microtainer ® tubes | BD Biosciences | 365967 |
| OptEIA ® mouse | BD Biosciences | 555248/1138318 |
| IgE ELISA set | | |
| EasySep ® Human | StemCell | 19554/1000042494 |
| Pan-B cell Isolation Kit | | |
| EasySep ® Mouse | StemCell | 19844A/10000054035 |
| Pan-B cell Isolation Kit | | |
| Streptavidin Conjugation Kit - | ABCAM | ab102921/GR3353137-1 |
| Lightning-Link ® | | and GR3394825 |
| Biotin Conjugation Kit - | ABCAM | Ab201795/GR3406889 |
| Lightning-Link ® | | |
| Bovine serum | Sigma | A9576/SLCH0670 |
| albumin (BSA) | | |
| Normal rat serum | StemCell | 13551/1000039221 |
| Ficoll ® density | GE Healthcare | 17-1440-03/10292123 |
| gradient medium | | |
| Dextran Sulfate | NA | NA |
| NHS-Biotin | Sigma | 20311/3526395 |
| ELISA Plate | Thermo Scientific | 80040LE/0910 |
| Carbonate-bicarbonate | Sigma | C3041-100/SLBW0243 |
| coating buffer | | |
| Distilled water | Gibco | 15230-170/2337487 |
| Tween ® 20 | Sigma | P1379/SLBS6187 |
| OptEIA ® | BD Biosciences, | 555213/0301707 |
| assay diluent | | |
| Dulbecco's phosphate- | Gibco | 14190/2276658 |
| buffered saline (DPBS) | | |
| OptEIA ® TMB substrate | BD Biosciences | 555214/9273407 |
| Stop solution | BDH VWR Analytical | BDH7500-1/21A1956065 |
| 4′,6-diamidino-2- | ThermoFisher | 62247/VG3030411 |
| phenylindole (DAPI) | | |
| Der p 1 antigen | IndoorBiotech | NA-DP1-1/44098 |
| Der p 2 antigen | IndoorBiotech | NA-DP2-1/41384 |
| Der f 1 antigen | IndoorBiotech | NA-DF1-1/44001 |
| Ole e 1 antigen | IndoorBiotech | RP-OE1-1/43138 |
| TotalSeq ® C0956 | BioLegend | 405283/B325046 |
| TotalSeq ® C0957 | BioLegend | 405285/B327470 |
| TotalSeq ® C0958 | BioLegend | 405293/B320055 |
| TotalSeq ® C0959 | BioLegend | 405159/B323806 |
| TotalSeq ® C0951 | Biolegend | 405261/B313967 |
| TotalSeq ® C0952 | Biolegend | 405263/B322438 |

### Biotinylation of Cell Surfaces

Cells were washed and resuspended in PBS containing 5% w/v BSA. Surface Fc was blocked with 1:10 diluted Fc Block (eBioscience) along with detection of surface BCR (for cell line assays) for fifteen minutes at 4° C. The cells were washed twice more with PBS, spun down at 500 g for 10 minutes, resuspended in 1 mL of freshly prepared NHS-biotin (Sigma, 0.5 mg/mL in PBS) and incubated at 37° C. for fifteen minutes. The biotinylated cells were washed three times in cold PBS containing 5% w/v BSA with a changed tube for each washing step.

### Preparation of Secreted IgE Capture Reagent

EctoFcεRIα was conjugated with streptavidin using a Lightning-Link® streptavidin conjugation kit to form the secreted IgE capture reagent. In brief, a solution of 0.5 mg/mL ectoFcεRIα containing 1 mg/mL streptavidin was prepared. One-hundred microliters of Modifier reagent was added to the solution with gentle mixing. The cap from the vial of Streptavidin Conjugation Mix was removed and the mixture, including the Modifier reagent, was added directly onto the lyophilized material. The material was resuspended gently by withdrawing and re-dispensing the added suspension once or twice using a pipette. The cap was replaced on the vial and the vial was stood for three hours in the dark at 20-25° C. Thereafter, 100 μL of Quencher reagent was added with gentle mixing. Thus, the secreted IgE capture reagent was generated.

Incubation of Cells Having Biotinylated Cell Surfaces with Secreted IgE Capture Reagent

Washed, biotinylated cells were resuspended at 1-3×10⁶ cells/mL in 1 mL of PBS containing 5% w/v BSA and 30 μg/mL purified secreted IgE capture reagent and placed in six-well plates. In some embodiments, a volume up to 3 mL in six-well plates can be used. The cells were incubated at 37° C. for one hour on a rotating platform to allow for capture of secreted IgE. The cells were then washed twice in PBS containing 5% w/v BSA.

### Lung-Cell Preparation

Tubes and reagents were prepared before lung samples were obtained. Ten milliliters of sterile water was added to 50 mg of Liberase® TH so as to have a final concentration of 26 collagenase Wunsch units/mL or 5.0 mg/mL collagenase. One milliliter aliquots were made into individual tubes and stored at −20° C. A Liberase® mix (digestion buffer) was prepared as needed:

| | Initial | Final | Volume |
|---|---|---|---|
| Reagent | Conc. | Conc. | to Add |
| Liberase TH Research Grade | 26 U/mL | 0.7 U/mL | 1.0 mL |
| DNase (in 20 mM Tris, | 10 mg/mL | 20 μg/mL | 74.4 μL |
| 1 mM MgCl₂, 50% glycerol) | | | |
| HBSS with Ca and Mg | — | — | 36.1 mL |

For each mouse, 1.5 mL of the Liberase® mix was pipetted into a separate 5 mL flow cytometry tube. The tubes were kept on ice or in a cool rack until digestion was required.

For each mouse, the largest lobe of the right lung or the whole lung was placed in a separate 2 mL Eppendorf® tube with 1 mL of the Liberase® mix. In those tubes, the lung material was chopped into small pieces, typically cubes of about 2-3 mm. This material was incubated at 37° C. for twenty minutes. The digesting material was added to gentleMACS™ C Tubes and the digestion stopped by the addition of 0.5 M EDTA to a final concentration of 10 mM. Two milliliters of MACS® buffer was also added. The caps were screwed on tightly and the capped tubes were run on a gentleMACS™ Octo Dissociator using the preloaded program m_lung_02_01. The resulting cells were centrifuged at 400 g for four minutes and the supernatant was decanted. Red-blood cells in the pellet were lysed with 1 mL of Red-Blood Cell Lysing Buffer for three minutes at ambient temperature. Thereafter, 10 mL of DPBS was added to deactivate the lysing buffer. The cells were centrifuged at 400 g for four minutes, the supernatant decanted, and the pellet resuspended in 1 mL of DPBS. The suspension was filtered through a 100 μm Millipore® plate filter into a 2 mL deep-well plate. The cells were centrifuged at 400 g for four minutes and resuspended in 500 μL DPBS. Fifty microliters from each sample were pooled with 100 μL from other samples and the 150 μL pooled samples were added to round-bottomed 96-well plates.

### Draining Lymph Node-Cell Preparation

To twelve-well, 70 μm cell strainer, plates 1-2 mL of RPMI media was added. The draining lymph nodes were collected and added to the plates. PCR plates were prepared and holes punched into each well with a 20 G needle and were placed on 2 mL deep-well plates.

The draining lymph nodes were mashed on a 74 μm cell strainer in 2 mL RPMI media using the back end of a 3 mL syringe. The preparation was filtered through a 100 μm Millipore® plate filter into a 2 mL deep-well plate and the filtered cells centrifuged at 400×g for four minutes. The pellet was resuspended in 500 μL DPBS and transferred to a 96-well deep-bottomed plate. This plate was centrifuged at 400×g for four minutes. The cells were resuspended in 200 μL of PBS. Into 96-well round-bottomed tubes were plated 200 μL of the suspension. The plate was centrifuged at 400×g for four minutes and the supernatant flicked off. The typical cell count from mice that had been administered intranasally 50 μg of HDM extract diluted in 20 μl of saline three times a week for fifteen weeks was 230×10⁶ cells.

### Bone Marrow-Cell Preparation

To 48-well plates, 200 μL of RPMI media was added. PCR plates were prepared and holes punched into each well with a 20 G needle and were placed on 2 mL deep-well plates.

The femurs of mice were extracted and the bones cleaned. Both ends of each bone were cut and placed in the PCR plate with holes. The PCR plate was placed on top of a 2 mL deep-well collection plate and centrifuged for four minutes at 500× g. The centrifugation step was repeated if any BM remained in the bones. The pellets were resuspended in 500 μL of red blood-cell lysis buffer was added. The suspension was incubated for three minutes at ambient temperature after which 1-2 mL of PBS was added to deactivate the lysis buffer. The resulting cells were centrifuged at 400×g for four minutes and the pellet resuspended in 1 mL DPBS. The suspension was filtered through a 100 μm Millipore plate filter into a 2 mL deep-well plate. From each sample, about 100-200 μL was taken for fluorescence minus one (FMO)s with the pool of the samples used for the FMO. Aliquoted, pooled samples were placed into twelve wells for the FMO. The remaining samples were centrifuged at 400×g for four minutes.

The bone-marrow samples were resuspended in 200 μL of PBS. Half of the bone-marrow sample was plated into 96-well round-bottomed plates. The plates were centrifuged at 400×g for four minutes and the supernatants flicked off. The typical cell count from mice that had been administered intranasally 50 μg of HDM extract diluted in 20 μl of saline three times a week for fifteen weeks was 116×10⁶ cells.

### Spleen-Cell Preparation

Spleens were extracted from mice and homogenized in 5 mL DPBS in gentleMACS™ tubes using the pre-loaded program TBA. Homogenized spleens were centrifuged at 400×g for four minutes and supernatant was discarded. Spleens were then re-suspended in 4 mL red blood cell lysis buffer and incubated at room temperature for 5 minutes, after which 10 mL DPBS was added to neutralize the lysis buffer. Spleens were then centrifuged at 400×g for four minutes and the supernatant was discarded. Spleens were then re-suspended in 10 mL DPBS and filtered through 70 μM filters for single cell suspensions. Cell counts and viability were determined and the spleens were then stained for antigen+ B cells.

### Pan-B Cell Isolation

B cells were isolated using an EasySep® Mouse Pan-B cell Isolation Kit. In brief, the samples were prepared at 1×10⁸ cells in 0.5-8 mL of PBS containing 5% w/v BSA. Fifty microliters of rat serum was added per mL of sample. The samples were added to 14 mL polystyrene round-bottom tubes (catalog no. 35205, lot no. 00421123). The cells were mixed and incubated for five minutes at ambient temperature. Fifty microliters of RapidSpheres® that had been vortexed for thirty seconds were added per milliliter of sample and the mixture was incubated at 2.5 minutes. Phosphate-buffered saline containing 5% w/v BSA was added to a volume of 5 mL and the tubes placed inside The Big Easy EasySep® magnet (StemCell catalog no. 18001) for 2.5 minutes at ambient temperature. The enriched cell suspension was poured into a new tube and counted. An overview of the procedure is set forth below:

| | | | 50 μL of Sample | | | |
|---|---|---|---|---|---|---|
| | | Resuspend | Rat | | | Pan-B |
| Cell | Cell | to 10⁸ | serum | Cocktail | | cells |
| type | Count | cells/mL | to add | to add | RapidSpheres ® | recovered |
| BM | 116 × 10⁶ | 1160 μL | 58 | 58 | 58 | 68 × 10⁶ |
| following | | | | | | |
| HDM | | | | | | |
| challenge | | | | | | |
| DLNs | 230 × 10⁶ | 2300 μL | 115 | 115 | 115 | 30 × 10⁶ |
| following | | | | | | |
| HDM | | | | | | |
| challenge | | | | | | |

### Example 1: Development of Capture Complexes

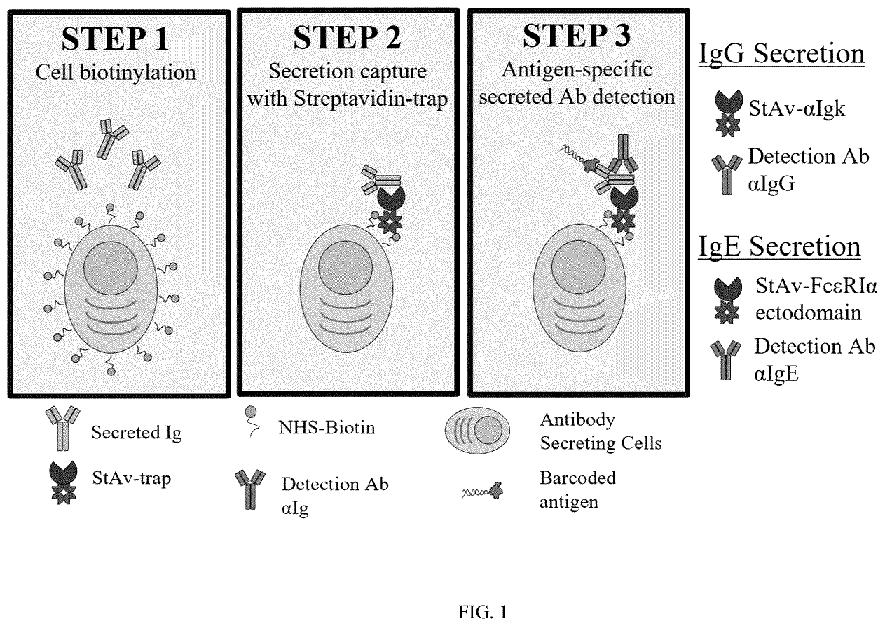

To pair secreted antibodies to the ASCs secreting them, immunoglobulin capture complexes were derived that can locally capture secreted antibodies on the surface of ASCs (FIG. 1). To achieve this, the cell surface was biotinylated using NHS-biotin, which has a hydrophilic active group that binds to cell surface primary amines (—NH2). An affinity matrix was then assembled around cells using streptavidin coupled to an Fc binding reagent (αlgk for IgG and FcεRIα ectodomain for IgE) to capture secreted immunoglobulins from ASCs (FIG. 1). To mark antigen specificity of PCs, this method was coupled with barcoded antigen and/or fluorescent-tagged antigen staining (FIG. 1). Single cell transcriptomes (scRNA-seq), barcoded antigen signal and VH:VL antibody sequences were generated using 10× Genomics reagents.

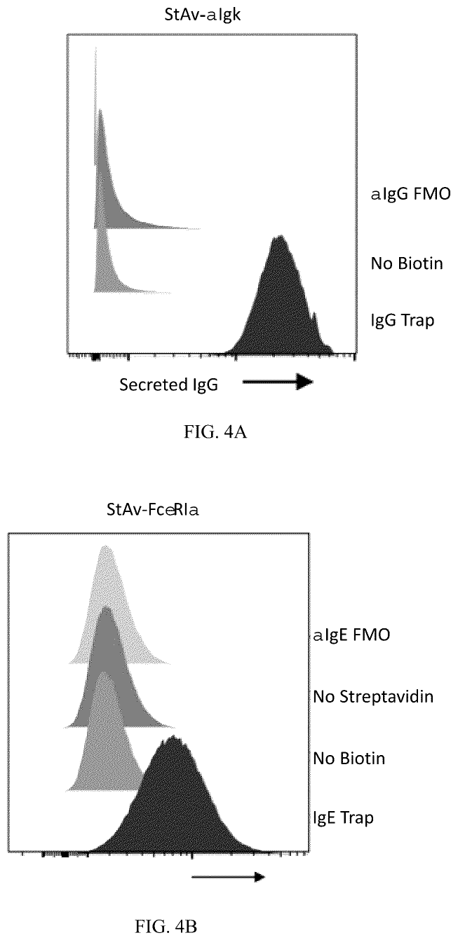

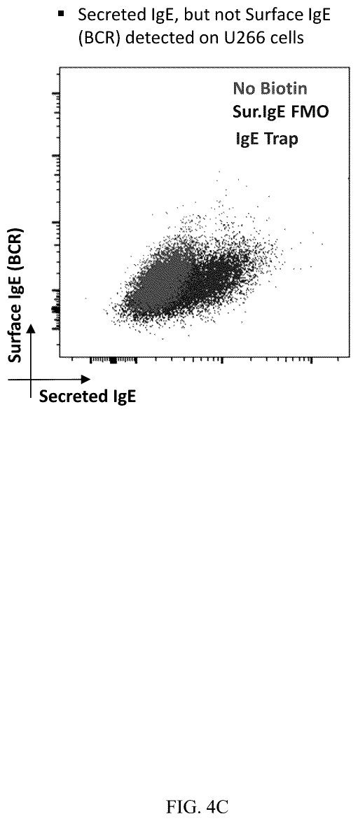

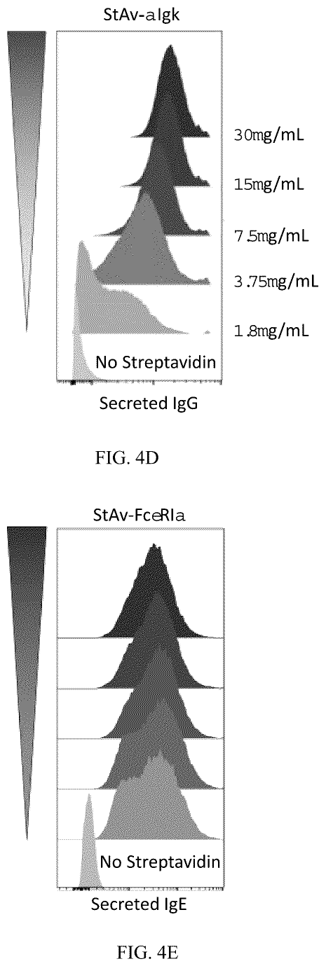

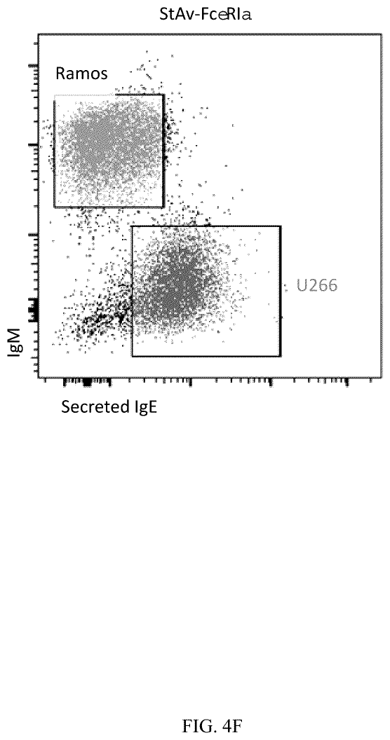

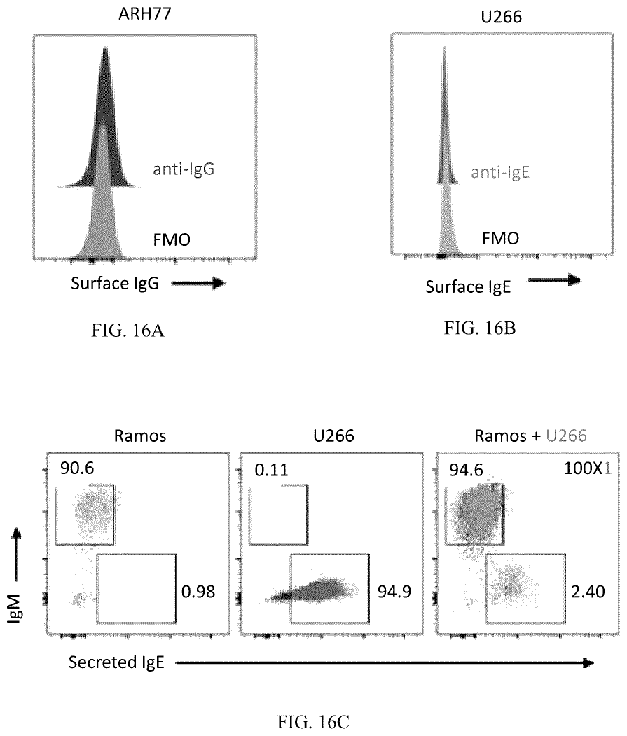

To determine functionality, sensitivity and specificity of IgG and IgE capture complexes, ARH77, an IgG (kappa light chain) secreting cell line, and U266, an IgE (lambda light chain) secreting myeloma line were used for proof of principle experiments (FIGS. 4A, 4B, 4D and 4E). A lack of BCR surface expression on both cell lines were first confirmed (FIGS. 16A and 16B). ARH77 and U266 cells were biotinylated using NHS-Biotin and a capture complex was assembled using streptavidin coupled to anti-human Igk for IgG (StAv-algk) and the ectodomain of the high affinity IgE receptor, FcεRIα (StAv-FcεRIa) for IgE. Sensitivity of capture was determined by staining for IgG and IgE (FIGS. 4A and 4B). While ARH77 and U266 cells missing any part of the affinity matrix lacked IgG and IgE on their surface, successful assembly of all components of the secretion capture complex allowed specific detection of secreted IgG and IgE on the surface of cells that are secreting them (FIGS. 4A and 4B). To further determine the minimal concentration of StAv-algk and StAv-FcεRIα required for binding secreted antibodies, dose titration of the capture complexes was performed and it was observed that StAv-algk saturated at 3.75 μg/mL while StAv-FcεRIα saturated between 15-30 μg/mL (FIGS. 4D and 4E).

To test specificity of the capture complex, IgM-expressing Ramos cells were mixed with U266 cells 1:1 (FIG. 4F) or 100:1 (FIG. 16C) and IgE secretion capture complex was assembled on the mixed population. No binding of secreted IgE was observed on the surface of Ramos cells suggesting there is minimal or no cross talk with neighboring cells at the dilution and culture conditions tested (FIG. 4F; FIG. 16C).

### Example 2: Proof of Concept for Secreted IgE Capture

### Capture of Secreted IgE: Experimental Procedure

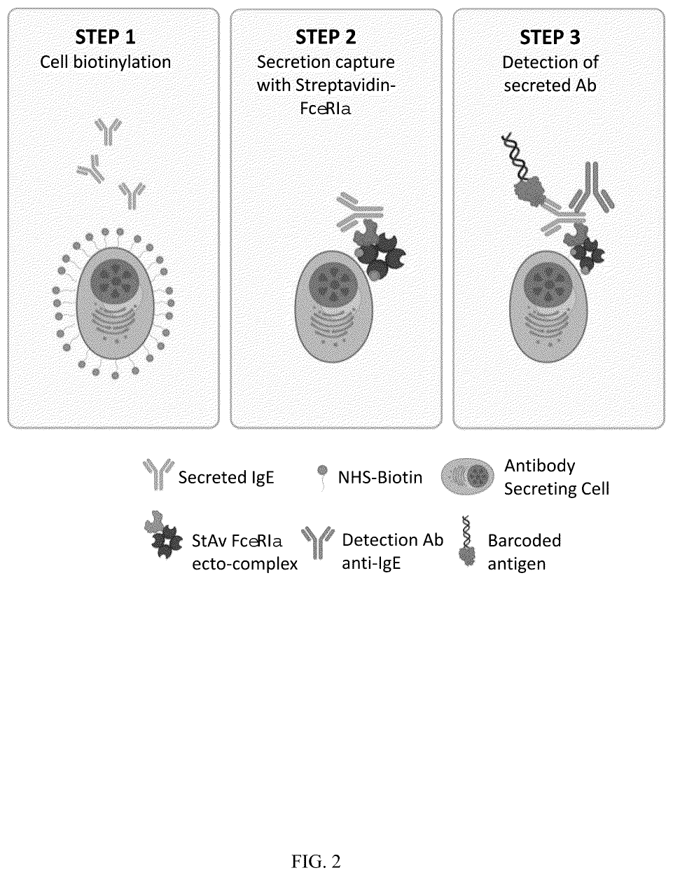

An approach was made to pair a given secreted IgE antibody with the PC that secreted the antibody. FIG. 2 (Step 1) shows the surface of a PC that has been biotinylated with NHS-biotin because the N-hydroxysuccinimido group of NHS-biotin has a hydrophilic active group that nonspecifically reacts with cell-surface primary amines (—NH2). Step 2 shows a secreted IgE capture reagent bound to the biotinylated PC. FIG. 2 (Step 2) also shows the ectoFceRla of the secreted IgE capture reagent capturing an IgE that the PC has secreted. FIG. 2 (Step 3) shows the captured IgE bound to a barcoded antigen and bound by an anti-IgE antibody.

### The Secreted IgE Capture Reagent Successfully Captured Secreted IgE on the Surface of U266 Cells

The surfaces of IgE-secreting U266 cells were biotinylated as described herein, washed three times in cold PBS with 5% w/v BSA with the tubes changed for every wash step, and incubated with the secreted IgE capture reagent as described herein. Thereafter, the cells were washed and stained for thirty minutes at 4° C. with Brilliant™ Violet 711-labeled anti-IgE (BD) in PBS with 5% w/v BSA, 10 mg/mL dextran sulfate, and 1:50 Fc block. The cells were then washed with 10× Brilliant™ Violet buffer. Thereafter, the cells were washed twice in PBS and fixed with BD Cytofix™. Subsequently, flow cytometric data was acquired.

The secreted IgE capture reagent was found to specifically capture secreted IgE antibody on the surface of U266 cells (FIG. 4B). Importantly, cell-surface IgE antibody was not detected in control cells that were not biotinylated, incubation with the secreted IgE capture reagent, or contacted with Brilliant™ Violet 711-labeled anti-IgE. This confirmed that each step is necessary for detecting captured secreted IgE. Moreover, IgE was not detected on U266 cells as a BCR. This confirmed that captured, secreted IgE antibody was detected on the surface of U266 cells rather than IgE as part of a BCR (FIG. 4C). To test specificity of the IgE secretion capture reagent, U266 cells were mixed in a 1:1 ratio with Ramos cells, which secrete human IgM. The mixed cells were incubated with the secreted IgE capture reagent as for the U266 cells alone. The cells were then washed in PBS and fixed with Cytofix™ as for the U266 cells alone. Flow cytometric data was again acquired. The secreted IgE capture reagent was found to capture secreted IgE antibody on the surface of U266 cells but not the IgM secreted by Ramos cells. This indicated specificity of the secreted IgE capture reagent (FIG. 4C).

### Example 3: Capture of Secreted IgE on the Surfaces of Primary Plasma Cells

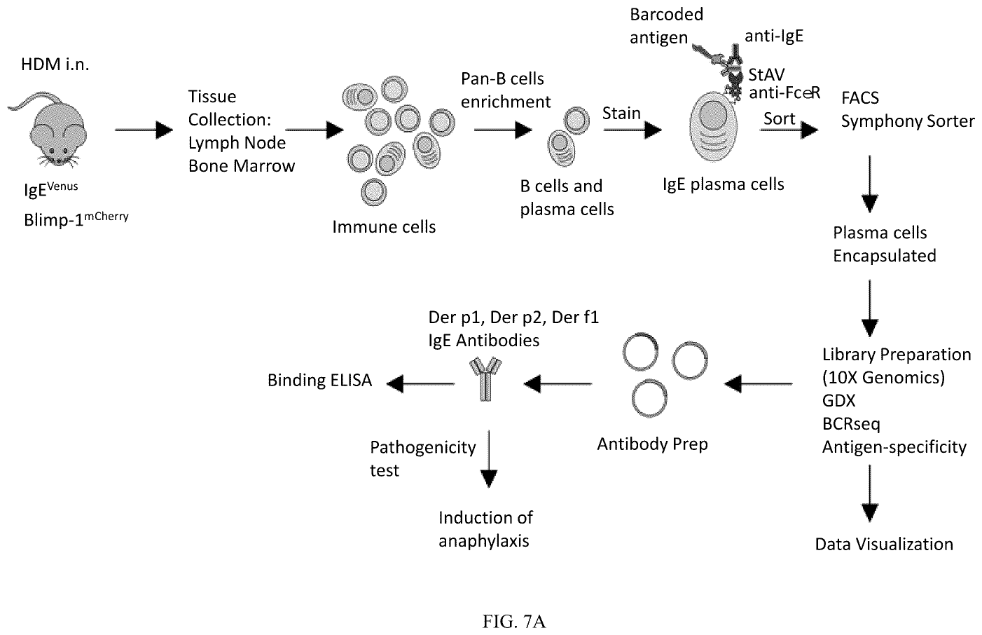

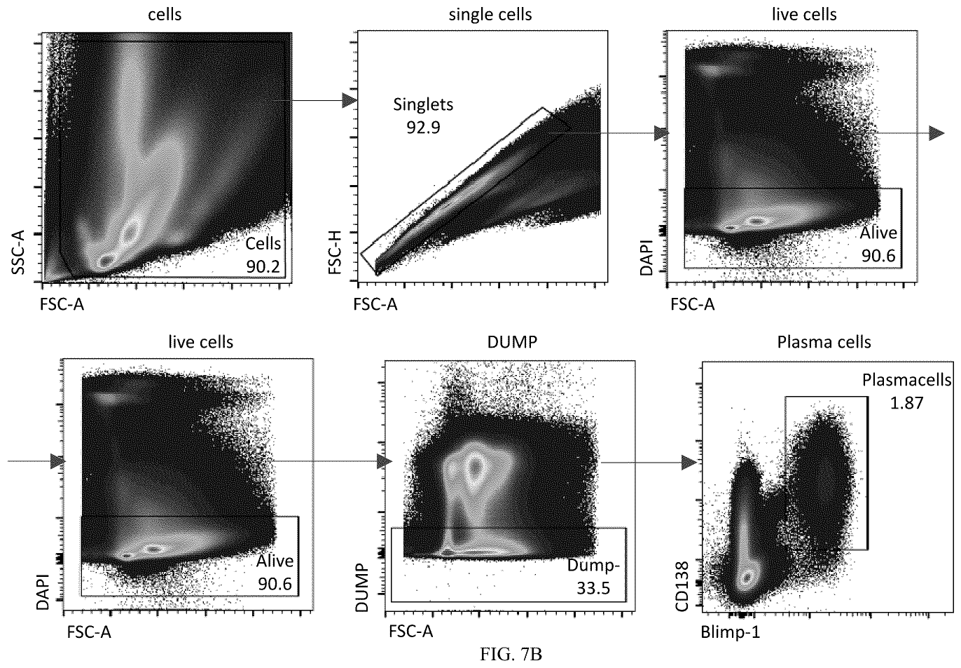

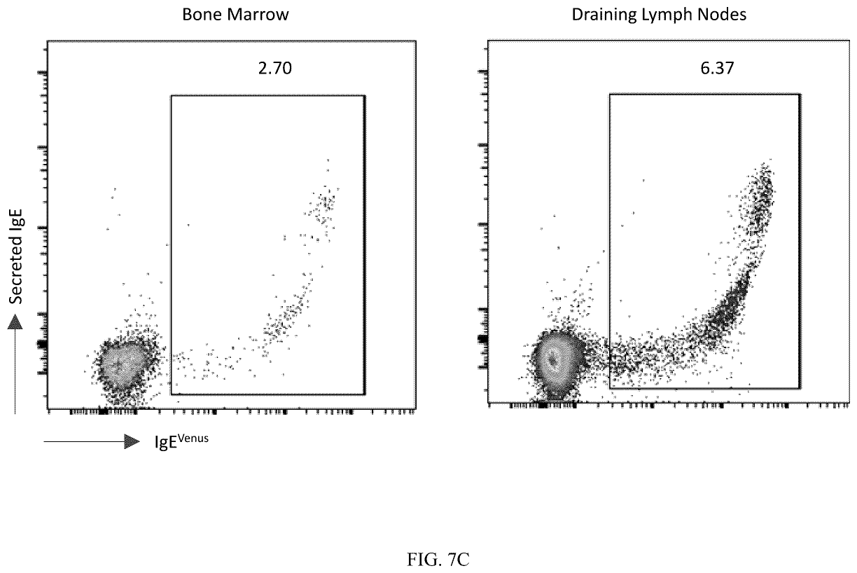

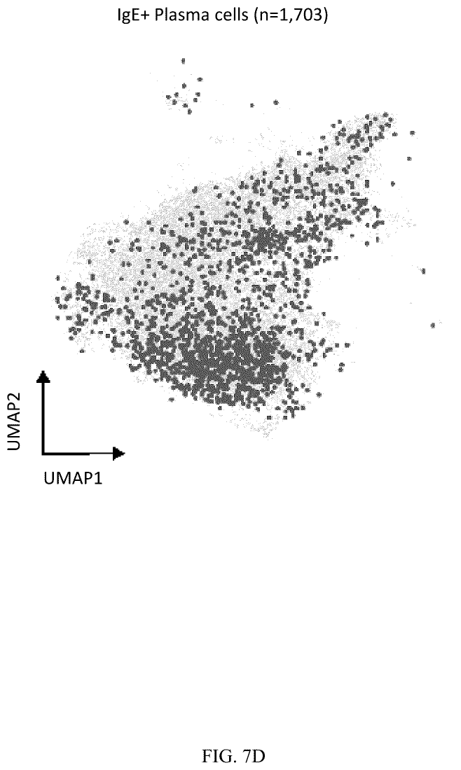

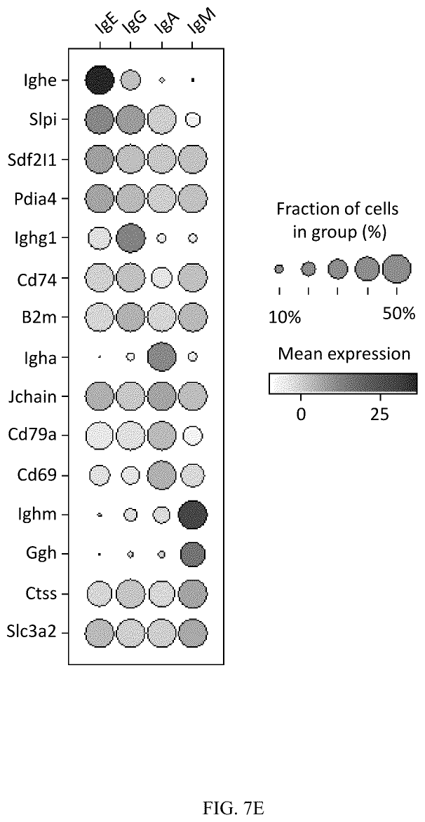

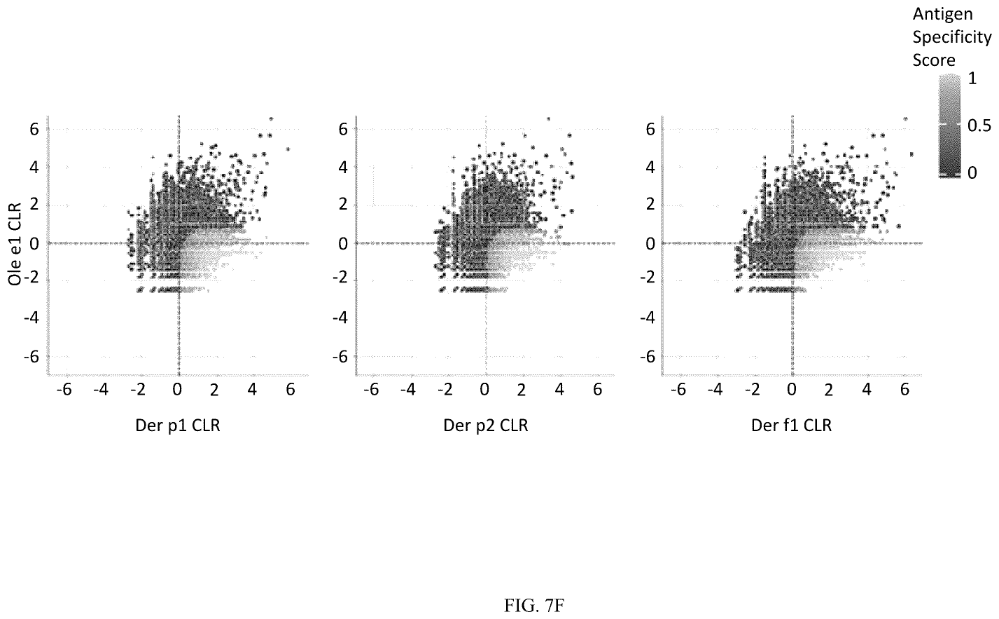

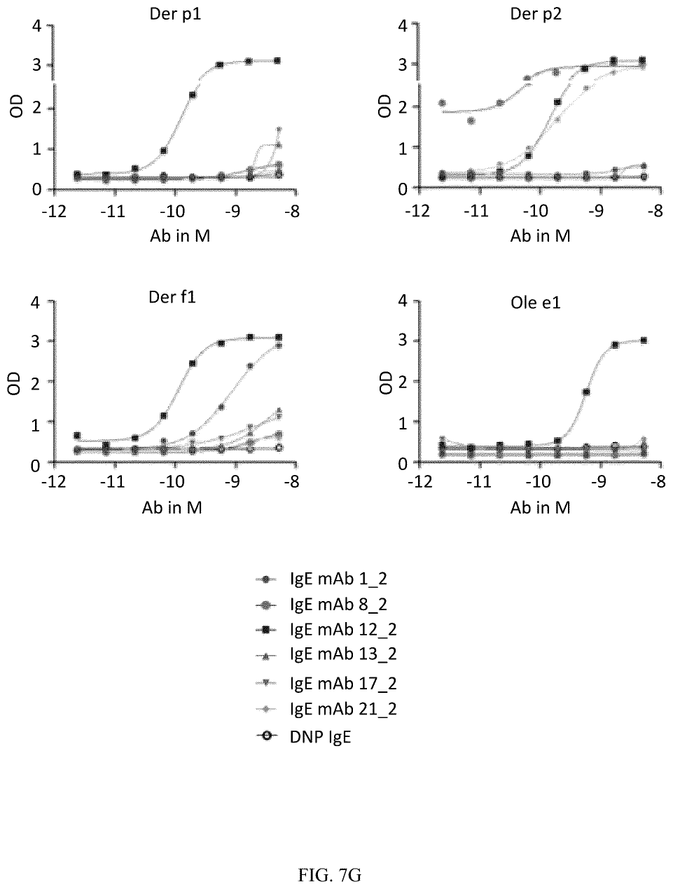

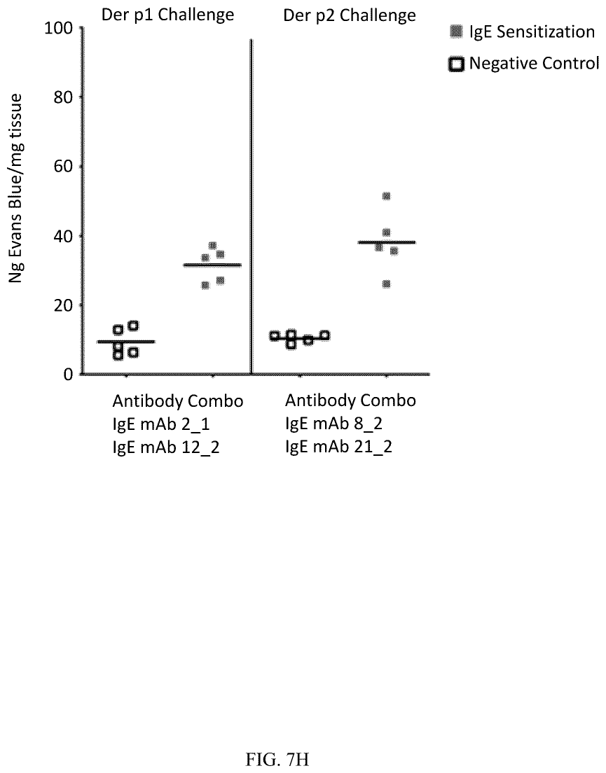

IgE^{Venus} and Blimp-1^{mCherry} reporter mice were used to determine if the secreted IgE capture reagent could capture secreted IgE on the surface of IgE-producing, primary plasma cells. Specifically, the Venus reporter, which had been inserted downstream of membrane IgE locus, identified IgE-secreting cells while the mCherry reporter, which had been inserted downstream of Blimp-1, identified PCs (Asrat et al., Sci. Immunol., 2020, 10, eaav8402). The mice were challenged with 50 μg of HDM extract diluted in 20 μl of saline solution administered intranasally three times a week for fifteen weeks. The mice were anesthetized and sacrificed as described herein. The BM cells were prepared as described herein and the LDLNs were prepared as described herein. BM from mice were pooled together and, separately, dLN from mice were pooled together. Pan-B cells were isolated and stained with an antibody mix comprising Brilliant™ Violet 711 labeled anti-IgE (BD) to detect secreted IgE in PBS with 5% w/v BSA for thirty minutes at 4° C. The labeled cells were washed in PBS with 5% w/v BSA, fixed with Cytofix™, and sorted with a BD FACSymphony® S6 flow cytometer (FIGS. 7A and 7B). Plasma cells were gated in the following order: Cells, single cells, live cells, DUMP⁻ cells, CD138⁺Blimp-1⁺ cells (FIGS. 7A and 7B). Dump is a collection of markers in the same channel, which includes CD3, CD11b, IgD, IgM, Ly6g, etc. Any cell that expressed these markers was excluded. CD138 was detected using anti-CD138 antibodies, and plasma cells were gated as Blimp1⁺CD138⁺ cells. IgE-secreting PCs were sorted as IgE^{Venus+} cells (FIG. 7C).

### Example 4: Single-Cell Library Preparation and Sequencing of Antigen-Specific, IgE-Expressing Plasma Cells

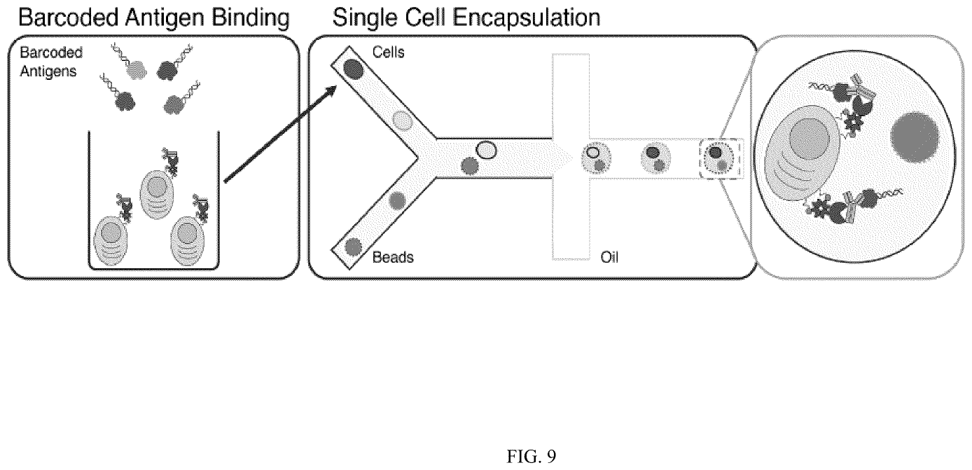

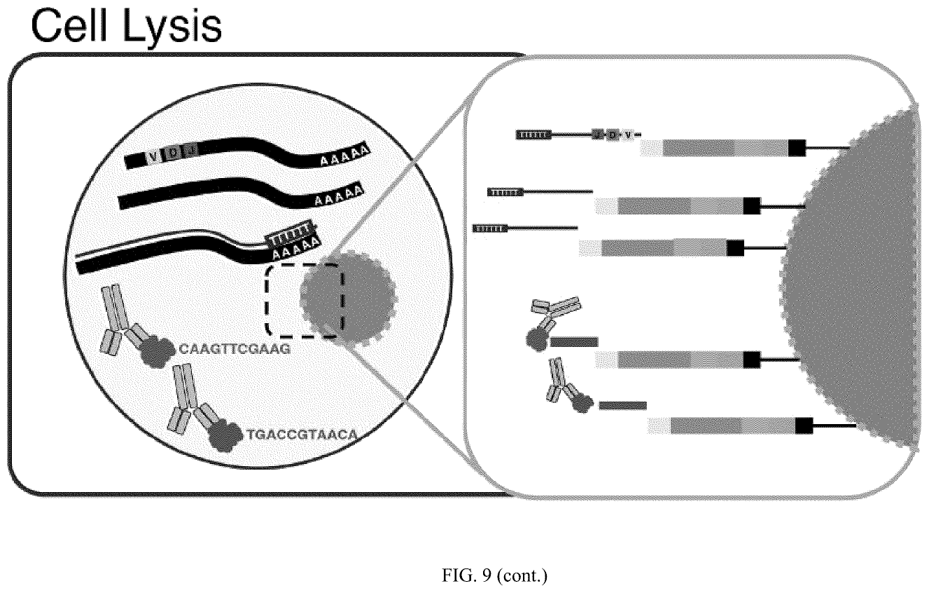

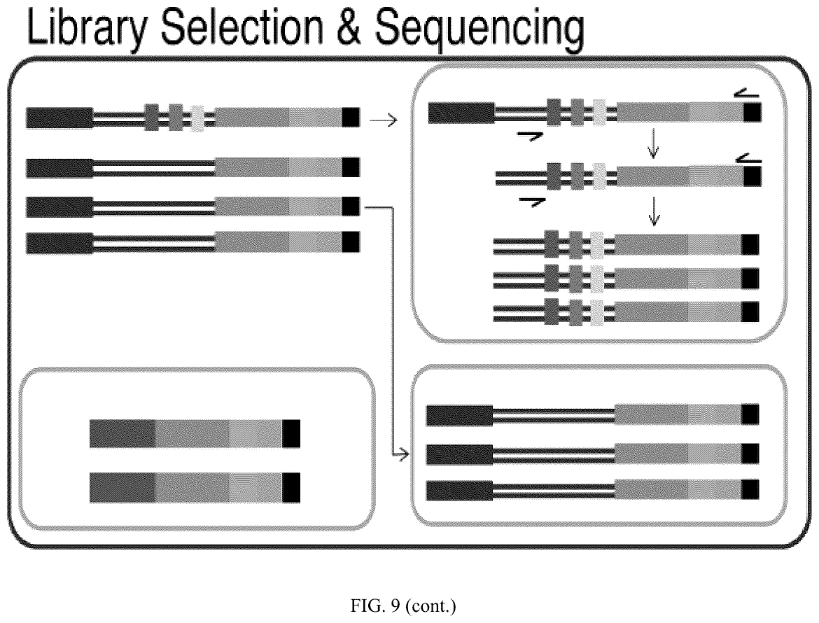

FIG. 7A shows a representative overview of the steps of Examples 3 to 5 while FIG. 9 shows the steps from the binding of barcode antigen to a secreted IgE captured on the surface of a PC to library selection and sequencing.

Plasma cells were prepared as described in Example 3. Single PCs were suspended in PBS with 0.04% w/v BSA and loaded onto a Chromium® Controller device (10× Genomics) at 15,000 cells per lane. Partitions of the PCs in barcoded beads were formed and the PCs lysed. Reverse transcription was performed followed by breakdown of the partition. RNA-seq, Feature Barcode, and V(D)J libraries were prepared using a Chromium® Next GEM Single Cell 5′ Kit, v.2 (10× Genomics). After amplification of the libraries, cDNA was split into separate RNA-seq, Feature Barcode, and V(D)J aliquots. To enrich the V(D)J aliquot for BCR sequences, the Chromium® Automated Single Cell Mouse and Human BCR Amplification & Library Construction Kit (10× Genomics) was used. Feature Barcode libraries for DNA-barcoded Antigens or DNA-barcoded cell surface proteins were prepared using a Chromium® 5′ Feature Barcode Library Construction kit (10× Genomics). Paired-end sequencing was performed on an Illumina NovaSeq® 6000 sequencing system for RNA-seq libraries with read 1 being 26 bp for unique molecular identifiers (UMIs) and cell barcodes and read 2 being 80 bp for transcript reads, with 10 bp i7 and 10 bp i5 reads. Pair-end sequencing was also performed feature barcode libraries with read 1 being 26 bp for UMIs and cell barcodes, read 2 being 35 bp for feature barcode read, and with 10 bp i7 and 10 bp i5 reads. Pair-end sequencing was also performed for V(D)J libraries with read 1 being 150 bp, 10 bp for i7, 10 bp for i5, and Read 2 being 150 bp. For RNA-seq and Feature Barcode libraries, the Cell Ranger Single-Cell Software Suite, version 6.1.1 (10× Genomics) was used to perform sample de-multiplexing, alignment, filtering, and UMI counting. The human GRCh38 and mouse mm10 genome assembly and RefSeq gene model for humans and mice, respectively, were used for alignment. For V(D)J libraries, the Cell Ranger software was used to perform sample de-multiplexing, to de novo assemble of read pairs into contigs, to align, and to annotate contigs against all V(D)J germline reference sequences from the germline mouse IMGT reference database.

### Example 5: Single Cell Analysis of Antigen-Specific, IgE-Expressing Plasma Cells

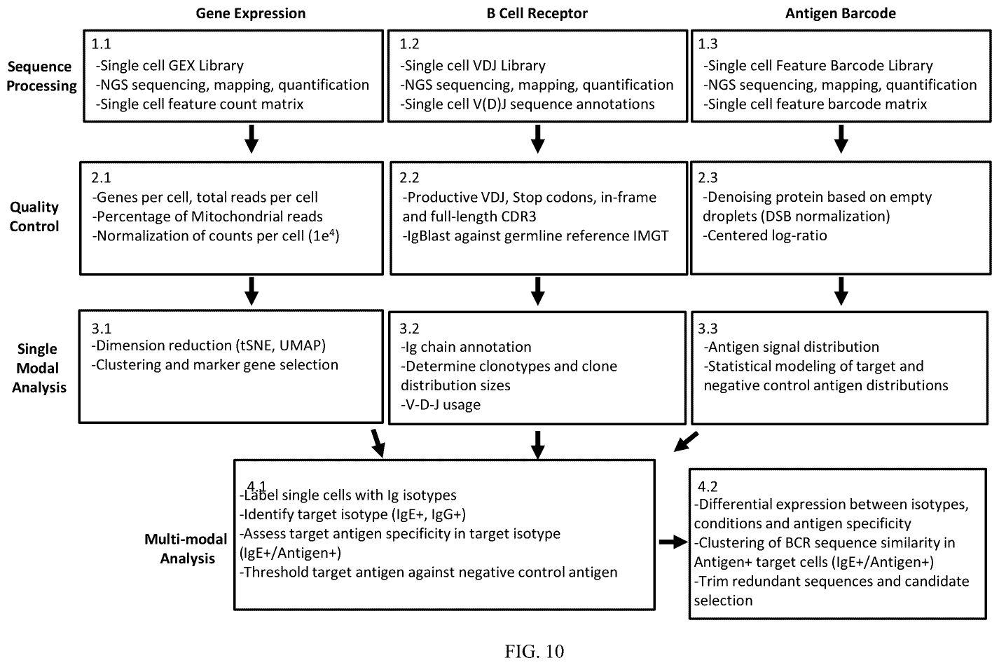

FIG. 10 shows a representative flowchart of the steps described in this Example. Following sequencing, the single cell gene expression library, the secreted antibody V(D)J region library, and the barcoded antigen library were analyzed simultaneously to obtain candidate antigen-positive secreted IgE antibody sequences.

The single cell gene expression library was mapped and aligned to the standard mouse reference genome Mm10. Subsequently, the unique mapped counts for each gene were determined by the CellRanger software. Reads were mapped to the standard reference genome with a single cell alignment software STAR, where each read comprises a unique molecular identifier which maps to annotated genes of the standard Mm10 reference genome. The mapped reads were binned and counted for each unique molecular identifier (UMI), thereby generating a single cell count matrix comprising of mapped counts for each annotated gene and a count for each UMI. The single cell-count matrix was then filtered for by calculating the ratio of the total number of annotated genes divided by the login of the total number of UMIs counted. Cells with a gene to UMI ratio below about 0.1 were filtered out. Cells with more than four times the interquartile range of the total number of UMIs were filtered out. Cells with more than about 80% of reads map to a mitochondrial gene were filtered out. High quality cells with a gene to UMI ratio above about 0.1, with less than about four times the interquartile range of the total number of UMIs counted, and with less than 80% of reads mapping to a mitochondrial gene were retained. The count matrices from each individual sample capture were normalized to a total count of 10,000 and batch corrected using the Harmony algorithm to generate a combined uniform manifold approximation and projection (UMAP). Unsupervised clustering by the Leiden algorithm was then used to determine cell type clusters and a Wilcox test was used to identify specific cell type and cluster marker genes.

The IgBlast sequence analysis tool (Ye et al., Nucleic Acids Res., 2013, 41, W34-W40) was carried out to align the V(D)J sequences against the germline mouse IMGT reference database. Valid V(D)J sequences were then filtered by productive in-frame alignments, full-length CDR3s, and the absence of stop codons within the sequence. The IgBlast tool also confirmed the immunoglobulin isotype, gene mappings for variable, diverse, and joining regions, and accurate full-length variable regions for both heavy and light chain sequences per cell.

The barcoded antigen library was mapped by the CellRanger software to a custom short-read reference that contained the DNA tag sequences of the four barcoded antigens, namely, Der p 1, Der p 2, Der f 1 and Ole e 1. These tag sequences were quantified across all cells and were normalized by taking the centered log-ratio (CLR) per barcoded antigen across each sample capture. In addition, background antigen signal for each barcoded antigen was removed by DSB (Denoised and Scaled by Background). Both the CLR and the DSB normalized values were used to quantify the target antigen signals, namely, those for Der p 1, Der p 2 and Der f 1, against the control signal, namely, that for Ole e 1.

Antigen specificity score (AgSS) for IgG were calculated by subtracting the quantile of hIL-6Rα barcoded UMI counts multiplied by a penalty factor (x) from the quantile of hIL-4Rα barcoded UMI counts, AgSS=Q_{hIL-4Rα}-Q_{hIL-6Rα}^{x}. AgSS for IgE were calculated with a similar formula for mouse and human single cell data. For IgE scores the target antigens were either Der p1, Der p2 or Der f1 and the control was always Ole e1.

Ig BCR sequences were clustered using scirpy v0.10 using the function scirpy.pp.ir_dist based on the amino acid sequence and with a hamming distance of less than 4. Clustered BCRs were collected into clonotypes with the function scirpy.tl.define_clonotypes based on the heavy chain CDR3 amino acid sequence based on the previous criteria of a hamming distance of less than 4. Scirpy documentation can be referenced at the world wide web at “scverse.org/scirpy/latest/api.html”.

Following processing of the three single-cell libraries, the gene expression and V(D)J region sequencing data were used to subset PCs with a valid IgE constant region and detectable expression of IgE and CD79a but lacking the expression of Ms4a1 and CD19 typically found in non-PC B cells. IgE-expressing PCs were then assessed for differential expression against additional IgA, IgG, and IgM plasma cell isotypes to further characterize the unique transcriptional signature of IgE-secreting PCs. IgE-secreting PCs were then assessed for antigen specificity by comparing the normalized levels of the target antigens, namely, Der p 1, Der p 2, or Der f 1, against the normalized level of the control antigen Ole e 1. This was performed by calculating an empirical score for each target antigen and subtracting the quantile rank of the target antigen from the quantile rank of the control antigen with a penalty factor. The formula used to do is set forth below:

Antigen specificity=qT−qC^{x}

where qT refers to the quantile of the target signal, qC refers to the quantile of the control signal, and x is a penalty factor.

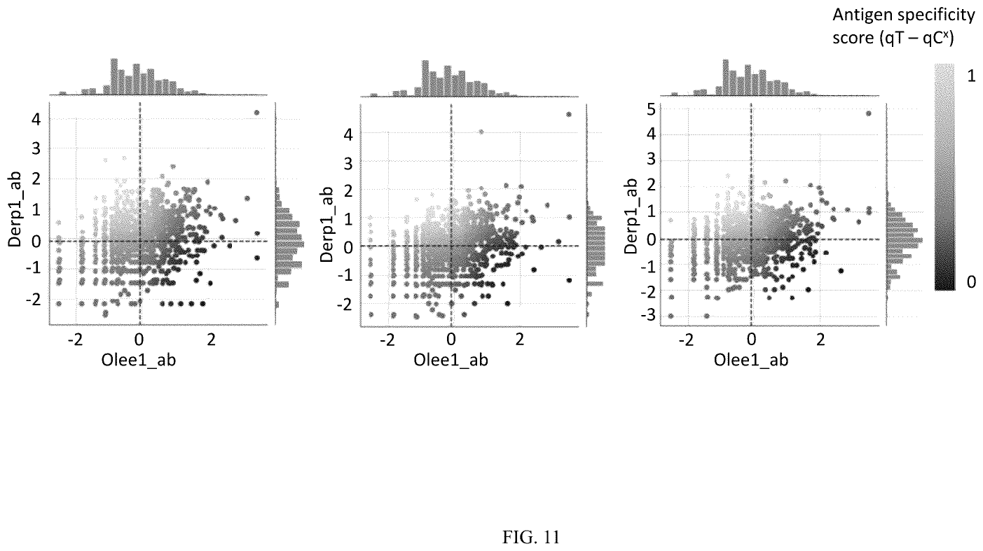

The antigen specificity score (AgSS) was determined per cell for each of the three target antigens and utilized to prioritize cells with the strongest signal of target antigen while minimizing the signal of the control antigen. Antibody candidates were selected from the ranked lists of target antigen signal using paired VH:VL antibody sequences from the cell in question. FIG. 11 shows visualization of antigen specificity.

### Example 6: IgE-Binding ELISAs

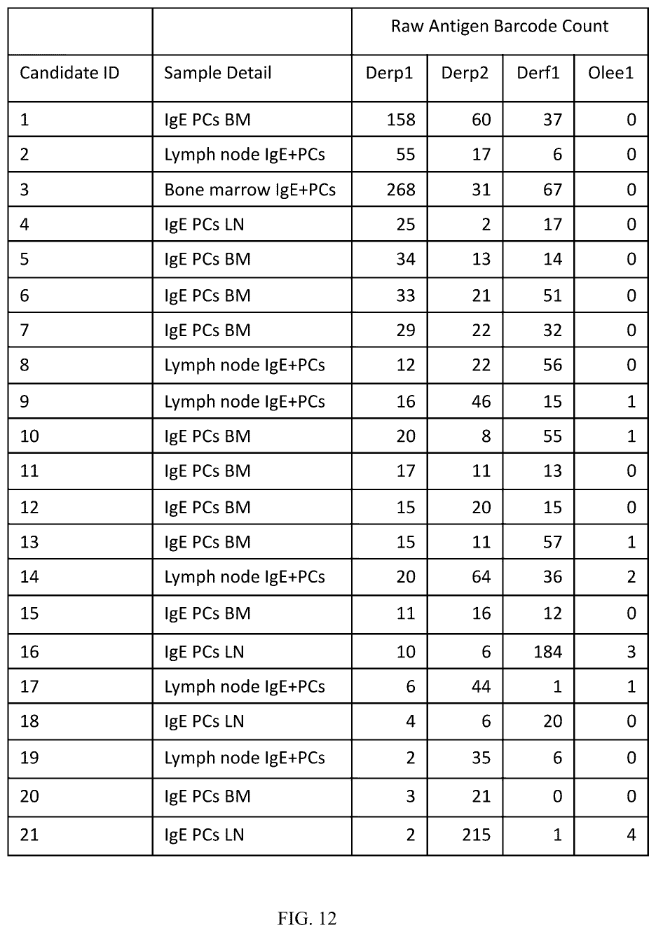

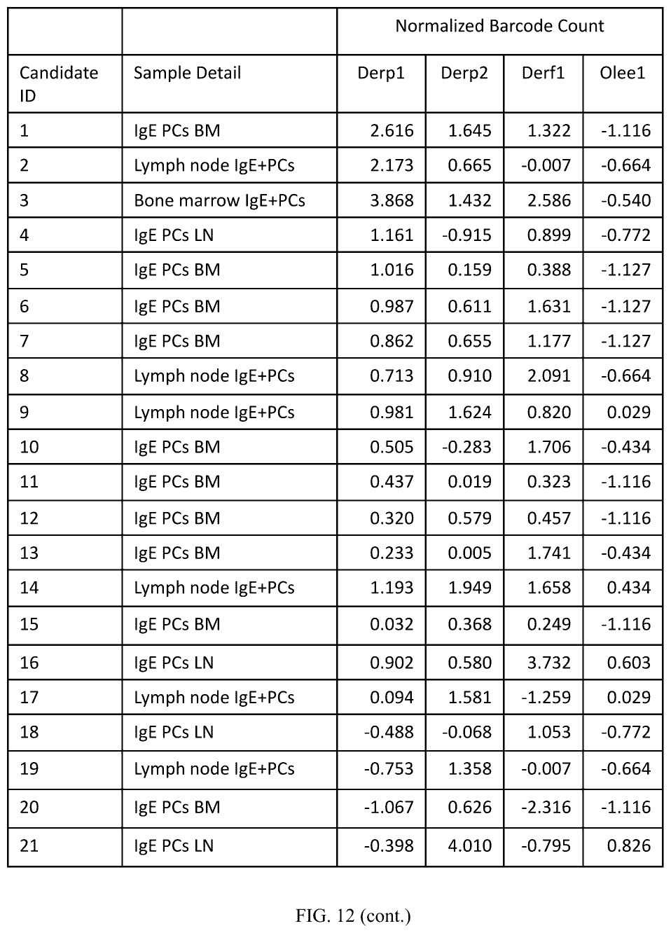

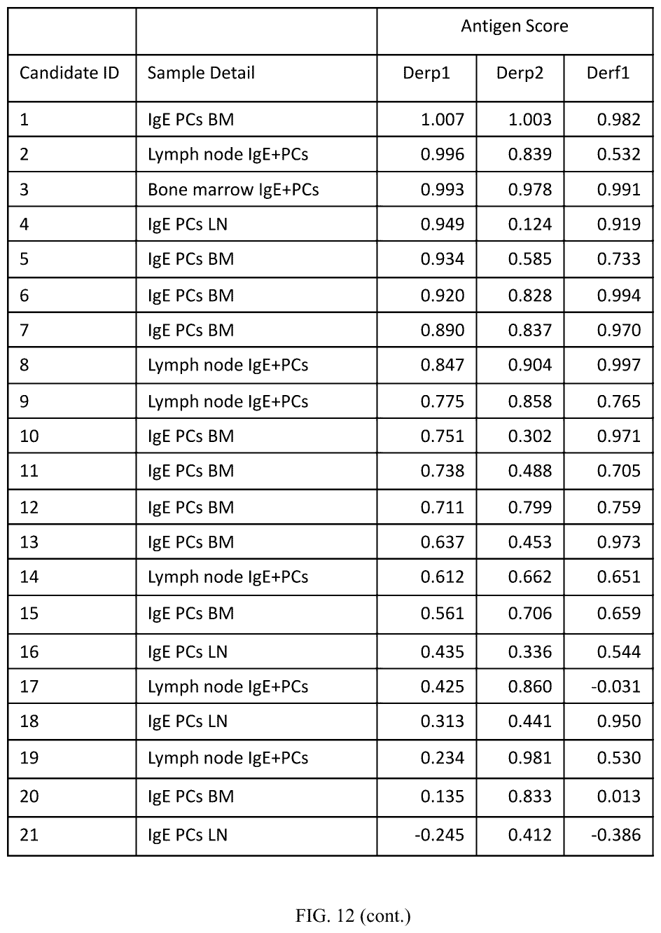

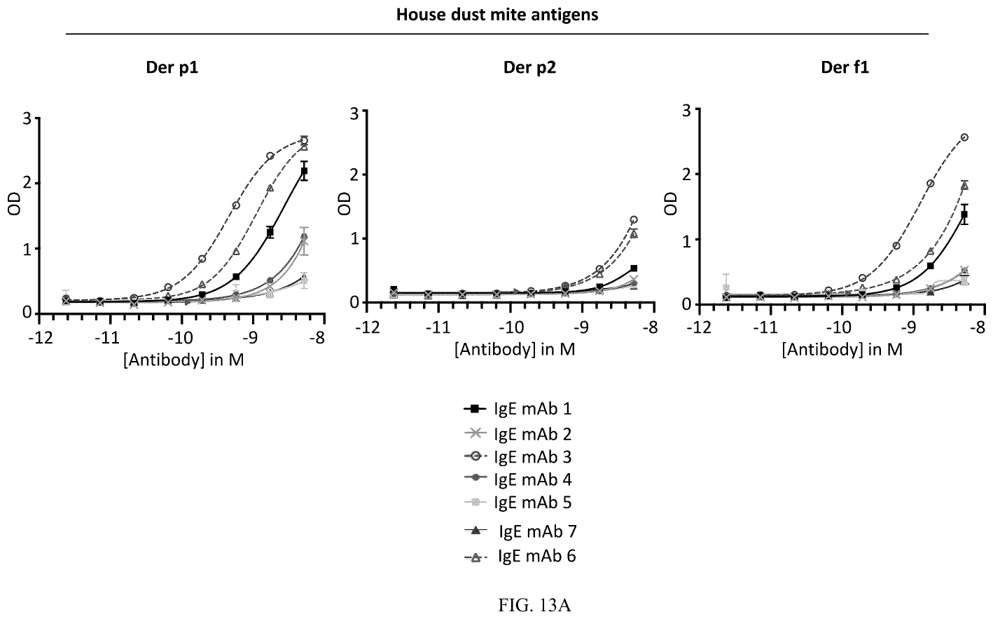

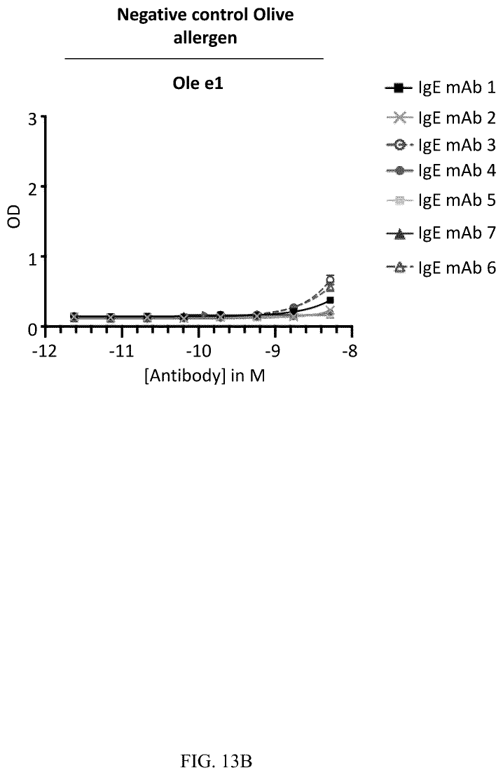

After selecting allergen-specific antibodies, relative binding to Der p 1, Der p 2, and Der f 1 were quantified by ELISA. FIG. 12 shows a table of IgE candidates based on antigen signal. Data were acquired for 1,069 IgE cells. Specifically, binding microtiter plates were coated with 100 μl of 2 μg/mL of natural Der p 1, Der p 2, Der f 1, and Ole e 1 in PBS overnight at 4′C. IgE antibodies were prepared at a concentration of 1 μg/mL and serially diluted three-fold in PBS with 0.5% w/v BSA. After washing with 0.05% v/v Tween® 20 in PBS and blocking in PBS with 0.5% w/v BSA, 100 μl of the diluted IgE antibodies were transferred to the plates and incubated for one hour at ambient temperature. Subsequently, 100 μL of anti-mouse IgE antibody conjugated to horse-radish peroxidase (HRP) diluted at 1:5000 was added. The plates were then incubated for one hour at ambient temperature, and washed in 0.05% v/v/Tween® 20 in PBS. Subsequently, the HRP substrate 3,3′,5,5′-tetramethylbenzidine (TMB) was added. The reaction was stopped (with 100 μL of sulfuric acid 2.0 N) and optical density values determined using a plate reader at 450 nm. IgEs mAb1, mAb3, and mAb6 were found to bind to Der p 1 and Der f 1 efficiently (FIG. 13A). However, these antibodies did not bind to the negative control allergen Ole e 1 (FIG. 13B). Compared to IgE mAb1, mAb4, and mAb6, the other IgE mAbs bound to Der p 1 and Der f 1 less efficiently. Overall, these data show that three IgE antibodies, which were captured by the secreted IgE capture reagent and for which the V(D)J regions were determined, bound specifically to Der p 1 and Der f 1.

### Example 7: Capture and Profile of IgE Plasma Cells In Vivo

For validation of IgE capture in vivo, the previously described IgE^{Venus} and Blimp-1^{mCherry} reporter mice (FIG. 7A) that were exposed to house dust mite (HDM) extract for 15 weeks and secreted IgE was captured using StAv-FcεRIα ectodomain as described in FIG. 1. Cells were stained with an antibody mix containing PC markers, αlgE for secreted IgE, and oligonucleotide-barcoded HDM-allergens, including Der p1, Der p2, Der f1 and an Olive allergen (Ole e1) as a negative control. PCs were gated using Blimp-1 signal (FIGS. 7B and 7C) and IgE PCs were sorted (FIGS. 7B and 7C) along with non-IgE PCs (FIGS. 7B and 7C) for comparison. Consistent with what was observed for U266 and Ramos (FIG. 4F), secreted IgE was only detected on IgE PCs, and not on other isotype (non-IgE PCs), confirming specificity of the trap in a mixed cell population (FIGS. 7B and 7C).

### Example 8: IgE Plasma Cells Sequenced from the Bone Marrow and Lymph Nodes of HDM Exposed Mice

To elucidate the biology of tissue resident IgE PCs, the single-cell transcriptomic profiles from lung dLN and BM were merged and cell clustering to compare IgE, and non-IgE PCs was performed. 12,646 IgG+ cells, 17,524 IgA+ cells, 3,503 IgM+ cells and 1,703 IgE+ cells were obtained (FIG. 7D). From single cell transcriptomes, immunoglobulin expression was confirmed and specific gene expression programs were identified for each isotype. IgE PCs expressed high levels of Ighe, Slpi, Sdf2l1, and Pdia4. IgG PCs expressed Ighg1 and modestly higher Cd74 and B2m while IgA PCs expressed Igha and higher levels of Cd79a and Cd69. IgM PCs expressed Ighm and higher SIc3a2, Ctss and Ghg (FIG. 7E).

Using-barcoded HDM allergens, it was possible to directly map IgE PCs to their specificity to Der p1, Der p2, Der f1 (HDM allergens) and Ole e1 (olive allergen, negative control) and compare their AgSS which were calculated as described in Example 4. The antigens were Der p1, Der p2 and Der f1, and the control for all was Ole e1 (olive antigen) (FIG. 7F).

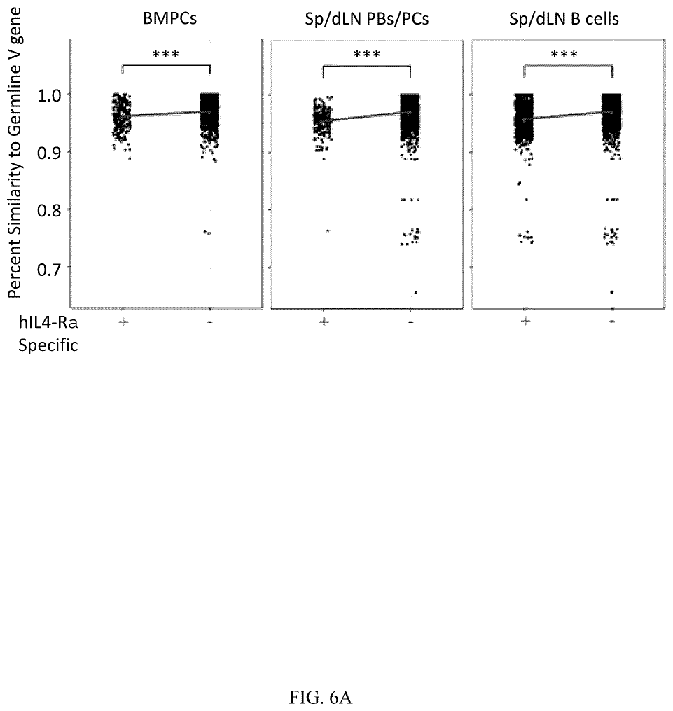

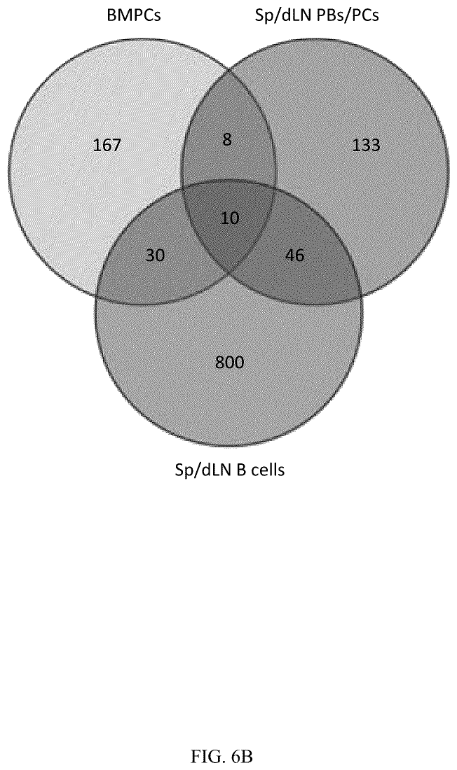

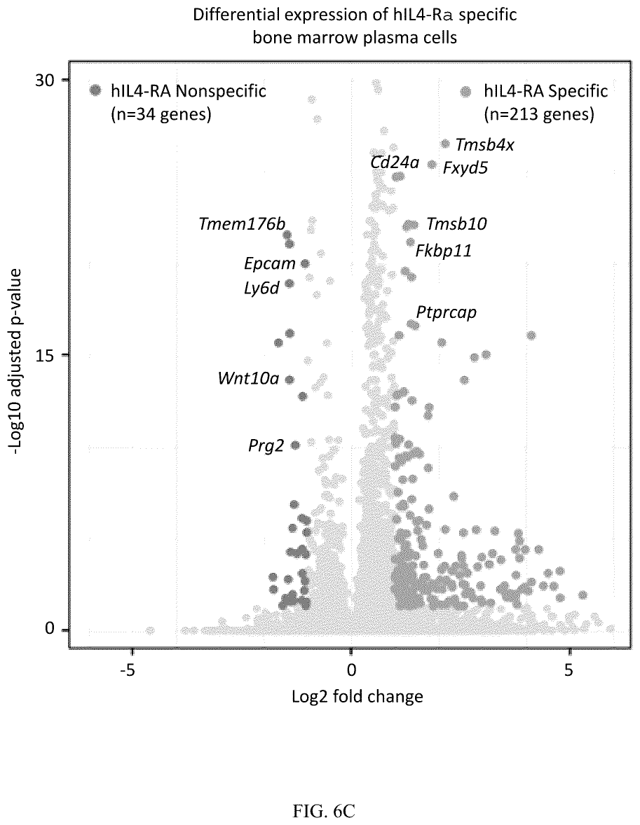

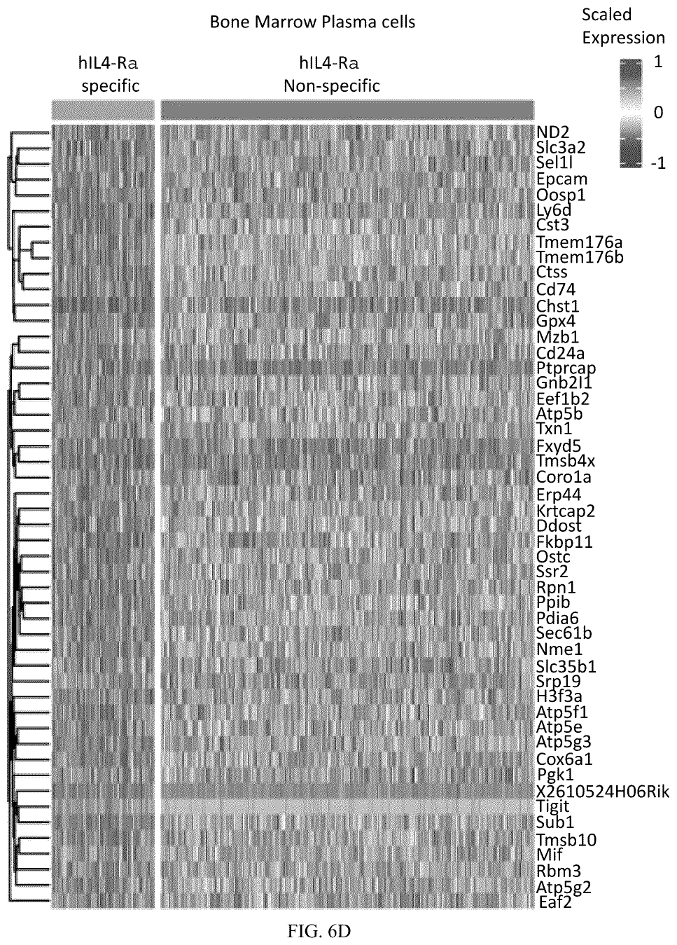

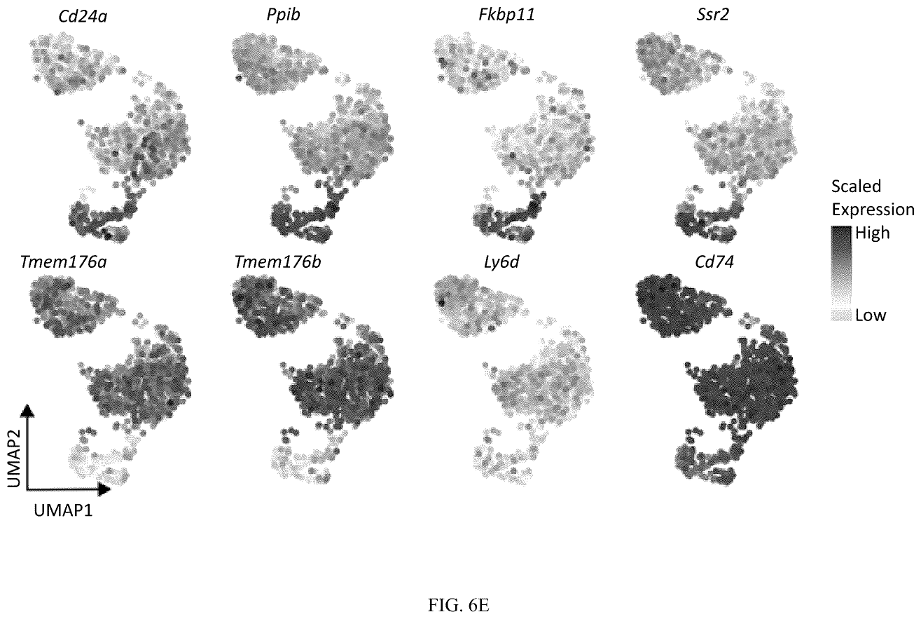

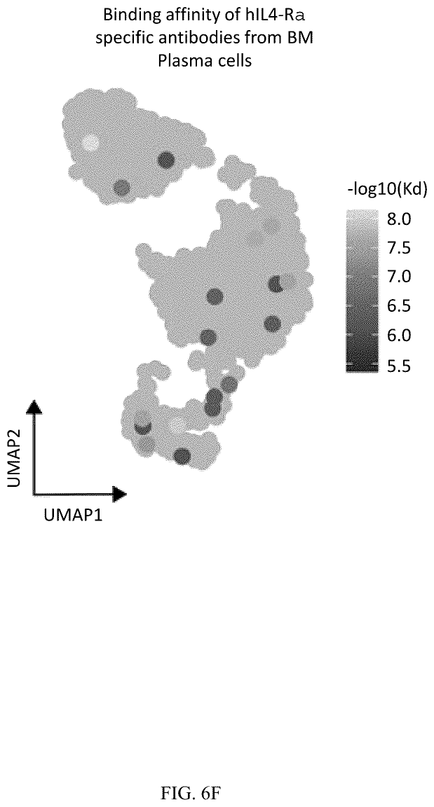

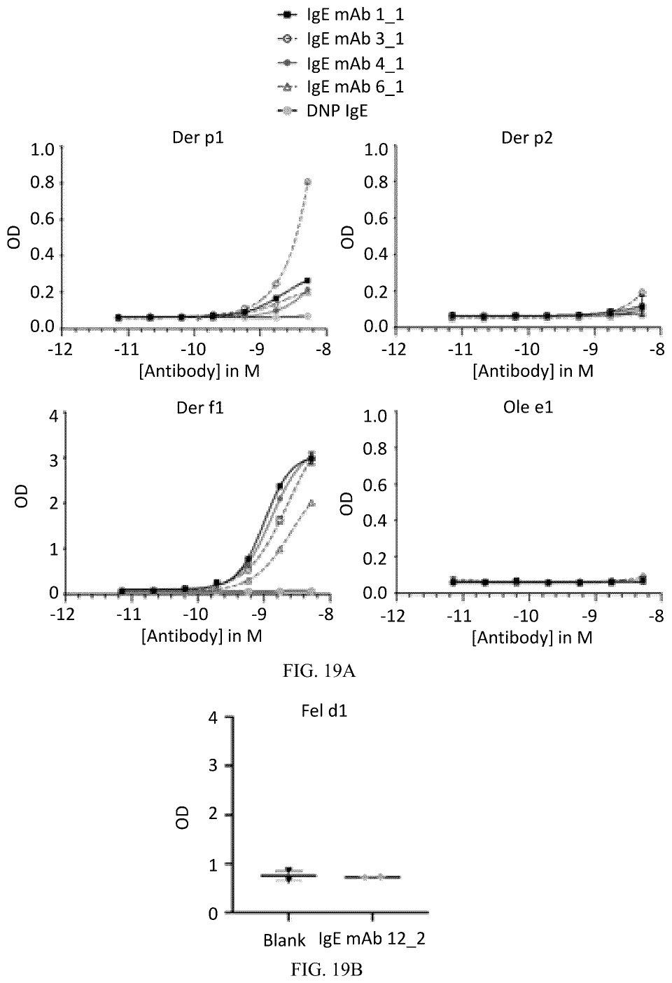

A few allergen-specific IgE antibodies were selected based on reactivity profile (see, Table 3) and their relative binding to Der p1, Der p2 and Der f1 as well as to the olive allergen, Ole e1, was tested by ELISA. 2 IgE antibodies (IgE mAb12_2 and mAb 3_1) bound to Der p1 (FIG. 6F, FIG. 19A), 3 IgEs bound to Der p2 (IgE mAb12_2, mAb8_2 and mAb21_2) and 6 IgEs bound to Der f1 (IgE mAb12_2, mAb1_2, mAb 1_1, mAb3_1, mAb4_1 and mAb 6_1). Ab12_2 bound to Ole e1 in addition to Der p1, Der p2 and Der f1 (FIG. 7G) but did not show any binding to Fel d1 (FIG. 19B).

To determine if the IgEs that showed HDM-allergen binding had anaphylactic potential, a passive cutaneous anaphylaxis (PCA) assay was performed. Mice were sensitized intradermally with a cocktail of Der p1 or Der p2 specific IgEs (FIG. 7G and FIG. 19A) in their left ear and an irrelevant DNP-IgE in the right ear as a negative control. After 24 h, mice were intravenously challenged with Evans blue containing Der p1 for group A or Der p2 for group B (FIG. 7H). Mast cell degranulation was observed only in the ears sensitized with Der p1 or Der p2 specific IgEs (left ear), as measured by Evan's blue dye leakage (FIG. 7H), demonstrating that the cocktail of IgE antibodies used induced crosslinking of FcεRIα and mast cell degranulation.

### Example 9: Human Primary Cells: Identification and Profiling of IgE PCs from the Bone Marrow of Allergic Donors

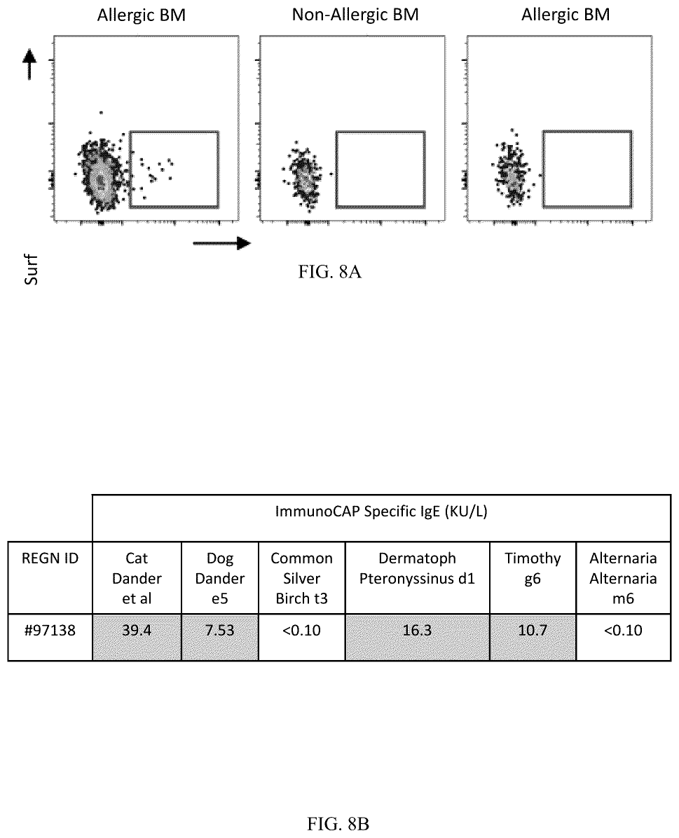

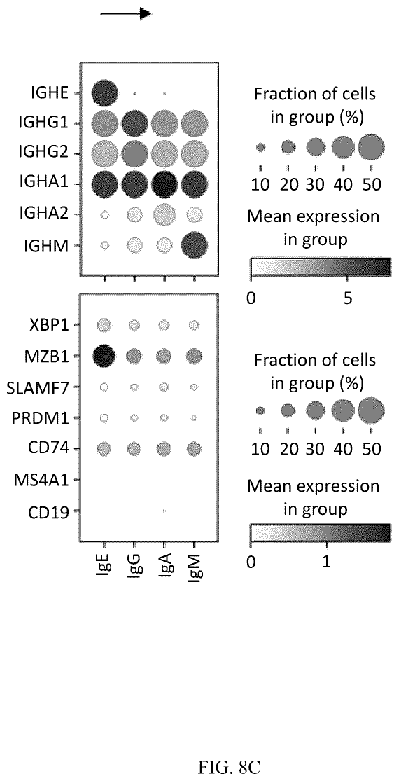

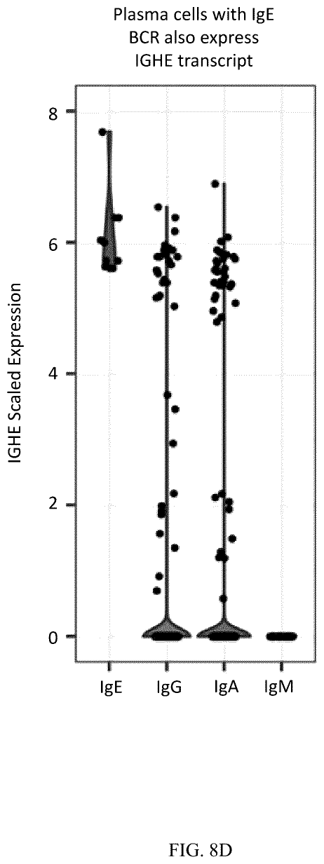

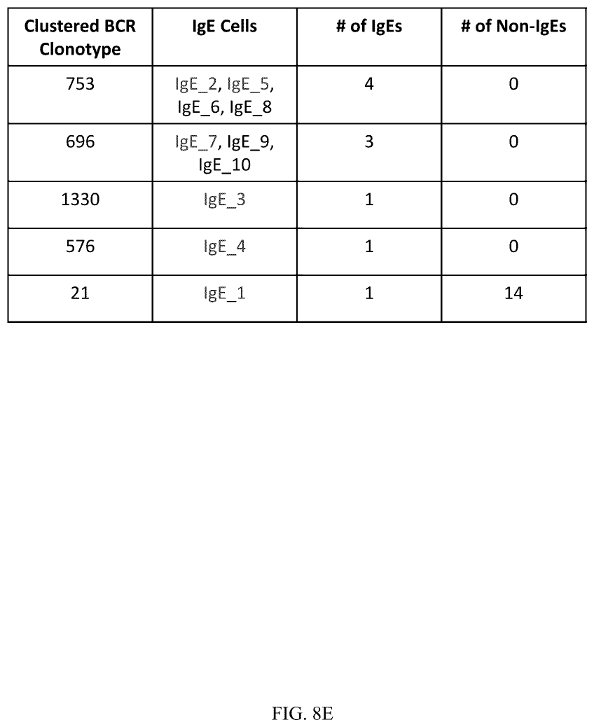

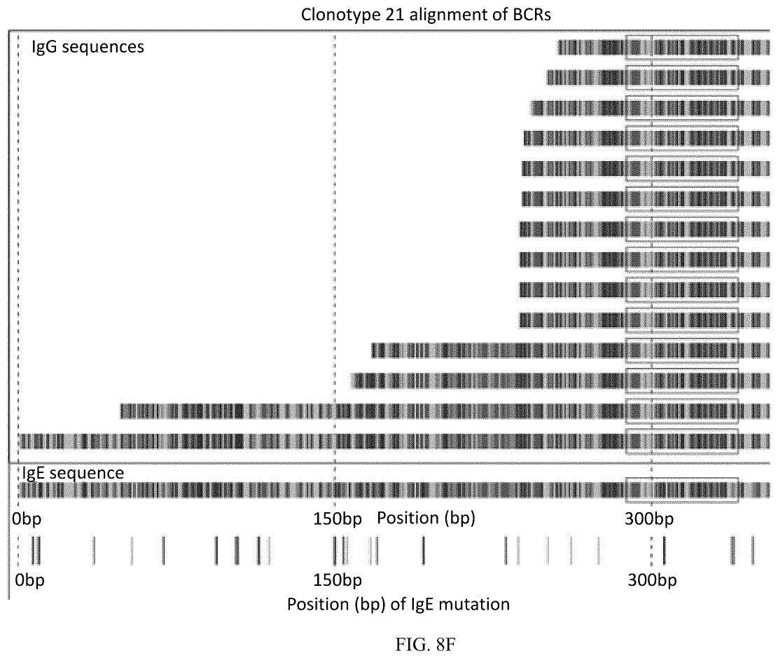

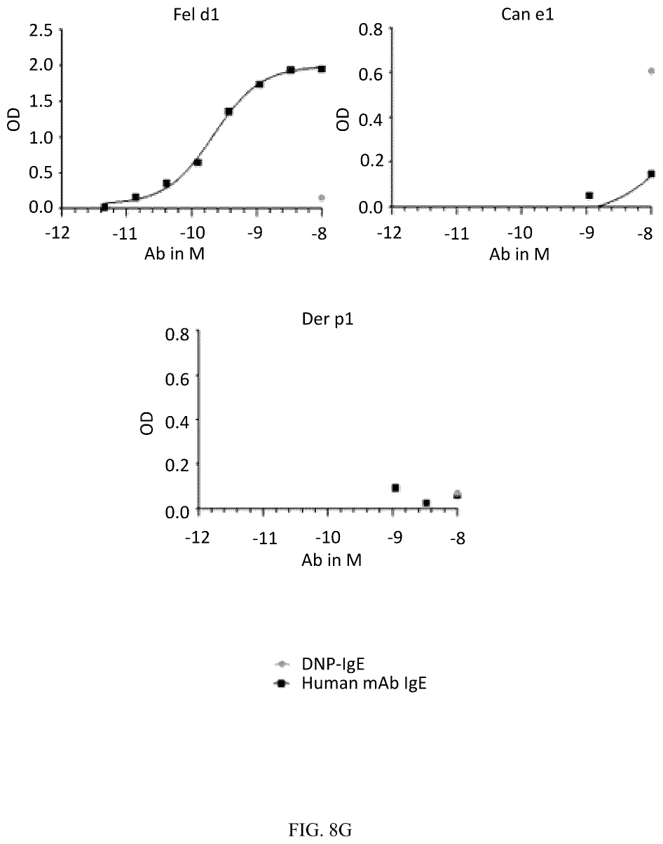

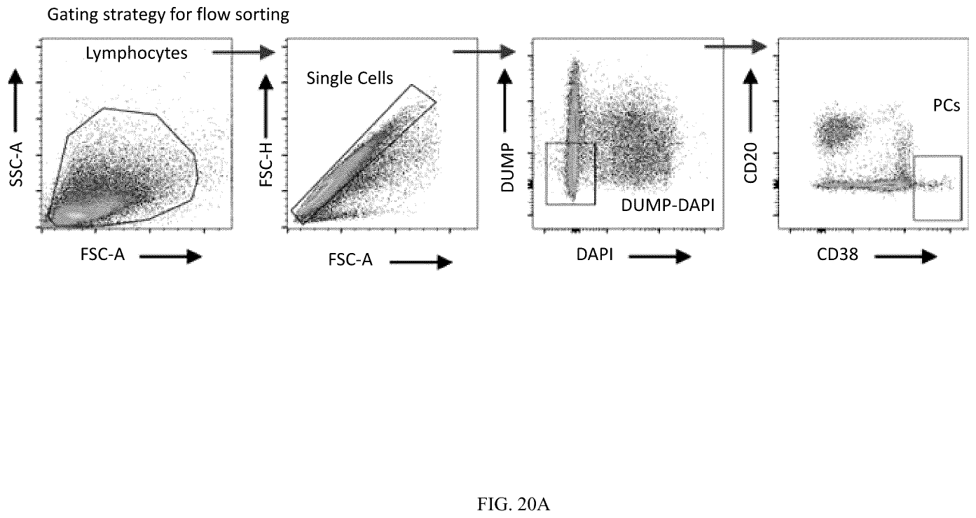

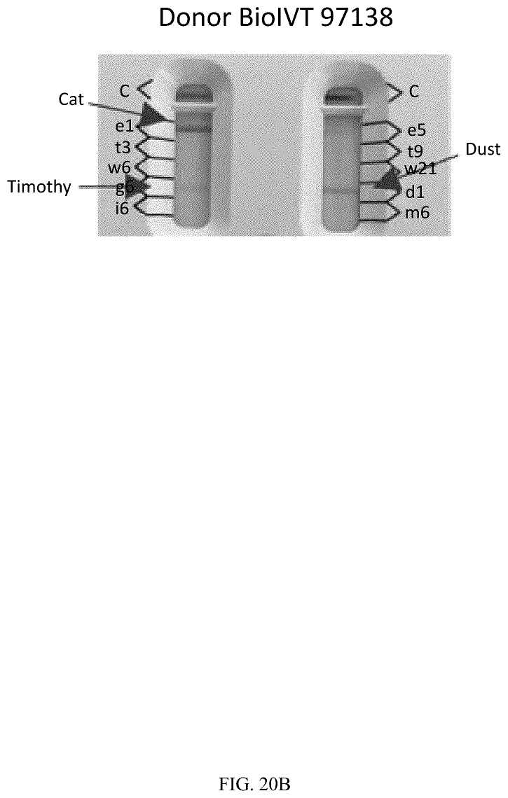

To determine if IgE secretion capture could be extended to human primary cells, we performed IgE-secretion capture on the BM of self-reported allergic donors. B lineage enriched cells were coated with NHS-biotin and secreted IgE was captured using StAv-FcεRIα ectodomain as previously described in FIG. 1. PCs were gated as CD38 high/CD20 low (FIG. 20A) and specificity and functionality of StAv-FcεRIα secretion capture was confirmed in non-allergic BM that had full Biotin-StAv-FcεRIα assembly (FIG. 8A) as well as allergic BM that lacked StAv-FcεRIα (FIG. 8A). To perform full validation of human IgE secretion capture, donors with confirmed cat, timothy grass and HDM reactive IgEs in sera were selected (FIG. 8B, FIG. 20B), the IgE secretion capture was performed, and all IgE PCs along with non-IgE PCs from the BM were sorted for comparison.

Following sorting, scRNA-seq recovered full transcriptomes and BCR sequences of 5,559 bone marrow PCs from the cat, grass, and dust allergic individual. The specificity of PC capture was confirmed based on the abundant expression of various immunoglobulin genes and PC markers such as XBP1, SLAMF7, and CD74, but B cell markers such as MS4A1 and CD19 were not observed (FIG. 8C). Ten IgE PCs were identified by VH:VL sequencing and alignment to the human germline database with IgBlast. These IgE PCs correlated very strongly with IGHE transcript expression along with a small fraction of cells from IgA and IgG isotypes which also expressed IGHE, possibly reflecting sequential class switching between these isotypes or sterile IGHE transcription in some IgA and IgG cells (FIGS. 8C and 8D). This suggests that despite the rarity of IgE PCs in human bone marrow, the capture method of the disclosure enables isolation and profiling of IgE PCs.

To investigate clonal evolution, VH sequences from all 5,559 cells were clustered based on the amino acid sequence of the heavy chain CDR3 region allowing for up to 3 amino acid mismatches, deletions, or insertions. The 10 IgE PCs clustered uniquely into 5 clonotypes, which were defined as a group of PCs with highly similar heavy chain amino acid CDR3s (see, Table 4). Two clonotypes, denoted 753 (n=4 cells) and 696 (n=3 cells), were made up exclusively of IgE cells and each clonotype displayed identical CDR3 sequences, respectively (FIG. 8E). Two other clonotypes denoted 1330 and 576 were single-cell clones from individual IgE cells (FIG. 8E). Interestingly, clonotype 21 contained 14 IgG cells and a single IgE (FIG. 8E). Closer inspection of the CDR3 nucleotide sequence of clonotype 21 revealed that the lone IgE sequence contains 3-point mutations within the CDR3 region while all IgG members of clonotype 21 carry an identical CDR3 region (FIG. 8F). Additionally, the IgE sequence harbored somatic hypermutations in the upstream V gene locus (FIG. 8F) compared to IgG clonotype members, suggesting additional rounds of affinity maturation.

After selecting the IgE clones, we tested binding of the purified IgEs and a control (DNP-IgE) to relevant allergens to which the donor was allergic to (Fel d1 for cats, Der p1 for dust or Can e1 for dog as a control) by ELISA (FIG. 8G). One of the IgE mAbs purified bound to Fel d1, but not Can e1 or Der p1 (FIG. 8G), suggesting that human IgE antibodies could successfully be isolated from allergic BM using the IgE capture method of the disclosure.

### Example 10: Proof of Concept for Secreted IgG Capture

### Preparation of Secreted IgG Capture Reagent

An anti-mouse IgK antibody was conjugated with streptavidin using a Lightning-Link® streptavidin conjugation kit to form the secreted IgG capture reagent. In brief, a solution of anti-mouse IgK antibody containing 1 mg/mL streptavidin was prepared. One-hundred microliters of Modifier reagent was added to the solution with gentle mixing. The cap from the vial of Streptavidin Conjugation Mix was removed and the mixture, including the Modifier reagent, was added directly onto the lyophilized material. The material was resuspended gently by withdrawing and re-dispensing the added suspension once or twice using a pipette. The cap was replaced on the vial and the vial stood for three hours in the dark at 20-25° C. Thereafter, 100 μL of Quencher reagent was added with gentle mixing. Thus, the secreted IgK capture reagent was generated.

### Capture of Secreted IgG: Experimental Procedure

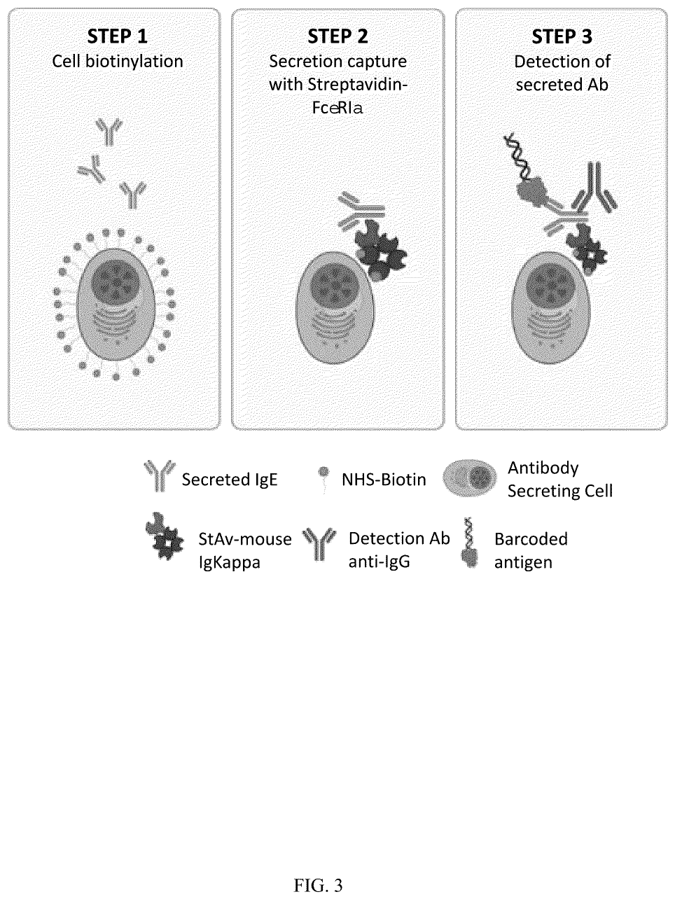

FIG. 3 (Step 1) shows the surface of a PC that has been biotinylated with NHS-biotin. FIG. 3 (Step 2) shows a secreted IgG capture reagent bound to the biotinylated PC. FIG. 3 (Step 2) also shows the anti-mouse IgK antibody of the secreted IgG capture reagent capturing an IgG that the PC has secreted. FIG. 3 (Step 3) shows the captured IgG bound to a barcoded antigen and bound by an anti-IgG antibody.

### Capture of Secreted IgG on the Surface of Primary Plasma Cells

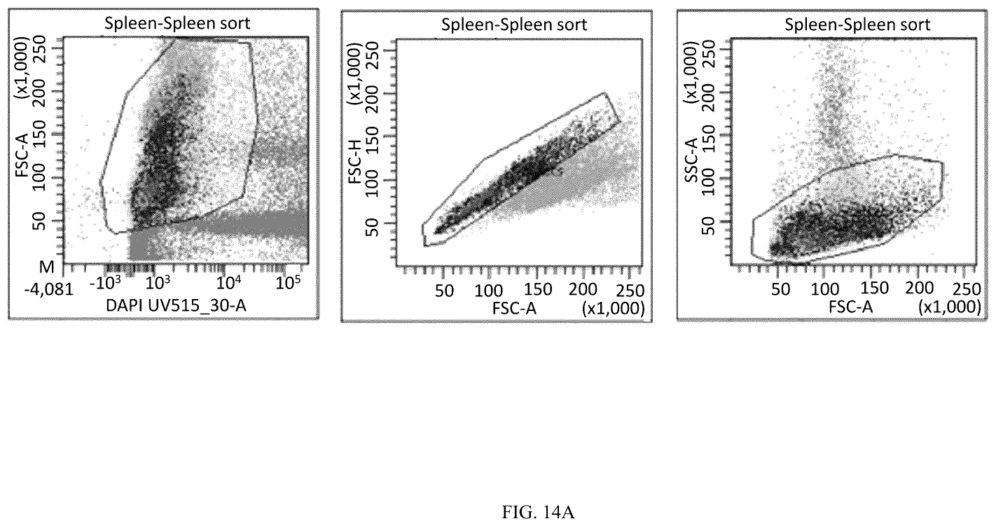

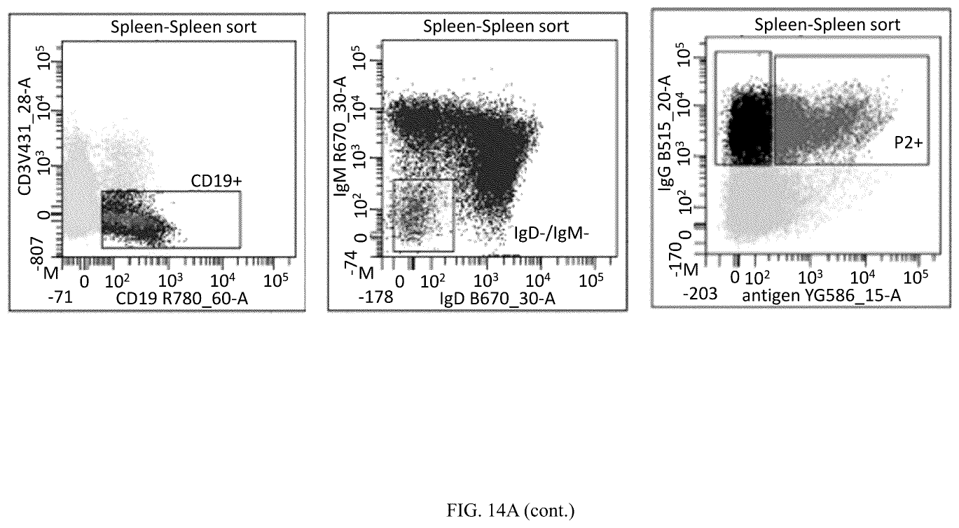

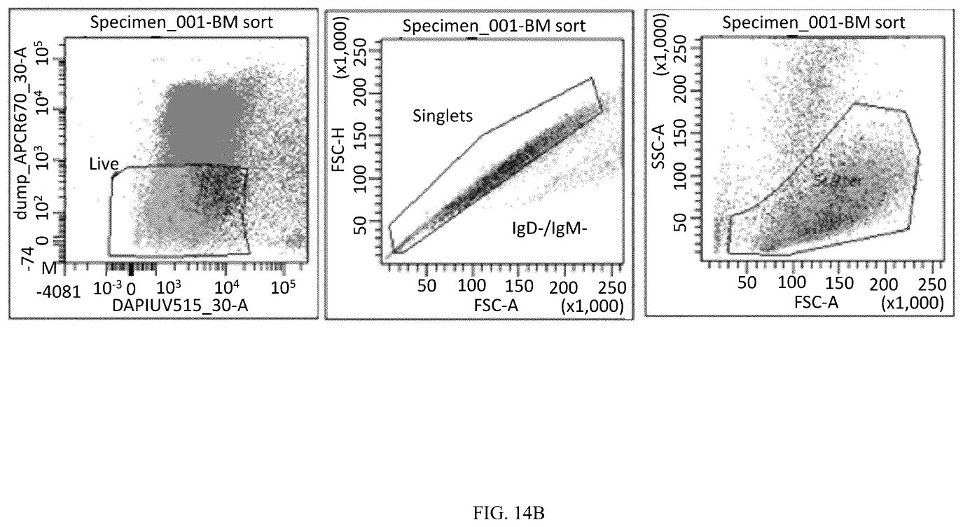

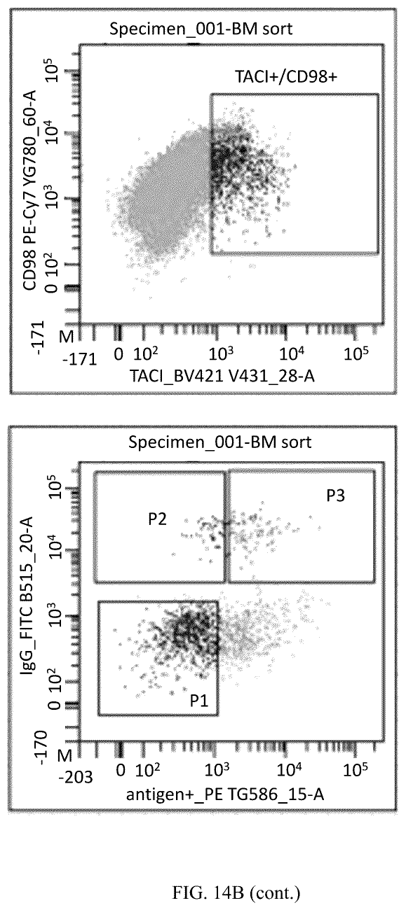

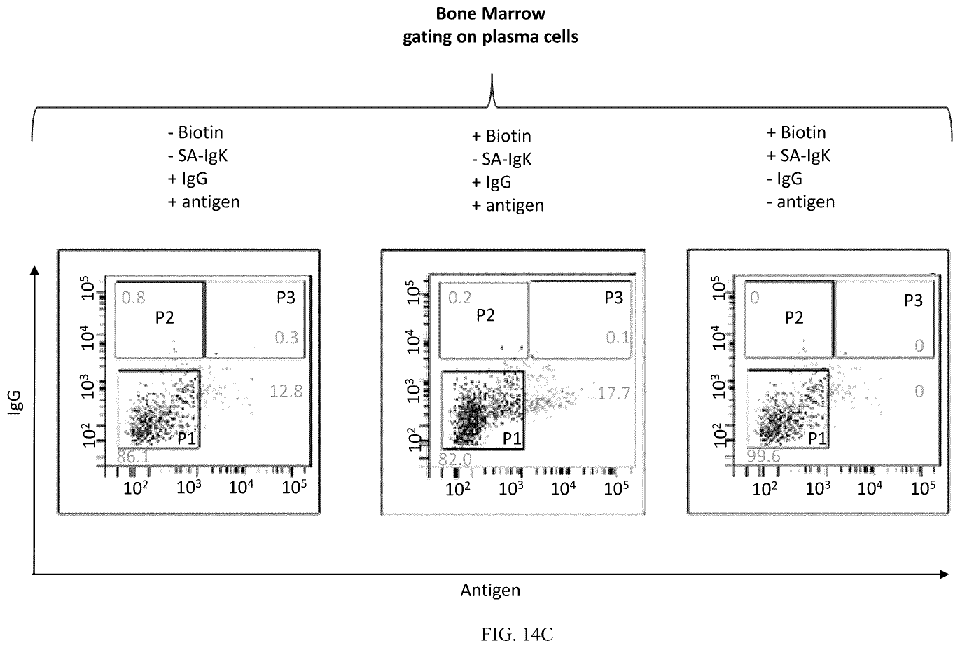

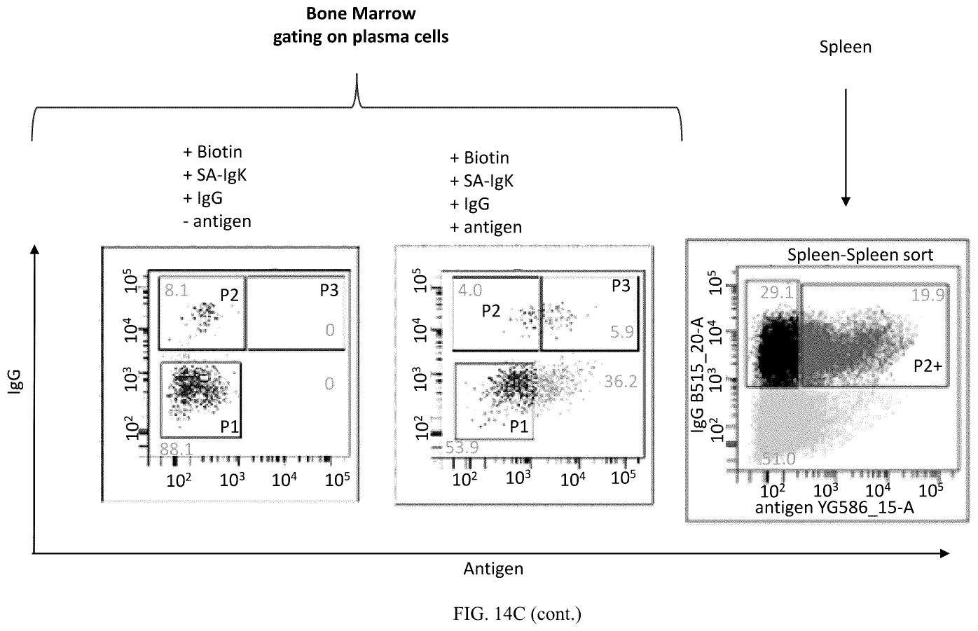

To detect antigen-specific IgG-secreting PCs, the footpads of VI3/VelocImmune mice were immunized twice a week for four weeks with an immunogen. The mice were rested for a month then boosted with immunogen four days before anesthesia, sacrifice, and procurement of the BM, Spleen, and Draining Lymph Nodes. Cells from the bone marrow, spleen, and draining-lymph nodes were prepared as described herein. Single-cell preparations from the spleen and lymph nodes were pooled and surface Fc receptors blocked with 1:10 diluted Fc Block for fifteen minutes at 4′C. The pooled preparations were then stained using an antibody cocktail to gate for antigen-specific IgG B cells (FIG. 14A). The antibody cocktail for the spleen and lymph nodes includes antibodies against murine IgG1, IgG2a, CD19, IgM, IgD, and CD3. The antibody cocktail for the BM includes antibodies against murine B220, CD138, TACI, CD98, TCRB, CD200R3, Ly6G, CD49b, CD11b, IgM, CD19, IgG1, IgG2a, and IgD. All antibodies are commercially available. The surfaces of the PCs were biotinylated as described herein, washed three times in cold PBS with 5% w/v BSA with the tubes changed for every wash step, and incubated with the secreted IgG capture reagent as described herein with the secreted IgG capture reagent present and the secreted IgE capture reagent absent. The cells were incubated at 37′C for one hour on a rotating platform to allow capture of secreted Ig antibody and stained with an antibody mix containing fluorescein isothiocyanate-labeled anti-IgG (BD Biosciences and Southern Biotech) for detection of captured IgG and barcoded, phycoerythrin-labeled immunogen in PBS with 5% BSA for thirty minutes at 4° C. Labeled PCs were sorted on a BD FACSSymphony® S6 flow cytometer and PCs identified upon which secreted IgG had been captured (FIG. 14B). Controls were used to detect an antigen-specific population with secreted IgG captured on the cell surface thereof (FIG. 14C).

### Example 11: Single-Cell Library Preparation and Sequencing of Plasma Cells with Secreted IgG Captured on the Surface Thereof

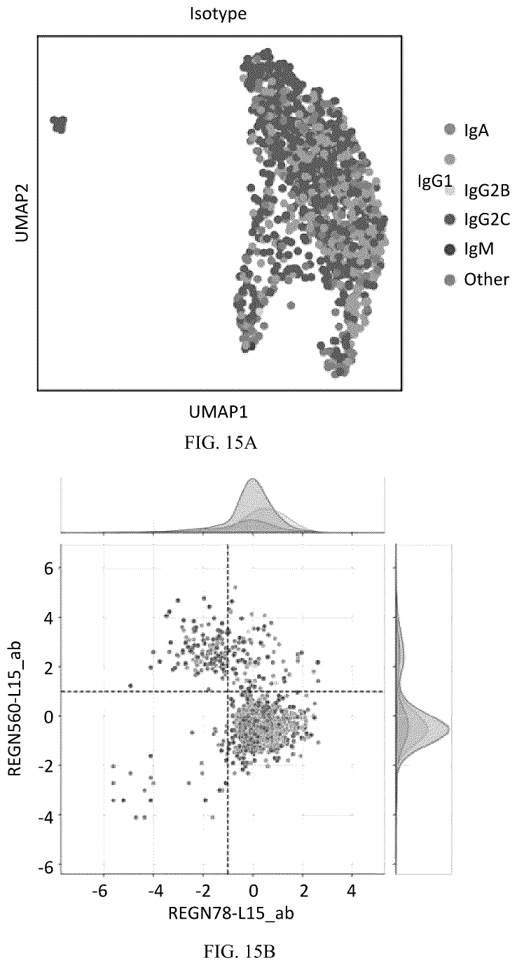

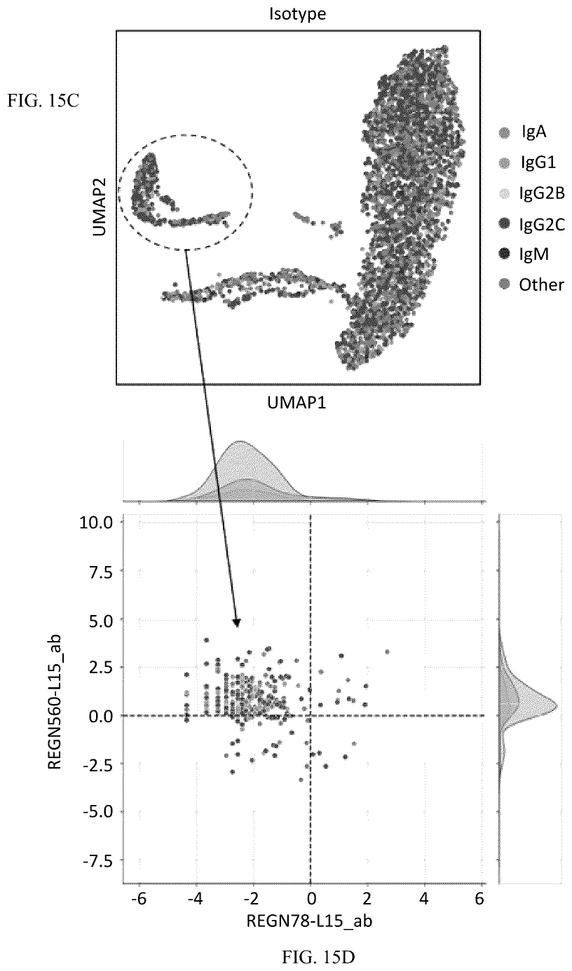

For IgG-secreting PCs for which the IgG had been captured, libraries for gene expression, the V(D)J regions of the secreted IgG, and barcoded antigens were prepared and sequenced in the manner described in Example 3. The individual libraries were also mapped, quantified, normalized, and processed in the manner described in Example 4. Owing to the humanized nature of the V(D)J region of the VelocImmune® mice, the V(D)J library was mapped to the standard human germline IMGT reference. Following processing of the three single-cell libraries, the gene expression and V(D)J region sequencing data were used to subset PCs with a valid IgG constant region and detectable expression of IgG and CD79a but lacking the expression of Ms4a1 and CD19 typically found in non-PC B cells. In order to select antigen-specific, IgG-expressing PCs, the antigen specificity score was calculated for the target immunogen against a control antigen (hIL-6Rα) as described in Example 4. Results are shown in FIG. 15, UMAP of the bone marrow in FIGS. 15A and 15B and the spleen in FIGS. 15C and 15D. The x axis in FIG. 15B and FIG. 15D represents the centered log-ratio of the immunogen antigen signal and the y-axis the centered log-ratio of the hIL-6Rα antigen signal. FIG. 15D only shows the plasma cells as determined by expression of CD79A, but lack of CD19 and MS4A1. Cells are colored by the plasma cell isotype as determined from the VDJ library sequencing.

### Example 12: IgG and Ag+ELISAs

To test antigen-specificity and IgG expression of BCR sequences from the IgG secretion trap, hIL-4Rα antigen or anti-human IgG (Jackson Labs) was plated at 1 ug/mL or 2 ug/mL, respectively, overnight at +4° C. The next day, plates were washed three times in PBS supplemented with 0.05% Tween20 (PBS-T) and blocked with PBS with 0.5% BSA for 1 hour at room temperature. Plates were subsequently washed three times with PBS-T, and diluted supernatants from Expi293F cells transfected with individual BCRs from the BMPC, PB/PC-like, or Spleen B-cell fractions were plated for 1 hour at room temperature. Plates were then washed three times with PBS-T and incubated with an anti-human IgG-HRP antibody (Jackson Labs) at 1:10,000 for 1 hour at room temperature. Plates were washed three times in PBS-T, developed using TMB substrate, stopped using 1N sulfuric acid, and read at 450 nm.

### Example 13: Identification and Isolation of Antigen-Specific IgG Antibody Secreting Cells from Immunized Mice

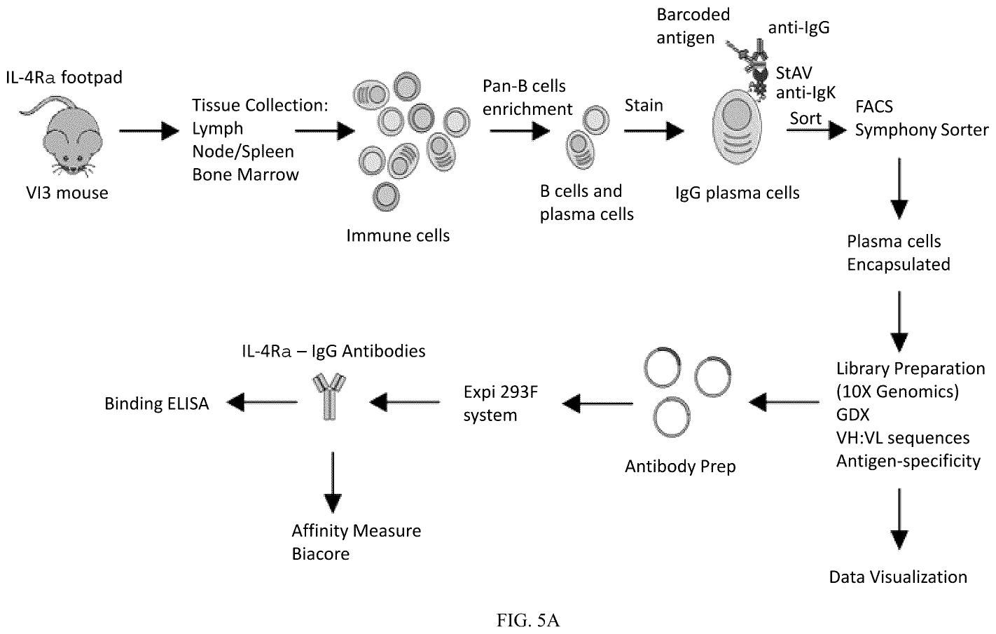

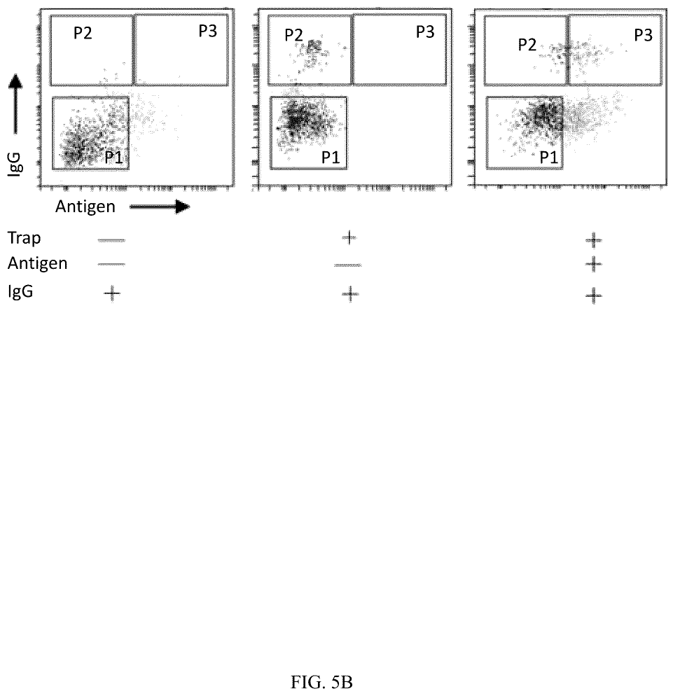

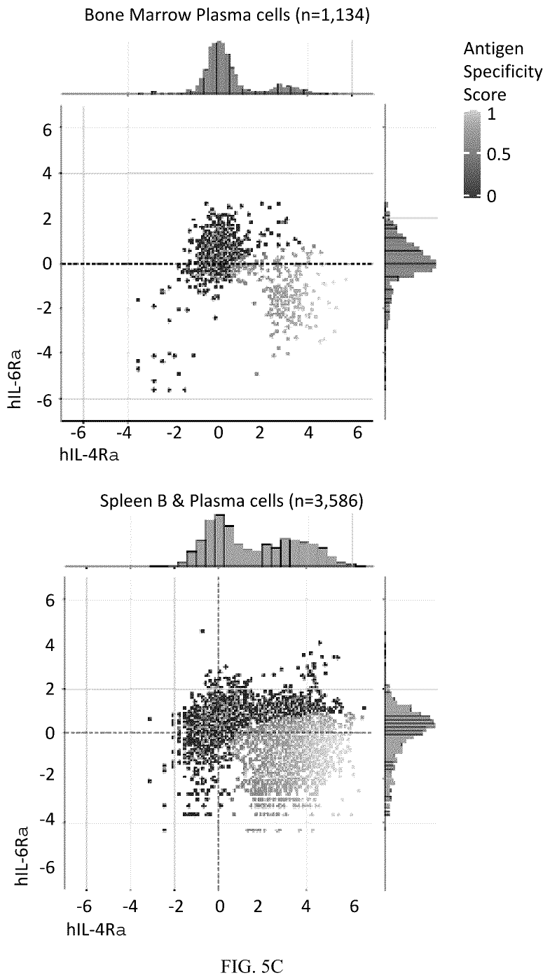

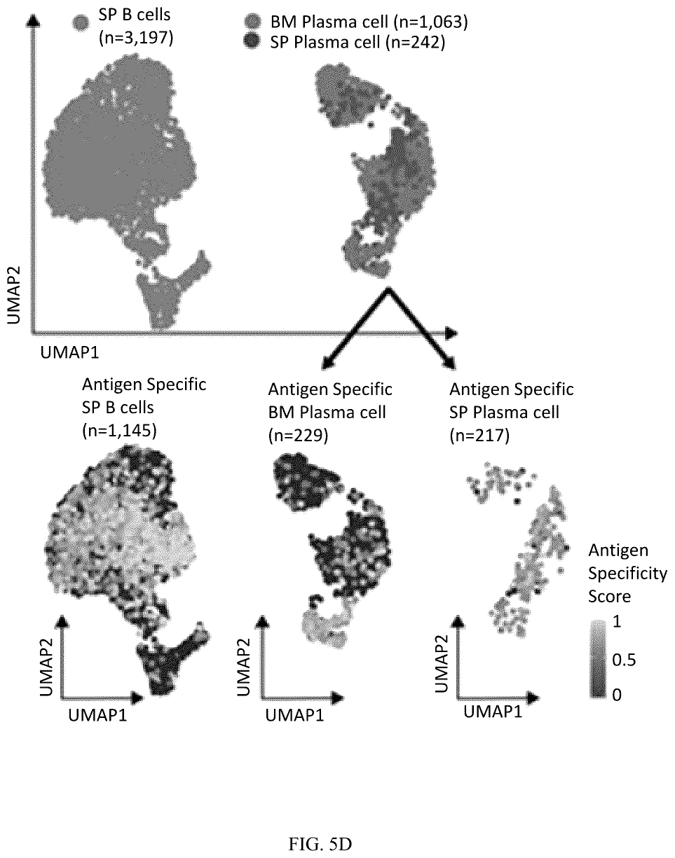

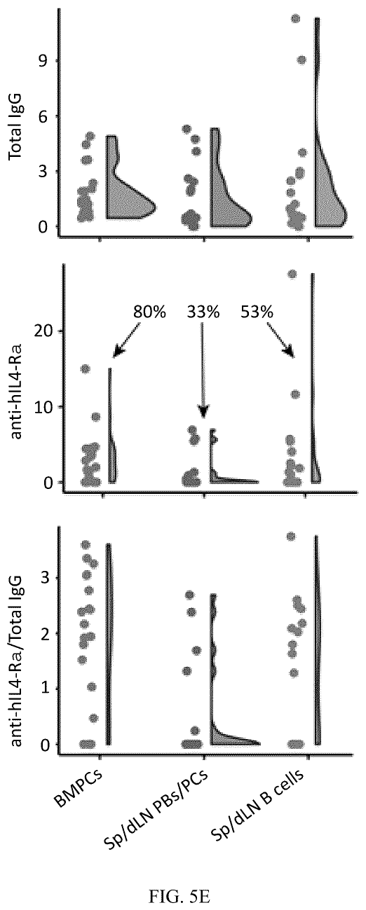

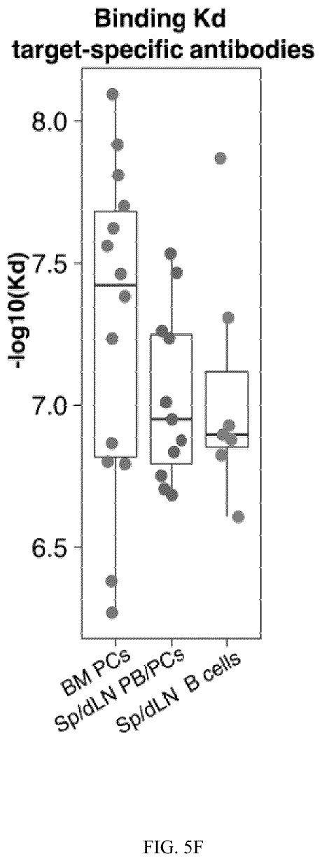

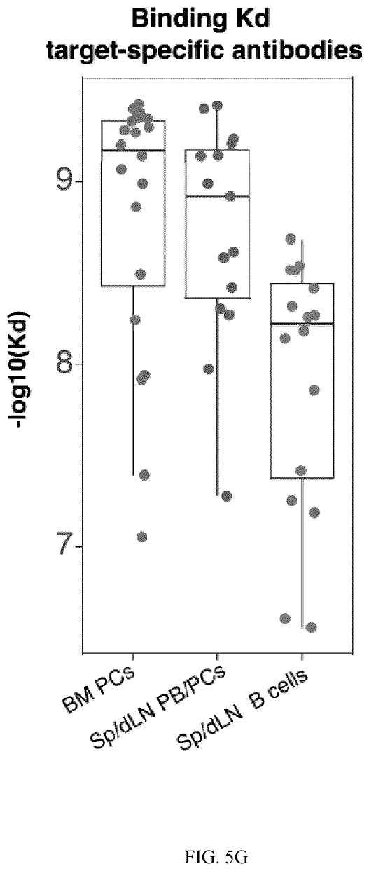

As described in FIG. 1, an algk antibody was conjugated to streptavidin to capture secreted antibodies using kappa light chain. The StAv-algk capture complex was then used on NHS-biotin coated ASCs from VelocImmune® mice immunized with human IL-4Rα (hIL-4Rα). Detection of Ag⁺/IgG⁺ secreting plasma cells was achieved by staining cells with anti-mouse IgG and barcoded hIL-4Rα antigen. Barcoded hIL-6Rα was used as a negative control (FIG. 5A).

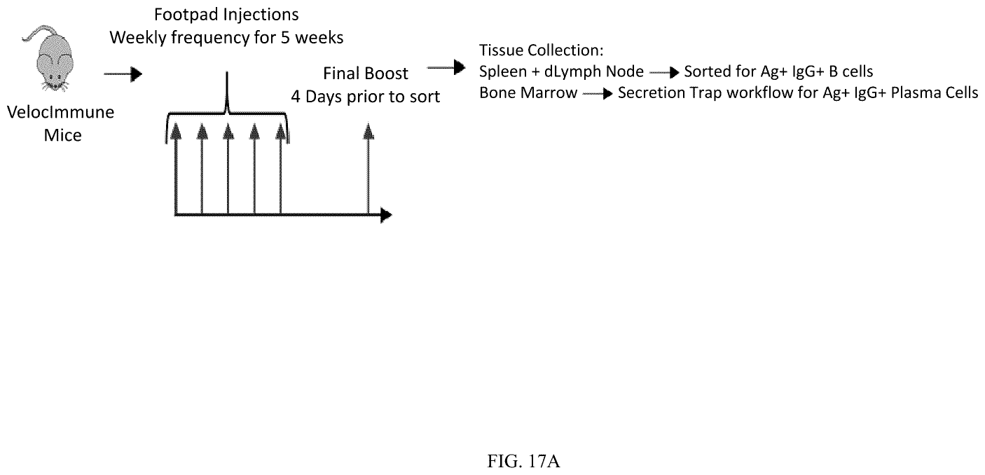

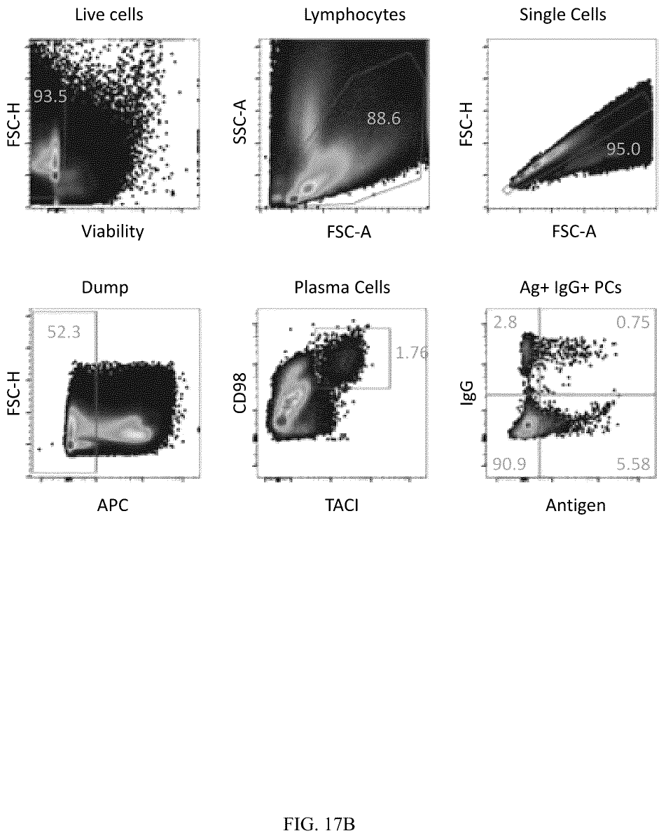

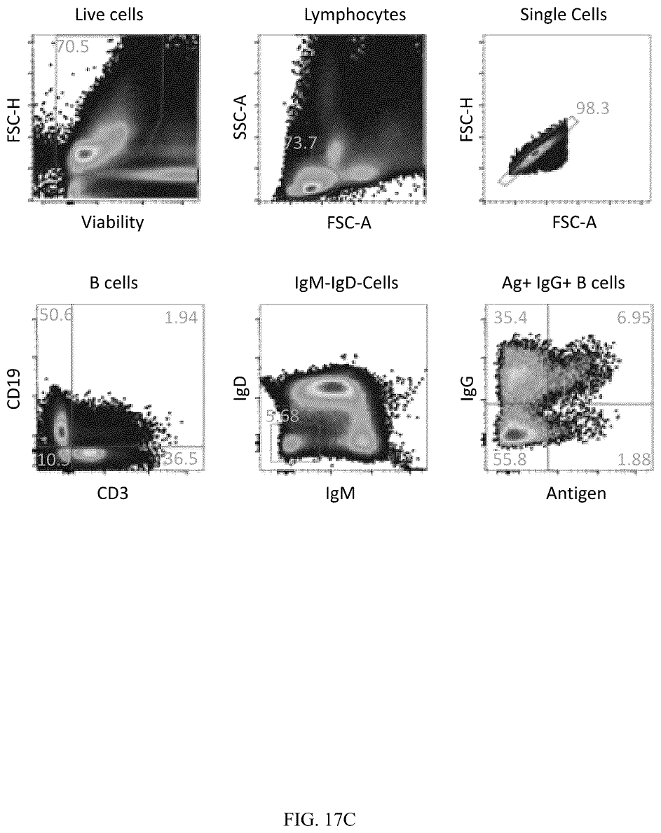

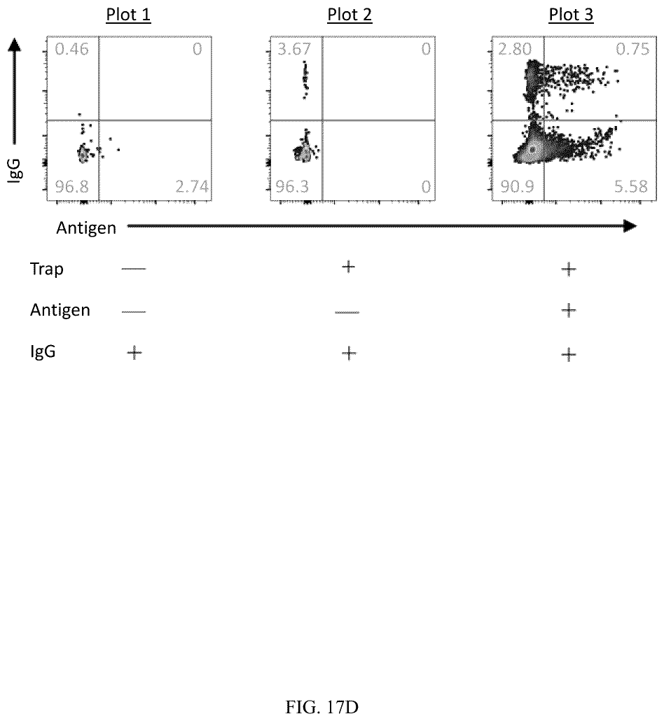

As shown in FIGS. 5 and 17, to detect Ag⁺/IgG⁺ ASCs, mice were immunized with hIL-4Rα monomeric protein via footpad (FIG. 5A and FIG. 17A). Single cell suspensions of BM cells were generated, and B lineage cells were enriched by negative selection (FIG. 5A). Cells were coated with NHS-biotin and secreted IgG captured with StAv-algk as described herein (FIG. 1). Following assembly of the biotin—StAv-algk complex on the surface, enriched B cells/ASCs were stained with an antibody mixture containing PC-specific markers, αlgG for detection of secreted IgG, barcoded hIL-4Rα for detection of Ag+ cells and barcoded hIL-6Rα as a negative control. Spleen and draining lymph nodes (dLNs) from the same mice were pooled and stained using a similar antibody cocktail for comparison of VH:VL sequences from Ag+ B cells and plasmablasts (PBs) to BMPCs (FIGS. 17B and 17C). Ag⁺/IgG⁺ PCs from the BM were sorted using staining controls along with B cells and ASCs expressing surface BCR (PBs/PCs) from spleen/dLN (FIG. 5B and FIGS. 17B, 17C, and 17D).

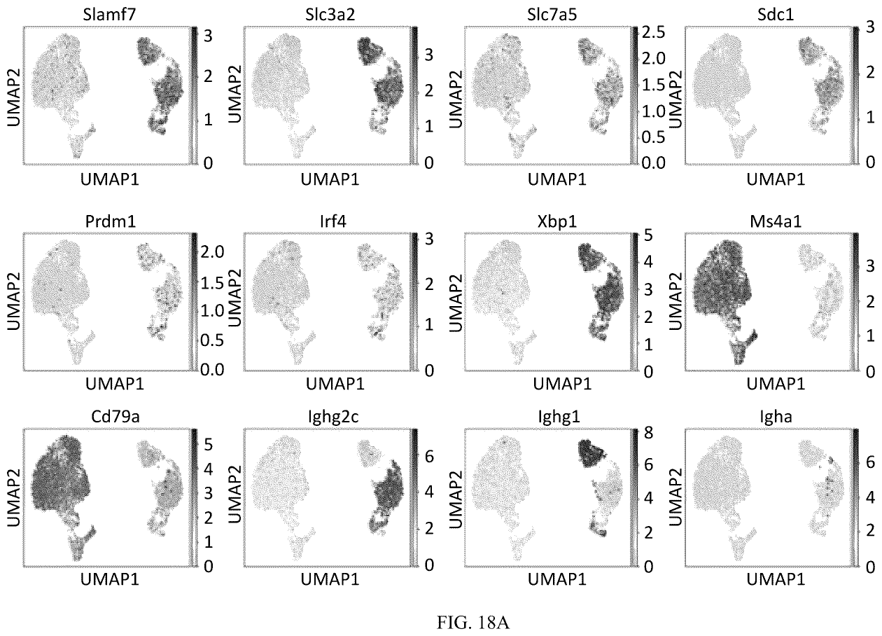

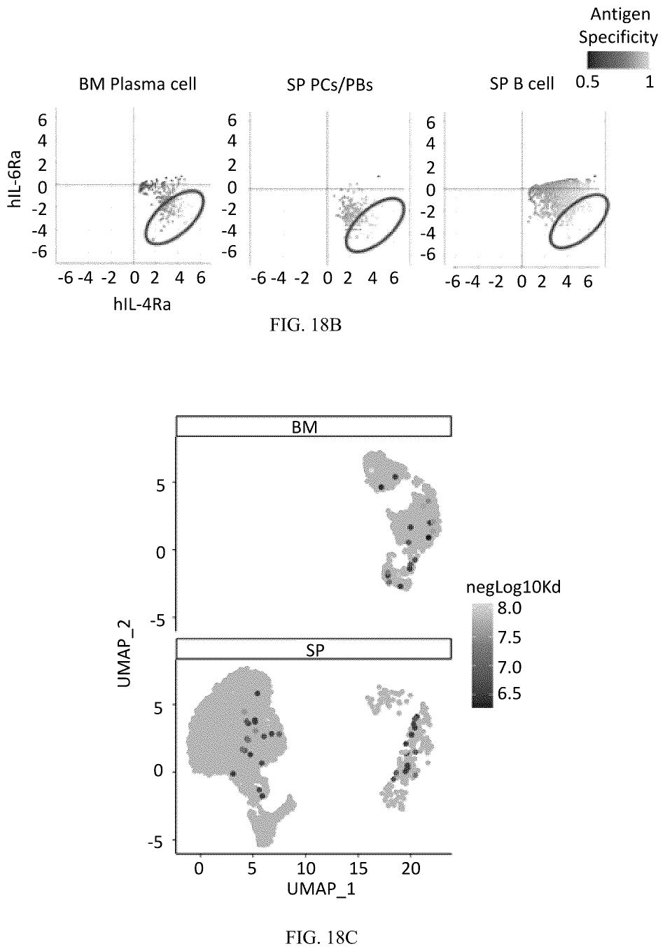

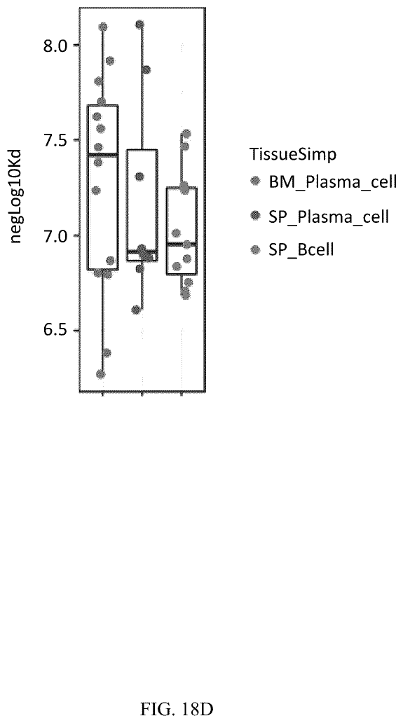

scRNA-seq was performed on sorted BMPCs, splenic/dLN B cells, and PBs/PCs to determine transcriptional differences, identify VH:VL repertoire changes and quantify antigen specificity at a single cell level. From barcoded antigen counts of hIL-4Rα and a negative control antigen hIL-6Rα, Ag⁺/IgG⁺ cells were verified (FIG. 5C). To prioritize specificity and minimize polyreactivity, an antigen specificity score (AgSS) was determined to selects cells with strong specificity for hIL-4Rα and minimal hIL-6Rα binding. Cells with high AgSS were prioritized for hIL-4Rα and had minimal barcoded UMI counts for the control antigen hIL-6Rα (FIG. 5C). Combined transcriptional analysis and unbiased clustering indicated that plasma cells from either spleen or BM clustered together and expressed classical PC markers such as Slamf7, Xbp1, Sdc1, Prdm1 and SIc3a2 (FIG. 5D, FIG. 18A). Splenic B cells, however, clustered very distinctly from plasma cells and expressed typical B cell markers such as Ms4a1, and Cd79a (FIG. 18A). Additionally, Ag+ cells with high AgSS (>0.5) from both the BM and spleen generally clustered together (FIG. 5D). In BM PCs especially, cells with high AgSS clustered together at the bottom of the UMAP (FIG. 5D).

### Example 14: Affinity of Antigen-Specific IgG Plasma Cells Isolated from Bone Marrow and Splenic/dLN B Cells

scRNA-seq yielded three distinct cell populations: bone marrow plasma cells (BMPCs), spleen/dLN plasmablast/plasma cell-like (Sp/dLN PBs/PCs), and spleen/dLN B cells (Sp/dLN B cells). From each of these populations, 20 unique VH:VL sequences from cells with the highest hIL-4Rα barcode signal were selected for cloning and expression analysis (FIGS. 18B and 18C). 20 heavy and light chain variable sequences were successfully cloned from BMPCs, 19 from Sp/dLN B cells and 19 from Sp/dLN PBs/PCs into vectors expressing human IgG4 and human Igk constants (see, Table 1).

Consistent IgG expression was observed across all three populations, but anti-hIL-4Rα activity was observed in 80% (16/20) of BMPCs compared to 32% (6/19) and 53% (10/19) of Sp/dLN PBs/PCs and Sp/dLN B cells, respectively (FIG. 5E; Table 1). When accounting for varying levels of IgG in the supernatants, BMPCs also had a higher median ratio of hIL-4Rα:total IgG binding, an indication that these sequences will have higher affinity (FIG. 5E). Biacore analysis of the supernatants and purified antibodies confirmed this, with antibodies from the BMPCs having the highest affinity antibodies as measured by negative log 10 of the binding Kds (FIG. 5F and FIG. 18D).

### Example 15: scRNA-Seq of Captured IgG Cells

Following validation of Ag⁺ cells, we investigated the unique BCR sequences obtained by scRNA-seq of the VDJ region to compare Ag⁺ BMPCs with B and PBs/PCs from the spleen/dLN. BCR sequences from all cells were mapped to specific V, D and J gene segments by IgBlast and the extent of somatic hypermutation was measured by percent similarity to germline V regions. Ag⁺ cells in all three compartments exhibited lower percent similarity to germline V regions and therefore more frequent mutations than non-Ag+ cells (FIG. 6A). The BCR sequences of Ag+ cells were further investigated by identifying BCR clonotypes in cells which shared the same heavy and light chain V gene segment and had identical CDR3 amino acid sequences in both the heavy and light chains (Table 2). 10 unique CDR3 sequence pairs were clonally shared among all 3 compartments, while the majority of each BCR repertoire was specific to each compartment (Table 2).

In addition to BCR sequence analysis, the transcriptomes of Ag+ and non-Ag+ cells in each of the three compartments were also compared to identify gene signatures related to hIL-4Rα specificity. In BMPCs, several gene expression programs differed based on hIL-4Ra specificity (FIGS. 6C and 6D). Tmsbx4, Cd24a, Tmsb10 and Fkbp11 all demonstrated significant upregulation in hIL-4Rα specific BMPCs. Non-Ag+ cells showed increases in Ly6d, Prg2, Tmem176a and Tmem176b (FIGS. 6C and 6D). Sub-clustering of BMPCs indicated Cd24a, Ppib, Fkbp11 and Ssr2 transcripts are all expressed highly toward the bottom of the UMAP projection (FIG. 6E), while gene programs marked by Tmem176a, Tmem176b, Ly6d and Cd74 expression cluster in the middle and top regions of the UMAP projection (FIG. 6E). However, despite significant transcriptional differences, BCR sequences cloned from either of the transcriptionally distinct regions resulted in high affinity hIL-4Rα specific antibodies as measured by negative log 10 of the binding Kds (FIG. 6F).

### Example 16: Expanding Trap-N-Seq to Human Bone Marrow to Characterize Short Versus Long-Lived Vaccine-Specific IgG Plasma Cells

In this Example, TRAPnSeq was used to isolate vaccine-specific IgG+ plasma cells (PCs) from human bone marrow. After enrichment for B cells and antibody secreting cells (ABCs), cells were biotinylated with NHS-biotin, and an affinity matrix was assembled using streptavidin coupled to anti-human Igk (StAv-algk) to capture secreted IgG. Following assembly of biotin—StAv-algk complex on the surface, enriched B cells/ASCs were stained with an antibody mix containing PC-specific markers, αlgG for detection of secreted IgG, and fluorescent-tagged antigens (listed in the table below) for detection of Ag+ cells.

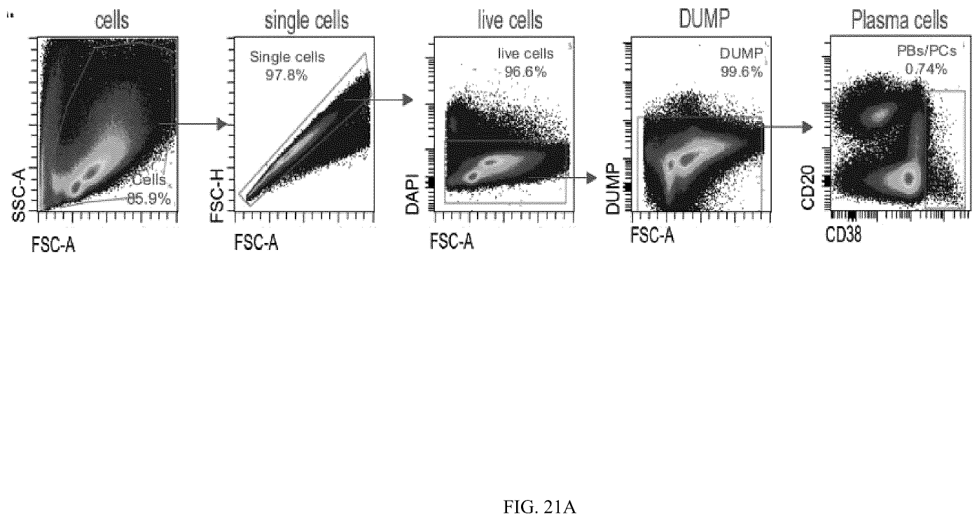

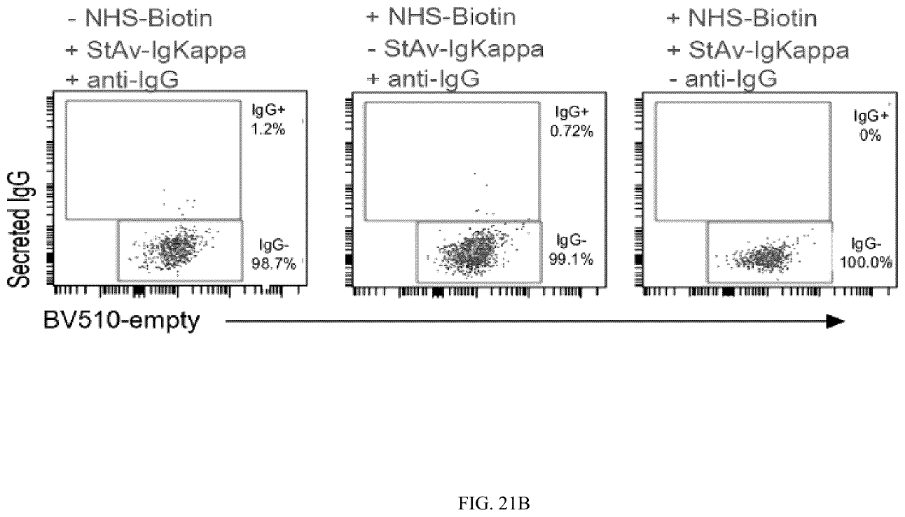

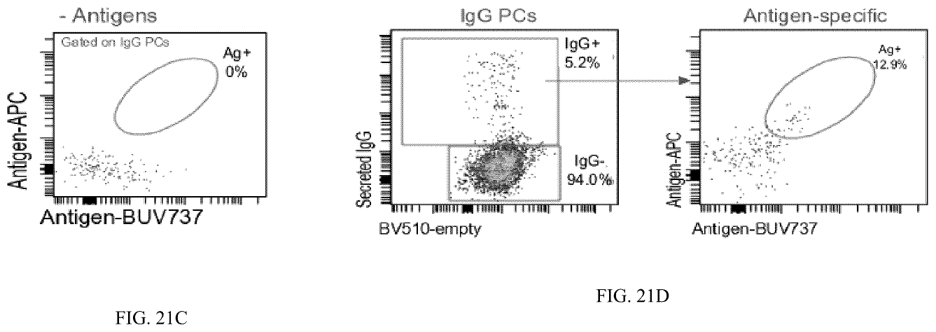

After gating on PCs as CD20-CD38++(FIG. 21A), the use of secretion trap to isolate vaccine-specific IgG+ PCs from human bone marrow was validated. The necessary components were demonstrated in FIG. 21B for the detection of secreted IgG on the surface of PCs, and in FIG. 21C for detection of antigen-specific IgG. Complete assembly of all components of the secretion trap allowed detection of secreted IgG antibodies (FIG. 21D, left), and detection of those that were vaccine-specific (FIG. 21D, right).

### Antigen List

| | | Supplier | Cat | Lot |
|---|---|---|---|---|
| 1 | Native VZV | Creative Diagnostics | DAG219 | VZ030066A1 |
| 2 | H1N1 ANTIGEN (A/New | Creative Diagnostics | DAG3690 | C1035 |
| | Caledonia/20/99) | | | |
| 3 | NATIVE IBV | Creative Diagnostics | DAGC081 | V2971 |
| | (B/Jiangsu/10/03) | | | |
| 4 | MUV GRADE 2 | Creative Diagnostics | DAG- | MU060020A3 |
| | | | H10369 | |
| 5 | MEASLES GRADE 2 | Creative Diagnostics | DAG- | ME040037A4 |
| | | | H10368 | |
| 6 | RUBELLA VIRUS STRAIN | Creative Diagnostics | DAGA- | C1664 |
| | HPV-77 | | 545 | |
| 7 | Diptheria Toxin | Abcam | ab188505 | GR3400744-7 |
| 8 | Tetanus Toxoid | EMD Millipore | 582231- | 3845630 |
| | | | 25ug | |

### Example 17: Secretion Trap in Combination with Antigen Chase

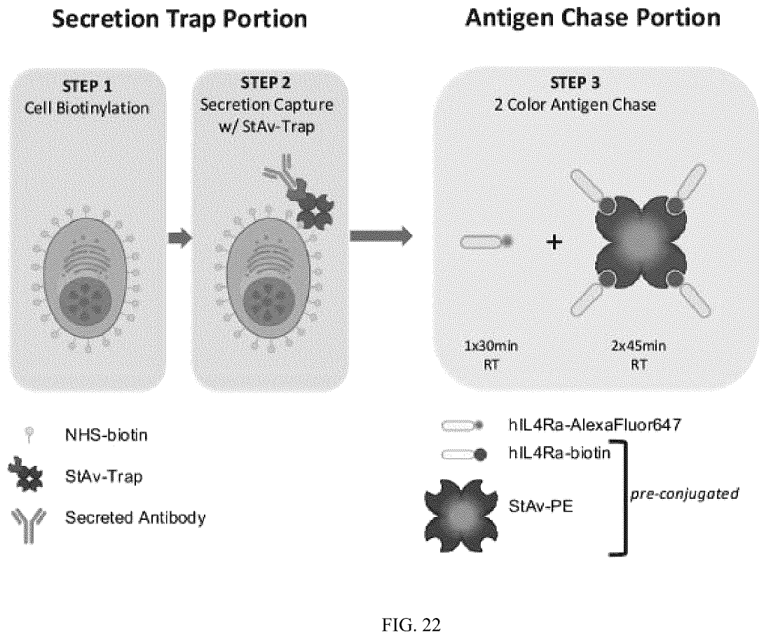

The workflow of a secretion trap combined with antigen chase is illustrated in FIG. 22.

Biotinylated human IL-4 Receptor a (hIL-4Rα) ectodomain was conjugated to PE-labeled streptavidin (“SA-PE”) tetramer overnight at 4′C. Separately, a trap antibody (mouse anti-human IgG (BD cat #555784, Clone #G18-145)) was conjugated to streptavidin overnight using a commercially available kit (abcam cat #ab102921).

A25 cells were suspended in NHS-biotin (Sigma cat #203112) at final concentration of 0.5 mg/mL in DPBS/FBS for 15 min at 37° C. A25 cells were derived from the commercially available A20 line by knocking out the endogenous surface BCR. Thus, A25 cells are mouse B cells without surface or secreted BCR.

After incubation, the cells were washed 3 times in DPBS/FBS, with transfer to a new conical tube after each wash to remove residual biotin.

Biotinylated cells were incubated with or without streptavidin conjugated trap antibody (or “SA-anti-hulgG”), performed at 1:100 dilution) at 10 million cells/mL in DPBS/FBS for about 15 minutes at 4° C. The 1:100 cell versus trap antibody ratio ensured that the trap reagent was bound to all biotinylated cells prior to spiking in the antibodies in the next step. After incubation, the cells were washed twice in DPBS/FBS and then resuspended in DPBS/FBS.

The following four antibodies differing in affinity were used. Each individual antibody was spiked into a cell suspension at about 1 mg antibody per 2 million cells. After incubation, the cells were washed twice in DPBS/FBS.

| | KD | t½ pH 7.4 |
|---|---|---|
| ab_1 | 1.68E−09 | >115.5 |
| ab_2 | 3.26E−09 | 21.7 |
| ab_3 | 1.42E−08 | 10.6 |
| ab_4 | neg control | |

Next, the cells were incubated with 5 nM hIL-4Rα labeled with AlexaFluor 647 (“AF647”) for 30 min at room temperature (RT) in DPBS/FBS. After incubation, the cells were washed twice in DPBS/FBS.

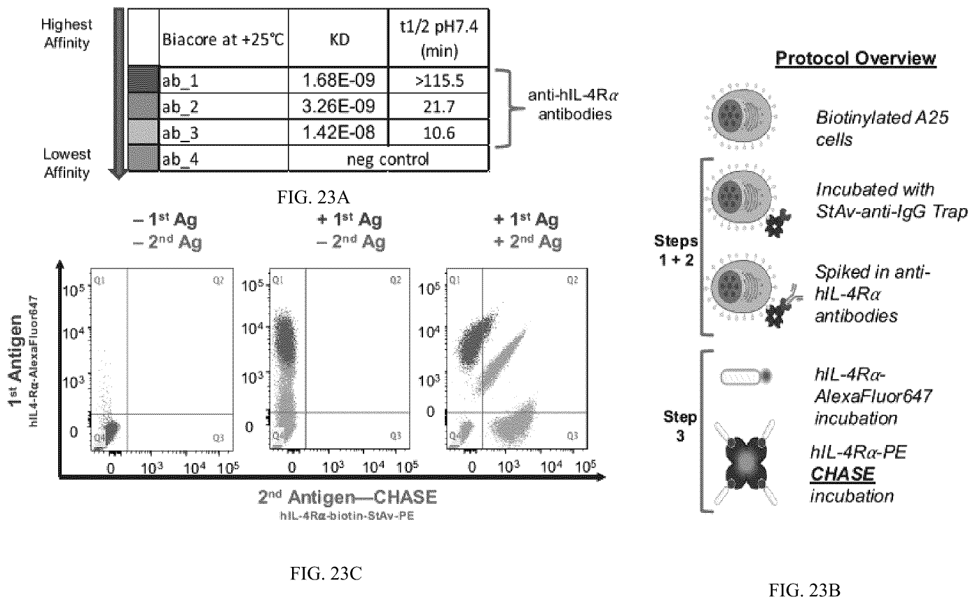

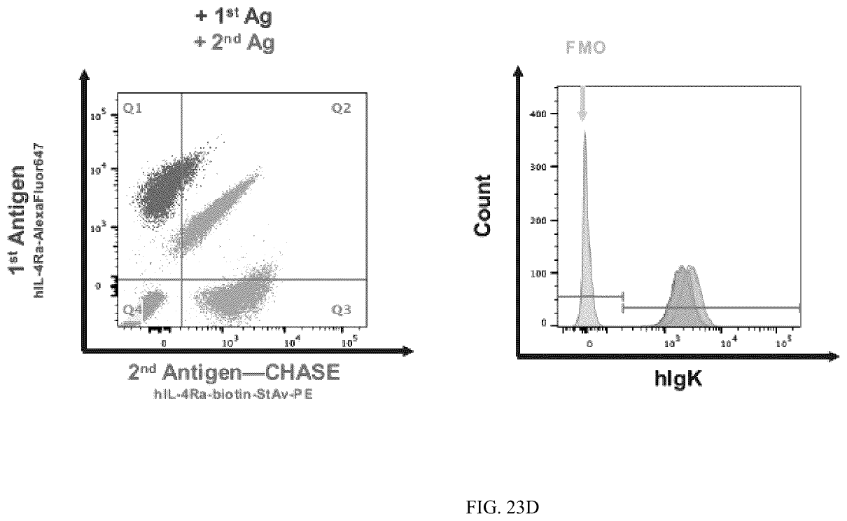

Afterwards, the cells were incubated with pre-conjugated hIL-4Rα biotin-StAv-PE twice for 45 min (or 2×45 min) at RT in DPBS/FBS. The concentration for antigen hIL-4Rα was nM. After incubation, the cells were washed twice in DPBS/FBS, and analyzed by flow cytometry. As shown in FIG. 23C, the four antibodies show clear separation by affinity.

As a control, after the chase antigen incubations (hIL-4Rα-biotin-StAv-PE), cells were washed twice in DPBS/FBS and then stained with an anti-hulgK-PE-Cy7 antibody to detect antibody on the surface of the cells. Ab 4/neg control did not stain for antigen, but it was positive for IgK, indicating that the antibody was still captured on the surface of the cells.

TABLE 1
Antigen-specific IgG plasma cell sequences isolated from the
bone marrow plasma cells and splenic plasma and B cells

| Line # | Cell Barcode | Heavy Sequence |
|---|---|---|
| 1 | SEQ ID NO: 1 | SEQ ID NO: 2 |
| 2 | SEQ ID NO: 3 | SEQ ID NO: 4 |
| 3 | SEQ ID NO: 5 | SEQ ID NO: 6 |
| 4 | SEQ ID NO: 7 | SEQ ID NO: 8 |
| 5 | SEQ ID NO: 9 | SEQ ID NO: 10 |
| 6 | SEQ ID NO: 11 | SEQ ID NO: 12 |
| 7 | SEQ ID NO: 13 | SEQ ID NO: 14 |
| 8 | SEQ ID NO: 15 | SEQ ID NO: 16 |
| 9 | SEQ ID NO: 17 | SEQ ID NO: 18 |
| 10 | SEQ ID NO: 19 | SEQ ID NO: 20 |
| 11 | SEQ ID NO: 21 | SEQ ID NO: 22 |
| 12 | SEQ ID NO: 23 | SEQ ID NO: 24 |
| 13 | SEQ ID NO: 25 | SEQ ID NO: 26 |
| 14 | SEQ ID NO: 27 | SEQ ID NO: 28 |
| 15 | SEQ ID NO: 29 | SEQ ID NO: 30 |
| 16 | SEQ ID NO: 31 | SEQ ID NO: 32 |
| 17 | SEQ ID NO: 33 | SEQ ID NO: 34 |
| 18 | SEQ ID NO: 35 | SEQ ID NO: 36 |
| 19 | SEQ ID NO: 37 | SEQ ID NO: 38 |
| 20 | SEQ ID NO: 39 | SEQ ID NO: 40 |
| 21 | SEQ ID NO: 41 | SEQ ID NO: 42 |
| 22 | SEQ ID NO: 43 | SEQ ID NO: 44 |
| 23 | SEQ ID NO: 45 | SEQ ID NO: 46 |
| 24 | SEQ ID NO: 47 | SEQ ID NO: 48 |
| 25 | SEQ ID NO: 49 | SEQ ID NO: 50 |
| 26 | SEQ ID NO: 51 | SEQ ID NO: 52 |
| 27 | SEQ ID NO: 53 | SEQ ID NO: 54 |
| 28 | SEQ ID NO: 55 | SEQ ID NO: 56 |
| 29 | SEQ ID NO: 57 | SEQ ID NO: 58 |
| 30 | SEQ ID NO: 59 | SEQ ID NO: 60 |
| 31 | SEQ ID NO: 61 | SEQ ID NO: 62 |
| 32 | SEQ ID NO: 63 | SEQ ID NO: 64 |
| 33 | SEQ ID NO: 65 | SEQ ID NO: 66 |
| 34 | SEQ ID NO: 67 | SEQ ID NO: 68 |
| 35 | SEQ ID NO: 69 | SEQ ID NO: 70 |
| 36 | SEQ ID NO: 71 | SEQ ID NO: 72 |
| 37 | SEQ ID NO: 73 | SEQ ID NO: 74 |
| 38 | SEQ ID NO: 75 | SEQ ID NO: 76 |
| 39 | SEQ ID NO: 77 | SEQ ID NO: 78 |
| 40 | SEQ ID NO: 79 | SEQ ID NO: 80 |
| 41 | SEQ ID NO: 81 | SEQ ID NO: 82 |
| 42 | SEQ ID NO: 83 | SEQ ID NO: 84 |
| 43 | SEQ ID NO: 85 | SEQ ID NO: 86 |
| 44 | SEQ ID NO: 87 | SEQ ID NO: 88 |
| 45 | SEQ ID NO: 89 | SEQ ID NO: 90 |
| 46 | SEQ ID NO: 91 | SEQ ID NO: 92 |
| 47 | SEQ ID NO: 93 | SEQ ID NO: 94 |
| 48 | SEQ ID NO: 95 | SEQ ID NO: 96 |
| 49 | SEQ ID NO: 97 | SEQ ID NO: 98 |
| 50 | SEQ ID NO: 99 | SEQ ID NO: 100 |
| 51 | SEQ ID NO: 101 | SEQ ID NO: 102 |
| 52 | SEQ ID NO: 103 | SEQ ID NO: 104 |
| 53 | SEQ ID NO: 105 | SEQ ID NO: 106 |
| 54 | SEQ ID NO: 107 | SEQ ID NO: 108 |

| Line # | Light Sequence | v_identity | IR_VJ_1_locus | IR_VDJ_1_locus |
|---|---|---|---|---|
| 1 | SEQ ID NO: 109 | | IGK | IGH |
| 2 | SEQ ID NO: 110 | 0.95973 | IGK | IGH |
| 3 | SEQ ID NO: 111 | 0.97987 | IGK | IGH |
| 4 | SEQ ID NO: 112 | 0.98658 | IGK | IGH |
| 5 | SEQ ID NO: 113 | 0.9726 | IGK | IGH |
| 6 | SEQ ID NO: 114 | 0.95302 | IGK | IGH |
| 7 | SEQ ID NO: 115 | 0.9697 | IGK | IGH |
| 8 | SEQ ID NO: 116 | 0.95638 | IGK | IGH |
| 9 | SEQ ID NO: 117 | 0.99317 | IGK | IGH |
| 10 | SEQ ID NO: 118 | 0.95302 | IGK | IGH |
| 11 | SEQ ID NO: 119 | 0.98653 | IGK | IGH |
| 12 | SEQ ID NO: 120 | 0.98649 | IGK | IGH |
| 13 | SEQ ID NO: 121 | 0.95946 | IGK | IGH |
| 14 | SEQ ID NO: 122 | 0.97987 | IGK | IGH |
| 15 | SEQ ID NO: 123 | 0.95623 | IGK | IGH |
| 16 | SEQ ID NO: 124 | 0.97651 | IGK | IGH |
| 17 | SEQ ID NO: 125 | 0.98311 | IGK | IGH |
| 18 | SEQ ID NO: 126 | 0.94898 | IGK | IGH |
| 19 | SEQ ID NO: 127 | 0.97306 | IGK | IGH |
| 20 | SEQ ID NO: 128 | 0.97973 | IGK | IGH |
| 21 | SEQ ID NO: 129 | 0.98986 | IGK | IGH |
| 22 | SEQ ID NO: 130 | 0.9698 | IGK | IGH |
| 23 | SEQ ID NO: 131 | 0.97635 | IGK | IGH |
| 24 | SEQ ID NO: 132 | 0.94631 | IGK | IGH |
| 25 | SEQ ID NO: 133 | 0.93919 | IGK | IGH |
| 26 | SEQ ID NO: 134 | 0.98355 | IGK | IGH |
| 27 | SEQ ID NO: 135 | 0.98316 | IGK | IGH |
| 28 | SEQ ID NO: 136 | 0.99324 | IGK | IGH |
| 29 | SEQ ID NO: 137 | 0.99658 | IGK | IGH |
| 30 | SEQ ID NO: 138 | 0.95638 | IGK | IGH |
| 31 | SEQ ID NO: 139 | 0.99327 | IGK | IGH |
| 32 | SEQ ID NO: 140 | 0.97651 | IGK | IGH |
| 33 | SEQ ID NO: 141 | 0.96949 | IGK | IGH |
| 34 | SEQ ID NO: 142 | 0.93581 | IGK | IGH |
| 35 | SEQ ID NO: 143 | 1 | IGK | IGH |
| 36 | SEQ ID NO: 144 | 0.95973 | IGK | IGH |
| 37 | SEQ ID NO: 145 | 0.96207 | IGK | IGH |
| 38 | SEQ ID NO: 146 | 0.94595 | IGK | IGH |
| 39 | SEQ ID NO: 147 | 0.95973 | IGK | IGH |
| 40 | SEQ ID NO: 148 | 0.97611 | IGK | IGH |
| 41 | SEQ ID NO: 149 | 0.96309 | IGK | IGH |
| 42 | SEQ ID NO: 150 | 0.95946 | IGK | IGH |
| 43 | SEQ ID NO: 151 | 0.95302 | IGK | IGH |
| 44 | SEQ ID NO: 152 | 0.97987 | IGK | IGH |
| 45 | SEQ ID NO: 153 | 0.9798 | IGK | IGH |
| 46 | SEQ ID NO: 154 | 0.94595 | IGK | IGH |
| 47 | SEQ ID NO: 155 | 0.93266 | IGK | IGH |
| 48 | SEQ ID NO: 156 | 0.97651 | IGK | IGH |
| 49 | SEQ ID NO: 157 | 0.92568 | IGK | IGH |
| 50 | SEQ ID NO: 158 | 0.96959 | IGK | IGH |
| 51 | SEQ ID NO: 159 | 0.98635 | IGK | IGH |
| 52 | SEQ ID NO: 160 | 0.95638 | IGK | IGH |
| 53 | SEQ ID NO: 161 | 0.9698 | IGK | IGH |
| 54 | SEQ ID NO: 162 | 0.98635 | IGK | IGH |

| Line # | IR_VJ_1_cdr3 | IR_VDJ_1_cdr3 | IR_VJ_1_cdr3_nt |
|---|---|---|---|
| 1 | SEQ ID NO: 163 | SEQ ID NO: 164 | SEQ ID NO: 165 |
| 2 | SEQ ID NO: 166 | SEQ ID NO: 167 | SEQ ID NO: 168 |
| 3 | SEQ ID NO: 169 | SEQ ID NO: 170 | SEQ ID NO: 171 |
| 4 | SEQ ID NO: 172 | SEQ ID NO: 173 | SEQ ID NO: 174 |
| 5 | SEQ ID NO: 175 | SEQ ID NO: 176 | SEQ ID NO: 177 |
| 6 | SEQ ID NO: 178 | SEQ ID NO: 179 | SEQ ID NO: 180 |
| 7 | SEQ ID NO: 181 | SEQ ID NO: 182 | SEQ ID NO: 183 |
| 8 | SEQ ID NO: 184 | SEQ ID NO: 185 | SEQ ID NO: 186 |
| 9 | SEQ ID NO: 187 | SEQ ID NO: 188 | SEQ ID NO: 189 |
| 10 | SEQ ID NO: 190 | SEQ ID NO: 191 | SEQ ID NO: 192 |
| 11 | SEQ ID NO: 193 | SEQ ID NO: 194 | SEQ ID NO: 195 |
| 12 | SEQ ID NO: 196 | SEQ ID NO: 197 | SEQ ID NO: 198 |
| 13 | SEQ ID NO: 199 | SEQ ID NO: 200 | SEQ ID NO: 201 |
| 14 | SEQ ID NO: 202 | SEQ ID NO: 203 | SEQ ID NO: 204 |
| 15 | SEQ ID NO: 205 | SEQ ID NO: 206 | SEQ ID NO: 207 |
| 16 | SEQ ID NO: 208 | SEQ ID NO: 209 | SEQ ID NO: 210 |
| 17 | SEQ ID NO: 211 | SEQ ID NO: 212 | SEQ ID NO: 213 |
| 18 | SEQ ID NO: 214 | SEQ ID NO: 215 | SEQ ID NO: 216 |
| 19 | SEQ ID NO: 217 | SEQ ID NO: 218 | SEQ ID NO: 219 |
| 20 | SEQ ID NO: 220 | SEQ ID NO: 221 | SEQ ID NO: 222 |
| 21 | SEQ ID NO: 223 | SEQ ID NO: 224 | SEQ ID NO: 225 |
| 22 | SEQ ID NO: 226 | SEQ ID NO: 227 | SEQ ID NO: 228 |
| 23 | SEQ ID NO: 229 | SEQ ID NO: 230 | SEQ ID NO: 231 |
| 24 | SEQ ID NO: 232 | SEQ ID NO: 233 | SEQ ID NO: 234 |
| 25 | SEQ ID NO: 235 | SEQ ID NO: 236 | SEQ ID NO: 237 |
| 26 | SEQ ID NO: 238 | SEQ ID NO: 239 | SEQ ID NO: 240 |
| 27 | SEQ ID NO: 241 | SEQ ID NO: 242 | SEQ ID NO: 243 |
| 28 | SEQ ID NO: 244 | SEQ ID NO: 245 | SEQ ID NO: 246 |
| 29 | SEQ ID NO: 247 | SEQ ID NO: 248 | SEQ ID NO: 249 |
| 30 | SEQ ID NO: 250 | SEQ ID NO: 251 | SEQ ID NO: 252 |
| 31 | SEQ ID NO: 253 | SEQ ID NO: 254 | SEQ ID NO: 255 |
| 32 | SEQ ID NO: 256 | SEQ ID NO: 257 | SEQ ID NO: 258 |
| 33 | SEQ ID NO: 259 | SEQ ID NO: 260 | SEQ ID NO: 261 |
| 34 | SEQ ID NO: 262 | SEQ ID NO: 263 | SEQ ID NO: 264 |
| 35 | SEQ ID NO: 265 | SEQ ID NO: 266 | SEQ ID NO: 267 |
| 36 | SEQ ID NO: 268 | SEQ ID NO: 269 | SEQ ID NO: 270 |
| 37 | SEQ ID NO: 271 | SEQ ID NO: 272 | SEQ ID NO: 273 |
| 38 | SEQ ID NO: 274 | SEQ ID NO: 275 | SEQ ID NO: 276 |
| 39 | SEQ ID NO: 277 | SEQ ID NO: 278 | SEQ ID NO: 279 |
| 40 | SEQ ID NO: 280 | SEQ ID NO: 281 | SEQ ID NO: 282 |
| 41 | SEQ ID NO: 283 | SEQ ID NO: 284 | SEQ ID NO: 285 |
| 42 | SEQ ID NO: 286 | SEQ ID NO: 287 | SEQ ID NO: 288 |
| 43 | SEQ ID NO: 289 | SEQ ID NO: 290 | SEQ ID NO: 291 |
| 44 | SEQ ID NO: 292 | SEQ ID NO: 293 | SEQ ID NO: 294 |
| 45 | SEQ ID NO: 295 | SEQ ID NO: 296 | SEQ ID NO: 297 |
| 46 | SEQ ID NO: 298 | SEQ ID NO: 299 | SEQ ID NO: 300 |
| 47 | SEQ ID NO: 301 | SEQ ID NO: 302 | SEQ ID NO: 303 |
| 48 | SEQ ID NO: 304 | SEQ ID NO: 305 | SEQ ID NO: 306 |
| 49 | SEQ ID NO: 307 | SEQ ID NO: 308 | SEQ ID NO: 309 |
| 50 | SEQ ID NO: 310 | SEQ ID NO: 311 | SEQ ID NO: 312 |
| 51 | SEQ ID NO: 313 | SEQ ID NO: 314 | SEQ ID NO: 315 |
| 52 | SEQ ID NO: 316 | SEQ ID NO: 317 | SEQ ID NO: 318 |
| 53 | SEQ ID NO: 319 | SEQ ID NO: 320 | SEQ ID NO: 321 |
| 54 | SEQ ID NO: 322 | SEQ ID NO: 323 | SEQ ID NO: 324 |

| Line # | IR_VDJ_1_cdr3_nt | IR_VJ_1_v_gene |
|---|---|---|
| 1 | SEQ ID NO: 325 | IGKV1-12*01 |
| 2 | SEQ ID NO: 326 | IGKV1-17*01 |
| 3 | SEQ ID NO: 327 | IGKV1-5*03 |
| 4 | SEQ ID NO: 328 | IGKV1-5*03 |
| 5 | SEQ ID NO: 329 | IGKV2-28*01, IGKV2D-28*01 |
| 6 | SEQ ID NO: 330 | IGKV1-17*01 |
| 7 | SEQ ID NO: 331 | IGKV1-27*01 |
| 8 | SEQ ID NO: 332 | IGKV1-17*01 |
| 9 | SEQ ID NO: 333 | IGKV2-28*01, IGKV2D-28*01 |
| 10 | SEQ ID NO: 334 | IGKV1-17*01 |
| 11 | SEQ ID NO: 335 | IGKV1-27*01 |
| 12 | SEQ ID NO: 336 | IGKV1-27*01 |
| 13 | SEQ ID NO: 337 | IGKV1-27*01 |
| 14 | SEQ ID NO: 338 | IGKV1-17*01 |
| 15 | SEQ ID NO: 339 | IGKV1-27*01 |
| 16 | SEQ ID NO: 340 | IGKV1-27*01 |
| 17 | SEQ ID NO: 341 | IGKV1-33*01, IGKV1D-33*01 |
| 18 | SEQ ID NO: 342 | IGKV3-20*01 |
| 19 | SEQ ID NO: 343 | IGKV1-5*03 |
| 20 | SEQ ID NO: 344 | IGKV1-39*01, IGKV1D-39*01 |
| 21 | SEQ ID NO: 345 | IGKV1-27*01 |
| 22 | SEQ ID NO: 346 | IGKV3-20*01 |
| 23 | SEQ ID NO: 347 | IGKV1-12*01, IGKV1-12*02, |
| | | IGKV1D-12*02 |
| 24 | SEQ ID NO: 348 | IGKV1-39*01, IGKV1D-39*01 |
| 25 | SEQ ID NO: 349 | IGKV1-27*01 |
| 26 | SEQ ID NO: 350 | IGKV3-15*01 |
| 27 | SEQ ID NO: 351 | IGKV1-17*01 |
| 28 | SEQ ID NO: 352 | IGKV1-12*01, IGKV1-12*02, |
| | | IGKV1D-12*02 |
| 29 | SEQ ID NO: 353 | IGKV1-12*01, IGKV1-12*02, |
| | | IGKV1D-12*02 |
| 30 | SEQ ID NO: 354 | IGKV1-39*01, IGKV1D-39*01 |
| 31 | SEQ ID NO: 355 | IGKV1-5*03 |
| 32 | SEQ ID NO: 356 | IGKV1-17*01 |
| 33 | SEQ ID NO: 357 | IGKV3-15*01 |
| 34 | SEQ ID NO: 358 | IGKV1-27*01 |
| 35 | SEQ ID NO: 359 | IGKV1-12*01, IGKV1-12*02, |
| | | IGKV1D-12*02 |
| 36 | SEQ ID NO: 360 | IGKV1-6*01 |
| 37 | SEQ ID NO: 361 | IGKV1-9*01 |
| 38 | SEQ ID NO: 362 | IGKV1-6*01, IGKV1-6*02 |
| 39 | SEQ ID NO: 363 | IGKV3-20*01 |
| 40 | SEQ ID NO: 364 | IGKV1-39*01, IGKV1D-39*01 |
| 41 | SEQ ID NO: 365 | IGKV1-17*01 |
| 42 | SEQ ID NO: 366 | IGKV2-24*01 |
| 43 | SEQ ID NO: 367 | IGKV1-17*01 |
| 44 | SEQ ID NO: 368 | IGKV1-17*01 |
| 45 | SEQ ID NO: 369 | IGKV3-20*01 |
| 46 | SEQ ID NO: 370 | IGKV1-27*01 |
| 47 | SEQ ID NO: 371 | IGKV1-39*01, IGKV1D-39*01 |
| 48 | SEQ ID NO: 372 | IGKV1-17*01 |
| 49 | SEQ ID NO: 373 | IGKV1-39*01, IGKV1D-39*01 |
| 50 | SEQ ID NO: 374 | IGKV1-12*01, IGKV1-12*02, |
| | | IGKV1D-12*02 |
| 51 | SEQ ID NO: 375 | IGKV3-20*01 |
| 52 | SEQ ID NO: 376 | IGKV1-39*01, IGKV1D-39*01 |
| 53 | SEQ ID NO: 377 | IGKV1-17*01 |
| 54 | SEQ ID NO: 378 | IGKV1-17*01 |

| Line # | IR_VDJ_1_v_gene | IR_VDJ_1_d_gene |
|---|---|---|
| 1 | IGHV3-21*01 | IGHD6-6*01 |
| 2 | IGHV3-9*01 | IGHD6-13*01, IGHD6-25*01, |
| | | IGHD6-6*01 |
| 3 | IGHV3-9*01 | IGHD6-13*01 |
| 4 | IGHV3-9*01 | IGHD6-13*01 |
| 5 | IGHV1-2*02 | IGHD1-14*01, IGHD6-25*01 |
| 6 | IGHV3-9*01 | IGHD1-26*01 |
| 7 | IGHV3-9*01 | IGHD6-19*01 |
| 8 | IGHV3-9*01 | IGHD6-13*01 |
| 9 | IGHV1-8*01 | IGHD1-1*01, IGHD4-11*01, |
| | | IGHD4-4*01 |
| 10 | IGHV3-9*01 | IGHD3-16*02 |
| 11 | IGHV3-9*01 | IGHD6-19*01 |
| 12 | IGHV3-30*18, IGHV3-30-5*01 | IGHD7-27*01 |
| 13 | IGHV3-20*01 | IGHD6-13*01 |
| 14 | IGHV3-9*01 | IGHD1-26*01 |
| 15 | IGHV3-9*01 | IGHD6-19*01 |
| 16 | IGHV3-9*01 | IGHD6-13*01 |
| 17 | IGHV3-48*03 | IGHD3-10*01 |
| 18 | IGHV5-51*01 | |
| 19 | IGHV3-9*01 | IGHD6-19*01 |
| 20 | IGHV3-21*01 | IGHD6-6*01 |
| 21 | IGHV3-20*01 | IGHD6-13*01 |
| 22 | IGHV3-9*01 | IGHD1-26*01 |
| 23 | IGHV3-23*01, IGHV3-23D*01 | IGHD6-6*01 |
| 24 | IGHV3-9*01 | IGHD1-26*01 |
| 25 | IGHV3-20*04 | IGHD6-13*01, IGHD6-19*01 |
| 26 | IGHV6-1*01 | IGHD1-7*01 |
| 27 | IGHV3-9*01 | IGHD6-13*01 |
| 28 | IGHV3-23*01, IGHV3-23D*01 | IGHD3-10*01, IGHD3-10*02 |
| 29 | IGHV3-23*01, IGHV3-23D*01 | IGHD1-7*01 |
| 30 | IGHV3-9*01 | IGHD3-16*01, IGHD3-16*02 |
| 31 | IGHV3-9*01 | IGHD6-19*01 |
| 32 | IGHV3-9*01 | IGHD1-26*01 |
| 33 | IGHV3-48*03 | IGHD1-7*01 |
| 34 | IGHV3-9*01 | IGHD6-13*01 |
| 35 | IGHV3-21*01 | IGHD6-13*01 |
| 36 | IGHV3-9*01 | IGHD6-19*01 |
| 37 | IGHV3-33*01, IGHV3-33*06 | IGHD1-7*01 |
| 38 | IGHV3-21*06 | IGHD4-17*01 |
| 39 | IGHV3-9*01 | IGHD1-26*01 |
| 40 | IGHV4-59*08 | IGHD3-16*01 |
| 41 | IGHV3-9*01 | IGHD2-15*01, IGHD6-13*01, |
| | | IGHD6-19*01 |
| 42 | IGHV3-23*01, IGHV3-23D*01 | IGHD1-26*01 |
| 43 | IGHV3-9*01 | IGHD2-15*01, IGHD6-13*01, |
| | | IGHD6-19*01 |
| 44 | IGHV3-9*01 | IGHD1-26*01 |
| 45 | IGHV3-9*01 | IGHD1-26*01 |
| 46 | IGHV3-9*01 | IGHD6-13*01 |
| 47 | IGHV3-9*01 | IGHD1-26*01 |
| 48 | IGHV3-9*01 | IGHD6-13*01 |
| 49 | IGHV3-23*01, IGHV3-23D*01 | IGHD1-26*01 |
| 50 | IGHV3-23*01, IGHV3-23D*01 | |
| 51 | IGHV3-64*07 | IGHD6-13*01 |
| 52 | IGHV3-9*01 | IGHD1-26*01 |
| 53 | IGHV3-9*01 | IGHD6-13*01 |
| 54 | IGHV1-18*01 | IGHD1-7*01 |

| Line # | IR_VJ_1_j_gene | IR_VDJ_1_j_gene | IR_VJ_1_c_gene | IR_VDJ_1_c_gene |
|---|---|---|---|---|
| 1 | IGKJ4*01 | IGHJ3*01, IGHJ3*02 | IGKC | IGHG1 |
| 2 | IGKJ4*01 | IGHJ6*02 | IGKC | IGHG2C |
| 3 | IGKJ1*01 | IGHJ2*01 | IGKC | IGHG2C |
| 4 | IGKJ1*01 | IGHJ2*01 | IGKC | IGHG2C |
| 5 | IGKJ4*01 | IGHJ6*02 | IGKC | IGHG2C |
| 6 | IGKJ4*01 | IGHJ6*02 | IGKC | IGHG2C |
| 7 | IGKJ1*01 | IGHJ4*02 | IGKC | IGHG1 |
| 8 | IGKJ3*01 | IGHJ5*02 | IGKC | IGHG1 |
| 9 | IGKJ3*01 | IGHJ2*01 | IGKC | IGHG1 |
| 10 | IGKJ4*01 | IGHJ6*02 | IGKC | IGHG2C |
| 11 | IGKJ1*01 | IGHJ4*02 | IGKC | IGHG2C |
| 12 | IGKJ1*01 | IGHJ4*02 | IGKC | IGHG2C |
| 13 | IGKJ1*01 | IGHJ4*02 | IGKC | IGHG2C |
| 14 | IGKJ4*01 | IGHJ6*02 | IGKC | IGHG2C |
| 15 | IGKJ1*01 | IGHJ4*02 | IGKC | IGHG2C |
| 16 | IGKJ1*01 | IGHJ2*01 | IGKC | IGHG1 |
| 17 | IGKJ3*01 | IGHJ3*02 | IGKC | IGHG2C |
| 18 | IGKJ5*01 | IGHJ5*02 | IGKC | IGHG2C |
| 19 | IGKJ1*01 | IGHJ4*02 | IGKC | IGHG2C |
| 20 | IGKJ5*01 | IGHJ3*02 | IGKC | IGHG2C |
| 21 | IGKJ1*01 | IGHJ1*01 | IGKC | IGHG2C |
| 22 | IGKJ2*01 | IGHJ4*02 | IGKC | IGHG2C |
| 23 | IGKJ1*01 | IGHJ3*02 | IGKC | IGHG2C |
| 24 | IGKJ3*01 | IGHJ4*02 | IGKC | IGHG2C |
| 25 | IGKJ1*01 | IGHJ4*02 | IGKC | IGHG1 |
| 26 | IGKJ2*01 | IGHJ4*02 | IGKC | IGHG2C |
| 27 | IGKJ5*01 | IGHJ3*01 | IGKC | IGHG2C |
| 28 | IGKJ3*01 | IGHJ4*02 | IGKC | IGHG2C |
| 29 | IGKJ4*01 | IGHJ4*02, IGHJ5*02 | IGKC | IGHG1 |
| 30 | IGKJ3*01 | IGHJ6*02 | IGKC | IGHG2C |
| 31 | IGKJ1*01 | IGHJ4*02 | IGKC | IGHG1 |
| 32 | IGKJ4*01 | IGHJ6*02 | IGKC | IGHG2C |
| 33 | IGKJ3*01 | IGHJ3*02 | IGKC | IGHG1 |
| 34 | IGKJ1*01 | IGHJ2*01 | IGKC | IGHG2C |
| 35 | IGKJ5*01 | IGHJ4*02 | IGKC | IGHG2C |
| 36 | IGKJ1*01 | IGHJ5*02 | IGKC | IGHG1 |
| 37 | IGKJ2*01 | IGHJ4*02 | IGKC | IGHG2C |
| 38 | IGKJ1*01 | IGHJ4*02 | IGKC | IGHG2C |
| 39 | IGKJ2*01 | IGHJ4*02 | IGKC | IGHG2C |
| 40 | IGKJ4*01 | IGHJ6*02 | IGKC | IGHG2C |
| 41 | IGKJ4*01 | IGHJ6*02 | IGKC | IGHG2C |
| 42 | IGKJ4*01 | IGHJ4*02 | IGKC | IGHG2C |
| 43 | IGKJ4*01 | IGHJ6*02 | IGKC | IGHG2C |
| 44 | IGKJ4*01 | IGHJ6*02 | IGKC | IGHG2C |
| 45 | IGKJ2*01 | IGHJ4*02 | IGKC | IGHG2C |
| 46 | IGKJ1*01 | IGHJ2*01 | IGKC | IGHG2C |
| 47 | IGKJ3*01 | IGHJ6*02 | IGKC | IGHG2C |
| 48 | IGKJ2*01 | IGHJ6*02 | IGKC | IGHG2C |
| 49 | IGKJ5*01 | IGHJ4*02 | IGKC | IGHG2C |
| 50 | IGKJ3*01 | IGHJ3*02 | IGKC | IGHG2C |
| 51 | IGKJ2*01 | IGHJ6*02 | IGKC | IGHG2C |
| 52 | IGKJ3*01 | IGHJ6*02 | IGKC | IGHG2C |
| 53 | IGKJ2*01 | IGHJ6*02 | IGKC | IGHG2C |
| 54 | IGKJ1*01 | IGHJ4*02 | IGKC | IGHG2C |

| Line # | REGN560.L15_ab_raw | REGN78.L15_ab_raw | Tissue | TissueCT |
|---|---|---|---|---|
| 1 | 11214 | 271 | BM | BM_Plasma_cell |
| 2 | 6538 | 26 | BM | BM_Plasma_cell |
| 3 | 6317 | 236 | BM | BM_Plasma_cell |
| 4 | 5046 | 95 | BM | BM_Plasma_cell |
| 5 | 4411 | 214 | BM | BM_Plasma_cell |
| 6 | 4144 | 18 | BM | BM_Plasma_cell |
| 7 | 4142 | 224 | BM | BM_Plasma_cell |
| 8 | 3780 | 524 | BM | BM_Plasma_cell |
| 9 | 3568 | 890 | BM | BM_Plasma_cell |
| 10 | 3476 | 16 | BM | BM_Plasma_cell |
| 11 | 3212 | 115 | BM | BM_Plasma_cell |
| 12 | 3180 | 348 | BM | BM_Plasma_cell |
| 13 | 2931 | 33 | BM | BM_Plasma_cell |
| 14 | 2828 | 50 | BM | BM_Plasma_cell |
| 15 | 2585 | 158 | BM | BM_Plasma_cell |
| 16 | 2366 | 69 | BM | BM_Plasma_cell |
| 17 | 2299 | 881 | BM | BM_Plasma_cell |
| 18 | 2174 | 55 | BM | BM_Plasma_cell |
| 19 | 2063 | 50 | BM | BM_Plasma_cell |
| 20 | 2058 | 681 | BM | BM_Plasma_cell |
| 21 | 17383 | 236 | SP | SP_B_cell |
| 22 | 13995 | 5 | SP | SP_B_cell |
| 23 | 10971 | 152 | SP | SP_B_cell |
| 24 | 10811 | 11 | SP | SP_B_cell |
| 25 | 10605 | 16 | SP | SP_B_cell |
| 26 | 10355 | 167 | SP | SP_B_cell |
| 27 | 9506 | 149 | SP | SP_B_cell |
| 28 | 9446 | 129 | SP | SP_B_cell |
| 29 | 9188 | 93 | SP | SP_B_cell |
| 30 | 9182 | 179 | SP | SP_B_cell |
| 31 | 8515 | 57 | SP | SP_B_cell |
| 32 | 7912 | 32 | SP | SP_B_cell |
| 33 | 7344 | 106 | SP | SP_B_cell |
| 34 | 6888 | 34 | SP | SP_B_cell |
| 35 | 6710 | 33 | SP | SP_B_cell |
| 36 | 6345 | 75 | SP | SP_B_cell |
| 37 | 6230 | 60 | SP | SP_B_cell |
| 38 | 6051 | 66 | SP | SP_B_cell |
| 39 | 3560 | 17 | SP | SP_B_cell |
| 40 | 5520 | 1 | SP | SP_Plasma_cell |
| 41 | 2457 | 227 | SP | SP_Plasma_cell |
| 42 | 2084 | 5 | SP | SP_Plasma_cell |
| 43 | 1980 | 2 | SP | SP_Plasma_cell |
| 44 | 1845 | 26 | SP | SP_Plasma_cell |
| 45 | 1775 | 25 | SP | SP_Plasma_cell |
| 46 | 1525 | 2 | SP | SP_Plasma_cell |
| 47 | 1522 | 6 | SP | SP_Plasma_cell |
| 48 | 1239 | 17 | SP | SP_Plasma_cell |
| 49 | 1199 | 2 | SP | SP_Plasma_cell |
| 50 | 924 | 0 | SP | SP_Plasma_cell |
| 51 | 897 | 3 | SP | SP_Plasma_cell |
| 52 | 819 | 19 | SP | SP_Plasma_cell |
| 53 | 818 | 5 | SP | SP_Plasma_cell |
| 54 | 815 | 26 | SP | SP_Plasma_cell |

| Line # | Kd | negLog10Kd |
|---|---|---|
| 1 | 4.16E−07 | 6.380906669 |
| 2 | 1.61E−07 | 6.793174124 |
| 3 | 5.37E−07 | 6.270025714 |
| 4 | 1.58E−07 | 6.801342913 |
| 5 | 2.38E−08 | 7.623423043 |
| 6 | 1.36E−07 | 6.866461092 |
| 7 | 1.55E−08 | 7.809668302 |
| 8 | 0 | NA |
| 9 | 0 | NA |
| 10 | 0 | NA |
| 11 | 2.75E−08 | 7.560667306 |
| 12 | 3.45E−08 | 7.462180905 |
| 13 | 5.82E−08 | 7.235077015 |
| 14 | 0 | NA |
| 15 | 1.99E−08 | 7.701146924 |
| 16 | 8.04E−09 | 8.094743951 |
| 17 | 0 | NA |
| 18 | 4.14E−08 | 7.382999659 |
| 19 | 1.21E−08 | 7.91721463 |
| 20 | 0 | NA |
| 21 | 1.77E−07 | 6.752026734 |
| 22 | 0 | NA |
| 23 | 0 | NA |
| 24 | 2.07E−07 | 6.684029655 |
| 25 | 9.76E−08 | 7.010550182 |
| 26 | 1.12E−07 | 6.950781977 |
| 27 | 0 | NA |
| 28 | 1.46E−07 | 6.835647144 |
| 29 | 5.48E−08 | 7.261219442 |
| 30 | 0 | NA |
| 31 | 1.33E−07 | 6.876148359 |
| 32 | 5.81E−08 | 7.235823868 |
| 33 | 0 | NA |
| 34 | 3.42E−08 | 7.465973894 |
| 35 | 2.93E−08 | 7.53313238 |
| 36 | 0 | NA |
| 37 | 1.97E−07 | 6.705533774 |
| 38 | 0 | NA |
| 39 | 0 | NA |
| 40 | 1.18E−07 | 6.928117993 |
| 41 | 1.50E−07 | 6.823908741 |
| 42 | 1.27E−07 | 6.896196279 |
| 43 | 0 | NA |
| 44 | 0 | NA |
| 45 | 0 | NA |
| 46 | 4.92E−08 | 7.308034897 |
| 47 | 0 | NA |
| 48 | 0 | NA |
| 49 | 1.32E−07 | 6.879426069 |
| 50 | 0 | NA |
| 51 | 0 | NA |
| 52 | 0 | NA |
| 53 | 2.47E−07 | 6.607303047 |
| 54 | 1.35E−08 | 7.869666232 |

TABLE 2
hIL-Ra IgG-specific BCR sequences shared among bone marrow plasma cells,
splenic B and Plasma cells

| Line | | |
|---|---|---|
| # | Cell_Barcode | heavy_sequence |
| 1 | Clonotype #1 (n = 10 cells) | |
| 2 | SEQ ID NO: 379 | SEQ ID NO: 380 |
| 3 | SEQ ID NO: 381 | SEQ ID NO: 382 |
| 4 | SEQ ID NO: 383 | SEQ ID NO: 384 |
| 5 | SEQ ID NO: 385 | SEQ ID NO: 386 |
| 6 | SEQ ID NO: 387 | SEQ ID NO: 388 |
| 7 | SEQ ID NO: 389 | SEQ ID NO: 390 |
| 8 | SEQ ID NO: 391 | SEQ ID NO: 392 |
| 9 | SEQ ID NO: 393 | SEQ ID NO: 394 |
| 10 | SEQ ID NO: 395 | SEQ ID NO: 396 |
| 11 | SEQ ID NO: 397 | SEQ ID NO: 398 |
| 12 | Clonotype #2 (n = 9 cells) | |
| 13 | SEQ ID NO: 399 | SEQ ID NO: 400 |
| 14 | SEQ ID NO: 401 | SEQ ID NO: 402 |
| 15 | SEQ ID NO: 403 | SEQ ID NO: 404 |
| 16 | SEQ ID NO: 405 | SEQ ID NO: 406 |
| 17 | SEQ ID NO: 407 | SEQ ID NO: 408 |
| 18 | SEQ ID NO: 409 | SEQ ID NO: 410 |
| 19 | SEQ ID NO: 411 | SEQ ID NO: 412 |
| 20 | SEQ ID NO: 413 | SEQ ID NO: 414 |
| 21 | SEQ ID NO: 415 | SEQ ID NO: 416 |
| 22 | Clonotype #3 (n = 9 cells) | |
| 23 | SEQ ID NO: 417 | SEQ ID NO: 418 |
| 24 | SEQ ID NO: 419 | SEQ ID NO: 420 |
| 25 | SEQ ID NO: 421 | SEQ ID NO: 422 |
| 26 | SEQ ID NO: 423 | SEQ ID NO: 424 |
| 27 | SEQ ID NO: 425 | SEQ ID NO: 426 |
| 28 | SEQ ID NO: 427 | SEQ ID NO: 428 |
| 29 | SEQ ID NO: 429 | SEQ ID NO: 430 |
| 30 | SEQ ID NO: 431 | SEQ ID NO: 432 |
| 31 | SEQ ID NO: 433 | SEQ ID NO: 434 |
| 32 | Clonotype #4 (n = 9 cells) | |
| 33 | SEQ ID NO: 435 | SEQ ID NO: 436 |
| 34 | SEQ ID NO: 437 | SEQ ID NO: 438 |
| 35 | SEQ ID NO: 439 | SEQ ID NO: 440 |
| 36 | SEQ ID NO: 441 | SEQ ID NO: 442 |
| 37 | SEQ ID NO: 443 | SEQ ID NO: 444 |
| 38 | SEQ ID NO: 445 | SEQ ID NO: 446 |
| 39 | SEQ ID NO: 447 | SEQ ID NO: 448 |
| 40 | SEQ ID NO: 449 | SEQ ID NO: 450 |
| 41 | SEQ ID NO: 451 | SEQ ID NO: 452 |
| 42 | Clonotype #5 (n = 6 cells) | |
| 43 | SEQ ID NO: 453 | SEQ ID NO: 454 |
| 44 | SEQ ID NO: 455 | SEQ ID NO: 456 |
| 45 | SEQ ID NO: 457 | SEQ ID NO: 458 |
| 46 | SEQ ID NO: 459 | SEQ ID NO: 460 |
| 47 | SEQ ID NO: 461 | SEQ ID NO: 462 |
| 48 | SEQ ID NO: 463 | SEQ ID NO: 464 |
| 49 | Clonotype #6 (n = 6 cells) | |
| 50 | SEQ ID NO: 465 | SEQ ID NO: 466 |
| 51 | SEQ ID NO: 467 | SEQ ID NO: 468 |
| 52 | SEQ ID NO: 469 | SEQ ID NO: 470 |
| 53 | SEQ ID NO: 471 | SEQ ID NO: 472 |
| 54 | SEQ ID NO: 473 | SEQ ID NO: 474 |
| 55 | SEQ ID NO: 475 | SEQ ID NO: 476 |
| 56 | Clonotype #7 (n = 5 cells) | |
| 57 | SEQ ID NO: 477 | SEQ ID NO: 478 |
| 58 | SEQ ID NO: 479 | SEQ ID NO: 480 |
| 59 | SEQ ID NO: 481 | SEQ ID NO: 482 |
| 60 | SEQ ID NO: 483 | SEQ ID NO: 484 |
| 61 | SEQ ID NO: 485 | SEQ ID NO: 486 |
| 62 | Clonotype #8 (n = 5 cells) | |
| 63 | SEQ ID NO: 487 | SEQ ID NO: 488 |
| 64 | SEQ ID NO: 489 | SEQ ID NO: 490 |
| 65 | SEQ ID NO: 491 | SEQ ID NO: 492 |
| 66 | SEQ ID NO: 493 | SEQ ID NO: 494 |
| 67 | SEQ ID NO: 495 | SEQ ID NO: 496 |
| 68 | Clonotype #9 (n = 4 cells) | |
| 69 | SEQ ID NO: 497 | SEQ ID NO: 498 |
| 70 | SEQ ID NO: 499 | SEQ ID NO: 500 |
| 71 | SEQ ID NO: 501 | SEQ ID NO: 502 |
| 72 | SEQ ID NO: 503 | SEQ ID NO: 504 |
| 73 | Clonotype #10 (n = 3 cells) | |
| 74 | SEQ ID NO: 505 | SEQ ID NO: 506 |
| 75 | SEQ ID NO: 507 | SEQ ID NO: 508 |
| 76 | SEQ ID NO: 509 | SEQ ID NO: 510 |

| Line | | |
|---|---|---|
| # | light_sequence | v_identity |
| 2 | SEQ ID NO: 511 | 0.95066 |
| 3 | SEQ ID NO: 512 | 0.9375 |
| 4 | SEQ ID NO: 513 | 0.96711 |
| 5 | SEQ ID NO: 514 | 0.96382 |
| 6 | SEQ ID NO: 515 | 0.95066 |
| 7 | SEQ ID NO: 516 | 0.95066 |
| 8 | SEQ ID NO: 517 | 0.95066 |
| 9 | SEQ ID NO: 518 | 0.95724 |
| 10 | SEQ ID NO: 519 | 0.95395 |
| 11 | SEQ ID NO: 520 | 0.95724 |
| 13 | SEQ ID NO: 521 | 0.95987 |
| 14 | EQ ID NO: 522 | 0.95987 |
| 15 | SEQ ID NO: 523 | 0.96321 |
| 16 | SEQ ID NO: 524 | 0.95318 |
| 17 | SEQ ID NO: 525 | 0.95652 |
| 18 | SEQ ID NO: 526 | 0.95652 |
| 19 | SEQ ID NO: 527 | 0.95318 |
| 20 | SEQ ID NO: 528 | 0.95652 |
| 21 | SEQ ID NO: 529 | 0.95987 |
| 23 | SEQ ID NO: 530 | 0.92491 |
| 24 | SEQ ID NO: 531 | 0.95222 |
| 25 | SEQ ID NO: 532 | 0.94198 |
| 26 | SEQ ID NO: 533 | 0.94198 |
| 27 | SEQ ID NO: 534 | 0.92491 |
| 28 | SEQ ID NO: 535 | 0.92833 |
| 29 | SEQ ID NO: 536 | 0.94881 |
| 30 | SEQ ID NO: 537 | 0.94198 |
| 31 | SEQ ID NO: 538 | 0.94539 |
| 33 | SEQ ID NO: 539 | 0.95563 |
| 34 | SEQ ID NO: 540 | 0.95222 |
| 35 | SEQ ID NO: 541 | 0.94539 |
| 36 | SEQ ID NO: 542 | 0.95904 |
| 37 | SEQ ID NO: 543 | 0.94198 |
| 38 | SEQ ID NO: 544 | 0.95222 |
| 39 | SEQ ID NO: 545 | 0.93515 |
| 40 | SEQ ID NO: 546 | 0.94198 |
| 41 | SEQ ID NO: 547 | 0.94539 |
| 43 | SEQ ID NO: 548 | 0.91156 |
| 44 | SEQ ID NO: 549 | 0.93532 |
| 45 | SEQ ID NO: 550 | 0.92177 |
| 46 | SEQ ID NO: 551 | 0.93197 |
| 47 | SEQ ID NO: 552 | 0.93537 |
| 48 | SEQ ID NO: 553 | 0.93197 |
| 50 | SEQ ID NO: 554 | 0.95638 |
| 51 | SEQ ID NO: 555 | 0.95302 |
| 52 | SEQ ID NO: 556 | 0.96309 |
| 53 | SEQ ID NO: 557 | 0.96309 |
| 54 | SEQ ID NO: 558 | 0.94595 |
| 55 | SEQ ID NO: 559 | 0.95302 |
| 57 | SEQ ID NO: 560 | 0.92282 |
| 58 | SEQ ID NO: 561 | 0.93289 |
| 59 | SEQ ID NO: 562 | 0.93289 |
| 60 | SEQ ID NO: 563 | 0.93289 |
| 61 | SEQ ID NO: 564 | 0.92953 |
| 63 | SEQ ID NO: 565 | 0.9215 |
| 64 | SEQ ID NO: 566 | 0.94539 |
| 65 | SEQ ID NO: 567 | 0.92491 |
| 66 | SEQ ID NO: 568 | 0.91126 |
| 67 | SEQ ID NO: 569 | 0.91126 |
| 69 | SEQ ID NO: 570 | 0.96246 |
| 70 | SEQ ID NO: 571 | 0.96587 |
| 71 | SEQ ID NO: 572 | 0.96587 |
| 72 | SEQ ID NO: 573 | 0.96246 |
| 74 | SEQ ID NO: 574 | 0.95066 |
| 75 | SEQ ID NO: 575 | 0.96053 |
| 76 | SEQ ID NO: 576 | 0.96053 |

| Line | | | | |
|---|---|---|---|---|
| # | IR_VJ_1_locus | IR_VDJ_1_locus | IR_VJ_1_cdr3 | IR_VDJ_1_cdr3 |
| 2 | IGK | IGH | SEQ ID NO: 577 | SEQ ID NO: 578 |
| 3 | IGK | IGH | SEQ ID NO: 577 | SEQ ID NO: 578 |
| 4 | IGK | IGH | SEQ ID NO: 577 | SEQ ID NO: 578 |
| 5 | IGK | IGH | SEQ ID NO: 577 | SEQ ID NO: 578 |
| 6 | IGK | IGH | SEQ ID NO: 577 | SEQ ID NO: 578 |
| 7 | IGK | IGH | SEQ ID NO: 577 | SEQ ID NO: 578 |
| 8 | IGK | IGH | SEQ ID NO: 577 | SEQ ID NO: 578 |
| 9 | IGK | IGH | SEQ ID NO: 577 | SEQ ID NO: 578 |
| 10 | IGK | IGH | SEQ ID NO: 577 | SEQ ID NO: 578 |
| 11 | IGK | IGH | SEQ ID NO: 577 | SEQ ID NO: 578 |
| 13 | IGK | IGH | SEQ ID NO: 250 | SEQ ID NO: 579 |
| 14 | IGK | IGH | SEQ ID NO: 250 | SEQ ID NO: 579 |
| 15 | IGK | IGH | SEQ ID NO: 250 | SEQ ID NO: 579 |
| 16 | IGK | IGH | SEQ ID NO: 250 | SEQ ID NO: 579 |
| 17 | IGK | IGH | SEQ ID NO: 250 | SEQ ID NO: 579 |
| 18 | IGK | IGH | SEQ ID NO: 250 | SEQ ID NO: 579 |
| 19 | IGK | IGH | SEQ ID NO: 250 | SEQ ID NO: 579 |
| 20 | IGK | IGH | SEQ ID NO: 250 | SEQ ID NO: 579 |
| 21 | IGK | IGH | SEQ ID NO: 250 | SEQ ID NO: 579 |
| 23 | IGK | IGH | SEQ ID NO: 580 | SEQ ID NO: 581 |
| 24 | IGK | IGH | SEQ ID NO: 580 | SEQ ID NO: 581 |
| 25 | IGK | IGH | SEQ ID NO: 580 | SEQ ID NO: 581 |
| 26 | IGK | IGH | SEQ ID NO: 580 | SEQ ID NO: 581 |
| 27 | IGK | IGH | SEQ ID NO: 580 | SEQ ID NO: 581 |
| 28 | IGK | IGH | SEQ ID NO: 580 | SEQ ID NO: 581 |
| 29 | IGK | IGH | SEQ ID NO: 580 | SEQ ID NO: 581 |
| 30 | IGK | IGH | SEQ ID NO: 580 | SEQ ID NO: 581 |
| 31 | IGK | IGH | SEQ ID NO: 580 | SEQ ID NO: 581 |
| 33 | IGK | IGH | SEQ ID NO: 582 | SEQ ID NO: 583 |
| 34 | IGK | IGH | SEQ ID NO: 582 | SEQ ID NO: 583 |
| 35 | IGK | IGH | SEQ ID NO: 582 | SEQ ID NO: 583 |
| 36 | IGK | IGH | SEQ ID NO: 582 | SEQ ID NO: 583 |
| 37 | IGK | IGH | SEQ ID NO: 582 | SEQ ID NO: 583 |
| 38 | IGK | IGH | SEQ ID NO: 582 | SEQ ID NO: 583 |
| 39 | IGK | IGH | SEQ ID NO: 582 | SEQ ID NO: 583 |
| 40 | IGK | IGH | SEQ ID NO: 582 | SEQ ID NO: 583 |
| 41 | IGK | IGH | SEQ ID NO: 582 | SEQ ID NO: 583 |
| 43 | IGK | IGH | SEQ ID NO: 584 | SEQ ID NO: 585 |
| 44 | IGK | IGH | SEQ ID NO: 584 | SEQ ID NO: 585 |
| 45 | IGK | IGH | SEQ ID NO: 584 | SEQ ID NO: 585 |
| 46 | IGK | IGH | SEQ ID NO: 584 | SEQ ID NO: 585 |
| 47 | IGK | IGH | SEQ ID NO: 584 | SEQ ID NO: 585 |
| 48 | IGK | IGH | SEQ ID NO: 584 | SEQ ID NO: 585 |
| 50 | IGK | IGH | SEQ ID NO: 586 | SEQ ID NO: 587 |
| 51 | IGK | IGH | SEQ ID NO: 586 | SEQ ID NO: 587 |
| 52 | IGK | IGH | SEQ ID NO: 586 | SEQ ID NO: 587 |
| 53 | IGK | IGH | SEQ ID NO: 586 | SEQ ID NO: 587 |
| 54 | IGK | IGH | SEQ ID NO: 586 | SEQ ID NO: 587 |
| 55 | IGK | IGH | SEQ ID NO: 586 | SEQ ID NO: 587 |
| 57 | IGK | IGH | SEQ ID NO: 169 | SEQ ID NO: 588 |
| 58 | IGK | IGH | SEQ ID NO: 169 | SEQ ID NO: 588 |
| 59 | IGK | IGH | SEQ ID NO: 169 | SEQ ID NO: 588 |
| 60 | IGK | IGH | SEQ ID NO: 169 | SEQ ID NO: 588 |
| 61 | IGK | IGH | SEQ ID NO: 169 | SEQ ID NO: 588 |
| 63 | IGK | IGH | SEQ ID NO: 589 | SEQ ID NO: 590 |
| 64 | IGK | IGH | SEQ ID NO: 589 | SEQ ID NO: 590 |
| 65 | IGK | IGH | SEQ ID NO: 589 | SEQ ID NO: 590 |
| 66 | IGK | IGH | SEQ ID NO: 589 | SEQ ID NO: 590 |
| 67 | IGK | IGH | SEQ ID NO: 589 | SEQ ID NO: 590 |
| 69 | IGK | IGH | SEQ ID NO: 232 | SEQ ID NO: 281 |
| 70 | IGK | IGH | SEQ ID NO: 232 | SEQ ID NO: 281 |
| 71 | IGK | IGH | SEQ ID NO: 232 | SEQ ID NO: 281 |
| 72 | IGK | IGH | SEQ ID NO: 232 | SEQ ID NO: 281 |
| 74 | IGK | IGH | SEQ ID NO: 591 | SEQ ID NO: 592 |
| 75 | IGK | IGH | SEQ ID NO: 591 | SEQ ID NO: 592 |
| 76 | IGK | IGH | SEQ ID NO: 591 | SEQ ID NO: 592 |

| Line | | | | |
|---|---|---|---|---|
| # | IR_VJ_1_cdr3_nt | IR_VDJ_1_cdr3_nt | IR_VJ_1_v_gene | IR_VDJ_1_v_gene |
| 2 | SEQ ID NO: 593 | SEQ ID NO: 594 | IGKV3-15*01 | IGHV6-1*01 |
| 3 | SEQ ID NO: 601 | SEQ ID NO: 594 | IGKV3-15*01 | IGHV6-1*01 |
| 4 | SEQ ID NO: 593 | SEQ ID NO: 594 | IGKV3-15*01 | IGHV6-1*01 |
| 5 | SEQ ID NO: 593 | SEQ ID NO: 594 | IGKV3-15*01 | IGHV6-1*01 |
| 6 | SEQ ID NO: 593 | SEQ ID NO: 594 | IGKV3-15*01 | IGHV6-1*01 |
| 7 | SEQ ID NO: 593 | SEQ ID NO: 594 | IGKV3-15*01 | IGHV6-1*01 |
| 8 | SEQ ID NO: 593 | SEQ ID NO: 594 | IGKV3-15*01 | IGHV6-1*01 |
| 9 | SEQ ID NO: 593 | SEQ ID NO: 594 | IGKV3-15*01 | IGHV6-1*01 |
| 10 | SEQ ID NO: 593 | SEQ ID NO: 594 | IGKV3-15*01 | IGHV6-1*01 |
| 11 | SEQ ID NO: 593 | SEQ ID NO: 594 | IGKV3-15*01 | IGHV6-1*01 |
| 13 | SEQ ID NO: 595 | SEQ ID NO: 596 | IGKV1-39*01, | IGHV4-39*01 |
| | | | IGKV1D-39*01 | |
| 14 | SEQ ID NO: 595 | SEQ ID NO: 596 | IGKV1-39*01, | IGHV4-39*01 |
| | | | IGKV1D-39*01 | |
| 15 | SEQ ID NO: 595 | SEQ ID NO: 596 | IGKV1-39*01, | IGHV4-39*01 |
| | | | IGKV1D-39*01 | |
| 16 | SEQ ID NO: 595 | SEQ ID NO: 596 | IGKV1-39*01, | IGHV4-39*01 |
| | | | IGKV1D-39*01 | |
| 17 | SEQ ID NO: 597 | SEQ ID NO: 596 | IGKV1-39*01, | IGHV4-39*01 |
| | | | IGKV1D-39*01 | |
| 18 | SEQ ID NO: 595 | SEQ ID NO: 596 | IGKV1-39*01, | IGHV4-39*01 |
| | | | IGKV1D-39*01 | |
| 19 | SEQ ID NO: 595 | SEQ ID NO: 596 | IGKV1-39*01, | IGHV4-39*01 |
| | | | IGKV1D-39*01 | |
| 20 | SEQ ID NO: 595 | SEQ ID NO: 596 | IGKV1-39*01, | IGHV4-39*01 |
| | | | IGKV1D-39*01 | |
| 21 | SEQ ID NO: 595 | SEQ ID NO: 596 | IGKV1-39*01, | IGHV4-39*01 |
| | | | IGKV1D-39*01 | |
| 23 | SEQ ID NO: 598 | SEQ ID NO: 599 | IGKV1-16*02 | IGHV4-34*01 |
| 24 | SEQ ID NO: 598 | SEQ ID NO: 600 | IGKV1-16*01 | IGHV4-34*01 |
| 25 | SEQ ID NO: 598 | SEQ ID NO: 602 | IGKV1-16*01 | IGHV4-34*01 |
| 26 | SEQ ID NO: 598 | SEQ ID NO: 600 | IGKV1-16*01 | IGHV4-34*01 |
| 27 | SEQ ID NO: 598 | SEQ ID NO: 603 | IGKV1-16*02 | IGHV4-34*01 |
| 28 | SEQ ID NO: 598 | SEQ ID NO: 603 | IGKV1-16*02 | IGHV4-34*01 |
| 29 | SEQ ID NO: 598 | SEQ ID NO: 600 | IGKV1-16*01 | IGHV4-34*01 |
| 30 | SEQ ID NO: 598 | SEQ ID NO: 600 | IGKV1-16*01 | IGHV4-34*01 |
| 31 | SEQ ID NO: 598 | SEQ ID NO: 600 | IGKV1-16*01 | IGHV4-34*01 |
| 33 | SEQ ID NO: 604 | SEQ ID NO: 605 | IGKV1-16*02 | IGHV4-34*01 |
| 34 | SEQ ID NO: 604 | SEQ ID NO: 605 | IGKV1-16*02 | IGHV4-34*01 |
| 35 | SEQ ID NO: 604 | SEQ ID NO: 606 | IGKV1-16*02 | IGHV4-34*01 |
| 36 | SEQ ID NO: 604 | SEQ ID NO: 605 | IGKV1-16*02 | IGHV4-34*01 |
| 37 | SEQ ID NO: 604 | SEQ ID NO: 606 | IGKV1-16*02 | IGHV4-34*01 |
| 38 | SEQ ID NO: 604 | SEQ ID NO: 605 | IGKV1-16*02 | IGHV4-34*01 |
| 39 | SEQ ID NO: 604 | SEQ ID NO: 606 | IGKV1-16*02 | IGHV4-34*01 |
| 40 | SEQ ID NO: 604 | SEQ ID NO: 605 | IGKV1-16*02 | IGHV4-34*01 |
| 41 | SEQ ID NO: 607 | SEQ ID NO: 606 | IGKV1-16*02 | IGHV4-34*01 |
| 43 | SEQ ID NO: 608 | SEQ ID NO: 609 | IGKV1-17*01 | IGHV3-23*01, |
| | | | | IGHV3-23D*01 |
| 44 | SEQ ID NO: 610 | SEQ ID NO: 609 | IGKV1-17*01 | IGHV3-23*01, |
| | | | | IGHV3-23*04, |
| | | | | IGHV3-23D*01 |
| 45 | SEQ ID NO: 611 | SEQ ID NO: 609 | IGKV1-17*01 | IGHV3-23*01, |
| | | | | IGHV3-23D*01 |
| 46 | SEQ ID NO: 611 | SEQ ID NO: 609 | IGKV1-17*01 | IGHV3-23*01, |
| | | | | IGHV3-23D*01 |
| 47 | SEQ ID NO: 611 | SEQ ID NO: 609 | IGKV1-17*01 | IGHV3-23*01, |
| | | | | IGHV3-23D*01 |
| 48 | SEQ ID NO: 611 | SEQ ID NO: 609 | IGKV1-17*01 | IGHV3-23*01, |
| | | | | IGHV3-23D*01 |
| 50 | SEQ ID NO: 612 | SEQ ID NO: 613 | IGKV1-27*01 | IGHV3-9*01 |
| 51 | SEQ ID NO: 612 | SEQ ID NO: 613 | IGKV1-27*01 | IGHV3-9*01 |
| 52 | SEQ ID NO: 612 | SEQ ID NO: 613 | IGKV1-27*01 | IGHV3-9*01 |
| 53 | SEQ ID NO: 612 | SEQ ID NO: 614 | IGKV1-27*01 | IGHV3-9*01 |
| 54 | SEQ ID NO: 612 | SEQ ID NO: 615 | IGKV1-27*01 | IGHV3-9*01 |
| 55 | SEQ ID NO: 612 | SEQ ID NO: 613 | IGKV1-27*01 | IGHV3-9*01 |
| 57 | SEQ ID NO: 171 | SEQ ID NO: 616 | IGKV1-5*03 | IGHV3-9*01 |
| 58 | SEQ ID NO: 171 | SEQ ID NO: 617 | IGKV1-5*03 | IGHV3-9*01 |
| 59 | SEQ ID NO: 171 | SEQ ID NO: 617 | IGKV1-5*03 | IGHV3-9*01 |
| 60 | SEQ ID NO: 171 | SEQ ID NO: 618 | IGKV1-5*03 | IGHV3-9*01 |
| 61 | SEQ ID NO: 171 | SEQ ID NO: 618 | IGKV1-5*03 | IGHV3-9*01 |
| 63 | SEQ ID NO: 619 | SEQ ID NO: 620 | IGKV6-21*01, | IGHV3-21*02 |
| | | | IGKV6D-21*01 | |
| 64 | SEQ ID NO: 619 | SEQ ID NO: 620 | IGKV6-21*01, | IGHV3-21*02 |
| | | | IGKV6D-21*01 | |
| 65 | SEQ ID NO: 619 | SEQ ID NO: 620 | IGKV6-21*01, | IGHV3-21*02 |
| | | | IGKV6D-21*01 | |
| 66 | SEQ ID NO: 619 | SEQ ID NO: 620 | IGKV6-21*01, | IGHV3-21*02 |
| | | | IGKV6D-21*01 | |
| 67 | SEQ ID NO: 619 | SEQ ID NO: 620 | IGKV6-21*01, | IGHV3-21*02 |
| | | | IGKV6D-21*01 | |
| 69 | SEQ ID NO: 234 | SEQ ID NO: 621 | IGKV1-39*01, | IGHV4-59*08 |
| | | | IGKV1D-39*01 | |
| 70 | SEQ ID NO: 234 | SEQ ID NO: 621 | IGKV1-39*01, | IGHV4-59*08 |
| | | | IGKV1D-39*01 | |
| 71 | SEQ ID NO: 622 | SEQ ID NO: 621 | IGKV1-39*01, | IGHV4-59*08 |
| | | | IGKV1D-39*01 | |
| 72 | SEQ ID NO: 623 | SEQ ID NO: 621 | IGKV1-39*01, | IGHV4-59*08 |
| | | | IGKV1D-39*01 | |
| 74 | SEQ ID NO: 624 | SEQ ID NO: 625 | IGKV1-12*01, | IGHV6-1*01 |
| | | | IGKV1-12*02, | |
| | | | IGKV1D-12*02 | |
| 75 | SEQ ID NO: 624 | SEQ ID NO: 626 | IGKV1-12*01, | IGHV6-1*01 |
| | | | IGKV1-12*02, | |
| | | | IGKV1D-12*02 | |
| 76 | SEQ ID NO: 624 | SEQ ID NO: 626 | IGKV1-12*01, | IGHV6-1*01 |
| | | | IGKV1-12*02, | |
| | | | IGKV1D-12*02 | |

| Line | | | | |
|---|---|---|---|---|
| # | IR_VDJ_1_d_gene | IR_VJ_1_i_gene | IR_VDJ_1_i_gene | IR_VJ_1_c_gene |
| 2 | IGHD7-27*01 | IGKJ4*01 | IGHJ5*02 | IGKC |
| 3 | IGHD7-27*01 | IGKJ4*01 | IGHJ5*02 | IGKC |
| 4 | IGHD7-27*01 | IGKJ4*01 | IGHJ5*02 | IGKC |
| 5 | IGHD7-27*01 | IGKJ4*01 | IGHJ5*02 | IGKC |
| 6 | IGHD7-27*01 | IGKJ4*01 | IGHJ5*02 | IGKC |
| 7 | IGHD7-27*01 | IGKJ4*01 | IGHJ5*02 | IGKC |
| 8 | IGHD7-27*01 | IGKJ4*01 | IGHJ5*02 | IGKC |
| 9 | IGHD7-27*01 | IGKJ4*01 | IGHJ5*02 | IGKC |
| 10 | IGHD7-27*01 | IGKJ4*01 | IGHJ5*02 | IGKC |
| 11 | IGHD7-27*01 | IGKJ4*01 | IGHJ5*02 | IGKC |
| 13 | IGHD3-10*01 | IGKJ3*01 | IGHJ4*02 | IGKC |
| 14 | IGHD3-10*01 | IGKJ3*01 | IGHJ4*02 | IGKC |
| 15 | IGHD3-10*01 | IGKJ3*01 | IGHJ4*02 | IGKC |
| 16 | IGHD3-10*01 | IGKJ3*01 | IGHJ4*02 | IGKC |
| 17 | IGHD3-10*01 | IGKJ3*01 | IGHJ4*02 | IGKC |
| 18 | IGHD3-10*01 | IGKJ3*01 | IGHJ4*02 | IGKC |
| 19 | IGHD3-10*01 | IGKJ3*01 | IGHJ4*02 | IGKC |
| 20 | IGHD3-10*01 | IGKJ3*01 | IGHJ4*02 | IGKC |
| 21 | IGHD3-10*01 | IGKJ3*01 | IGHJ4*02 | IGKC |
| 23 | IGHD2-21*01, | IGKJ1*01 | IGHJ4*02 | IGKC |
| | IGHD2-21*02 | | | |
| 24 | IGHD2-21*01, | IGKJ1*01 | IGHJ4*02 | IGKC |
| | IGHD2-21*02 | | | |
| 25 | IGHD2-21*01, | IGKJ1*01 | IGHJ4*02 | IGKC |
| | IGHD2-21*02 | | | |
| 26 | IGHD2-21*01, | IGKJ1*01 | IGHJ4*02 | IGKC |
| | IGHD2-21*02 | | | |
| 27 | IGHD2-21*01, | IGKJ1*01 | IGHJ4*02 | IGKC |
| | IGHD2-21*02 | | | |
| 28 | IGHD2-21*01, | IGKJ1*01 | IGHJ4*02 | IGKC |
| | IGHD2-21*02 | | | |
| 29 | IGHD2-21*01, | IGKJ1*01 | IGHJ4*02 | IGKC |
| | IGHD2-21*02 | | | |
| 30 | IGHD2-21*01, | IGKJ1*01 | IGHJ4*02 | IGKC |
| | IGHD2-21*02 | | | |
| 31 | IGHD2-21*01, | IGKJ1*01 | IGHJ4*02 | IGKC |
| | IGHD2-21*02 | | | |
| 33 | IGHD2-21*01 | IGKJ1*01 | IGHJ4*02 | IGKC |
| 34 | IGHD2-21*01 | IGKJ1*01 | IGHJ4*02 | IGKC |
| 35 | IGHD2-21*01 | IGKJ1*01 | IGHJ4*02 | IGKC |
| 36 | IGHD2-21*01 | IGKJ1*01 | IGHJ4*02 | IGKC |
| 37 | IGHD2-21*01 | IGKJ1*01 | IGHJ4*02 | IGKC |
| 38 | IGHD2-21*01 | IGKJ1*01 | IGHJ4*02 | IGKC |
| 39 | IGHD2-21*01 | IGKJ1*01 | IGHJ4*02 | IGKC |
| 40 | IGHD2-21*01 | IGKJ1*01 | IGHJ4*02 | IGKC |
| 41 | IGHD2-21*01 | IGKJ1*01 | IGHJ4*02 | IGKC |
| 43 | IGHD3-16*02 | IGKJ3*01 | IGHJ4*02 | IGKC |
| 44 | IGHD3-16*02 | IGKJ3*01 | IGHJ4*02 | IGKC |
| 45 | IGHD3-16*02 | IGKJ3*01 | IGHJ4*02 | IGKC |
| 46 | IGHD3-16*02 | IGKJ3*01 | IGHJ4*02 | IGKC |
| 47 | IGHD3-16*02 | IGKJ3*01 | IGHJ4*02 | IGKC |
| 48 | IGHD3-16*02 | IGKJ3*01 | IGHJ4*02 | IGKC |
| 50 | IGHD6-13*01 | IGKJ1*01 | IGHJ2*01 | IGKC |
| 51 | IGHD6-13*01 | IGKJ1*01 | IGHJ2*01 | IGKC |
| 52 | IGHD6-13*01 | IGKJ1*01 | IGHJ2*01 | IGKC |
| 53 | IGHD6-13*01 | IGKJ1*01 | IGHJ2*01 | IGKC |
| 54 | IGHD6-13*01 | IGKJ1*01 | IGHJ2*01 | IGKC |
| 55 | IGHD6-13*01 | IGKJ1*01 | IGHJ2*01 | IGKC |
| 57 | IGHD6-13*01 | IGKJ1*01 | IGHJ4*02 | IGKC |
| 58 | IGHD6-13*01 | IGKJ1*01 | IGHJ4*02 | IGKC |
| 59 | IGHD6-13*01 | IGKJ1*01 | IGHJ4*02 | IGKC |
| 60 | IGHD6-13*01 | IGKJ1*01 | IGHJ4*02 | IGKC |
| 61 | IGHD6-13*01 | IGKJ1*01 | IGHJ4*02 | IGKC |
| 63 | IGHD6-6*01 | IGKJ3*01 | IGHJ4*02 | IGKC |
| 64 | IGHD6-6*01 | IGKJ3*01 | IGHJ4*02 | IGKC |
| 65 | IGHD6-6*01 | IGKJ3*01 | IGHJ4*02 | IGKC |
| 66 | IGHD6-6*01 | IGKJ3*01 | IGHJ4*02 | IGKC |
| 67 | IGHD6-6*01 | IGKJ3*01 | IGHJ4*02 | IGKC |
| 69 | IGHD1-26*01 | IGKJ3*01 | IGHJ6*02 | IGKC |
| 70 | IGHD1-26*01 | IGKJ3*01 | IGHJ6*02 | IGKC |
| 71 | IGHD1-26*01 | IGKJ3*01 | IGHJ6*02 | IGKC |
| 72 | IGHD1-26*01 | IGKJ3*01 | IGHJ6*02 | IGKC |
| 74 | IGHD1-20*01, | IGKJ4*01 | IGHJ5*02 | IGKC |
| | IGHD1-7*01, | | | |
| | IGHD1/OR15-1a*01 | | | |
| 75 | IGHD1-7*01 | IGKJ4*01 | IGHJ5*02 | IGKC |
| 76 | IGHD1-7*01 | IGKJ4*01 | IGHJ5*02 | IGKC |

| Line | | | |
|---|---|---|---|
| # | IR_VDJ_1_c_Gene | TissueCT | CDR3L_CDR3H |
| 2 | IgG2C | BM_Plasma_cell | SEQ ID NO: 577-SEQ ID NO: 578 |
| 3 | IgG2C | SP_Bcell | SEQ ID NO: 577-SEQ ID NO: 578 |
| 4 | IgG2C | SP_Plasma_cell | SEQ ID NO: 577-SEQ ID NO: 578 |
| 5 | IgG2C | SP_Plasma_cell | SEQ ID NO: 577-SEQ ID NO: 578 |
| 6 | IgG2C | SP_Bcell | SEQ ID NO: 577-SEQ ID NO: 578 |
| 7 | IgG2C | SP_Bcell | SEQ ID NO: 577-SEQ ID NO: 578 |
| 8 | IgG2C | SP_Bcell | SEQ ID NO: 577-SEQ ID NO: 578 |
| 9 | IgG2C | SP_Bcell | SEQ ID NO: 577-SEQ ID NO: 578 |
| 10 | IgG2C | SP_Bcell | SEQ ID NO: 577-SEQ ID NO: 578 |
| 11 | IgG2C | SP_Bcell | SEQ ID NO: 577-SEQ ID NO: 578 |
| 13 | IgG2C | BM_Plasma_cell | SEQ ID NO: 250-SEQ ID NO: 579 |
| 14 | IgG2C | SP_Plasma_cell | SEQ ID NO: 250-SEQ ID NO: 579 |
| 15 | IgG2B | SP_Bcell | SEQ ID NO: 250-SEQ ID NO: 579 |
| 16 | IgG2C | SP_Plasma_cell | SEQ ID NO: 250-SEQ ID NO: 579 |
| 17 | IgG2C | SP_Bcell | SEQ ID NO: 250-SEQ ID NO: 579 |
| 18 | IgG2C | SP_Plasma_cell | SEQ ID NO: 250-SEQ ID NO: 579 |
| 19 | IgG2C | SP_Plasma_cell | SEQ ID NO: 250-SEQ ID NO: 579 |
| 20 | IgG2B | SP_Bcell | SEQ ID NO: 250-SEQ ID NO: 579 |
| 21 | IgG2C | SP_Plasma_cell | SEQ ID NO: 250-SEQ ID NO: 579 |
| 23 | IgG1 | BM_Plasma_cell | SEQ ID NO: 580-SEQ ID NO: 581 |
| 24 | IgG1 | BM_Plasma_cell | SEQ ID NO: 580-SEQ ID NO: 581 |
| 25 | IgG2C | SP_Bcell | SEQ ID NO: 580-SEQ ID NO: 581 |
| 26 | IgG1 | SP_Plasma_cell | SEQ ID NO: 580-SEQ ID NO: 581 |
| 27 | IgG1 | SP_Plasma_cell | SEQ ID NO: 580-SEQ ID NO: 581 |
| 28 | IgG1 | SP_Bcell | SEQ ID NO: 580-SEQ ID NO: 581 |
| 29 | IgG1 | SP_Plasma_cell | SEQ ID NO: 580-SEQ ID NO: 581 |
| 30 | IgG1 | SP_Bcell | SEQ ID NO: 580-SEQ ID NO: 581 |
| 31 | | | SEQ ID NO: 580-SEQ ID NO: 581 |
| 33 | IgG1 | BM_Plasma_cell | SEQ ID NO: 582-SEQ ID NO: 583 |
| 34 | IgG1 | SP_Plasma_cell | SEQ ID NO: 582-SEQ ID NO: 583 |
| 35 | IgG1 | SP_Plasma_cell | SEQ ID NO: 582-SEQ ID NO: 583 |
| 36 | IgG1 | SP_Plasma_cell | SEQ ID NO: 582-SEQ ID NO: 583 |
| 37 | IgG1 | SP_Plasma_cell | SEQ ID NO: 582-SEQ ID NO: 583 |
| 38 | IgG1 | SP_Bcell | SEQ ID NO: 582-SEQ ID NO: 583 |
| 39 | IgG1 | SP_Plasma_cell | SEQ ID NO: 582-SEQ ID NO: 583 |
| 40 | IgG1 | SP_Plasma_cell | SEQ ID NO: 582-SEQ ID NO: 583 |
| 41 | IgG2C | SP_Plasma_cell | SEQ ID NO: 582-SEQ ID NO: 583 |
| 43 | IgG2C | BM_Plasma_cell | SEQ ID NO: 584-SEQ ID NO: 585 |
| 44 | IgG2C | SP_Plasma_cell | SEQ ID NO: 584-SEQ ID NO: 585 |
| 45 | IgG2C | SP_Bcell | SEQ ID NO: 584-SEQ ID NO: 585 |
| 46 | IgG2C | SP_Bcell | SEQ ID NO: 584-SEQ ID NO: 585 |
| 47 | IgG2C | SP_Plasma_cell | SEQ ID NO: 584-SEQ ID NO: 585 |
| 48 | IgG2C | SP_Plasma_cell | SEQ ID NO: 584-SEQ ID NO: 585 |
| 50 | IgG2C | BM_Plasma_cell | SEQ ID NO: 586-SEQ ID NO: 587 |
| 51 | IgG2C | SP_Bcell | SEQ ID NO: 586-SEQ ID NO: 587 |
| 52 | IgG2C | SP_Plasma_cell | SEQ ID NO: 586-SEQ ID NO: 587 |
| 53 | IgG2C | SP_Bcell | SEQ ID NO: 586-SEQ ID NO: 587 |
| 54 | IgG2C | SP_Bcell | SEQ ID NO: 586-SEQ ID NO: 587 |
| 55 | IgA | SP_Bcell | SEQ ID NO: 586-SEQ ID NO: 587 |
| 57 | IgG2C | BM_Plasma_cell | SEQ ID NO: 169-SEQ ID NO: 588 |
| 58 | IgG2C | BM_Plasma_cell | SEQ ID NO: 169-SEQ ID NO: 588 |
| 59 | IgG2C | BM_Plasma_cell | SEQ ID NO: 169-SEQ ID NO: 588 |
| 60 | IgG2C | SP_Plasma_cell | SEQ ID NO: 169-SEQ ID NO: 588 |
| 61 | IgG2C | SP_Bcell | SEQ ID NO: 169-SEQ ID NO: 588 |
| 63 | IgG1 | BM_Plasma_cell | SEQ ID NO: 589-SEQ ID NO: 590 |
| 64 | IgG1 | BM_Plasma_cell | SEQ ID NO: 589-SEQ ID NO: 590 |
| 65 | IgG1 | SP_Bcell | SEQ ID NO: 589-SEQ ID NO: 590 |
| 66 | IgG2C | SP_Plasma_cell | SEQ ID NO: 589-SEQ ID NO: 590 |
| 67 | IgG2C | SP_Plasma_cell | SEQ ID NO: 589-SEQ ID NO: 590 |
| 69 | IgG2C | BM_Plasma_cell | SEQ ID NO: 232-SEQ ID NO: 281 |
| 70 | IgG2C | SP_Bcell | SEQ ID NO: 232-SEQ ID NO: 281 |
| 71 | IgG1 | SP_Bcell | SEQ ID NO: 232-SEQ ID NO: 281 |
| 72 | IgG1 | SP_Plasma_cell | SEQ ID NO: 232-SEQ ID NO: 281 |
| 74 | IgG1 | BM_Plasma_cell | SEQ ID NO: 591-SEQ ID NO: 592 |
| 75 | IgG1 | SP_Bcell | SEQ ID NO: 591-SEQ ID NO: 592 |
| 76 | IgG2C | SP_Plasma_cell | SEQ ID NO: 591-SEQ ID NO: 592 |

TABLE 3
IgE HDM-specific BCR sequences metadata

| Line | | | |
|---|---|---|---|
| # | Cell_Barcode | Ab_name | meta_tissue |
| 1 | SEQ ID NO: 627 | IgE mAb 1_2 | BoneMarrow |
| 2 | SEQ ID NO: 628 | IgE mAb 8_2 | BoneMarrow |
| 3 | SEQ ID NO: 629 | IgE mAb 12_2 | BoneMarrow |
| 4 | SEQ ID NO: 630 | IgE mAb 13_2 | LymphNode |
| 5 | SEQ ID NO: 631 | IgE mAb 17_2 | LymphNode |
| 6 | SEQ ID NO: 632 | IgE mAb 21_2 | LymphNode |

| Line | | |
|---|---|---|
| # | heavy_sequence | light_sequence |
| 1 | SEQ ID NO: 633 | SEQ ID NO: 634 |
| 2 | SEQ ID NO: 635 | SEQ ID NO: 636 |
| 3 | SEQ ID NO: 637 | SEQ ID NO: 638 |
| 4 | SEQ ID NO: 639 | SEQ ID NO: 640 |
| 5 | SEQ ID NO: 641 | SEQ ID NO: 642 |
| 6 | SEQ ID NO: 643 | SEQ ID NO: 644 |

| Line | | | | |
|---|---|---|---|---|
| # | v_identity | IR_VJ_1_locus | IR_VDJ_1_locus | IR_VJ_1_cdr3 |
| 1 | 0.9966 | IGK | IGH | SEQ ID NO: 645 |
| 2 | 0.97578 | IGK | IGH | SEQ ID NO: 646 |
| 3 | 0.9932 | IGK | IGH | SEQ ID NO: 647 |
| 4 | 1 | IGK | IGH | SEQ ID NO: 648 |
| 5 | 0.89796 | IGK | IGH | SEQ ID NO: 649 |
| 6 | 0.95918 | IGK | IGH | SEQ ID NO: 650 |

| Line | | | |
|---|---|---|---|
| # | IR_VDJ_1_cdr3 | IR_VJ_1_cdr3_nt | IR_VDJ_1_cdr3_nt |
| 1 | SEQ ID NO: 651 | SEQ ID NO: 652 | SEQ ID NO: 653 |
| 2 | SEQ ID NO: 654 | SEQ ID NO: 655 | SEQ ID NO: 656 |
| 3 | SEQ ID NO: 657 | SEQ ID NO: 658 | SEQ ID NO: 659 |
| 4 | SEQ ID NO: 660 | SEQ ID NO: 661 | SEQ ID NO: 662 |
| 5 | SEQ ID NO: 663 | SEQ ID NO: 664 | SEQ ID NO: 665 |
| 6 | SEQ ID NO: 666 | SEQ ID NO: 667 | SEQ ID NO: 668 |

| Line | | | | | |
|---|---|---|---|---|---|
| # | IR_VJ_1_v_gene | IR_VDJ_1_v_gene | IR_VDJ_1_d_gene | IR_VJ_1_i_gene | IR_VDJ_1_i_gene |
| 1 | IGKV3-10*01 | IGHV9-3*01 | IGHD1-1*01 | IGKJ2*01 | IGHJ1*03 |
| 2 | IGKV1-117*01 | IGHV2-3*01 | IGHD1-1*01 | IGKJ4*01 | IGHJ3*01 |
| 3 | IGKV12-46*01 | IGHV1-12*01 | IGHD1-1*01, | IGKJ1*01 | IGHJ3*01 |
| | | | IGHD1-1*02 | | |
| 4 | IGKV3-12*01 | IGHV1-26*01 | IGHD1-1*01 | IGKJ1*01 | IGHJ3*01 |
| 5 | IGKV6-32*01 | IGHV1-42*01 | IGHD3-3*01 | IGKJ1*01 | IGHJ2*01 |
| 6 | IGKV8-24*01 | IGHV1-82*01 | IGHD4-1*01, | IGKJ5*01 | IGHJ1*03 |
| | | | IGHD4-1*02 | | |
| Line | | | | | |
| # | IR_VJ_1_c_gene | IR_VDJ_1_c_gene | Derp1_ab_raw | Derp2_ab_raw | Derf1_ab_raw |
| 1 | IGKC | IGHE | 158 | 60 | 37 |
| 2 | IGKC | IGHE | 29 | 22 | 32 |
| 3 | IGKC | IGHE | 15 | 11 | 57 |
| 4 | IGKC | IGHE | 16 | 46 | 15 |
| 5 | IGKC | IGHE | 10 | 6 | 184 |
| 6 | IGKC | IGHE | 2 | 215 | 1 |

| Line | | | | |
|---|---|---|---|---|
| # | Olee1_ab_raw | Derp1_ab_clr | Derp2_ab_clr | Derf1_ab_clr |
| 1 | 0 | 2.615831 | 1.6454458 | 1.3217044 |
| 2 | 0 | 0.8618727 | 0.65546227 | 1.1766725 |
| 3 | 1 | 0.23326397 | 0.004874706 | 1.7406077 |
| 4 | 1 | 0.98130155 | 1.6244271 | 0.8201184 |
| 5 | 3 | 0.901677 | 0.5801703 | 3.7320528 |
| 6 | 4 | −0.39760602 | 4.0095387 | −0.7951561 |
| Line | | | | |
| # | Olee1_ab_clr | Derp1_ab_rank | Derp2_ab_rank | Derf1_ab_rank |
| 1 | −1.1156448 | 1.006892564 | 1.002825161 | 0.982488148 |
| 2 | −1.1272509 | 0.889548671 | 0.836672436 | 0.969734609 |
| 3 | −0.43410373 | 0.636937195 | 0.45274196 | 0.972788444 |
| 4 | 0.028710008 | 0.775289528 | 0.858380754 | 0.76541155 |
| 5 | 0.6026533 | 0.434538481 | 0.33633976 | 0.544358354 |
| 6 | 0.8257969 | −0.245443457 | 0.411732604 | −0.386059378 |

TABLE 4
HDM-specific clustered BCR sequences Containing an IgE clone from human
bone marrow

| Line | | | | |
|---|---|---|---|---|
| # | Cell_Barcode | heavy_sequence | | |
| 1 | SEQ ID NO: 669 | SEQ ID NO: 670 | | |
| 2 | SEQ ID NO: 671 | SEQ ID NO: 672 | | |
| 3 | SEQ ID NO: 673 | SEQ ID NO: 674 | | |
| 4 | SEQ ID NO: 675 | SEQ ID NO: 676 | | |
| 5 | SEQ ID NO: 677 | SEQ ID NO: 678 | | |
| 6 | SEQ ID NO: 679 | SEQ ID NO: 680 | | |
| 7 | SEQ ID NO: 681 | SEQ ID NO: 682 | | |
| 8 | SEQ ID NO: 683 | SEQ ID NO: 684 | | |
| 9 | SEQ ID NO: 685 | SEQ ID NO: 686 | | |
| 10 | SEQ ID NO: 687 | SEQ ID NO: 688 | | |
| 11 | SEQ ID NO: 689 | SEQ ID NO: 690 | | |
| 12 | SEQ ID NO: 691 | SEQ ID NO: 692 | | |
| 13 | SEQ ID NO: 693 | SEQ ID NO: 694 | | |
| 14 | SEQ ID NO: 695 | SEQ ID NO: 696 | | |
| 15 | SEQ ID NO: 697 | SEQ ID NO: 698 | | |
| 16 | SEQ ID NO: 699 | SEQ ID NO: 700 | | |
| 17 | SEQ ID NO: 701 | SEQ ID NO: 702 | | |
| 18 | SEQ ID NO: 703 | SEQ ID NO: 704 | | |
| 19 | SEQ ID NO: 705 | SEQ ID NO: 706 | | |
| 20 | SEQ ID NO: 707 | SEQ ID NO: 708 | | |
| 21 | SEQ ID NO: 709 | SEQ ID NO: 710 | | |
| 22 | SEQ ID NO: 711 | SEQ ID NO: 712 | | |
| 23 | SEQ ID NO: 713 | SEQ ID NO: 714 | | |
| 24 | SEQ ID NO: 715 | SEQ ID NO: 716 | | |
| Line | | | | |
| # | light_sequence | v_identity | | |
| 1 | SEQ ID NO: 717 | 0.89492 | | |
| 2 | Nan | 0.92982 | | |
| 3 | SEQ ID NO: 718 | 0.92982 | | |
| 4 | SEQ ID NO: 719 | 0.92857 | | |
| 5 | Nan | 0.925 | | |
| 6 | SEQ ID NO: 720 | 0.89153 | | |
| 7 | Nan | 0.89855 | | |
| 8 | SEQ ID NO: 721 | 0.93103 | | |
| 9 | SEQ ID NO: 722 | 0.87854 | | |
| 10 | Nan | 0.93103 | | |
| 11 | Nan | 0.92453 | | |
| 12 | SEQ ID NO: 723 | 0.93333 | | |
| 13 | nan | 0.90625 | | |
| 14 | nan | 0.93103 | | |
| 15 | nan | 0.93103 | | |
| 16 | nan | 0.88889 | | |
| 17 | nan | 0.90722 | | |
| 18 | SEQ ID NO: 724 | 0.90102 | | |
| 19 | SEQ ID NO: 725 | 0.90722 | | |
| 20 | nan | 0.924 | | |
| 21 | nan | 0.94643 | | |
| 22 | nan | 0.91818 | | |
| 23 | SEQ ID NO: 726 | 0.925 | | |
| 24 | Nan | 0.88618 | | |
| Line | IR_VJ_ | IR_VDJ_1 | IR_VJ_ | IR_VDJ_ |
| # | 1_locus | locus | 1_cdr3 | 1_cdr3 |
| 1 | IGL | IGH | SEQ ID NO: 727 | SEQ ID NO: 728 |
| 2 | | IGH | None | SEQ ID NO: 729 |
| 3 | IGK | IGH | SEQ ID NO: 730 | SEQ ID NO: 729 |
| 4 | IGL | IGH | SEQ ID NO: 731 | SEQ ID NO: 729 |
| 5 | | IGH | None | SEQ ID NO: 729 |
| 6 | IGL | IGH | SEQ ID NO: 732 | SEQ ID NO: 729 |
| 7 | | IGH | None | SEQ ID NO: 729 |
| 8 | IGL | IGH | SEQ ID NO: 733 | SEQ ID NO: 729 |
| 9 | IGK | IGH | SEQ ID NO: 734 | SEQ ID NO: 729 |
| 10 | | IGH | None | SEQ ID NO: 729 |
| 11 | | IGH | None | SEQ ID NO: 729 |
| 12 | IGL | IGH | CAIWHDSETGWVF | SEQ ID NO: 729 |
| | | | (SEQ ID NO: 735) | |
| 13 | | IGH | None | SEQ ID NO: 729 |
| 14 | | IGH | None | SEQ ID NO: 729 |
| 15 | | IGH | None | SEQ ID NO: 729 |
| 16 | | IGH | None | SEQ ID NO: 736 |
| 17 | | IGH | None | SEQ ID NO: 737 |
| 18 | IGL | IGH | CAAWDDRLNGWVF | SEQ ID NO: 737 |
| | | | (SEQ ID NO: 738) | |
| 19 | IGL | IGH | CAAWDDRLNGWVF | SEQ ID NO: 737 |
| | | | (SEQ ID NO: 738) | |
| 20 | | IGH | None | SEQ ID NO: 739 |
| 21 | | IGH | None | SEQ ID NO: 739 |
| 22 | | IGH | None | SEQ ID NO: 739 |
| 23 | IGK | IGH | CQQYDKWLITF | SEQ ID NO: 739 |
| | | | (SEQ ID NO: 740) | |
| 24 | | IGH | None | SEQ ID NO: 741 |
| Line | IR_VJ_1_ | IR_VDJ_1_ | IR_VJ_1_ | IR_VDJ_ |
| # | cdr3_nt | cdr3_nt | v_gene | 1_v_gene |
| 1 | SEQ ID NO: 742 | SEQ ID NO: 743 | IGLV2-14*01 | IGHV3-9*01 |
| 2 | None | SEQ ID NO: 744 | | IGHV3- |
| | | | | 9*01, |
| | | | | IGHV3-9*02 |
| 3 | SEQ ID NO: 745 | SEQ ID NO: 744 | IGKV3-20*01 | IGHV3- |
| | | | | 9*01, |
| | | | | IGHV3-9*02 |
| 4 | SEQ ID NO: 746 | SEQ ID NO: 744 | IGLV3- | IGHV3- |
| | | | 21*02, | 9*01, |
| | | | IGLV3- | IGHV3-9*02 |
| | | | 21*03, | |
| | | | IGLV3- | |
| | | | 21*04 | |
| 5 | None | SEQ ID NO: 744 | | IGHV3- |
| | | | | 9*01, |
| | | | | IGHV3-9*02 |
| 6 | SEQ ID NO: 747 | SEQ ID NO: 744 | IGLV2-14*01 | IGHV3-9*01 |
| 7 | None | SEQ ID NO: 744 | | IGHV3- |
| | | | | 9*01, |
| | | | | IGHV3-9*02 |
| 8 | SEQ ID NO: 748 | SEQ ID NO: 744 | IGLV3-21*03 | IGHV3- |
| | | | | 9*01, |
| | | | | IGHV3-9*02 |
| 9 | SEQ ID NO: 749 | SEQ ID NO: 744 | IGKV3- | IGHV3-9*01 |
| | | | 11*01, | |
| | | | IGKV3D- | |
| | | | 11*02 | |
| 10 | None | SEQ ID NO: 744 | | IGHV3- |
| | | | | 9*01, |
| | | | | IGHV3-9*02 |
| 11 | None | SEQ ID NO: 744 | | IGHV3- |
| | | | | 9*01, |
| | | | | IGHV3-9*02 |
| 12 | SEQ ID NO: 750 | SEQ ID NO: 744 | IGLV1-47*01 | IGHV3- |
| | | | | 9*01, |
| | | | | IGHV3-9*02 |
| 13 | None | SEQ ID NO: 744 | | IGHV3- |
| | | | | 9*01, |
| | | | | IGHV3-9*02 |
| 14 | None | SEQ ID NO: 744 | | IGHV3- |
| | | | | 9*01, |
| | | | | IGHV3-9*02 |
| 15 | None | SEQ ID NO: 744 | | IGHV3- |
| | | | | 9*01, |
| | | | | IGHV3-9*02 |
| 16 | None | SEQ ID NO: 751 | | IGHV4-59*10 |
| 17 | None | SEQ ID NO: 752 | | IGHV4- |
| | | | | 59*01, |
| | | | | IGHV4- |
| | | | | 59*02, |
| | | | | IGHV4- |
| | | | | 59*11 |
| 18 | SEQ ID NO: 753 | SEQ ID NO: 752 | IGLV1-44*01 | IGHV4-59*01 |
| 19 | SEQ ID NO: 753 | SEQ ID NO: 752 | IGLV1-44*01 | IGHV4- |
| | | | | 59*01, |
| | | | | IGHV4- |
| | | | | 59*02, |
| | | | | IGHV4- |
| | | | | 59*11 |
| 20 | None | SEQ ID NO: 754 | | IGHV3-7*01 |
| 21 | None | SEQ ID NO: 754 | | IGHV3- |
| | | | | 21*01, |
| | | | | IGHV3- |
| | | | | 21*02, |
| | | | | IGHV3- |
| | | | | 21*05 |
| 22 | None | SEQ ID NO: 754 | | IGHV3-7*01 |
| 23 | SEQ ID NO: 755 | SEQ ID NO: 754 | IGKV1D- | IGHV3-7*01 |
| | | | 8*02, | |
| | | | IGKV1D- | |
| | | | 8*03 | |
| 24 | None | SEQ ID NO: 756 | | IGHV3-23*05 |
| Line | IR_VDJ_1_ | IR_VJ_1_j | IR_VDJ_1_j | IR_VJ _1_c_ |
| # | d_gene | _gene | gene | gene |
| 1 | IGHD3-10*02, | IGLJ1*01 | IGHJ6*02 | IGLC1 |
| | IGHD4/OR15- | | | |
| | 4a*01, | | | |
| | IGHD4/ | | | |
| | OR15-4b*01 | | | |
| 2 | IGHD4-17*01 | | IGHJ6*01 | |
| 3 | IGHD4-17*01 | IGKJ3*01 | IGHJ6*01 | IGKC |
| 4 | IGHD4-17*01 | IGLJ3*02 | IGHJ6*01 | IGLC2 |
| 5 | IGHD4-17*01 | | IGHJ6*01 | |
| 6 | IGHD4-17*01 | IGLJ1*01 | IGHJ6*01 | IGLC1 |
| 7 | IGHD4-17*01 | | IGHJ6*01 | |
| 8 | IGHD4-17*01 | IGLJ3*02 | IGHJ6*01 | IGLC2 |
| 9 | IGHD4-17*01 | IGKJ4*01 | IGHJ6*01 | IGKC |
| 10 | IGHD4-17*01 | | IGHJ6*01 | |
| 11 | IGHD4-17*01 | | IGHJ6*01 | |
| 12 | IGHD4-17*01 | IGLJ3*02 | IGHJ6*01 | IGLC2 |
| 13 | IGHD4-17*01 | | IGHJ6*01 | |
| 14 | IGHD4-17*01 | | IGHJ6*01 | |
| 15 | IGHD4-17*01 | | IGHJ6*01 | |
| 16 | IGHD5-24*01 | | IGHJ6*02 | |
| 17 | IGHD2-15*01, | | IGHJ3*01 | |
| | IGHD2-21*01, | | | |
| | IGHD2- | | | |
| | 21*02 | | | |
| 18 | IGHD2-15*01, | IGLJ3*02 | IGHJ3*01 | IGLC2 |
| | IGHD2-21*01, | | | |
| | IGHD2- | | | |
| | 21*02 | | | |
| 19 | IGHD2-15*01, | IGLJ3*02 | IGHJ3*01 | IGLC2 |
| | IGHD2-21*01, | | | |
| | IGHD2- | | | |
| | 21*02 | | | |
| 20 | IGHD2-2*02 | | IGHJ2*01 | |
| 21 | IGHD2-2*02 | | IGHJ2*01 | |
| 22 | IGHD2-2*02 | | IGHJ2*01 | |
| 23 | IGHD2-2*02 | IGKJ5*01 | IGHJ2*01 | IGKC |
| 24 | IGHD2-21*01, | | IGHJ5*02 | |
| | IGHD2-21*02 | | | |
| Line | IR_VDJ_1_ | | cc_aa_ | cc_aa_ |
| # | c_gene | Ig_chain | hamming | hamming_size |
| 1 | IGHE | IgE | 21 | 15 |
| 2 | IGHG4 | IgG | 21 | 15 |
| 3 | IGHG4 | IgG | 21 | 15 |
| 4 | IGHG4 | IgG | 21 | 15 |
| 5 | IGHG2 | IgG | 21 | 15 |
| 6 | IGHG4 | IgG | 21 | 15 |
| 7 | IGHG4 | IgG | 21 | 15 |
| 8 | IGHG4 | IgG | 21 | 15 |
| 9 | IGHG3 | IgG | 21 | 15 |
| 10 | IGHG4 | IgG | 21 | 15 |
| 11 | IGHG3 | IgG | 21 | 15 |
| 12 | IGHG4 | IgG | 21 | 15 |
| 13 | IGHG4 | IgG | 21 | 15 |
| 14 | IGHG4 | IgG | 21 | 15 |
| 15 | IGHG4 | IgG | 21 | 15 |
| 16 | IGHE | IgE | 576 | 1 |
| 17 | IGHE | IgE | 696 | 3 |
| 18 | IGHE | IgE | 696 | 3 |
| 19 | IGHE | IgE | 696 | 3 |
| 20 | IGHE | IgE | 753 | 4 |
| 21 | IGHE | IgE | 753 | 4 |
| 22 | IGHE | IgE | 753 | 4 |
| 23 | IGHE | IgE | 753 | 4 |
| 24 | IGHE | IgE | 1330 | 1 |

The disclosure provides the following illustrative embodiments:
1. An antibody capture complex comprising a first component of a binding pair linked to an antibody-capture molecule;
   wherein the first component of the binding pair is capable of binding a second component of the binding pair;
   wherein the antibody-capture molecule is capable of binding to a target antibody, wherein the target antibody is secreted by an antibody secreting cell;
   wherein when the second component of the binding pair is biotin and the first component of the binding pair is streptavidin, the antibody-capture molecule does not bind to a kappa light chain of the target antibody.
2. The antibody capture complex of embodiment 1, wherein:
   the first component of the binding pair comprises avidin, streptavidin, or anti-biotin and the second component of the binding pair comprises biotin;
   the first component of the binding pair comprises one of jun and fos and the second component of the binding pair comprises the other of jun and fos;
   the first component of the binding pair comprises one of mad and max and the second component of the binding pair comprises the other of mad and max;
   the first component of the binding pair comprises one of myc and max and the second component of the binding pair comprises the other of myc and max;
   the first component of the binding pair comprises one of an azide and an alkyne and the second component of the binding pair comprises the other of an azide and an alkyne; or
   the first component of the binding pair comprises one of an azide and a phosphine and the second component of the binding pair comprises the other of an azide and a phosphine.
3. The antibody capture complex of embodiment 1, wherein the first component of the binding pair comprises avidin or streptavidin and the second component of the binding pair comprises biotin.
4. The antibody capture complex of embodiment 1, wherein the second component of the binding pair comprises a surface marker of the antibody secreting cell and the first component of the binding pair comprises an antibody that binds to the surface marker.
5. The antibody capture complex of embodiment 4, wherein the cell surface marker comprises CD27, CD38, CD45, CD138, CD98, CD78, CD319, CXCR4, BCMA, GPRC5D, FCRL5, CD19, Ly6D, CD52, or transmembrane activator calcium modulator and cyclophilin ligand interactor (TACI).
6. The antibody capture complex of embodiment 1, wherein the second component of the binding pair comprises an interleukin-6 receptor (IL-6R), and the first component of the binding pair comprises an anti-IL-6R antibody or interleukin-6 (IL-6).
7. The antibody capture complex of embodiment 1, wherein the second component of the binding pair comprises CD27, and the first component of the binding pair comprises an anti-CD27 antibody or CD70.
8. The antibody capture complex of embodiment 1, wherein:
   the second component of the binding pair comprises fluorescein isothiocyanate (FITC), and the first component of the binding pair comprises an anti-FITC antibody;
   the second component of the binding pair comprises phycoerythrin (PE), and the first component of the binding pair comprises an anti-PE antibody;
   the second component of the binding pair comprises APC, and the first component of the binding pair comprises an anti-APC antibody;
   the second component of the binding pair comprises BV421, and the first component of the binding pair comprises an anti-BV421 antibody;
   the second component of the binding pair comprises BV510, and the first component of the binding pair comprises an anti-BV510 antibody;
   the second component of the binding pair comprises BV605, and the first component of the binding pair comprises an anti-BV605 antibody;
   the second component of the binding pair comprises BV650, and the first component of the binding pair comprises an anti-BV650 antibody;
   the second component of the binding pair comprises BV711, and the first component of the binding pair comprises an anti-BV711 antibody; or
   the second component of the binding pair comprises BV786, and the first component of the binding pair comprises an anti-BV786 antibody.
9. The antibody capture complex of any one of embodiments 1 to 8, wherein the antibody-capture molecule comprises a capture antibody.
10. The antibody capture complex of embodiment 9, wherein the capture antibody comprises an anti-Fc antibody, an anti-light chain kappa antibody, or an anti-light chain lambda antibody.
11. The antibody capture complex of embodiment 10, wherein the anti-Fc antibody comprises an anti-Fcγ antibody, an anti-Fcα antibody, or an anti-Fcε antibody.
12. The antibody capture complex of embodiment 11, wherein the anti-Fcγ antibody comprises an anti-FcγRI antibody, an anti-FcγRIIA antibody, an anti-FcγRIIB antibody, an anti-FcγRIIB1 antibody, an anti-FcγRIIB2 antibody, an anti-FcγRIIIA antibody, or an anti-FcγRIIIB antibody.
13. The antibody capture complex of embodiment 11, wherein the anti-Fcα antibody comprises an anti-FcαRI antibody.
14. The antibody capture complex of embodiment 11, wherein the anti-Fcε antibody comprises an anti-FcεRIantibody or an anti-FcεRII antibody.
15. The antibody capture complex of embodiment 9, wherein the capture antibody comprises an anti-IgM antibody.
16. The antibody capture complex of embodiment 9, wherein the capture antibody comprises an anti-IgG antibody.
17. The antibody capture complex of embodiment 16, wherein the anti-IgG antibody comprises an anti-IgG1 antibody, an anti-IgG2 antibody, an anti-IgG2a antibody, an anti-IgG2b antibody, an anti-IgG3 antibody, or an anti-IgG4 antibody.
18. The antibody capture complex of embodiment 9, wherein the capture antibody comprises an anti-IgA antibody.
19. The antibody capture complex of embodiment 18, wherein the anti-IgA antibody comprises an anti-IgA1 antibody, an anti-IgA2 antibody, an anti-secretory IgA antibody, or a polymeric anti-IgA antibody.
20. The antibody capture complex of embodiment 9, wherein the capture antibody comprises an anti-IgE antibody.
21. The antibody capture complex of any one of embodiments 1 to 8, wherein the antibody-capture molecule comprises an Fc receptor or an ectodomain of the Fc receptor.
22. The antibody capture complex of embodiment 21, wherein the Fc receptor comprises an Fcγ receptor, an Fcα receptor, or an Fcε receptor.
23. The antibody capture complex of embodiment 22, wherein the Fcγ receptor comprises an FcγRI receptor, an FcγRIIA receptor, an FcγRIIB receptor, an FcγRIIB1 receptor, an FcγRIIB2 receptor, an FcγRIIIA receptor, an FcγRIIIB receptor, or an FcRn receptor.
24. The antibody capture complex of embodiment 22, wherein the Fcα receptor comprises an FcαRI receptor or an Fcα/μR receptor.
25. The antibody capture complex of embodiment 22, wherein the Fcε receptor comprises an FcεRI receptor or an FcεRII receptor.
26. The antibody capture complex of any one of embodiments 1 to 8, wherein the antibody-capture molecule comprises protein A or protein G.
27. The antibody capture complex of any one of embodiments 1 to 8, wherein the antibody-capture molecule comprises protein L.
28. The antibody capture complex of embodiment 1, wherein the antibody capture complex comprises avidin or streptavidin linked to an ectodomain of a high affinity IgE receptor (FcεRIa).
29. A method of capturing a target antibody secreted by an antibody secreting cell, the method comprising:
   contacting a population of antibody secreting cells with a second component of a binding pair to allow the second component of the binding pair to bind to the cell surface of the population of antibody secreting cells, wherein the population of antibody secreting cells comprises the antibody secreting cell that secretes the target antibody;
   contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the antibody secreting cells, and whereby the antibody-capture molecule captures the target antibody secreted by the antibody secreting cell; and
   contacting the population of antibody secreting cells with an antigen, whereby the target antibody secreted by the antibody secreting cell and captured by the antibody capture complex binds the antigen.
30. The method of embodiment 29, wherein:
   the first component of the binding pair comprises avidin, streptavidin, or anti-biotin and the second component of the binding pair comprises biotin;
   the first component of the binding pair comprises one of jun and fos and the second component of the binding pair comprises the other of jun and fos;
   the first component of the binding pair comprises one of mad and max and the second component of the binding pair comprises the other of mad and max;
   the first component of the binding pair comprises one of myc and max and the second component of the binding pair comprises the other of myc and max;
   the first component of the binding pair comprises one of an azide and an alkyne and the second component of the binding pair comprises the other of an azide and an alkyne; or
   the first component of the binding pair comprises one of an azide and a phosphine and the second component of the binding pair comprises the other of an azide and a phosphine.
31. The method of embodiment 29, wherein the first component of the binding pair comprises avidin or streptavidin and the second component of the binding pair comprises biotin.
32. The method of embodiment 29, wherein:
   the second component of the binding pair comprises fluorescein isothiocyanate (FITC), and the first component of the binding pair comprises an anti-FITC antibody;
   the second component of the binding pair comprises phycoerythrin (PE), and the first component of the binding pair comprises an anti-PE antibody;
   the second component of the binding pair comprises APC, and the first component of the binding pair comprises an anti-APC antibody;
   the second component of the binding pair comprises BV421, and the first component of the binding pair comprises an anti-BV421 antibody;
   the second component of the binding pair comprises BV510, and the first component of the binding pair comprises an anti-BV510 antibody;
   the second component of the binding pair comprises BV605, and the first component of the binding pair comprises an anti-BV605 antibody;
   the second component of the binding pair comprises BV650, and the first component of the binding pair comprises an anti-BV650 antibody;
   the second component of the binding pair comprises BV711, and the first component of the binding pair comprises an anti-BV711 antibody; or
   the second component of the binding pair comprises BV786, and the first component of the binding pair comprises an anti-BV786 antibody.
33. A method of capturing a target antibody secreted by an antibody secreting cell, the method comprising:
   contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell; and
   contacting the population of antibody secreting cells with an antigen, whereby the target antibody secreted by the antibody secreting cell and captured by the antibody capture complex binds the antigen.
34. The method of embodiment 33, wherein the second component of the binding pair comprises a surface marker of the population of antibody secreting cells and the first component of the binding pair is an antibody that binds to the surface marker.
35. The method of embodiment 34, wherein the surface marker comprises CD27, CD38, CD45, CD138, CD98, CD78, CD319, CXCR4, BCMA, GPRC5D, FCRL5, CD19, Ly6D, CD52, or transmembrane activator calcium modulator and cyclophilin ligand interactor (TACI).
36. The method of embodiment 33, wherein the second component of the binding pair comprises an interleukin-6 receptor (IL-6R), and the first component of the binding pair comprises an anti-IL-6R antibody or interleukin-6 (IL-6).
37. The method of embodiment 33, wherein the second component of the binding pair comprises CD27, and the first component of the binding pair comprises an anti-CD27 antibody or CD70.
38. The method of any one of embodiments 29 to 37, wherein the antibody-capture molecule comprises a capture antibody.
39. The method of embodiment 38, wherein the capture antibody comprises an anti-Fc antibody, an anti-light chain kappa antibody, or an anti-light chain lambda antibody.
40. The method of embodiment 39, wherein the anti-Fc antibody comprises an anti-Fcγ antibody, an anti-Fcα antibody, or an anti-Fcε antibody.
41. The method of embodiment 40, wherein the anti-Fcγ antibody comprises an anti-FcγRI antibody, an anti-FcγRIIA antibody, an anti-FcγRIIB antibody, an anti-FcγRIIB1 antibody, an anti-FcγRIIB2 antibody, an anti-FcγRIIIA antibody, or an anti-FcγRIIIB antibody.
42. The method of embodiment 40, wherein the anti-Fcα antibody comprises an anti-FcαRI antibody.
43. The method of embodiment 40, wherein the anti-Fcε antibody comprises an anti-FcεRI antibody or an anti-FcεRII antibody.
44. The method of embodiment 38, wherein the capture antibody comprises an anti-IgM antibody.
45. The method of embodiment 38, wherein the capture antibody comprises an anti-IgG antibody.
46. The method of embodiment 45, wherein the anti-IgG antibody comprises an anti-IgG1 antibody, an anti-IgG2 antibody, an anti-IgG2a antibody, an anti-IgG2b antibody, an anti-IgG3 antibody, or an anti-IgG4 antibody.
47. The method of embodiment 38, wherein the capture antibody comprises an anti-IgA antibody.
48. The method of embodiment 47, wherein the anti-IgA antibody comprises an anti-IgA1 antibody, an anti-IgA2 antibody, an anti-secretory IgA antibody, or a polymeric anti-IgA antibody.
49. The method of embodiment 38, wherein the capture antibody comprises an anti-IgE antibody.
50. The method of any one of embodiments 29 to 37, wherein the antibody-capture molecule comprises an Fc receptor or the ectodomain of the Fc receptor.
51. The method of embodiment 50, wherein the Fc receptor comprises an Fcγ receptor, an Fcα receptor, or an Fcε receptor.
52. The method of embodiment 51, wherein the Fcγ receptor comprises an FcγRI receptor, an FcγRIIA receptor, an FcγRIIB receptor, an FcγRIIB1 receptor, an FcγRIIB2 receptor, an FcγRIIIA receptor, an FcγRIIIB receptor, or an FcRn receptor.
53. The method of embodiment 51, wherein the Fcα receptor comprises an FcαRI receptor or an Fcα/μR receptor.
54. The method of embodiment 51, wherein the Fcε receptor comprises an FcεRI receptor or an FcεRII receptor.
55. The method of any one of embodiments 29 to 37, wherein the antibody-capture molecule comprises protein A or protein G.
56. The method of any one of embodiments 29 to 37, wherein the antibody-capture molecule comprises protein L.
57. The method of any one of embodiments 29-56, further comprising contacting the population of antibody secreting cells with a secondary anti-Ig antibody, whereby the target antibody secreted by the antibody secreting cell and captured by the antibody capture complex binds the secondary anti-Ig antibody.
58. The method of embodiment 57, wherein the secondary anti-Ig antibody is anti-IgE or anti-IgG.
59. The method of embodiment 57 or 58, wherein the secondary anti-Ig antibody is detectably labeled.
60. The method of embodiment 59, wherein the secondary anti-Ig antibody is labeled with a radioactive compound, a fluorescent compound, or an enzyme.
61. The method of embodiment 60, wherein the radioactive compound comprises ³H, ¹⁴C, ¹⁹F, ³⁵S, ¹²⁵I, ¹³¹I, ¹¹¹In, or ⁹⁹Tc.
62. The method of embodiment 60, wherein the fluorescent compound comprises fluorescein, fluorescein isothiocyanate, rhodamine, 5-dimethylamine-1-naphthalenesulfonyl chloride, phycoerythrin, or a fluorescent protein.
63. The method of embodiment 60, wherein the enzyme comprises alkaline phosphatase, horseradish peroxidase, luciferase, or glucose oxidase.
64. The method of any one of embodiments 29-63, wherein in the step of contacting the population of antibody secreting cells with an antigen, the antigen is a barcoded antigen.
65. The method of any one of embodiments 29 to 64, wherein in the step of contacting the population of antibody secreting cells with an antigen, the population of antibody secreting cells is contacted with a plurality of barcoded antigens, wherein each barcoded antigen of the plurality of barcoded antigens comprises a unique antigen linked to a unique nucleic acid molecule.
66. The method of embodiment 65, wherein the unique nucleic acid molecule comprises DNA.
67. The method of embodiment 65, wherein the unique nucleic acid molecule comprises RNA.
68. The method of any one of embodiments 65 to 67, wherein each unique nucleic acid molecule comprises from about 10 nucleotides to about 500 nucleotides.
69. The method of any one of embodiments 65 to 68, wherein each barcoded antigen is detectably labeled.
70. The method of embodiment 69, wherein each barcoded antigen is labeled with a radioactive compound, a fluorescent compound, or an enzyme.
71. The method of embodiment 70, wherein the radioactive compound comprises ³H, ¹⁴C, ¹⁹F, ³⁵S, ¹²⁵I, ¹³¹I, ¹¹¹In, or ⁹⁹Tc.
72. The method of embodiment 70, wherein the fluorescent compound comprises fluorescein, fluorescein isothiocyanate, rhodamine, 5-dimethylamine-1-naphthalenesulfonyl chloride, phycoerythrin, or a fluorescent protein.
73. The method of embodiment 70, wherein the enzyme comprises alkaline phosphatase, horseradish peroxidase, luciferase, or glucose oxidase.
74. The method of any one of embodiments 64 to 73, wherein the barcoded antigen comprises an allergen.
75. The method of any one of embodiments 29 to 74, wherein the antibody secreting cell is a plasma cell or plasmablast.
76. The method of any one of embodiments 29 to 75, wherein the population of antibody secreting cells is obtained by enriching a population of cells obtained from a human for immune cells.
77. The method of embodiment 76, wherein the population of cells obtained from the human is enriched for plasma cells or plasmablasts.
78. The method of embodiment 77, wherein the population of enriched immune cells is enriched for plasma cells or plasmablasts using pan-B cell enrichment.
79. The method of any one of embodiments 29 to 78, wherein the population of antibody secreting cells is obtained from the lymph node, lung, bone marrow, or blood of a human.
80. The method of any one of embodiments 29 to 79, further comprising detecting the antigen bound by the target antibody.
81. The method of any one of embodiments 29 to 80, further comprising sorting the population of antibody secreting cells to collect a pool of antibody secreting cells, wherein the antibody secreting cells in the pool each secrete an antibody that is captured by the antibody capture molecule and is bound by the antigen, wherein the collected pool of antibody secreting cells comprises the antibody secreting cell that secretes the target antibody.
82. The method of embodiment 81, wherein separating the collected pool of antibody secreting cells into single cells and isolating the antibody secreting cell that secretes the target antibody.
83. The method of any one of embodiments 57 to 80, further comprising detecting the secondary anti-Ig antibody.
84. The method of embodiment 83, further comprising sorting the population of antibody secreting cells to collect a pool of antibody secreting cells, wherein the antibody secreting cells in the pool each secrete an antibody that is captured by the antibody capture molecule and is bound by the antigen and the secondary anti-Ig antibody, wherein the collected pool of antibody secreting cells comprises the antibody secreting cell that secretes the target antibody bound by the antigen and the secondary anti-Ig antibody.
85. The method of embodiment 84, further comprising separating the collected pool of antibody secreting cells into single cells and isolating the antibody secreting cell that secretes the target antibody.
86. The method of any one of embodiments 29 to 85, further comprising contacting the population of antibody secreting cells with an Fc block prior to contacting the population of antibody secreting cells with the second component of the binding pair.
87. A method of capturing an IgE antibody secreted by an antibody secreting cell, wherein the IgE antibody is directed to an allergen, the method comprising:
   contacting a population of antibody secreting cells with NHS-biotin to allow biotin to bind to the cell surface, wherein the population of antibody secreting cells comprises the antibody secreting cell that secretes the IgE antibody;
   contacting the population of antibody secreting cells with an antibody capture complex, wherein the antibody capture complex comprises streptavidin linked to an ectodomain of a high affinity IgE receptor (FcεRIa), whereby the antibody-capture molecule binds to the IgE antibody secreted by the antibody secreting cell; and
   contacting the population of antibody secreting cells with an antigen, wherein the antigen is an antigenic portion or a mixture of a plurality of antigenic portions of the allergen, whereby the IgE antibody captured by the antibody capture complex binds to the antigen.
88. The method of embodiment 87, wherein the antigen comprises a plurality of barcoded antigens, wherein each of the plurality of barcoded antigens is a different antigenic portion of the allergen.
89. The method of embodiment 87 or 88, further comprising sorting the population of antibody secreting cells to collect a pool of antibody secreting cells, wherein the antibody secreting cells in the pool each secrete an IgE antibody that is captured by the antibody capture complex and is bound by the antigen, wherein the collected pool of antibody secreting cells comprises the antibody secreting cell that secretes the IgE antibody.
90. The method of embodiment 87 or 88, further comprising contacting the population of antibody secreting cells with an anti-IgE antibody to allow the IgE antibody secreted by the antibody secreting cell and captured by the antibody capture complex to bind to the anti-IgE antibody.
91. The method of embodiment 90, further comprising sorting the population of antibody secreting cells to collect a pool of antibody secreting cells, wherein the antibody secreting cells in the pool each secrete an IgE antibody that is captured by the antibody capture complex and is bound by the antigen and the anti-IgE antibody, wherein the collected pool of antibody secreting cells comprises the antibody secreting cell that secretes the IgE antibody.
92. The method of embodiment 89 or 91, further comprising separating the collected pool of antibody secreting cells into single cells and isolating the antibody secreting cell that secretes the IgE antibody.
93. The method of any one of embodiments 29-92, wherein the step of contacting the population of antibody secreting cells with an antigen comprises:
   (a) contacting the population of antibody secreting cells with a first labeled form of the antigen to allow the antigen to bind to antibodies including the target antibody captured on the cell surface of the population of antibody secreting cells, wherein the antigen of the first labeled form is conjugated to a first detectable label;
   (b) washing the population of antibody secreting cells to remove unbound antigen;
   (c) contacting the population of antibody secreting cells with
      (i) an unlabeled form of the antigen,
      (ii) a second labeled form of the antigen, or
      (iii) the unlabeled form of the antigen and the second labeled form of the antigen;
   (d) washing the population of antibody secreting cells to remove unbound antigen, and
   (e) collecting a pool of antibody secreting cells remaining bound to the first labeled form of the antigen, wherein the collected pool of antibody secreting cells comprises the antibody secreting cell that secretes the target antibody, wherein the target antibody secreted by the antibody secreting cell and captured by the antibody capture complex remains bound to the first labeled form of the antigen.
94. The method of embodiment 93, wherein the first labeled form of the antigen is at a concentration between 0.001 nM and 1 uM.
95. The method of embodiment 93, wherein the first labeled form of the antigen is at a concentration between 0.1 and 7.5 nM.
96. The method according to any one of embodiments 93-95, wherein the first detectable label is a first fluorescent label.
97. The method according to any one of embodiments 93-96, wherein the antigen is a protein in a monomeric form.
98. The method according to any one of embodiments 93-96, wherein the antigen is a protein in a multimeric form.
99. The method according to any one of embodiments 93-96, wherein the antigen is a protein present in both monomeric and multimeric forms.
100. The method according to any one of embodiments 97-99, wherein the first labeled form of the antigen is a monovalent form of the antigen.
101. The method according to any one of embodiments 97-100, wherein the unlabeled form of the antigen is a monovalent form of the antigen.
102. The method according to any one of embodiments 97-100, wherein the unlabeled form of the antigen is a multivalent form of the antigen.
103. The method of embodiment 102, wherein the multivalent form of the antigen is provided by a multivalent molecule to which the antigen is bound.
104. The method of embodiment 103, wherein the multivalent molecule is selected from a streptavidin multimer such as tetramer, a dimer of an immunoglobulin Fc fragment, or a trimer of a trimerization molecule such as foldon.
105. The method according to any one of embodiments 97-104, wherein the second labeled form of the antigen is a monovalent form of the antigen.
106. The method according to any one of embodiments 99-104, wherein the second labeled form of the antigen is a multivalent form of the antigen.
107. The method of embodiment 106, wherein the multivalent form of the antigen is provided by a multivalent molecule to which the antigen is bound or linked.
108. The method of embodiment 107, wherein the multivalent molecule is a streptavidin multimer such as tetramer, a dimer of an immunoglobulin Fc fragment, or a trimer of a trimerization molecule such as foldon.
109. The method according to any of embodiments 106-108, wherein the multivalent form of the antigen is labeled with a second detectable label.
110. The method of embodiment 109, wherein the second detectable label is a second fluorescent label.
111. The method of embodiment 100, wherein in step (c) the population of antibody secreting cells is contacted with the unlabeled form of the antigen.
112. The method of embodiment 111, wherein the unlabeled form of the antigen is a monovalent form of the antigen.
113. The method of embodiment 111 or 112, wherein the antigen in the unlabeled form is at least 2 to 4 fold in molar ratio relative to the first labeled form of the antigen used in step (a).
114. The method of embodiment 100, wherein in step (c) the population of antibody secreting cells is contacted with the second labeled form of the antigen.
115. The method of embodiment 114, wherein the second labeled form of the antigen is a multivalent form of the antigen.
116. The method of embodiment 114 or 115, wherein the antigen in the second labeled form is at least 2 to 4-fold in molar ratio relative to the first labeled form of the antigen used in step (a).
117. The method according to embodiments 111-116, wherein the contacting in step (c) and the washing in step (d) are repeated at least once before collecting the cells in step (e).
118. The method of embodiment 100, wherein in step (c) the population of antibody secreting cells is contacted with an unlabeled form of the antigen and a second labeled form of the antigen.
119. The method of embodiment 118, wherein the unlabeled form is a monovalent form of the antigen, and the second labeled form of the antigen is a multivalent form of the antigen.
120. The method of embodiment 118, wherein the antigen is a monomeric protein, the unlabeled form is a monovalent form of the antigen, and the second labeled form of the antigen is a multivalent form of the antigen.
121. The method of embodiment 118, wherein the antigen is a multimeric protein, the unlabeled form is a monovalent form of the antigen, and the second labeled form of the antigen is a multivalent form of the antigen.
122. The method of embodiment 118, wherein the antigen is a protein present in both monomeric and multimeric forms, the unlabeled form is a monovalent form of the antigen, and the second labeled form of the antigen is a multivalent form of the antigen.
123. The method of any one of embodiments 118-122, wherein the cells are contacted with the unlabeled form of the antigen and the second labeled form of the antigen at the same time.
124. The method according to any one of embodiments 118-123, wherein the unlabeled form of the antigen is at least 2 to 4-fold in molar ratio relative to the first labeled form of the antigen used in step (a).
125. The method according to any embodiments 93-124, wherein the first detectable label is a fluorescent label, and wherein fluorescence-activated cell sorting is used to collect cells that remain bound to the first labeled form of the antigen.
126. The method according to any one of embodiments 111-124, wherein the first detectable label is a first fluorescent label, and the second detectable label is a second fluorescent label that differentiates from the first fluorescent label, wherein two-dimensional fluorescence-activated cell sorting is performed to collect the pool of antibody secreting cells that remain bound to the first labeled form of the antigen, and wherein the collected pool of antibody secreting cells comprises the antibody secreting cell that secretes the target antibody which remains bound to the first labeled form of the antigen.
127. The method according to embodiments 93-126, wherein the target antibody has an affinity in the range of from about 0.1 pM to about 25 nM (KD).
128. The method of embodiment 127, wherein the affinity is less than about 10 nM (KD).
129. The method according to any one of embodiments 93-128, further comprising separating the pool of antibody secreting cells collected in step (e) into single cells and isolating the antibody secreting cell that secretes the target antibody.
130. The method according to embodiment 82, 85, 92 or 129, further comprising isolating antibody-encoding nucleic acids from the isolated antibody secreting cell that secretes the target antibody.
131. A method of identifying a region of a gene encoding an antigen-binding fragment of a target antibody; wherein the target antibody is secreted by a cell comprising a modified cell surface, and wherein the target antibody is subsequently captured on the modified cell surface, the method comprising:
   a) providing an antibody secreting cell comprising a modified cell surface, wherein the antibody secreting cell secretes a target antibody, wherein the target antibody is subsequently captured on the modified cell surface, wherein the target antibody captured on the modified cell surface is bound to an antigen, wherein the antigen is linked to a barcode nucleic acid molecule, and wherein the antibody secreting cell further comprises a nucleic acid molecule comprising the region of the gene encoding the antigen-binding fragment of the target antibody captured on the modified cell surface;
   b) hybridizing a portion of the barcode nucleic acid molecule to a portion of a first nucleic acid molecule attached to a solid surface;
   c) hybridizing a portion of the nucleic acid molecule comprising the region of the gene encoding the antigen-binding fragment of the target antibody captured on the modified cell surface to a portion of a second nucleic acid molecule attached to the solid surface;
   d) preparing a first library of amplicons of gene expression in the antibody secreting cell, a second library of amplicons of variable regions, diversity regions, and joining regions (VDJ) in the antibody secreting cell, and a third library of amplicons of antigen barcode nucleic acid molecules;
   e) sequencing each of the three libraries of amplicons; and
   f) identifying the region of the gene encoding the antigen-binding fragment of the target antibody captured on the modified cell surface.
132. The method of embodiment 131, wherein the portion of the barcode nucleic acid molecule is complementary to a first template switch oligonucleotide (TSO), wherein the portion of the barcode nucleic acid molecule terminates at the 3′ terminus of the barcode nucleic acid molecule.
133. The method of embodiment 131 or embodiment 132, wherein the portion of the first nucleic acid molecule attached to the solid surface comprises a first template switch oligo (TSO).
134. The method of any one of embodiments 131-133, wherein the 5′ terminus of the first nucleic acid molecule attached to the solid surface comprises a first sequence of three contiguous riboguanosine residues.
135. The method of any one of embodiments 131 to 134, wherein the first nucleic acid molecule attached to the solid surface further comprises a first unique molecular identifier (UMI).
136. The method of any one of embodiments 131 to 135, wherein the first nucleic acid molecule attached to the solid surface further comprises a first surface barcode.
137. The method of any one of embodiments 131 to 136, wherein the first nucleic acid molecule attached to the solid surface further comprises a first sequencing primer.
138. The method of any of embodiments 131 to 137, wherein the first nucleic acid molecule attached to the solid surface comprises, from 5′ to 3′, the first sequence of three riboguanosine residues, the first TSO, the first UMI, the first surface barcode, and the first sequencing primer.
139. The method of any one of embodiments 131 to 138, wherein the first nucleic acid molecule attached to the solid surface is attached by the 3′ terminus of the first nucleic acid molecule attached to the solid surface.
140. The method of any one of embodiments 131-139, wherein the first nucleic acid molecule attached to the solid surface comprises a first DNA molecule attached to the solid surface.
141. The method of embodiment 140, wherein the first DNA molecule attached to the solid surface comprises a first single-stranded DNA molecule attached to the solid surface.
142. The method of embodiment 141, wherein the portion of the first single-stranded DNA molecule attached to the solid surface comprises a first TSO, wherein the portion of the barcode nucleic acid molecule hybridized to the first single-stranded DNA molecule is complementary to the first TSO, and wherein the method further comprises reverse transcribing the first single-stranded DNA molecule attached to the solid surface beginning from the 3′ terminus of the portion of the barcode nucleic acid molecule which is complementary to the first TSO.
143. The method of any one of embodiments 131-142, wherein the barcode nucleic acid molecule is a single-stranded DNA barcode nucleic acid molecule, and wherein the portion of the first nucleic acid molecule attached to the solid surface comprises a first TSO, and the method further comprises reverse transcribing the single-stranded DNA barcode nucleic acid molecule beginning from the 5′ terminus of the first TSO.
144. The method of any one of embodiments 131-143, wherein the 3′ terminus of the portion of the barcode nucleic acid molecule comprises three contiguous cytidine or ribocytidine residues.
145. The method of embodiment 144, further comprising reverse transcribing an mRNA molecule encoding the region of the gene encoding the antigen-binding fragment of the target antibody captured on the modified cell surface, thereby generating a single-stranded cDNA molecule encoding the region of the gene encoding the antigen-binding fragment of the target antibody captured on the modified cell surface, wherein the nucleotide sequence of the single-stranded cDNA molecule encoding the region of the gene encoding the antigen-binding fragment of the target antibody captured on the modified cell surface differs from a DNA complement of the mRNA molecule encoding the region of the gene encoding the antigen-binding fragment of the target antibody captured on the modified cell surface in that the 3′ terminus of the single-stranded DNA molecule encoding the region of the gene encoding the antigen-binding fragment of the target antibody captured on the modified cell surface comprises three additional, contiguous cytidine or ribocytidine residues.
146. The method of embodiment 145, wherein the 3′ terminus of the mRNA molecule encoding the region of the gene encoding the antigen-binding fragment of the target antibody captured on the modified cell surface comprises a plurality of contiguous adenine residues.
147. The method of embodiment 145 or embodiment 146, wherein the 5′ terminus of the single-stranded DNA molecule encoding the region of the gene encoding the antigen-binding fragment of the target antibody captured on the modified cell surface comprises a plurality of contiguous thymine residues.
148. The method of any one of embodiments 131 to 147, wherein the 5′ terminus of the second nucleic acid molecule attached to the solid surface comprises a second sequence of three contiguous riboguanosine residues.
149. The method of any one of embodiments 131 to 148, wherein the second nucleic acid molecule attached to the solid surface further comprises a second UMI.
150. The method of any one of embodiments 131 to 149, wherein the second nucleic acid molecule attached to the solid surface further comprises a second surface barcode, wherein the nucleotide sequences of the first and second surface barcode nucleic acid molecules are the same.
151. The method of any one of embodiments 131 to 150, wherein the second nucleic acid molecule attached to the solid surface further comprises a second sequencing primer.
152. The method of any of embodiments 131 to 151, wherein the second nucleic acid molecule attached to the solid surface comprises, from 5′ to 3′, the second sequence of three riboguanosine residues, the second TSO, the second UMI, the second surface barcode, and the second sequencing primer, wherein the nucleotide sequences of the first and second surface barcode nucleic acid molecules are the same.
153. The method of any one of embodiments 131 to 152, wherein the second nucleic acid molecule attached to the solid surface is attached by the 3′ terminus of the second nucleic acid molecule attached to the solid surface.
154. The method of any one of embodiments 131 to 153, wherein the second nucleic acid molecule attached to the solid surface comprises a second DNA molecule attached to the solid surface.
155. The method of embodiment 154, wherein the second DNA molecule attached to the solid surface comprises a second single-stranded DNA molecule attached to the solid surface.
156. The method of embodiment 155, wherein the 5′ terminus of the portion of the second single-stranded DNA molecule attached to the solid surface comprises a second sequence of three contiguous riboguanosine residues, and wherein the 3′ terminus of the portion of the nucleic acid molecule encoding the region of the gene encoding the antigen-binding fragment of the target antibody captured on the modified cell surface comprises three contiguous cytidine or ribocytidine residues, wherein the method further comprises reverse transcribing the second single-stranded DNA molecule attached to the solid surface beginning from the 3′ terminus of the three contiguous cytidine or ribocytidine residues.
157. The method of any one of embodiments 131 to 156, wherein the nucleic acid molecule encoding the region of the gene encoding the antigen-binding fragment of the target antibody captured on the modified cell surface comprises a second single-stranded DNA molecule encoding the region of the gene encoding the antigen-binding fragment of the target antibody captured on the modified cell surface.
158. The method of embodiment 156, wherein the 3′ terminus of the portion of the single-stranded DNA molecule encoding the region of the gene encoding the antigen-binding fragment of the target antibody captured on the modified cell surface comprises three contiguous cytidine or ribocytidine residues, and wherein the 5′ terminus of the portion of the second single-stranded DNA molecule attached to the solid surface comprises a second sequence of three contiguous riboguanosine residues, wherein the method further comprises reverse transcribing the second single-stranded DNA molecule attached to the solid surface beginning from the 3′ terminus of the portion of the single-stranded DNA molecule encoding the region of the gene encoding the antigen-binding fragment of the target antibody captured on the modified cell surface.
159. The method of any one of embodiments 131 to 158, wherein the antibody secreting cell is disposed within a partition with the solid surface.
160. The method of any one of embodiments 131 to 159, wherein the solid surface comprises a bead.
161. The method of embodiment 159 or 160, further comprising lysing the antibody secreting cell within the partition.
162. The method of any one of embodiments 131 to 161, wherein the barcoded nucleic acid molecule further comprises a third sequencing primer, wherein the third sequencing primer begins at the 5′ terminus of the barcoded nucleic acid molecule.
163. The method of any one of embodiments 131 to 162, wherein each amplicon of the first library of amplicons comprises a gene, wherein each amplicon does not comprise a nucleic acid molecule encoding the region of the gene encoding the antigen-binding fragment of the target antibody captured on the modified cell surface.
164. The method of embodiment 163, the method further comprising sample de-multiplexing, aligning, filtering, and UMI counting the first library of amplicons.
165. The method of any one of embodiments 131 to 164, wherein the sequencing of the first library of amplicons comprises next-generation sequencing.
166. The method of any one of embodiments 131 to 165, the method further comprising mapping and aligning each amplicon of the first library of amplicons to a standard reference genome.
167. The method of embodiment 166, wherein the standard reference genome comprises a human standard reference genome.
168. The method of embodiment 167, wherein the human standard reference genome is GRCh38.
169. The method of embodiment 166, wherein the standard reference genome comprises a murine standard reference genome.
170. The method of embodiment 169, wherein the murine standard reference genome is mm10.
171. The method of any one of embodiments 166-170, wherein the mapping and aligning comprises mapping each amplicon of the first library of amplicons to the standard reference genome with a splice aware aligner (STAR), wherein each amplicon of the first library of amplicons comprises a unique molecular identifier, wherein all amplicons of the first library of amplicons which map to annotated genes of the standard reference genome are binned, and wherein the binned amplicons are counted for each unique molecular identifier (UMI), thereby generating a single cell count matrix comprising mapped count for each annotated gene and a count for each UMI.
172. The method of embodiment 171, the method further comprising filtering the single cell count matrix for high quality cells and extensively profiled genes by calculating the ratio of the total number of annotated genes divided by the login of the total number of UMIs counted, wherein cells with a ratio below about 0.1 are filtered out, wherein cells with more than about four times the interquartile range of the total number of UMIs counted are filtered out, and cells with more than about 80% of reads that map to a mitochondrial gene are filtered out.
173. The method of embodiment 172, the method further comprising normalizing the single cell count matrix such that the total number of UMIs counted is about 10,000.
174. The method of embodiment 172 or 173, the method further comprising computing the PCA embeddings of the single cell count matrix, and performing iterative clustering of major cell types to identify similarities across batches, thereby generating a uniform manifold approximation and projection (UMAP).
175. The method of any of embodiments 172 to 174, the method further comprising using cluster-specific centroids to determine a linear adjustment function per cell, and applying the linear adjustment function per cell to correct for differences in batches, thereby generating batch corrected embeddings, and using the batch corrected embeddings to generate a uniform manifold approximation and projection (UMAP) in two dimensions.
176. The method of embodiment 174 or 175, the method further comprising determining a weighted k-neighbor graph and applying the Leiden algorithm to the weighted k-neighbor graph with resolution values of about 0.1, about 0.2, about 0.5, and about 1.0 to determine cell type clusters in an unsupervised manner.
177. The method of embodiment 176, the method further comprising performing a pairwise Wilcox test to all of the cells in one cluster and comparing the result of the first pairwise Wilcox test, and performing another pairwise Wilcox test to all of the cells in every other cluster to quantify a p-value and fold-change for each gene in each cluster, wherein when a gene has the low p-value and the high fold-change, the cells are labeled by a common marker of antibody secreting cells and the cells are labeled by clustered subtypes that are named by the top genes from the differential expression result.
178. The method of any of embodiments 131 to 177, the method further comprising performing sample de-multiplexing, de novo assembly of read pairs into contigs, and aligning and annotating the contigs against germline segment V(D)J reference sequences for the second family of amplicons.
179. The method of embodiment 178, wherein alignment of the contigs against germline segment V(D)J reference sequences comprises aligning against a human germline reference database or a murine germline reference database.
180. The method of embodiment 179, wherein the human germline reference database comprises the IMGT database of human germline immunoglobulin sequences.
181. The method of embodiment 179, wherein the murine germline reference database comprises the IMGT germline reference database.
182. The method of any one of embodiments 179 to 181, the method further comprising comparing VDJ sequences against sequences in the human germline reference database or the murine germline reference database, thereby labeling the VDJ sequences for quality alignments by checking for in-frame alignments, length of the CDR3, and the absence of stop codons in the variable region sequence, and filtering the VDJ sequences based on the labels.
183. The method of embodiment 182, the method further comprising mapping the VDJ sequences to a reference database of immunoglobulin chains.
184. The method of embodiment 179, wherein the immunoglobulin isotype, the variable region mapping, the joining region mapping, the diversity region mapping, the accuracy of full-length variable regions for both heavy and light chain sequence per cell are confirmed by the alignment.
185. The method of any of embodiments 131 to 184, the method further comprising sample de-multiplexing, aligning, filtering, and UMI counting the third library of amplicons.
186. The method of any one of embodiments 131 to 185, the method further comprising mapping the third library of amplicons to a custom short-read reference which comprises the barcode nucleic acid molecule reference associated with each antigen, wherein the counts for each uniquely mapped barcode nucleic acid molecule are summed for each cell in a barcoded antigen single cell matrix.
187. The method of embodiment 186, the method further comprising quantifying the barcode nucleic acid molecules across all cells and normalizing the quantification by taking the centered log-ratio of each barcode nucleic acid molecule of the plurality of barcode nucleic acid molecules across each sample capture.
188. The method of embodiment 187, the method further comprising removing background antigen signal by denoising and scaling by background (DSB) for each barcode nucleic acid molecule of the plurality of barcoded nucleic acid molecules.
189. The method of embodiment 188, the method further comprising determining an antigen signal distribution, wherein when there is a well separated bimodal distribution in the antigen signal distribution, z-transformed values are used to compute antigen specificity, and wherein when there is not a well separated bimodal distribution in the antigen signal distribution, a quantile value is used.
190. The method of embodiment 189, the method further comprising statistically modeling the target antigen and negative control antigen distributions.
191. The method of any one of embodiments 131 to 190, wherein the analysis of the first library of amplicons, the second library of amplicons, and the third library of amplicons are analyzed simultaneously to obtain at least one candidate sequence of the antigen-positive target antibody captured on the modified cell surface.
192. The method of embodiment 191, the method further comprising subsetting antibody secreting cells with a valid antibody constant region using the analysis of the first library of amplicons and the analysis of the second library of amplicons.
193. The method of embodiment 192, wherein the antibody constant region comprises an IgE constant region.
194. The method of embodiment 192 or 193, wherein the subsetted antibody secreting cell comprises detectable levels of IgE heavy chain and CD79a but lacks detectable levels of Ms4a1 and CD19, wherein the subsetted antibody secreting cell is an IgE antibody secreting cell.
195. The method of embodiment 194, the method further comprising assessing the differential expression of the IgE antibody secreting cell against an IgA-secreting cell isotype, an IgG-secreting cell isotype, and/or an IgM-secreting cell isotype.
196. The method of embodiment 195, wherein the assessment of differential expression further characterizes the unique transcriptional signature of the IgE antibody secreting cell.
197. The method of embodiment 196, the method further comprising assessing the antigen specificity of the IgE antibody secreting cell.
198. The method of embodiment 197, wherein the assessment comprises comparing the antigen specificity of the IgE antibody secreting cell against a plurality of antigens and a control antigen, and wherein the plurality of antigens comprises the target antigen.
199. The method of embodiment 198, wherein the comparing comprises calculating an empirical score for each antigen of the plurality of antigens and the control antigen by subtracting a quantile value of a signal associated with the antigen (qT) from a quantile value of a signal associated with the control antigen (qC) with a penalty factor (x) to determine an antigen specificity score where the antigen specificity score=qT−qC^{x}.
200. The method of embodiment 199, wherein the penalty factor is the same for all antigens.
201. The method of embodiment 199, wherein the penalty factor is different for different antibody isotypes.
202. The method of embodiment 199, wherein the penalty factor is different when other antigens are used.
203. The method of embodiment 199, the method further comprising determining the antigen specificity score for each of the plurality of antigens and the control antigen.
204. The method of embodiment 203, the method further comprising calculating a control antigen specificity score, wherein when the antigen specificity score is high and the control antigen specificity score is low, selecting the cell.
205. The method of embodiment 204, wherein the VDJ regions identify antigen-specific antibodies.
206. The method of embodiment 204, the method further comprising selecting antibody candidates from a ranked list of antigen signals using the paired V_{H}:V_{L} antibody sequence of the cell.
207. The method of any one of embodiments 191 to 206, the method further comprising determining differential expression between isotypes, conditions, and/or antigen specificity, wherein determining the differential expression between isotypes comprises performing a pairwise Wilcox test for all of the cells in one isotype and performing a pairwise Wilcox test for all of the cells in another isotype, thereby quantifying a p-value and fold-change of each gene for each cluster; and wherein determining the differential expression between antigen specificity comprises performing a pairwise Wilcox test for all of the cells in one isotype and performing a pairwise Wilcox test for all of the cells in another isotype, thereby quantifying a p-value and fold-change of each gene for each cluster.
208. The method of any one of embodiments 191 to 207, the method further comprising clustering VDJ sequence similarity in an antigen positive target cell, wherein the clustering VDJ sequence similarity comprises sequence-based alignment, grouping of similar variable regions by amino acid sequence.
209. The method of any one of embodiments 191 to 208, wherein the antigen positive target cell is IgE⁺.
210. The method of any one of embodiments 191 to 209, the method further comprising trimming redundant sequences.
211. A method of identifying a region of a gene encoding an antigen-binding fragment of a target antibody, wherein the target antibody is secreted by an antibody secreting cell, the method comprising:
   a) contacting a population of antibody secreting cells with a second component of a binding pair, wherein the population of antibody secreting cells comprises the antibody secreting cell that secretes the target antibody, whereby the second component of the binding pair binds to the cell surface of the population of antibody secreting cells;
   b) contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the population of antibody secreting cells, and whereby the antibody-capture molecule captures the target antibody secreted by the antibody secreting cell;
   c) contacting the population of antibody secreting cells with a secondary anti-Ig antibody to allow the target antibody secreted by the antibody secreting cell and captured by the antibody capture complex to bind the secondary anti-Ig antibody;
   d) contacting the population of antibody secreting cells with an antigen comprising a barcode nucleic acid molecule to allow the target antibody secreted by the antibody secreting cell and captured by the antibody capture complex to bind the antigen;
   e) collecting a pool of antibody secreting cells, wherein each cell in the pool secretes an antibody that is captured by the antibody capture complex, bound by the secondary anti-Ig antibody and by the antigen comprising the barcode nucleic acid molecule, and wherein the pool of antibody secreting cells comprises the antibody secreting cell that secretes the target antibody;
   f) separating the pool of antibody secreting cells into single antibody secreting cells, and for each single antibody secreting cell:
   1) hybridizing a portion of the barcode nucleic acid molecule to a portion of a first nucleic acid molecule attached to a solid surface, wherein the solid surface and the first nucleic acid molecule attached thereto are unique to each single antibody secreting cell;
   2) hybridizing a portion of the nucleic acid molecule comprising the region of the gene encoding the antigen-binding fragment of the target antibody to a portion of a second nucleic acid molecule attached to the solid surface;
   3) preparing a first library of amplicons of gene expression, a second library of amplicons of variable regions, diversity regions, and joining regions (VDJ), and a third library of amplicons of antigen barcode nucleic acid molecules; and
   4) sequencing each of the three libraries of amplicons; and
   g) identifying the region of the gene encoding the antigen-binding fragment of the target antibody.
212. The method of embodiment 211, wherein:
   the first component of the binding pair comprises avidin, streptavidin, or anti-biotin and the second component of the binding pair comprises biotin;
   the first component of the binding pair comprises one of jun and fos and the second component of the binding pair comprises the other of jun and fos;
   the first component of the binding pair comprises one of mad and max and the second component of the binding pair comprises the other of mad and max;
   the first component of the binding pair comprises one of myc and max and the second component of the binding pair comprises the other of myc and max;
   the first component of the binding pair comprises one of an azide and an alkyne and the second component of the binding pair comprises the other of an azide and an alkyne; or
   the first component of the binding pair comprises one of an azide and a phosphine and the second component of the binding pair comprises the other of an azide and a phosphine.
213. The method of embodiment 211, wherein the first component of the binding pair comprises avidin or streptavidin and the second component of the binding pair comprises biotin.
214. The method of embodiment 211, wherein:
   the second component of the binding pair comprises fluorescein isothiocyanate (FITC), and the first component of the binding pair comprises an anti-FITC antibody;
   the second component of the binding pair comprises phycoerythrin (PE), and the first component of the binding pair comprises an anti-PE antibody;
   the second component of the binding pair comprises APC, and the first component of the binding pair comprises an anti-APC antibody;
   the second component of the binding pair comprises BV421, and the first component of the binding pair comprises an anti-BV421 antibody;
   the second component of the binding pair comprises BV510, and the first component of the binding pair comprises an anti-BV510 antibody;
   the second component of the binding pair comprises BV605, and the first component of the binding pair comprises an anti-BV605 antibody;
   the second component of the binding pair comprises BV650, and the first component of the binding pair comprises an anti-BV650 antibody;
   the second component of the binding pair comprises BV711, and the first component of the binding pair comprises an anti-BV711 antibody; or
   the second component of the binding pair comprises BV786, and the first component of the binding pair comprises an anti-BV786 antibody.
215. A method of identifying a region of a gene encoding an antigen-binding fragment of a target antibody, wherein the target antibody is secreted by an antibody secreting cell, the method comprising:
   a) contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell;
   b) contacting the population of antibody secreting cells with a secondary anti-Ig antibody to allow the target antibody secreted by the antibody secreting cell and captured by the antibody capture complex to bind the secondary anti-Ig antibody;
   c) contacting the population of antibody secreting cells with an antigen comprising a barcode nucleic acid molecule, whereby the target antibody secreted by the antibody secreting cell and captured by the antibody capture complex binds the antigen;
   d) collecting a pool of antibody secreting cells, wherein each cell in the pool secretes an antibody that is captured by the antibody capture complex, bound by the secondary anti-Ig antibody and by the antigen comprising the barcode nucleic acid molecule, and wherein the pool of antibody secreting cells comprises the antibody secreting cell that secretes the target antibody;
   e) separating the pool of antibody secreting cells into single antibody secreting cells, and for each single antibody secreting cell:
   1) hybridizing a portion of the barcode nucleic acid molecule to a portion of a first nucleic acid molecule attached to a solid surface, wherein the solid surface and the first nucleic acid molecule attached thereto are unique to each single antibody secreting cell;
   2) hybridizing a portion of the nucleic acid molecule encoding the region of the gene encoding the antigen-binding fragment of the target antibody to a portion of a second nucleic acid molecule attached to the solid surface;
   3) preparing a first library of amplicons of gene expression in the antibody secreting cell, a second library of amplicons of variable regions, diversity regions, and joining regions (VDJ) in the antibody secreting cell, and a third library of amplicons of antigen barcode nucleic acid molecules; and
   4) sequencing each of the three libraries of amplicons; and
   f) identifying the region of the gene encoding the antigen-binding fragment of the target antibody.
216. The method of embodiment 215, wherein the second component of the binding pair comprises a surface marker of the antibody secreting cell and the first component of the binding pair is an antibody that binds to the surface marker.
217. The method of embodiment 216, wherein the surface marker comprises CD27, CD38, CD45, CD138, CD98, CD78, CD319, CXCR4, BCMA, GPRC5D, FCRL5, CD19, Ly6D, CD52, or transmembrane activator calcium modulator and cyclophilin ligand interactor (TACI).
218. The method of embodiment 215, wherein the second component of the binding pair comprises an interleukin-6 receptor (IL-6R), and the first component of the binding pair comprises an anti-IL-6R antibody or interleukin-6 (IL-6).
219. The method of embodiment 215, wherein the second component of the binding pair comprises CD27, and the first component of the binding pair comprises an anti-CD27 antibody or CD70.
220. The method of any one of embodiments 211 to 219, wherein the antibody-capture molecule comprises a capture antibody.
221. The method of embodiment 220, wherein the capture antibody comprises an anti-Fc antibody, an anti-light chain kappa antibody, or an anti-light chain lambda antibody.
222. The method of embodiment 221, wherein the anti-Fc antibody comprises an anti-Fcγ antibody, an anti-Fcα antibody, or an anti-Fcε antibody.
223. The method of embodiment 222, wherein the anti-Fcγ antibody comprises an anti-FcγRI antibody, an anti-FcγRIIA antibody, an anti-FcγRIIB antibody, an anti-FcγRIIB1 antibody, an anti-FcγRIIB2 antibody, an anti-FcγRIIIA antibody, or an anti-FcγRIIIB antibody.
224. The method of embodiment 222, wherein the anti-Fcα antibody comprises an anti-FcαRI antibody.
225. The method of embodiment 222, wherein the anti-Fcε antibody comprises an anti-FcεRI antibody or an anti-FcεRII antibody.
226. The method of embodiment 220, wherein the capture antibody comprises an anti-IgM antibody.
227. The method of embodiment 220, wherein the capture antibody comprises an anti-IgG antibody.
228. The method of embodiment 227, wherein the anti-IgG antibody comprises an anti-IgG1 antibody, an anti-IgG2 antibody, an anti-IgG2a antibody, an anti-IgG2b antibody, an anti-IgG3 antibody, or an anti-IgG4 antibody.
229. The method of embodiment 220, wherein the capture antibody comprises an anti-IgA antibody.
230. The method of embodiment 229, wherein the anti-IgA antibody comprises an anti-IgA1 antibody, an anti-IgA2 antibody, an anti-secretory IgA antibody, or a polymeric anti-IgA antibody.
231. The method of embodiment 220, wherein the capture antibody comprises an anti-IgE antibody.
232. The method of any one of embodiments 211 to 219, wherein the antibody-capture molecule comprises an Fc receptor or an ectodomain of the Fc receptor.
233. The method of embodiment 232, wherein the Fc receptor comprises an Fcγ receptor, an Fcα receptor, or an Fcε receptor.
234. The method of embodiment 233, wherein the Fcγ receptor comprises an FcγRI receptor, an FcγRIIA receptor, an FcγRIIB receptor, an FcγRIIB1 receptor, an FcγRIIB2 receptor, an FcγRIIIA receptor, an FcγRIIIB receptor, or an FcRn receptor.
235. The method of embodiment 233, wherein the Fcα receptor comprises an FcαRI receptor or an Fcα/μR receptor.
236. The method of embodiment 233, wherein the Fcε receptor comprises an FcεRI receptor or an FcεRII receptor.
237. The method of any one of embodiments 211 to 219, wherein the antibody-capture molecule comprises protein A or protein G.
238. The method of any one of embodiments 211 to 219, wherein the antibody-capture molecule comprises protein L.
239. The method of any one of embodiments 211 to 238, wherein the secondary anti-Ig antibody is anti-IgE or anti-IgG.
240. The method of any one of embodiments 211 to 239, wherein the secondary anti-Ig antibody is detectably labeled.
241. The method of embodiment 240, wherein the secondary anti-Ig antibody is labeled with a radioactive compound, a fluorescent compound, or an enzyme.
242. The method of embodiment 241, wherein the radioactive compound comprises ³H, ¹⁴C ¹⁹F, ³⁵S, ¹²⁵I, ¹³¹I, ¹¹¹In, or ⁹⁹Tc.
243. The method of embodiment 241, wherein the fluorescent compound comprises fluorescein, fluorescein isothiocyanate, rhodamine, 5-dimethylamine-1-naphthalenesulfonyl chloride, phycoerythrin, or a fluorescent protein.
244. The method of embodiment 241, wherein the enzyme comprises alkaline phosphatase, horseradish peroxidase, luciferase, or glucose oxidase.
245. The method of any one of embodiments 211 to 244, wherein the antibody secreting cell is contacted with a plurality of barcoded antigens, wherein each barcoded antigen of the plurality of barcoded antigens comprises a unique antigen linked to a unique nucleic acid molecule.
246. The method of embodiment 245, wherein the unique nucleic acid molecule comprises DNA.
247. The method of embodiment 245, wherein the unique nucleic acid molecule comprises RNA.
248. The method of any one of embodiments 245 to 247, wherein each unique nucleic acid molecule comprises from about 10 nucleotides to about 500 nucleotides.
249. The method of any one of embodiments 245 to 248, wherein each barcoded antigen is detectably labeled.
250. The method of embodiment 249, wherein each barcoded antigen is labeled with a radioactive compound, a fluorescent compound, or an enzyme.
251. The method of embodiment 250, wherein the radioactive compound comprises ³H, ¹⁴C ¹⁹F, ³⁵S, ¹²⁵I, ¹³¹I, ¹¹¹In, or ⁹⁹Tc.
252. The method of embodiment 250, wherein the fluorescent compound comprises fluorescein, fluorescein isothiocyanate, rhodamine, 5-dimethylamine-1-naphthalenesulfonyl chloride, phycoerythrin, or a fluorescent protein.
253. The method of embodiment 250, wherein the enzyme comprises alkaline phosphatase, horseradish peroxidase, luciferase, or glucose oxidase.
254. The method of any one of embodiments 211 to 253, wherein the barcoded antigen comprises an allergen.
255. The method of any one of embodiments 211 to 254, wherein the antibody secreting cell is a plasma cell or plasmablast.
256. The method of any one of embodiments 211 to 255, the method further comprising enriching a population of cells obtained from a human for immune cells prior to contacting the surface of the population of antibody secreting cells with the second component of a binding pair or prior to contacting the surface of the population of antibody secreting cells with the antibody capture complex.
257. The method of embodiment 256, wherein the population of cells obtained from the human is enriched for plasma cells or plasmablasts.
258. The method of embodiment 257, wherein the population of enriched immune cells is enriched for plasma cells or plasmablasts using pan-B cell enrichment.
259. The method of any one of embodiments 211 to 258, wherein the population of antibody secreting cells is obtained from the lymph node, lung, bone marrow, or blood of a human.
260. The method of any one of embodiments 211 to 259, the method further comprising detecting the secondary anti-Ig antibody and/or one or more of the barcoded antigens.
261. The method of any one of embodiments 211 to 260, the method further comprising sorting the population of antibody secreting cells to obtain the pool of antibody secreting cells that are bound by secondary anti-Ig antibody and/or one or more of the barcoded antigens.
262. The method of any one of embodiments 211 to 261, the method further comprising contacting the population of antibody secreting cells with an Fc block prior to contacting the population of antibody secreting cells with the second component of the binding pair.
263. The method of any one of embodiments 211 to 262, wherein the portion of the barcode nucleic acid molecule is complementary to a first template switch oligonucleotide (TSO).
264. The method of any one of embodiments 211 to 263, wherein the portion of the first nucleic acid molecule attached to the solid surface comprises a first template switch oligo (TSO).
265. The method of any one of embodiments 211 to 264, wherein the 5′ terminus of the first nucleic acid molecule attached to the solid surface comprises a first sequence of three contiguous riboguanosine residues.
266. The method of any one of embodiments 211 to 265, wherein the first nucleic acid molecule attached to the solid surface further comprises a first unique molecular identifier (UMI).
267. The method of any one of embodiments 211 to 266, wherein the first nucleic acid molecule attached to the solid surface further comprises a first surface barcode.
268. The method of any one of embodiments 211 to 267, wherein the first nucleic acid molecule attached to the solid surface further comprises a first sequencing primer.
269. The method of any of embodiments 211 to 268, wherein the first nucleic acid molecule attached to the solid surface comprises, from 5′ to 3′, the first sequence of three riboguanosine residues, the first TSO, the first UMI, the first surface barcode, and the first sequencing primer.
270. The method of any one of embodiments 211 to 269, wherein the first nucleic acid molecule attached to the solid surface is attached by the 3′ terminus of the first nucleic acid molecule attached to the solid surface.
271. The method of any one of embodiments 211 to 270, wherein the first nucleic acid molecule attached to the solid surface comprises a first DNA molecule attached to the solid surface.
272. The method of embodiment 271, wherein the first DNA molecule attached to the solid surface comprises a first single-stranded DNA molecule attached to the solid surface.
273. The method of embodiment 272, wherein the portion of the first single-stranded DNA molecule attached to the solid surface comprises a first TSO and the method further comprises reverse transcribing the first single-stranded DNA molecule attached to the solid surface beginning from the 3′ terminus of the portion of the barcode nucleic acid molecule which is complementary to the first TSO.
274. The method of any one of embodiments 211 to 273, wherein the barcode nucleic acid molecule is a single-stranded DNA barcode nucleic acid molecule, and wherein the portion of the first nucleic acid molecule attached to the solid surface comprises a first TSO, and the method further comprises reverse transcribing the single-stranded DNA barcode nucleic acid molecule beginning from the 3′ terminus of the first TSO.
275. The method of any one of embodiments 211 to 274, wherein the 3′ terminus of the portion of the barcode nucleic acid molecule comprises three contiguous cytidine residues.
276. The method of embodiment 275, further comprising reverse transcribing an mRNA molecule encoding the region of the gene encoding the antigen-binding fragment of the target antibody, thereby generating a single-stranded DNA molecule encoding the region of the gene encoding the antigen-binding fragment of the target antibody, and wherein the nucleotide sequence of the single-stranded DNA molecule encoding the region of the gene encoding the antigen-binding fragment of the target antibody differs from a complement of the mRNA molecule encoding the region of the gene encoding the antigen-binding fragment of the target antibody in that the 3′ terminus of the single-stranded DNA molecule encoding the region of the gene encoding the antigen-binding fragment of the target antibody comprises three additional, contiguous cytidine residues.
277. The method of embodiment 276, wherein the 3′ terminus of the mRNA molecule encoding the region of the gene encoding the antigen-binding fragment of the target antibody comprises a plurality of contiguous adenine residues.
278. The method of embodiment 276 or embodiment 277, wherein the 5′ terminus of the single-stranded DNA molecule encoding the region of the gene encoding the antigen-binding fragment of the target antibody comprises a plurality of contiguous thymine residues.
279. The method of any one of embodiments 211 to 278, wherein the 5′ terminus of the second nucleic acid molecule attached to the solid surface comprises a second sequence of three contiguous riboguanosine residues.
280. The method of any one of embodiments 211 to 279, wherein the second nucleic acid molecule attached to the solid surface further comprises a second UMI.
281. The method of any one of embodiments 211 to 280, wherein the second nucleic acid molecule attached to the solid surface further comprises a second surface barcode.
282. The method of any one of embodiments 211 to 281, wherein the second nucleic acid molecule attached to the solid surface further comprises a second sequencing primer.
283. The method of any of embodiments 211 to 282, wherein the second nucleic acid molecule attached to the solid surface comprises, from 5′ to 3′, the second sequence of three riboguanosine residues, the second TSO, the second UMI, the second surface barcode, and the second sequencing primer.
284. The method of any one of embodiments 211 to 283, wherein the second nucleic acid molecule attached to the solid surface is attached by the 3′ terminus of the second nucleic acid molecule attached to the solid surface.
285. The method of any one of embodiments 211 to 284, wherein the second nucleic acid molecule attached to the solid surface comprises a second DNA molecule attached to the solid surface.
286. The method of embodiment 285, wherein the second DNA molecule attached to the solid surface comprises a second single-stranded DNA molecule attached to the solid surface.
287. The method of embodiment 286, wherein the 5′ terminus of the portion of the second single-stranded DNA molecule attached to the solid surface comprises a second sequence of three contiguous riboguanosine residues, and wherein the 3′ terminus of the portion of the nucleic acid molecule encoding the region of the gene encoding the antigen-binding fragment of the target antibody comprises three contiguous cytidine residues, wherein the method further comprises reverse transcribing the second single-stranded DNA molecule attached to the solid surface beginning from the 3′ terminus of the three contiguous cytidine residues.
288. The method of any one of embodiments 211 to 287, wherein the nucleic acid molecule encoding the region of the gene encoding the antigen-binding fragment of the target antibody comprises a second single-stranded DNA molecule encoding the region of the gene encoding the antigen-binding fragment of the target antibody.
289. The method of embodiment 287, wherein the 3′ terminus of the portion of the single-stranded DNA molecule encoding the region of the gene encoding the antigen-binding fragment of the target antibody comprises three contiguous cytidine residues, and wherein the 5′ terminus of the portion of the second single-stranded DNA molecule attached to the solid surface comprises a second sequence of three contiguous riboguanosine residues, wherein the method further comprises reverse transcribing the second single-stranded DNA molecule attached to the solid surface beginning from the 3′ terminus of the portion of the single-stranded DNA molecule encoding the region of the gene encoding the antigen-binding fragment of the target antibody.
290. The method of any one of embodiments 211 to 289, wherein the single antibody secreting cells are each disposed within a partition with a solid surface.
291. The method of any one of embodiments 211 to 290, wherein the solid surface comprises a bead.
292. The method of embodiment 290 or embodiment 291, further comprising lysing the single antibody secreting cells within the partitions.
293. The method of any one of embodiments 211 to 292, wherein the barcoded nucleic acid molecule further comprises a third sequencing primer.
294. The method of any one of embodiments 211 to 293, wherein each amplicon of the first library of amplicons comprises a gene, wherein each amplicon does not comprise a nucleic acid molecule comprising the region of the gene encoding the antigen-binding fragment of the target antibody.
295. The method of embodiment 294, the method further comprising sample de-multiplexing, aligning, filtering, and UMI counting the first library of amplicons.
296. The method of any one of embodiments 211 to 295, wherein the sequencing of the first library of amplicons comprises next-generation sequencing.
297. The method of any one of embodiments 211 to 296, the method further comprising mapping and aligning each amplicon of the third library of amplicons to a standard reference genome.
298. The method of embodiment 297, wherein the standard reference genome comprises a human standard reference genome.
299. The method of embodiment 298, wherein the human standard reference genome is GRCh38.
300. The method of embodiment 297, wherein the standard reference genome comprises a murine standard reference genome.
301. The method of embodiment 300, wherein the murine standard reference genome is mm10.
302. The method of any one of embodiments 297 to 301, wherein the mapping and aligning comprises mapping each amplicon of the first library of amplicons to the standard reference genome with a splice aware aligner (STAR), wherein each amplicon of the first library of amplicons comprises a unique molecular identifier, wherein all amplicons of the first library of amplicons which map to annotated genes of the standard reference genome are binned, and wherein the binned amplicons are counted for each unique molecular identifier (UMI), thereby generating a single cell count matrix comprising a mapped count for each annotated gene and a count for each UMI.
303. The method of embodiment 302, the method further comprising filtering the single cell count matrix for high quality single antibody secreting cells and extensively profiled genes by calculating the ratio of the total number of annotated genes divided by the login of the total number of UMIs counted, wherein single antibody secreting cells with a ratio below about 0.1 are filtered out, wherein single antibody secreting cells with more than about four times the interquartile range of the total number of UMIs counted are filtered out, and single antibody secreting cells with more than about 80% of reads map to a mitochondrial gene are filtered out.
304. The method of embodiment 303, the method further comprising normalizing the single cell count matrix such that the total number of UMIs counted is about 10,000.
305. The method of embodiment 303 or embodiment 304, the method further comprising computing the PCA embeddings of the single cell count matrix, performing iterative clustering of major cell types to identify similarities across batches, thereby generating a uniform manifold approximation and projection (UMAP).
306. The method of any of embodiments 303 to 305, the method further comprising using cluster-specific centroids to determine a linear adjustment function per cell, applying the linear adjustment function per cell to correct for differences in batches, thereby generating batch corrected embeddings, and using the batch corrected embeddings to generate a uniform manifold approximation and projection (UMAP) in two dimensions.
307. The method of embodiment 305 or embodiment 306, the method further comprising determining a weighted k-neighbor graph and applying the Leiden algorithm to the weighted k-neighbor graph with resolution values of 0.1, 0.2, 0.5, and 1.0 to determine cell type clusters in an unsupervised manner.
308. The method of embodiment 305, the method further comprising performing a pairwise Wilcox test to all of the single antibody secreting cells in one cluster and comparing the result of the first pairwise Wilcox test, and performing another pairwise Wilcox test to all of the single antibody secreting cells in every other cluster to quantify a p-value and fold-change for each gene in each cluster, wherein when a gene has the low p-value and the high fold-change, the single antibody secreting cells are labeled by a common marker of plasma cells, plasmablasts, or B cells and the single antibody secreting cells are labeled by clustered subtypes that are named by the top genes from the differential expression result.
309. The method of any of embodiments 211 to 308, the method further comprising performing sample de-multiplexing, de novo assembly of read pairs into contigs, and aligning and annotating the contigs against germline segment V(D)J reference sequences for the second family of amplicons.
310. The method of embodiment 309, wherein alignment of the contigs against germline segment V(D)J reference sequences comprises aligning against a human germline reference database or a murine germline reference database.
311. The method of embodiment 310, wherein the human germline reference database comprises the IMGT database of human germline immunoglobulin sequences.
312. The method of embodiment 310, wherein the murine germline reference database comprises the IMGT germline reference database.
313. The method of any one of embodiments 310 to 312, the method further comprising comparing VDJ sequences against sequences in the human germline reference database or the murine germline reference database, thereby labeling the VDJ sequences for quality alignments by checking for in-frame alignments, length of the CDR3, and the absence of stop codons in the variable region sequence, and filtering the VDJ sequences based on the labels.
314. The method of embodiment 313, the method further comprising mapping the VDJ sequences to a reference database of immunoglobulin chains.
315. The method of embodiment 310, wherein the immunoglobulin isotype, the variable region mapping, the joining region mapping, the diversity region mapping, the accuracy of full-length variable regions for both heavy and light chain sequence per cell are confirmed by the alignment.
316. The method of any of embodiments 211 to 315, the method further comprising sample de-multiplexing, aligning, filtering, and UMI counting the third library of amplicons.
317. The method of any one of embodiments 211 to 316, the method further comprising mapping the third library of amplicons to a custom short-read reference which comprises the barcode nucleic acid molecule reference associated with each antigen, wherein the counts for each uniquely mapped barcode nucleic acid molecule are summed for each cell in a barcoded antigen single cell matrix.
318. The method of embodiment 317, the method further comprising quantifying the barcode nucleic acid molecules across all single antibody secreting cells and normalizing the quantification by taking the centered log-ratio of each barcode nucleic acid molecule of the plurality of barcode nucleic acid molecules across each sample capture.
319. The method of embodiment 318, the method further comprising removing background antigen signal by denoising and scaling by background (DSB) for each barcode nucleic acid molecule of the plurality of barcoded nucleic acid molecules.
320. The method of embodiment 319, the method further comprising determining an antigen signal distribution, wherein when there is a well separated bimodal distribution in the antigen signal distribution, z-transformed values are used to compute antigen specificity, and wherein when there is not a well separated bimodal distribution in the antigen signal distribution, a quantile value is used.
321. The method of embodiment 320, the method further comprising statistically modeling the target antigen and negative control antigen distributions.
322. The method of any one of embodiments 211 to 321, wherein the analysis of the first library of amplicons, the second library of amplicons, and the third library of amplicons are analyzed simultaneously to obtain at least one candidate sequence of the antigen-positive target antibody.
323. The method of embodiment 322, the method further comprising subsetting plasma cells or plasmablasts with a valid antibody constant region using the analysis of the first library of amplicons and the analysis of the second library of amplicons.
324. The method of embodiment 323, wherein the antibody constant region comprises an IgE constant region.
325. The method of embodiment 323 or embodiment 324, wherein the subsetted plasma cell or plasmablast comprises detectable levels of IgE heavy chain and CD79a but lacking detectable levels of Ms4a1 and CD19, wherein the subsetted plasma cell or plasmablast is an IgE⁺ plasma cell or plasmablast.
326. The method of embodiment 325, the method further comprising assessing the differential expression of the IgE⁺ plasma cell or plasmablast against an IgA-secreting plasma cell or plasmablast isotype, an IgG-secreting plasma cell or plasmablast isotype, and/or an IgM-secreting plasma cell or plasmablast isotype.
327. The method of embodiment 326, wherein the assessment of differential expression further characterizes the unique transcriptional signature of the IgE plasma cell or plasmablast.
328. The method of embodiment 327, the method further comprising assessing the antigen specificity of the IgE plasma cell or plasmablast.
329. The method of embodiment 328, wherein the assessment comprises comparing the antigen specificity of the IgE plasma cell or plasmablast against a plurality of antigens and a control antigen, and wherein the plurality of antigens comprises the target antigen.
330. The method of embodiment 329, wherein the comparing comprises calculating an empirical score for each antigen of the plurality of antigens and the control antigen by subtracting a quantile value of a signal associated with the antigen (qT) from a quantile value of a signal associated with the control antigen (qC) with a penalty factor (x) to determine an antigen specificity score where the antigen specificity score=qT−qC^{x}.
331. The method of embodiment 330, wherein the penalty factor is the same for all antigens.
332. The method of embodiment 330, wherein the penalty factor is different for different antibody isotypes.
333. The method of embodiment 330, wherein the penalty factor is different when other antigens are used.
334. The method of embodiment 331, the method further comprising determining the antigen specificity score for each of the plurality of antigens and the control antigen.
335. The method of embodiment 334, the method further comprising calculating a control antigen specificity score, wherein when the antigen specificity score is high and the control antigen specificity score is low, selecting the cell.
336. The method of embodiment 335, wherein the VDJ regions identify antigen-specific antibodies.
337. The method of embodiment 335, the method further comprising selecting antibody candidates from a ranked list of antigen signals using the paired V_{H}:V_{L} antibody sequence of the cell.
338. The method of any one of embodiments 322 to 337, the method further comprising determining differential expression between isotypes, conditions, and/or antigen specificity, wherein determining the differential expression between isotypes comprises performing a pairwise Wilcox test for all of the single antibody secreting cells in one isotype and performing a pairwise Wilcox test for all of the single antibody secreting cells in another isotype, thereby quantifying a p-value and fold-change of each gene for each cluster; and wherein determining the differential expression between antigen specificity comprises performing a pairwise Wilcox test for all of the single antibody secreting cells in one isotype and performing a pairwise Wilcox test for all of the single antibody secreting cells in another isotype, thereby quantifying a p-value and fold-change of each gene for each cluster.
339. The method of any one of embodiments 320 to 338, the method further comprising clustering VDJ sequence similarity in an antigen positive target cell, wherein the clustering VDJ sequence similarity comprises sequence-based alignment, grouping of similar variable regions by amino acid sequence.
340. The method of any one of embodiments 320 to 339, wherein the antigen positive target cell is IgE⁺.
341. The method of any one of embodiments 320 to 340, the method further comprising trimming redundant sequences.

Various modifications of the described subject matter, in addition to those described herein, will be apparent to those skilled in the art from the foregoing description. Such modifications are also intended to fall within the scope of the appended claims.

1. An antibody capture complex comprising a first component of a binding pair linked to an antibody-capture molecule;
wherein the first component of the binding pair is capable of binding a second component of the binding pair;
wherein the antibody-capture molecule is capable of binding to a target antibody, wherein the target antibody is secreted by an antibody secreting cell;
wherein when the second component of the binding pair is biotin and the first component of the binding pair is streptavidin, the antibody-capture molecule does not bind to a kappa light chain of the target antibody.

2-28. (canceled)

29. A method of capturing a target antibody secreted by an antibody secreting cell, the method comprising:
contacting a population of antibody secreting cells with a second component of a binding pair to allow the second component of the binding pair to bind to the cell surface of the population of antibody secreting cells, wherein the population of antibody secreting cells comprises the antibody secreting cell that secretes the target antibody;
contacting the population of antibody secreting cells with an antibody capture complex, wherein the antibody capture complex comprises a first component of the binding pair linked to an antibody-capture molecule, whereby the first component of the binding pair binds to the second component of the binding pair on the cell surface of the antibody secreting cells, and whereby the antibody-capture molecule captures the target antibody secreted by the antibody secreting cell; and
contacting the population of antibody secreting cells with an antigen, whereby the target antibody secreted by the antibody secreting cell and captured by the antibody capture complex binds the antigen.

30. The method of claim 29, wherein:
the first component of the binding pair comprises avidin, streptavidin, or anti-biotin and the second component of the binding pair comprises biotin;
the first component of the binding pair comprises one of jun and fos and the second component of the binding pair comprises the other of jun and fos;
the first component of the binding pair comprises one of mad and max and the second component of the binding pair comprises the other of mad and max;
the first component of the binding pair comprises one of myc and max and the second component of the binding pair comprises the other of myc and max;
the first component of the binding pair comprises one of an azide and an alkyne and the second component of the binding pair comprises the other of an azide and an alkyne; or
the first component of the binding pair comprises one of an azide and a phosphine and the second component of the binding pair comprises the other of an azide and a phosphine.

31. The method of claim 29, wherein the first component of the binding pair comprises avidin or streptavidin and the second component of the binding pair comprises biotin.

32. The method of claim 29, wherein:
the second component of the binding pair comprises fluorescein isothiocyanate (FITC), and the first component of the binding pair comprises an anti-FITC antibody;
the second component of the binding pair comprises phycoerythrin (PE), and the first component of the binding pair comprises an anti-PE antibody;
the second component of the binding pair comprises APC, and the first component of the binding pair comprises an anti-APC antibody;
the second component of the binding pair comprises BV421, and the first component of the binding pair comprises an anti-BV421 antibody;
the second component of the binding pair comprises BV510, and the first component of the binding pair comprises an anti-BV510 antibody;
the second component of the binding pair comprises BV605, and the first component of the binding pair comprises an anti-BV605 antibody;
the second component of the binding pair comprises BV650, and the first component of the binding pair comprises an anti-BV650 antibody;
the second component of the binding pair comprises BV711, and the first component of the binding pair comprises an anti-BV711 antibody; or
the second component of the binding pair comprises BV786, and the first component of the binding pair comprises an anti-BV786 antibody.

33. A method of capturing a target antibody secreted by an antibody secreting cell, the method comprising:
contacting a population of antibody secreting cells with an antibody capture complex, wherein the population of antibody secreting cells comprises the antibody secreting cell that secretes the target antibody, wherein the antibody capture complex comprises a first component of a binding pair linked to an antibody-capture molecule, whereby the first component of the binding pair binds to a second component of the binding pair on the cell surface of the population of antibody secreting cells, and whereby the antibody-capture molecule captures the target antibody secreted by the antibody secreting cell; and
contacting the population of antibody secreting cells with an antigen, whereby the target antibody secreted by the antibody secreting cell and captured by the antibody capture complex binds the antigen.

34. The method of claim 33, wherein the second component of the binding pair comprises a surface marker of the population of antibody secreting cells and the first component of the binding pair is an antibody that binds to the surface marker.

35-37. (canceled)

38. The method of claim 29, wherein the antibody-capture molecule comprises a capture antibody.

39. The method of claim 38, wherein the capture antibody comprises an anti-Fc antibody, an anti-light chain kappa antibody, or an anti-light chain lambda antibody.

40. The method of claim 39, wherein the anti-Fc antibody comprises an anti-Fcγ antibody, an anti-Fcα antibody, or an anti-Fcε antibody.

41-43. (canceled)

44. The method of claim 38, wherein the capture antibody comprises an anti-IgM antibody, an anti-IgG antibody, an anti-IgA antibody, or an anti-IgE antibody.

45-54. (canceled)

55. The method of claim 29, wherein the antibody-capture molecule comprises protein A, protein G or protein L.

56-63. (canceled)

64. The method of claim 29, wherein in the step of contacting the population of antibody secreting cells with an antigen, the antigen is a barcoded antigen.

65. The method of claim 29, wherein in the step of contacting the population of antibody secreting cells with an antigen, the population of antibody secreting cells is contacted with a plurality of barcoded antigens, wherein each barcoded antigen of the plurality of barcoded antigens comprises a unique antigen linked to a unique nucleic acid molecule.

66-68. (canceled)

69. The method of claim 65, wherein each barcoded antigen is detectably labeled.

70-73. (canceled)

74. The method of claim 64, wherein the barcoded antigen comprises an allergen.

75-79. (canceled)

80. The method of claim 29, further comprising detecting the antigen bound by the target antibody.

81. The method of claim 29, further comprising sorting the population of antibody secreting cells to collect a pool of antibody secreting cells, wherein the antibody secreting cells in the pool each secrete an antibody that is captured by the antibody capture molecule and is bound by the antigen, wherein the collected pool of antibody secreting cells comprises the antibody secreting cell that secretes the target antibody.

82. The method of claim 81, wherein separating the collected pool of antibody secreting cells into single cells and isolating the antibody secreting cell that secretes the target antibody.

83-85. (canceled)

86. The method of claim 29, further comprising contacting the population of antibody secreting cells with an Fc block prior to contacting the population of antibody secreting cells with the second component of the binding pair.

87. A method of capturing an IgE antibody secreted by an antibody secreting cell, wherein the IgE antibody is directed to an allergen, the method comprising:
contacting a population of antibody secreting cells with NHS-biotin to allow biotin to bind to the cell surface, wherein the population of antibody secreting cells comprises the antibody secreting cell that secretes the IgE antibody;
contacting the population of antibody secreting cells with an antibody capture complex, wherein the antibody capture complex comprises streptavidin linked to an ectodomain of a high affinity IgE receptor (FcεRIa), whereby the antibody-capture molecule binds to the IgE antibody secreted by the antibody secreting cell; and
contacting the population of antibody secreting cells with an antigen, wherein the antigen is an antigenic portion or a mixture of a plurality of antigenic portions of the allergen, whereby the IgE antibody captured by the antibody capture complex binds to the antigen.

88-92. (canceled)

93. The method of claim 29, wherein the step of contacting the population of antibody secreting cells with an antigen comprises:
(a) contacting the population of antibody secreting cells with a first labeled form of the antigen to allow the antigen to bind to antibodies including the target antibody captured on the cell surface of the population of antibody secreting cells, wherein the antigen of the first labeled form is conjugated to a first detectable label;
(b) washing the population of antibody secreting cells to remove unbound antigen;
(c) contacting the population of antibody secreting cells with
(i) an unlabeled form of the antigen,
(ii) a second labeled form of the antigen, or
(iii) the unlabeled form of the antigen and the second labeled form of the antigen;
(d) washing the population of antibody secreting cells to remove unbound antigen, and
(e) collecting a pool of antibody secreting cells remaining bound to the first labeled form of the antigen, wherein the collected pool of antibody secreting cells comprises the antibody secreting cell that secretes the target antibody, wherein the target antibody secreted by the antibody secreting cell and captured by the antibody capture complex remains bound to the first labeled form of the antigen.

94-341. (canceled)
